(19) United States
(12) Patent Application Publication
(10) Pub. No.: US 2025/0012817 A1
(43) Pub. Date: Jan. 9, 2025

(54) SELECTION BIOMARKERS FOR PATIENT STRATIFICATION IN BODILY FLUIDS AND APPLYING PRECISION MEDICINE THROUGH NOVEL DIAGNOSTIC BIOMARKERS

(71) Applicant: Lifestory Health Inc., BROOKLINE, MA (US)

(72) Inventor: Anna Villarreal, Brookline, MA (US)

(73) Assignee: Lifestory Health Inc., BROOKLINE, MA (US)

(21) Appl. No.: 18/624,423

(22) Filed: Apr. 2, 2024

Related U.S. Application Data
(63) Continuation of application No. 16/753,418, filed on Apr. 3, 2020, now Pat. No. 11,946,940.
(60) Provisional application No. 62/569,998, filed on Oct. 9, 2017.

(51) Int. Cl. G01N 33/72; G01N 33/68

(52) U.S. Cl. CPC G01N 33/723; G01N 33/6848; G01N 2333/805; G01N 2560/00; G01N 2800/042

(57) ABSTRACT

The present invention relates to, in part, methods of improved healthcare in female subjects that, for example, rely on menstrual fluid sampling for applying selection biomarkers.

### RELATED APPLICATIONS

This application is a continuation of U.S. patent application Ser. No. 16/753,418 filed Apr. 3, 2020, which is a 35 U.S.C. § 371 U.S. National Phase entry of International Application No. PCT/US2018/055059 filed Oct. 9, 2018, which claims priority to U.S. Provisional Application No. 62/569,998, titled “DIABETES AND PREDIABETES DETECTION IN MENSTRUAL FLUID” and filed Oct. 9, 2017, the entire contents of which are hereby incorporated by reference for all purposes.

### INCORPORATION BY REFERENCE

In accordance with 37 CFR 1.831 (a), 37 CFR 1.835 (a) (2) and/or 1.835 (b) (2), Applicant states that the Sequence Listing is entitled 068036-00009_replacement_sequence_listing_st25_9_30_24.xml, was created on Sep. 30, 2024 and is 1.40 MB in size, and the contents of the sequence listing are hereby incorporated by reference in its entirety for all purposes.

### FIELD OF THE INVENTION

This invention relates to, in part, methods and devices that are useful for the treatment, prevention, and/or diagnosis, of various diseases in females, as well as female stratification through application of selection biomarkers and precision medicine using health-related biomarkers.

### BACKGROUND

Healthcare is estimated to account for nearly twenty percent of the U.S. Gross Domestic Product (GDP)—an amount of almost three trillion US dollars. Considering the increased demand for healthcare the magnitude of this industry is expected to rise. With increased demand comes less access to health care practitioners either in the form of shorter visits or longer wait times to schedule an appointment. Further, the increased reliance on biomarkers for monitoring patient health and prescribing pharmaceuticals make accurate biomarker measurements a critical component for effective health care. New methods, devices, and systems capable of providing accurate biomarker measurements, without increasing demand on the current health care system, are needed. A three-fold higher likelihood of approval from Phase One was calculated for programs that utilized selection biomarkers (25.9%, n=512) versus programs that did not (8.4%, n=9,012). All four phase transition success rates were much higher for programs incorporating selection biomarkers versus those not incorporating selection biomarkers. Phase three transition success rates for selection biomarker programs were 76.5% (n=132) vs. only 55.0% (n=1,254) for nonselection biomarker programs-the largest percentage difference among the four phases of development. Patient enrollment at the molecular level is a more successful strategy than heterogeneous enrollment especially for female inclusion as the female biology is largely unknown and females remain excluded from many clinical studies.

Millions of women worldwide have a variety of healthcare needs largely unmet for many reasons. Some reasons are based on sociocultural, political and/or socioeconomical determinants, where women's health remains a minority focus for mainstream funding, discussion and solutions in nearly all global economic geographies.

Other reasons are as basic as the uniqueness of females and their biology. Unlike males, females will have different healthcare needs driven by various biological changes that occur throughout her life. It starts with pre-puberty, continues through puberty and adolescence, into early and mid-childbearing years and through menopause and beyond. Many researchers have claimed that this amount of variability throughout a female's life makes it difficult to study, innovate and/or improve diagnostics and therapeutics specific to female health needs; and hence, we still have very little insight about the specifics of female biology to this day. In fact, it was only twenty-five years ago when the US Food & Drug Administration (US FDA) changed their policy from excluding women of child-bearing age from clinical studies to including them into clinical studies, and yet we still see instances where male biology (even in pre-clinical studies) remains the proxy for a drug's safety and efficacy.

To underscore the unknown and therefore the un-met needs for female patients, as well as costs incurred from current-day methods and approaches some of the most common diseases, we provide two examples:

### More Women are Impacted by Cancer

The American Cancer Society estimates 1.7 MM new cases of cancer in 2018, of which 879K (52%) will be women-a majority because nearly 80K more women than men are impacted by female-prevalent breast and endocrine cancers. Further, there is a hypothesis that cancer develops at greater rates in women than in men possibly due to the cyclical nature of menstruation in combination with the effects of estrogen signaling which rapidly causes the biological development of a tumor-network.

Additionally, −3.2 MM women between the ages of 18 and 45 were diagnosed or living with some of the most prevalent female-specific cancers (Breast, Endometrial, Lung, Ovarian, Cervical), representing a $15B annual cost in healthcare for this age group.

Early detection is the best detection, but not always easy, available or affordable. When we think about the nearly 900K women who will be diagnosed with cancer this year, and the million others who are living with cancer, we wonder: what if there were a regular, easy, affordable way for physicians to detect or even predict potential cancers, starting in our teenage years?

### More Women are Impacted by Degenerative Diseases

Female-prevalent degenerative disease has twice the magnitude outlook as cancer, with −6.5 MM women between the ages of 18 and 45 diagnosed or living with some of the most prevalent female-specific degenerative diseases (such as Type II Diabetes, Heart Disease, Early-Onset Alzheimer's), representing a $27B annual cost in healthcare for this age group. Diagnostic devices targeted for gynecological use are expected to display the highest future growth rate in North America. In 2015, the available market for diagnostics in women's health was-$33B. By 2020 of the market growth is valued at $40.6B with a CAGR value of 4.2% over this time period. The American Cancer Society estimates that 51% of new cancer cases in 2018 will be women vs 49% men due to female-prevalent cancers such as endocrine, in which there is no screening test for women. Diagnostic solutions are not personalized enough for women; female biology is not widely understood yet.

### SUMMARY OF THE INVENTION

This disclosure relates to methods and devices that enables physicians, healthcare providers, pharmaceutical companies and female patients to engage in patient stratification, apply precision medicine to therapeutic use, treatment, diagnosis and/or prognosis, and/or short and long-term health monitoring of important disease-or health-related biomarkers in a reliable, cost effective, and non-invasive manner.

In one aspect, the invention provides a method for evaluating one or more disease- or health-related biomarkers in a female subject, comprising: (a) obtaining a sample of the female subject's menstrual fluid; (b) measuring the presence, absence, or level of one or more disease- or health-related biomarkers in the sample; and (c) repeating with subsequent menstrual fluid. The trend and/or average levels of the biomarker(s) can be evaluated by the healthcare provider or patient to inform healthcare or lifestyle decisions, including in some embodiments the diagnosis of early stage disease, the state of chronic disease (e.g., controlled or uncontrolled), and/or patient stratification and precision medicine to the individual patient.

In some embodiments, the present methods inform a healthcare provider to make healthcare decisions and/or providing improved health care and/or informs a female subject to make improved health/lifestyle decisions.

In various embodiments, the method prevents unnecessary medical care visits, reduces or eliminates unnecessary diagnostic tests, reduces or eliminates unnecessary administration of therapeutic agents, improves the selection of diagnostic tests, and improves the selection of therapeutic agents.

Further, in various embodiments, the present methods provide baseline biomarker levels for the patient, as well as long term and short term trends in biomarker levels. Such baseline information or trends allow for more accurate and interpretable diagnostic and/or prognostic tests including, for example, when the baseline or trend health information is used to compare to a biomarker measurement at a single point of time (including, by way of non-limiting example, at a point of care, e.g., upon visit to a healthcare profession presenting symptoms of a disease or disorder). Additionally, baseline information may be used to apply precision medicine at a given point in time of the female patient's menstruating life, for example, between the onset of menses and menopause, a female's biology will significantly change and one can use such baseline information to properly dose medication, monitor the state of chronic disease, etc. In various embodiments, the methods provided herein comprise measurement of various disease- or health-related biomarkers that are used to direct healthcare decisions and/or personal health decisions. In particular, the present invention provides for biomarker measurements for which patient stratification through selection biomarkers and precision medicine is desired across a wide range of health states and various diseases.

Provided herein is a method of stratifying human female subject, comprising obtaining a sample of the female subject's menstrual fluid; separating proteins of the menstrual fluid; measuring the level of glycation of the hemoglobin subunit alpha in the sample; and assigning female subject to a category and using said category for clinical trial enrollment and medication dosing. A category is defined by the number of hemoglobin subunit alpha modifications present in the female subject's menstrual blood. Categories may also be confirmed by the number of modifications on other proteins present in the female subject's menstrual blood. Categories are numerically assigned and each subject within a category has specific and similar attributes related to the biological health of the female subjects in each category. For instance, a category of one, subjects will be dosed in similar quantities for medications as compared to other categories in which the dosing may be more or less. In some aspects, category assignments will rate the risk of future and current disease exacerbation within female subjects of that category. For instance, a category of four, subjects will be of higher risk for increased disease progression, as compared to a category one, in which female subjects will have a lower risk of disease progression.

Also provided herein is a method for evaluating hemoglobin subunit alpha in a female subject, comprising: obtaining a sample of the female subject's menstrual fluid; separating proteins of the menstrual fluid; measuring the presence, absence, or level of glycation of the hemoglobin subunit alpha in the sample; and repeating with subsequent menstrual fluid. A post-translation modification on any protein may be detected and correlated to various diseases. The hemoglobin subunit alpha may be positively identified from the sample through MS analysis. A post-translation modification on the hemoglobin subunit alpha may be detected. The post-translation modification on any protein may be detected and correlated to various diseases. The post-translation modification may be an advanced glycation event or glutathionylation. The menstrual fluid may be collected using a 903 dried blood spot collection card. The hemoglobin subunit alpha may be detected using LC-MS/MS peptide sequencing. The post-translational modification may be detected using LC-MS/MS peptide sequencing. The protein may be identified based on mass and site of modification may be identified based on residue. Multiple glycation events may be observed on the hemoglobin subunit alpha. The multiple glycation events may be indicative of the presence of diabetes or prediabetes. The female subject's menstrual fluid may be obtained using a device comprising a disposable cartridge, optionally inserted or insertable into a wireless enabled device. The device may be a home instrument. The menstrual blood may be collected and analyzed for about 3 months, or about 6 months, or about 9 months, or about 1 year, or about 2 years, or about 3 years, or about 4years, or about 5 years, or about 6 years, or about 7 years, or about 8 years, or about 9 years, or about 10 years, or about 20 years, or about 30 years, or about 40 years, or about 50 years.

A method of detecting glycated hemoglobin on the alpha subunit in a female patient is provided, the method comprising: obtaining a menstrual fluid sample from a human female patient; and detecting whether glycation of hemoglobin alpha subunit is present in the menstrual fluid sample by using LC-MS/MS peptide sequencing.

A method of detecting modifications of any protein in a female patient is provided, the method comprising: obtaining a menstrual fluid sample from the female patient; and detecting whether modification on one or more proteins is present in the menstrual fluid sample by using LC-MS/MS peptide sequencing.

A method of diagnosing pre-diabetes or diabetes in a female patient is provided, the method comprising: obtaining a menstrual fluid sample from the female patient; detecting whether glycation of hemoglobin alpha subunit is present in the menstrual fluid sample by using LC-MS/MS peptide sequencing; and diagnosing the female patient with pre-diabetes or diabetes when the presence of one or more glycation events in the menstrual fluid sample is detected.

A method of diagnosing and treating pre-diabetes or diabetes in a female patient is provided, the method comprising: obtaining a menstrual fluid sample from the female patient; detecting whether glycation of hemoglobin alpha subunit is present in the menstrual fluid sample by using LC-MS/MS peptide sequencing; and diagnosing the female patient with pre-diabetes or diabetes when the presence of one or more glycation events in the menstrual fluid sample is detected; and administering an effective amount of insulin to the diagnosed female patient.

A method of medication dosing in a female patient is provided, the method comprising: obtaining a menstrual fluid sample from the female patient; measuring glycation of hemoglobin subunit alpha in the menstrual fluid sample; assigning the female patient to a category, as described in the claims; and administering an effective amount of medication to the patient.

### BRIEF DESCRIPTION OF THE FIGURES

FIG. 1 is a graph showing intact mass spectrum of menstrual blood sample 1.

FIG. 2 is a graph showing deconvoluted hemoglobin subunit alpha with post translational glycation.

FIG. 3 is a graph showing intact mass spectrum of menstrual blood sample 2.

FIG. 4 is a graph showing deconvoluted hemoglobin subunit alpha with post translational glycation.

FIGS. 5A-5C are graphs illustrating the analysis of hemoglobin Ale standard (96%). FIG. 5A shows total ion chromatogram, intact mass spectrum showing P1A, identified as hemoglobin subunit alpha and P2A, identified as hemoglobin subunit beta. FIG. 5B shows deconvoluted P1A shows P1B as unmodified hemoglobin subunit beta and P2B as singly glycated hemoglobin (S.9%). FIG. 5C is deconvoluted P2A shows P1C as unmodified hemoglobin subunit beta and P2c as singly glycated hemoglobin (9S.4%).

FIGS. 6A-6C are graphs illustrating the analysis of a menstrual blood sample. FIG. 6A shows intact mass spectrum showing P1A, identified as hemoglobin subunit alpha and P2A, identified as hemoglobin subunit beta. FIG. 6B is a graph of deconvoluted P1A showing P1B as unmodified hemoglobin subunit beta, P2B as singly glycated hemoglobin (3.S%), P3B as doubly glycated (3.S%), P4B as triply glycated (3.8%), and PSB as quadruply glycated (1.3%). FIG. 6C is a graph of deconvoluted P2A showing P1C as unmodified hemoglobin subunit beta and P2C as singly glycated hemoglobin (3.2%), P3C as doubly glycated (3.7%), P4C as triply glycated (2.7%).

FIGS. 7A-7C are graphs illustrating the analysis of a fingerprick blood sample. FIG. 7A shows intact mass spectrum showing P1A, identified as hemoglobin subunit alpha and P2A, identified as hemoglobin subunit beta. FIG. 7B is a graph of deconvoluted P1A showing P1B as unmodified hemoglobin subunit beta, P2B as singly glycated hemoglobin (5.8). FIG. 7C is a graph of deconvoluted P2A showing P1C as unmodified hemoglobin subunit beta and P2C as singly glycated hemoglobin (4.7%).

FIG. 8 illustrates a series of modifications found in menstrual blood and the location of the amino acid with the post-translational modifications (PTM's).

FIG. 9 represents data from 389 patients ascribing each patient to a category based on the level of glycations on the hemoglobin subunit alpha found in menstrual blood as set forth in the claims.

FIG. 10 is a graph of a blank dried blood spot collection card to illustrate the absence of any proteins without blood and/or menstrual fluid.

FIGS. 11A-11D are graphs illustrating the analysis of a fingerprick blood sample compared to a menstrual blood sample and the contents of hemoglobin subunit alpha glycation.

FIG. 11A shows intact mass spectrum from fingerpick blood of a single patient. FIG. 11B shows intact mass analysis from fingerpick blood of a singly glycated hemoglobin subunit alpha. FIG. 11C shows intact mass spectrum from menstrual blood taken from the same patient as represented in FIG. 11A and FIG. 11B. FIG. 11D shows intact mass spectrum showing an additional hemoglobin subunit alpha that is not found in the blood from the same patient's fingerprick sample.

FIGS. 12A-12D are graphs illustrating the analysis of a fingerpick blood sample compared to a menstrual blood sample and the contents of alpha glycation, in a second female patient. FIG. 12A shows intact mass spectrum from fingerpick blood of a single patient. FIG. 12B shows intact mass analysis from fingerpick blood of a singly glycated hemoglobin subunit alpha. FIG. 12C shows intact mass spectrum from menstrual blood taken from the same patient as represented in FIG. 12A and FIG. 12B. FIG. 12D shows intact mass spectrum showing an additional hemoglobin subunit alpha that is not found in the blood from the same patient's fingerpick sample.

FIGS. 13A-13F are mass spectrometry reports from a single patient that illustrates hemoglobin subunit beta modified position as lysine eight (8), which is unreported in literature. FIG. 13A illustrates the authentic HbAlc standard hemoglobin subunit beta glycan modifications and the detection of a previously unreported glycation on Lysine-8. FIGS. 13BA and 13BB show the sequence coverage map in which 87% of the hemoglobin subunit beta is reported (Seq. Id. No. 2). FIG. 13C shows the fragment coverage map in which the average structural resolutions is equal to 1.6 residues. FIG. 13D shows both the deconvoluted scan spectra and the full scan spectra of the number of residues from FIG. 13C. FIG. 13E shows the fragment coverage map in which the average structural resolutions is equal to 8.0 residues and a sequence of VHLTPEEK. FIG. 13F shows both the deconvoluted scan spectra and the full scan spectra of the number of residues from FIG. 13E.

FIGS. 14A-14F are mass spectrometry reports from a second, and separate patient from FIG. 13A-F, that illustrates hemoglobin subunit beta modified position as lysine eight (8), which is unreported in literature. FIG. 14A illustrates the authentic HbAlc standard hemoglobin subunit beta glycan modifications and the detection of a previously unreported glycation on Lysine-8. FIGS. 14BA and 14BB show the sequence coverage map in which 86.5% of the hemoglobin subunit beta is reported (Seq. Id. No. 2). FIG. 14C shows the fragment coverage map in which the average structural resolutions is equal to 1.6 residues. FIG. 14D shows both the deconvoluted scan spectra and the full scan spectra of the number of residues from FIG. 14C. FIG. 14E shows the fragment coverage map in which the average structural resolutions is equal to 8.0 residues and a sequence of VHLTPEEK (SEQ ID No. 140). FIG. 14F shows both the deconvoluted scan spectra and the full scan spectra of the number of residues from FIG. 14E.

FIGS. 15AA-15F are mass spectrometry reports from three separate patients that illustrates that no glycations are found on hemoglobin subunit beta with between 75%-87% sequence coverage. FIGS. 15AA and 15AB show the sequence coverage map in which 75.3% of the hemoglobin subunit beta is reported (Seq. Id. No. 2). FIG. 15B shows the fragment coverage map in which the average structural resolutions is equal to 1.1 residues from the patient in FIG. 15AA. FIG. 15C shows the sequence coverage map from a separate female subject in which 76.7% of the hemoglobin subunit beta is reported (Seq. Id. No. 2). FIG. 15D shows the fragment coverage map in which the average structural resolutions is equal to 1.1 residues from the patient in FIG. 15C. FIG. 15E shows the sequence coverage map from a separate female subject in which 76.7% of the hemoglobin subunit beta is reported (Seq. Id. No. 2). FIG. 15F shows the fragment coverage map in which the average structural resolutions is equal to 1.0 residues from the patient in FIG. 15E.

FIGS. 16AA-16CD are a table representing the unique peptide sequences for the modified hemoglobin subunit alpha, including, the site of modification on the amino acid from the menstrual blood samples of multiple female subjects over a period. Seq. Id. Nos. 3-140 are shown in FIGS. 16AA-16CD.

FIGS. 17AA-17GD are a table representing the unique peptide sequences for five (5) additional modified biomarkers (Seq. Id. Nos. 141-886), including, the site of modification on the amino acid from the menstrual blood samples of multiple female subjects over a period. FIGS. 17AA-17GD serve as a proxy for the data for all other biomarkers.

FIGS. 18A-18D illustrates mass spectrometry reports from two patients in which alpha-1 antitrypsin was detected by monitoring a characteristic peptide, observed with a range from 0.0166% to 0.047% of total peptide detected. FIG. 18A shows intact mass spectrum from the menstrual blood of a single female subject. FIG. 18B shows intact mass analysis from the female subjects menstrual represented in FIG. 18A as having an observed mass of −820 Da and an observed retention time of 36.21 minutes. FIG. 18C illustrates the unique peptide as ITPNLAEFAFSLYR (Seq. Id. No. 887). FIG. 18D shows intact mass spectrum from the menstrual blood of a single patient as different from the female subject in FIG. 18A.

FIGS. 19A-19D are graphs representing the analysis of menstrual blood samples. The graphs are samples from four separate female subjects in which glutathionylation is detected and observed with a range from 5.7% to 12.1% glutathionylation of hemoglobin subunit beta. FIG. 19A shows intact mass deconvoluted spectrum from the menstrual blood of a single female subject with 5.7% glutathionylation on hemoglobin subunit beta. FIG. 19B shows intact mass deconvoluted spectrum from the menstrual blood of a single female subject, separate from the female subject in FIG. 19A, with 12.1% glutathionylation on hemoglobin subunit beta. FIG. 19C shows intact mass deconvoluted spectrum from the menstrual blood of a single female subject, separate from the female subject in FIG. 19A & 19B, with 6.3% glutathionylation on hemoglobin subunit beta. FIG. 19D shows intact mass deconvoluted spectrum from the menstrual blood of a single female subject, separate from the female subject in FIGS. 19A, 19B, 19C, with 8.2% glutathionylation on hemoglobin subunit beta.

FIG. 20A-20B are graphs representing the analysis of menstrual blood samples. The graphs are samples from two separate patients in which glycated albumin is detected. FIG. 20A shows intact mass spectrum from the menstrual blood of a single female with 34 times glycated albumin. FIG. 20B shows intact mass spectrum from the menstrual blood of a single female subject, separate from the female subject in FIG. 20A, with 34 times glycated albumin.

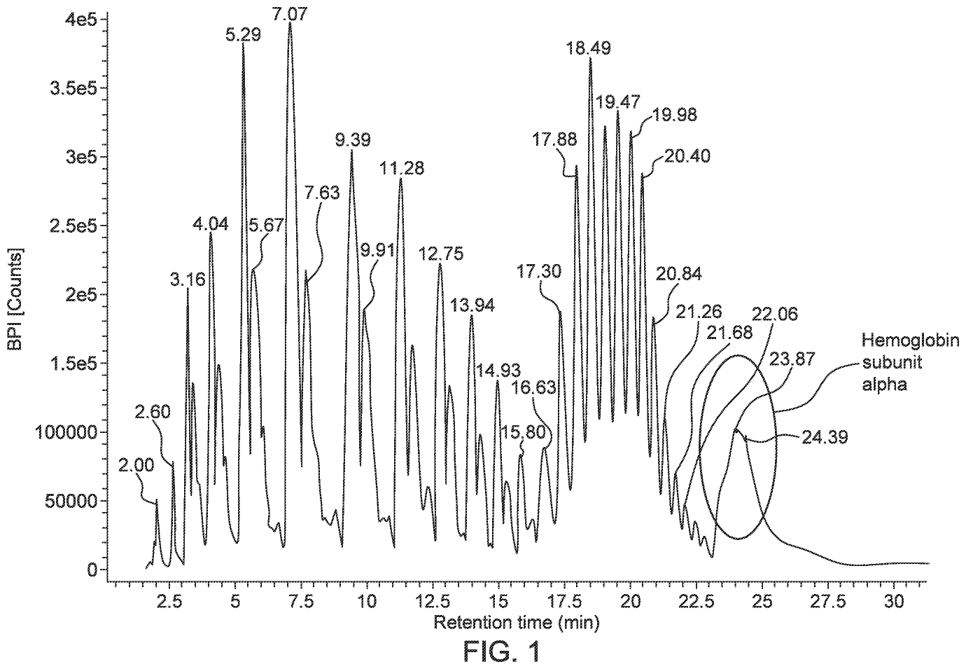

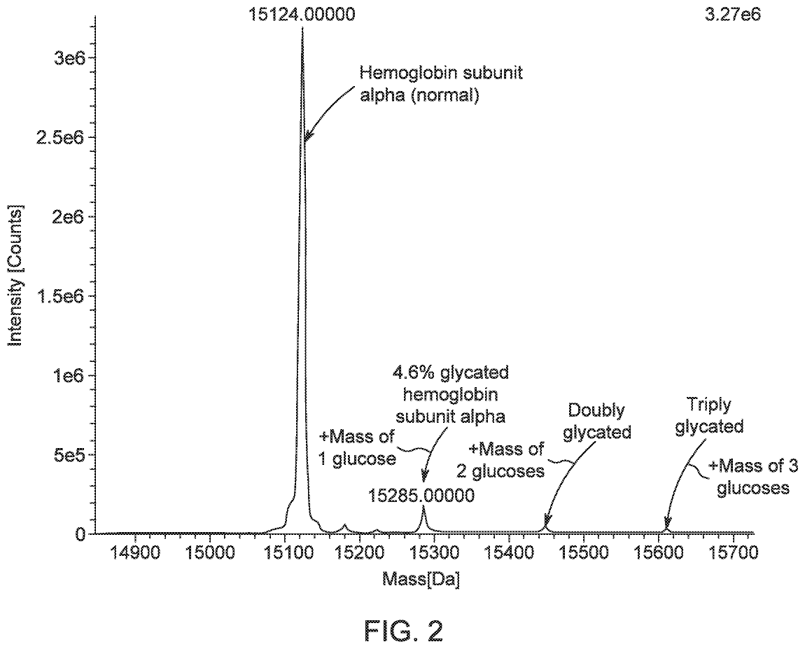

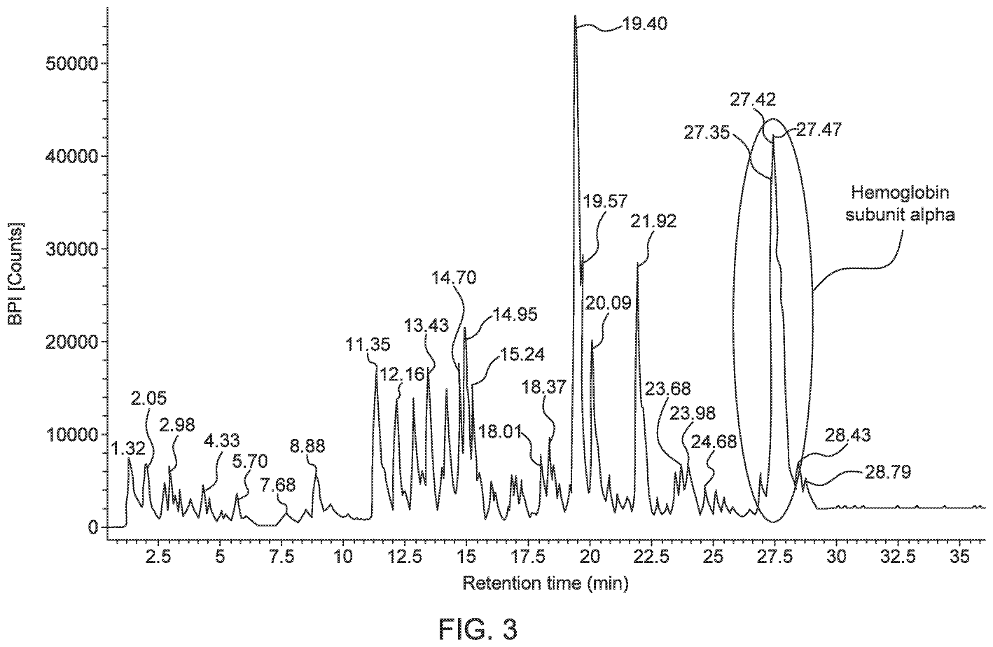

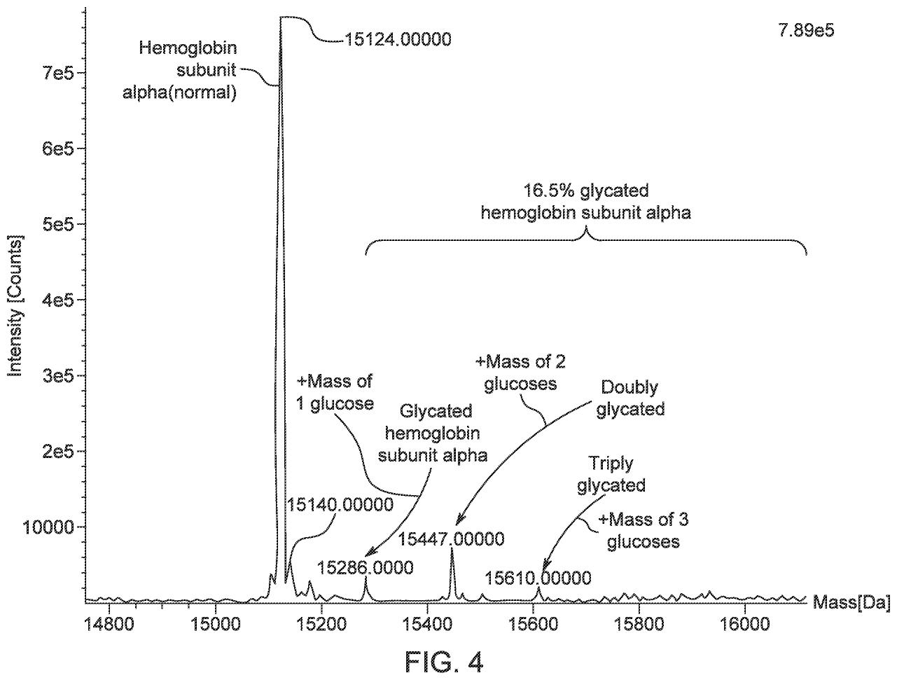

### DETAILED DESCRIPTION

The genomic, proteomic and bio-informatic interplay has a central theme in disease progression and sex-bias that revolves around estrogen and cyclical spikes each month at menses.

The present invention is based, in part, on the discovery of novel detection systems and methods for detecting PTM's on proteins specifically, glycation, oxidation, carboxymethyl, deamidation and glutathionylation of the hemoglobin subunit a (alpha), alpha 1 antitrypsin, hemoglobin subunit beta, Lon protease homolog 2, peroxisomal, serotransferrin, peroxiredoxin-2, TANC1, Teashirt homolog 2, Glycodelin, FE65-Amyloid-beta A4 precursor protein-binding family B member 1, Albumin, and Bovine Casine. These unique methods described herein provide for a non-invasive and robust method of early and continuous detection of novel disease biomarkers for personalized medicine and patient stratification. The systems described herein solve the problem of early and easy detection, tracking, and treatment of women's diseases.

The presence of multiple PTM's on biomarkers in menstrual fluid are quite different from circulating blood. The menstrual cycle provides a unique and monthly trackable, yet non-invasive biopsy for menstruating women. Thus, monitoring menstrual fluid allows for the possibility of an easily assessed diagnosis of cancer, specifically, endometrial, breast and lung cancer, early detection of degenerative disease such as early on-set Alzheimer's and prediabetes, all with potentially global utility.

In addition to disease detection, the methods described herein allow for application of precision based on patient stratification techniques as well as selection biomarker usage. A new method of detecting biomarkers with multiple modification events, with positions on specific lysine residues is described herein. Novel modification patterns on proteins that appear to be unique to the blood found in menstrual fluid have been discovered.

The menstrual blood milieu presents an environment that can be analyzed quite differently than that from circulating blood. For example, the mean pH of the vagina is typically 4.0 to 4.5 during menstruation, rising slightly to blood's pH of 7.4 when menstrual blood is present. The exact effects of this unique environment on modified proteins in menstrual blood and the indications it may predict have previously gone uninvestigated without baseline studies such as those presented herein. Monitoring this information and correlating it to clinical information provides valuable data for diagnosis and/or treatment of patients in various states of disease progression, management and treatment.

In one aspect, the method comprises the steps of obtaining a sample of a human female's menstrual fluid, separating the proteins of the menstrual fluid, detecting protein profiles, identifying individual proteins, and elucidating biomarkers. Proteins can be positively identified from a complex menstrual fluid sample (e.g., dried menstrual fluid) through MS analysis. Unique post-translational modifications (e.g., advanced glycation events) can also be observed on the proteins that compare with standard tests for monitoring disease.

The proteins of the menstrual fluid can be collected using any commercially available blood sampling device that provides a consistent, uniform, and rapid collection of fluid. Health profiles are completed by all patients and where applicable and possible, patient medical records, health tracking device data, etc. will be included. Samples are collected in the patient's home according to LSH's Collection Protocol using EBF-Inc.'s 903 dried blood spot (DBS) collection device. Up to one-hundred and fifty microliters (150 uL) of menstrual blood is collected.

A multitude of proteins can be detected from the processed menstrual fluid including but not limited to, actin-binding, albumin, chaperone proteins, enzymes, antibodies, ribosomal proteins, structural proteins, transcription factors, and hemoglobin, using, for example, LC-MS/MS peptide sequencing.

The analyzation of unique modifications that have not been detected by current techniques are outlined herein.

First, the observed mass of the proteins is matched against its intensity output (e.g. mass-to-charge (mlz) ratio) versus intensity (e.g. current generated from multiplier) which generates the mass spectrum. The presence of ions are identified in a particular mass which enables the count of all amino acids in a protein element to be identified. The difference in peaks of these amino acids provide a residue in which we can compare masses with standard weights of amino acids to identify the presence of modification in different molecular samples. Through this process the ability to analyse biomarkers is achieved in order to gain overall health knowledge of the patient for example, presence of certain amino acids are related to human disease which ultimately points out the protein causing the disease.

Once analysed, precision medicine can be applied using a 1, 2, or 50 or more combined biomarkers per female subject to both early detect various diseases as well as apply treatment in a more precise manner to female patients. As described herein, each protein represents a single biomarker and a combination of biomarkers can be combined to provide more accurate measures including, diagnosis, prognosis, patient stratification and/or precision medicine.

In one aspect, hemoglobin and its subunits are detected. Post-translation modifications (e.g., glycation, glutathionylation) are detected during protein profiling experiments. For example, PTMs and hemoglobin identifications may be observed based on mass. Without wishing to be bound by theory, glutathionylation may be observed as a result of oxidative stress and/or glycation may be observed when high concentrations of sugars are present in the menstrual fluid sample. In some aspects, multiple glycation events (e.g., 1 to 4 or more events) are observed on the alpha subunits of hemoglobin.

In one aspect, PIP5K1a is detected, specifically observing the LEVAESEFTH peptide sequence (Seq. Id. No. 888) consistently. Increases in PIP5K1a in blood increase the risk of cancer due to cell proliferation, survival, invasion (Citation doi: 10.1073/pnas.1107808109), and creation of invododia (Citation https://doi.org/10.1111/j.1349-7006.2010.01574.x). In addition to identification of PIP5K1a, post-translational modifications have been identified, including, but not limited to, glycyl lysine isopeptide at position 103 and phosphoserine at position 486. In one aspect, GRP-78 is detected and observed in (nine) 9 unique, reproducible, peptides.

Increases in this protein in blood are consistently linked to breast cancer, endometrial cancer, familial breast and ovarian cancer and pre-eclampsia. PTM's reported, including, but not limited to, Signal peptide—between positions 1 and 18; Polypeptide chain between positions 19 and 654; Modified residue at positions 86, 125, 160,213, 326,353,447, 518, 585, 591, 643 and 648; Cross-link at positions 352 and 353.

In one aspect, alpha 1 antitrypsin is detected. In two separate patients, alpha-1 antitrypsin, is detected by monitoring a characteristic peptide, observed in a range from 0.0166% to 0.047% of total peptide detected. In the first patient, 12 unique peptide sequences were matched in which one was confidently followed as ITPNLAEFAFSLYR (Seq. Id. No.887) and exhibits 0.047% of entire peptide content analyzed in sample. In the second patient, 15 unique peptide sequences were matched in which one was confidently followed as ITPNLAEFAFSLYR (Seq. Id. No. 887) and exhibits 0.0166% of entire peptide content analyzed in sample. In one aspect, hemoglobin subunit beta is detected. Post-translation modifications (e.g. glutathionylation) are detected during protein profiling experiments. 28% of patient samples demonstrated modifications and 19 unique peptide sequences.

In one aspect, Ion protease homolog 2, peroxisomal is detected. In some aspects, upregulation is observed as a result of linkage to poor prognosis in cancer patients and tumor growth. Observed with one unique peptide sequence in seven samples.

In one aspect, serotransferrin is detected in 42% of patients with 14 unique peptide sequences and a signal peptide position of 1-19 and chain positions 20-698.

In one aspect, peroxiredoxin-2 is detected with four unique sequences and PTM's such as: Initiator methionine; Polypeptide chain-between positions 2 and 198; Modified residue at position 2, 112 and 182; Disulfide bond at position 51 and 172.

In one aspect, TANC-1 is detected in 57% of patients with three unique sequences and PTM's such as: Phosphorylated by MINK1 and TNIK upon stimulation by RAP2A.

In one aspect, teashirt homolog 2 is detected in 29% of patients with four unique sequences.

In one aspect, glycodelin is detected with four unique sequences and PTM's on signal peptide—position 1-18 and Glycosylations at positions 46 and 81; Disulfide bonds between positions 84 and 178 and between positions 124 and 137; Four distinct glycoforms A, C, F and S arise from different N-linked oligosaccharide chains at amino acid residues Asn-46 and Asn-81.

In one aspect, FE65-amyloid-beta A4 precursor protein-binding family B member 1 is detected with PTM's on polypeptide chain between positions 1 and 710; Modified residue at positions 517, 547 and 610.

In one aspect, albumin is detected with PTM's on Signal peptide positions 1-18; Propeptide 19-24; Glycosylations positions 36, 75, 161, 186, 223, 249, 257, 300, 305, 337, 341, 342, 347,375,402,437,463,468, 518, 549, 558, 560, 569, 597 (plus additional reported). Disulfide bonds between positions: 77-86, 99-115, 114-125, 148-193, 192-201, 224-270, 269-277, 289-303, 302-313, 340-385, 384-393, 416-462, 461-472, 485-501, 500-511, 538-583, 582-591; Modified amino acid residues: Phosphoserine positions 29, 82, 89,297,443,513; Phosphothreonine positions 107, 444, 446; N6-succinyllysine positions 229, 460, 543, 588; N6-methyllysine position 558.

In various embodiments, the present methods provide for protein combinations based on unique signatures based on PTM's found in menstrual blood that can be applied to improve personal healthcare and/or health decisions. Such protein profiling based on unique signatures found in each female patient is used to form a more complete subject's health history than standard point of care testing.

In one aspect, the method of detecting modified proteins and novel biomarkers in a female patient, comprises obtaining a menstrual fluid sample from a human female patient; and detecting whether modifications are present in the menstrual fluid sample by using LC-MS/MS peptide sequencing and analyzation techniques.

In various embodiments, the application of precision medicine is applied by using the female biology as the method of diagnosing various diseases and treating the female patient, which comprises obtaining a menstrual fluid sample from a human female patient; by first stratifying the patient based on the number of glycation of hemoglobin alpha subunit present in the menstrual fluid sample by using LC-MS/MS peptide sequencing; diagnosing the patient with disease when the presence of a set number of proteins and modification events in the menstrual fluid sample are detected; alerting patient to a lifestyle change and/or prompting the administering of an effective amount of medication to the diagnosed patient.

In one aspect, the method of diagnosing prediabetes and/or diabetes in its nascent stage in a female patient, comprises obtaining a menstrual fluid sample from a human female patient; detecting whether glycation of hemoglobin alpha subunit is present in the menstrual fluid sample by using LC-MS/MS peptide sequencing; and diagnosing the patient with prediabetes and/or diabetes when the presence of a set number of glycation events in the menstrual fluid sample is detected; alerting patient to a lifestyle change and/or prompting the administering of an effective amount of insulin to the diagnosed patient.

In one aspect, the method of diagnosing well characterized liver and/or lung disorders in its nascent stage in a female patient, comprises obtaining a menstrual fluid sample from a human female patient; detecting whether post translational modifications on Alpha 1-Antitrypsin are present in the menstrual fluid sample by using LC-MS/MS peptide sequencing; and diagnosing the patient with liver and/or lung disorders when the presence of a set number of modification events in the menstrual fluid sample are detected; alerting patient to a lifestyle change and/or prompting the administering of an effective amount of medication to the diagnosed patient.

In one aspect, the method of diagnosing any early stage disease and/or providing prognosis in a female patient, comprises obtaining menstrual fluid sample from a human female patient; detecting whether post-translational modifications are present in the menstrual fluid sample by using LC-MS/MS peptide sequencing;

In one aspect, the method of diagnosing overall health in a female patient, comprises obtaining a menstrual fluid sample from a human female patient; detecting whether glycation of hemoglobin alpha subunit is present in the menstrual fluid sample by using LC-MS/MS peptide sequencing; stratifying the patient based on the presence of a set number of glycation events in the menstrual fluid is detected; and assigning the patient into a category when the presence of a set number of glycation events in the menstrual fluid sample is detected; alerting patient to a lifestyle change and/or prompting the diagnosis of latent disease, potentially at risk of exacerbation, and/or prompting of an reevaluation of effective amount of medication to the diagnosed patient. As used herein, a category is defined by the number of hemoglobin subunit alpha modifications present in the female subject's menstrual blood. Categories may also be confirmed by the number of modifications on other proteins present in the female subject's menstrual blood. Categories are numerically assigned and each subject within a category has specific and similar attributes related to the biological health of the female subjects in each category. For instance, a category of one, subjects will be dosed in similar quantities for medications as compared to other categories in which the dosing may be more or less. In some aspects, category assignments will rate the risk of future and current disease exacerbation within female subjects of that category. For instance, a category of four, subjects will be of higher risk for increased disease progression, as compared to a category one, in which female subjects will have a lower risk of disease progression.

In one aspect, the application of precision medicine is applied by using the female biology as the method of diagnosing and treating pre-diabetes and/or diabetes in a patient, comprises obtaining a menstrual fluid sample from a human female patient; detecting whether glycation of hemoglobin alpha subunit is present in the menstrual fluid sample by using LC-MS/MS peptide sequencing; diagnosing the patient with diabetes when the presence of a set number of glycation events in the menstrual fluid sample is detected; alerting patient to a lifestyle change and/or prompting the administering of an effective amount of insulin to the diagnosed patient.

In various aspects, the application of precision medicine is applied by using the female biology as the method of diagnosing early stage disease in a patient, comprises obtaining a menstrual fluid sample from a human female patient; detecting whether protein modifications are present in the menstrual fluid sample by using LC-MS/MS peptide sequencing; diagnosing the patient with disease when the presence of a set number of modification events in the menstrual fluid sample are detected; alerting patient to a lifestyle change and/or prompting the administering of an effective amount of medication to the diagnosed patient.

In various embodiments, the present methods provide for repeated sampling of a female subject's menstrual fluid to allow for an accumulation of data over a period of weeks, months or years. Such data is used to form a more complete subject's health history than standard point of care testing. As described herein, such data allows for an improvement in personal healthcare and/or health decisions.

Also, in some embodiments, the present invention provides for a non-invasive method of monitoring one's health. For example, in various embodiments, the collection of menstrual fluid provides biomarker information (e.g., glycation and glutathionylation of hemoglobin subunit alpha) without the need for blood draws, etc. In some embodiments, the present methods allow for long term health monitoring without various deleterious side effects of standard monitoring including, by way of illustration, excessive bleeding, fainting, lightheadedness, hematoma, infection, pricking or stinging sensations, bruising, pain, throbbing, etc. In some embodiments, the non-invasive nature of the sample collection improves patient compliance and allows for a more complete set of data.

In various embodiments, the evaluation informs a healthcare provider to provide improved health care and/or informs the female subject to make improved health decisions. For example, subtle alterations (e.g., glycation and glutathionylation of hemoglobin subunit alpha) in one or more disease-or health-related biomarkers over time, away from a normal level, may provide an earlier indication of a disease or disorder than a test at a single point of time (including, by way of non-limiting example, at a point of care) test and before symptoms arise. Further, the repeated evaluations of the present methods allow for early detection of a disease or disorder as the evaluation is not driven by a symptom or sign on the subject part.

For instance, the repeated evaluation of menstrual fluid allows for increased healthcare vigilance and largely eliminates the need for reactive medical interventions. In some embodiments, the subject has a chronic disease such as diabetes and the state (e.g., controlled or uncontrolled) is monitored over time.

In some embodiments, the evaluation comprises any one of diagnosis, prognosis, and response to treatment. Diagnosis refers to the process of attempting to determine or identify a possible disease or disorder. Prognosis refers to the predicting of a likely outcome of a disease or disorder. A complete prognosis often includes the expected duration, the function, and a description of the course of the disease, such as progressive decline, intermittent crisis, or sudden, unpredictable crisis. Response to treatment is a prediction of a patient's medical outcome when receiving a treatment (e.g. response to a therapeutic agent). Responses to treatment can be, by way of non-limiting example, pathological complete response, survival, and remission.

In various embodiments, the present invention pertains to the generation of a long term health history record that informs care. Accordingly, in various embodiments, the menstrual sample is obtained periodically. In some embodiments, the menstrual sample is obtained on a regular basis. For instance, sampling may occur about once every month, or about once every other month, or about once every 3 months, or about once every 6 months, or about once every 9 months, or about once every year. In some embodiments, about 1 to about 12, or about 2 to about 10, or about 3 to about 8 samples are evaluated per year. Furthermore, in some embodiments the present methods are repeated long term to generate a large data set. For example, in some embodiments, the evaluation is repeated for about 3 months, or about 6 months, or about 9 months, or about 1 year, or about 2 years, or about 3 years, or about 4 years, or about 5 years, or about 6 years, or about 7 years, or about 8 years, or about 9 years, or about 10 years, or about 20 years, or about 30 years, or about 40 years, or about 50 years.

In various embodiments, the female subject's biomarker information (e.g., glycation and glutathionylation of hemoglobin subunit alpha) provides baseline health information, as well as long term and short term trends in biomarker levels. In various embodiments, the baseline or trend health information is used to compare to a biomarker measurement at a single point in time (e.g. at the point of care). Accordingly, in some embodiments, the present methods prevent or mitigate incorrect or missed diagnosis. In various embodiments, the present methods allow for one or more of reducing or eliminating unnecessary medical care visits, reducing or eliminating unnecessary diagnostic tests, reducing or eliminating unnecessary administration of therapeutic agents, improving the selection of appropriate diagnostic tests, and improving the selection of appropriate therapeutic agents.

In various embodiments, the present invention relates to various disease- or health-related biomarkers that are available in menstrual fluid. For example, the present invention provides for disease- or health-related biomarkers for which long term data is desirable. Further, in some embodiments, the disease-or health-related biomarkers of the present invention are those which are hindered by inconsistency when measured in a single point of time (including, by way of non-limiting example, at a point of care) scenario. Further still in some embodiments, the present disease-or health-related biomarkers include those which are surrogates for slowly developing and/or relatively symptom-free and/or chronic diseases. In various embodiments, the evaluation is of glycation and glutathionylation of hemoglobin subunit alpha and is useful to screen for, diagnose, and monitor diabetes and pre-diabetes. For instance, a female subject at risk for type II diabetes may have repeated evaluation of these disease-or health-related biomarkers and such readings may direct lifestyle changes (e.g. increased exercise, improved diet) and/or treatments to avoid an onset or worsening of diabetes (e.g. administration of metformin to control blood glucose levels).

Further, the present methods, at least in situations of relatively controlled blood glucose, obviate a need for inconvenient blood glucose monitors that suffer from poor patient compliance due to, in part, their invasiveness (e.g. monitors requiring finger pricking for blood sampling). In some embodiments, the present methods show a gradual increase in glycation and glutathionylation of hemoglobin subunit alpha and allow for lifestyle changes or medical intervention to prevent, for example, the evaluation from normal to pre-diabetes or pre-diabetes to diabetes. Further, it is suggested that diabetic subjects maintain an HbAlc level of less than about 7% and the present methods allow for a non-invasive manner to monitor this. For example, if the periodic evaluations show HbAlc levels rising to 7% or above, medical intervention of lifestyle change may be ordered to mitigate detrimental effects (e.g. increased likelihood or onset of one or more of eye disease, heart disease, kidney disease, nerve damage, and stroke).

The methods described herein allow for the personalized and routine monitoring of menstrual fluid that could lead to the early detection and treatment of disease.

### Assays

In one aspect, the invention relates to an assay (e.g., a diagnostic assay). The assay may utilize mass spectrometry to identify biomarkers and other major proteins (e.g., hemoglobin).

The assay described herein is capable of documenting multiple glycation events on the alpha subunits of hemoglobin. This stands in unique juxtaposition to the standard test for diabetes management, the HbAlc, which measures the concentration of singly glycated beta subunits of hemoglobin.

Intact mass analysis may be used to identify the alpha and beta subunits of a protein such as hemoglobin. Detailed analysis of the hemoglobin samples may show one or more glycations of alpha subunit, for example 1, 2, 3, 4, or more glycations, 1 to 4, 1 to 3, or 2 to 4 glycations may be observed. This assay may serve as a diagnostic assay for disease (e.g., diabetes) with routine testing of menstrual blood, or may serve as an assay for pre-diagnosis of diseases such as pre-diabetes and/or diabetes.

In some embodiments, the present invention relates to a device for collection of a female subject's menstrual fluid sample and uses thereof. In some embodiments, the device is a disposable cartridge which may be inserted into a wireless enabled device. In various embodiments, the device is a home instrument. In various embodiments, the device is operated by the patient, without the need for intervention by a medical professional.

Accordingly, in various embodiments, the patient is spared the inconvenience of scheduling an appointment in a medical clinical and may be able to institute sample collection at her convenience and without scheduling delays.

In various embodiments, the device is or comprises a sampling implement that provides a means to collect a sample from a subject. The sampling implement may be connected to a collection chamber via a sampling implement holder. In some embodiments, the sampling implement is disposed at the distal end of a shaft, which shaft can be solid, hollow or semi-permeable. In some embodiments, the sampling implement is a swab, a comb, a brush, a spatula, a rod, a foam, a flocculated substrate or a spun substrate.

In various embodiments, the device is associated with and/or integrated into one or more of a tampon, pad (menstrual napkin) or menstrual cup (see, e.g., International Patent Publication Nos. WO/2002/080827 and WO/2006/058409, the contents of which are hereby incorporated by reference).

In various embodiments, the collection of menstrual fluid may take place on one of the heaviest days of the donor's menstrual period which may be the first or second day. In various embodiments, the general area around the vagina may be cleansed with an aseptic cleaning pad prior to collection.

In various embodiments, a single sample or multiple samples may be collected. The sample or samples may be maintained at room temperature (about 15° C. to about 25° C.). In various embodiments, samples may be shipped to a laboratory so long as the sample or samples arrive at the laboratory within about 24 hours to about 72 hours of collection. Alternatively, samples may be refrigerated at about 1° C. to about 10° C.

In various embodiments, the sample may be subjected to centrifugation and either the supernatant or pellet may be analyzed.

In some embodiments, the present methods comprise contacting an agent that specifically binds a biomarker (e.g., glycated hemoglobin) with the menstrual sample. For example, such an agent may be an antibody. Illustrative, but non-limiting methods for evaluation include one or more of immunohistochemical staining, western blotting, in cell western, immunofluorescent staining, ELISA, and fluorescent activating cell sorting (FACS), or any other method described herein or known in the art.

There are generally two strategies used for detection of epitopes on antigens in body fluids or tissues, direct methods and indirect methods. The direct method comprises a one-step staining, and may involve a labeled antibody (e.g. FITC conjugated antiserum) reacting directly with the antigen in a body fluid or tissue sample. The indirect method comprises an unlabeled primary antibody that reacts with the body fluid or tissue antigen, and a labeled secondary antibody that reacts with the primary antibody. Labels can include radioactive labels, fluorescent labels, hapten labels such as, biotin, or an enzyme such as horse radish peroxidase or alkaline phosphatase. Methods of conducting these assays are well known in the art. See, e.g., Harlow et al. (Antibodies, Cold Spring Harbor Laboratory, NY, 1988), Harlow et al. (Using Antibodies, A Laboratory Manual, Cold Spring Harbor Laboratory, NY, 1999), Virella (Medical Immunology, 6th edition, Informa HealthCare, New York, 2007), and Diamandis et al. (Immunoassays, Academic Press, Inc., New York, 1996). Kits for conducting these assays are commercially available from, for example, Clontech Laboratories, LLC. (Mountain View, Calif.).

In various embodiments, antibodies include whole antibodies and/or any antigen binding fragment (e.g., an antigen-binding portion) and/or single chains of these (e.g. an antibody comprising at least two heavy (H) chains and two light (L) chains inter-connected by disulfide bonds, an Fab fragment, a monovalent fragment consisting of the VL, VH, CL and CHI domains; a F(ab)2 fragment, a bivalent fragment including two Fab fragments linked by a disulfide bridge at the hinge region; a Pd fragment consisting of the VH and CHI domains; a Fv fragment consisting of the VL and VH domains of a single arm of an antibody; and the like). In various embodiments, polyclonal and monoclonal antibodies are useful, as are isolated human or humanized antibodies, or functional fragments thereof.

Standard assays to evaluate the binding ability of the antibodies toward the target of various species are known in the art, including for example, ELISAs, western blots and RIAs. The binding kinetics (e.g., binding affinity) of antibodies also can be assessed by standard assays known in the art, such as by Biacore analysis.

A person skilled in the art will appreciate that a number of methods can be used to detect or quantify the DNA/RNA levels of various disease-or health-related biomarkers (e.g., hemoglobin).

Gene expression can be measured using, for example, low-to-mid-plex techniques, including but not limited to reporter gene assays, Northern blot, fluorescent in situ hybridization (FISH), and reverse transcription PCR (RT-PCR). Gene expression can also be measured using, for example, higher-plex techniques, including but not limited, serial analysis of gene expression (SAGE), DNA microarrays. Tiling array, RNA-Seq/whole transcriptome shotgun sequencing (WTSS), high-throughput sequencing, multiplex PCR, multiplex ligation-dependent probe amplification (MLPA), DNA sequencing by ligation, and Luminex/XMAP.

A person skilled in the art will appreciate that a number of methods can be used to detect or quantify the level of RNA products of the disease- or health-related biomarkers within a sample, including arrays, such as microarrays, RT-PCR (including quantitative PCR), nuclease protection assays and Northern blot analyses.

In various embodiments, the female subject is menstruating and thus generating sample for evaluation. In some embodiments, the female subject is non-menopausal or recently menopausal. In some embodiments, the female subject may be repeatedly evaluated until pregnancy and resume evaluation post-partum. In these embodiments, the female subject may be monitored for post-partum complications. For instance, the pre-pregnancy data may be used in comparison with post-partum data to monitor a restoration of pre-pregnancy health baselines.

In some embodiments, the female subject has an age in a range of from about 13 years to about 60 years. In some embodiments, the female subject is about 10 years old, or about 15 years old, or about 20 years old, or about 25 years old, or about 30 years old, or about 35 years old, or about 40 years old, or about 45 years old, or about 50 years old, or about 55 years old, or about 60 years old, or about 65 years old.

### EXEMPLIFICATION

The following results are applicable to any of the other proteins and/or combination of proteins and modifications described herein.

### Example 1—Mass Spectrometry Based Assay

Menstrual blood was analyzed for biomarkers using a shot gun proteomics approach. Eleven major proteins were identified and list above. By intact mass analysis, we were able to identify unique modifications to the 11 proteins. Upon detailed analysis of certain modifications such as the hemoglobin samples, we observed multiple glycations of the alpha subunit (1-4 glycans observed) in at least 389 patient samples. In comparison to a glycated hemoglobin control, we only observed glycation on the beta subunit and only a single glycation on the alpha subunit. Thus, the observation of the alpha-subunit of glycation was a unique finding in menstrual blood, in addition to the several other unique findings described herein. Glutathionylation on subunit beta of hemoglobin was also observed in at least 40 patient samples. Glutathionylation is sometimes seen in diabetic patients with microangiopathy.

### Example 2—Immunological Assay for Biomarker Quantitation in MB

After identification of the modified proteins, we digested the protein using enzymes and subjected it to peptide mapping and post-translational analysis using mass spectrometry. The discovery of modified proteins are unique finds (described above), and further identification of the exact sites of modifications was used in a separate process described herein.

This procedure was employed to identity the location of particular modification events on 130 proteins, found in the menstrual fluid sample. Using this method, multiple specific locations of modification were identified.

Extraction of menstrual fluid from collection paper: Menstrual fluid-soaked filter paper portion (a spiral 6 cm in diameter) was extracted with 200 ul of 100 mM ammonium bicarbonate in a 1.5 ml Eppendorf tube. It was shaken at room temperature, 1000 RPM for 45 min.

Trypsin digest: 20 ng of lyophilized trypsin was prepared by adding 20 uL of buffer solution and mixing by pipetting. This solution was added to 20 uL of sample and incubated for 2 hours at 37 C. This samples was then subjected to MS/MS analysis.

Glu-C digest: 10 ng of lyophilized Glu-C was prepared by adding 10 uL of buffer solution and mixing by pipetting. This solution was added to 10 uL of sample and incubated for 2 hours at 37 C. This samples was then subjected to MS/MS analysis.

UPLC-MS method: Samples were separated on a C-18 reverse phase column over the course of 90 minutes. They were then injected into the ESI-Q-Tof Mass Spectrometer using standard parameters for MS analysis. The mass spec data collected in an entire sample run was analyzed via the UNIFI software package.

Data interpretation: All MS/MS peptide fragmentation data was interpreted using UNIFI software. Identification of peptide fragments containing K-linked modified events were identified and cross matched between the alternative digest methods.

Following the identification of proteoform(s) of interest using bottom-up and top-down MS proteomics workflows, we develop highly sensitive immunological assays for simple quantitation in MB.

To accomplish this, using the process described herein, a full protein purification regimen was used to isolate the proteoform(s) in sufficiently high concentrations for polyclonal antibody production. The exact steps of the regimen are informed by the results of each proteomics study but might include: size exclusion chromatography (SEC), ion exchange chromatography (IEC), preparative high-performance liquid chromatography (HPLC), isoelectric focusing (IEF), and/or affinity chromatography techniques (e.g. lectin affinity chromatography for glycosylated proteins).

After the proteoform(s) of interest are isolated in sufficient purity as determined by MS and quantities (−250-1000 μg), using standardized custom animal monospecific antibody production protocols offered by an assortment of companies such as Thermo Fisher Scientific (Waltham, MA), Abzena (Cambridge, UK), and GenScript (Piscataway, NJ), are used.

Monospecific antibody production is tailored to target proteoform-specific post-translational modifications to ensure singular binding of the developed antibody to the proteoform of interest in situ (aberrant post-translational modifications, such as glycosylation, have been well characterized in some cancer types). Finally, custom antibodies are chemically or enzymatically labelled as necessary for detection purposes (via colorimetry or fluorometry) in the immunological assay. The resulting assay is designed to be highly specific, easy-to-use, and produce rapid quantitative results.

Performance of developed assays through parallel ultrahigh resolution mass spectrometry/ion mobility studies.

An antibody raised to the modified peptide on the protein will serve as a robust, inexpensive, diagnostic assay for reliable biomarkers for detection of disease, stratification, and assay kit(s).

### Example 3—Glycated Hemoglobin Standard Analysis

This example shall be used as proxy for all proteins and modifications described herein as applying to all industry standard tests for various diseases and the usage of selection biomarkers.

The ability to observe multiple glycation events on menstrual fluid hemoglobin could be a promising diagnostic for prediabetes, as poor control of blood sugar would lead to such glycation events.

The goal of this experiment was to determine the position of the glycation events. For comparison, authenticated 96% natural HbAlc sample (abeam ab98306) was obtained and compared with a fingerpick sample from a patient. Under LC-MS conditions, separate alpha and beta subunits were observed. The hemoglobin Ale is a native protein and prepared from packed red blood cells, the protein arrives intact. The tetramer molecular weight was −64 KDa, consisting of four subunits of approximately 16 KDa each. Analysis showed that this standard exhibited a level of 5.88% glycated hemoglobin subunit alpha. No signs of multiple glycation sites were observed. It also exhibited 95.4% glycated subunit beta, as authenticated.

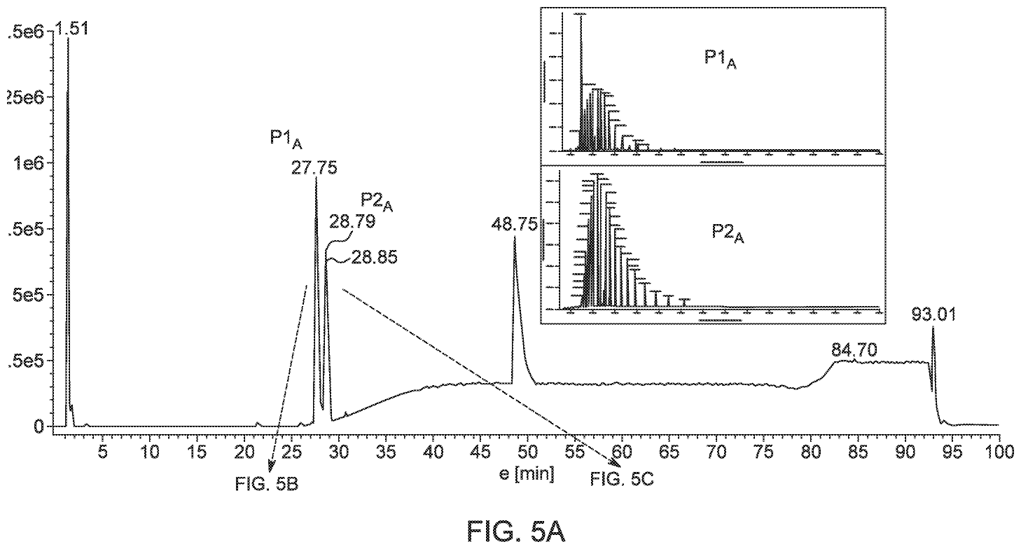

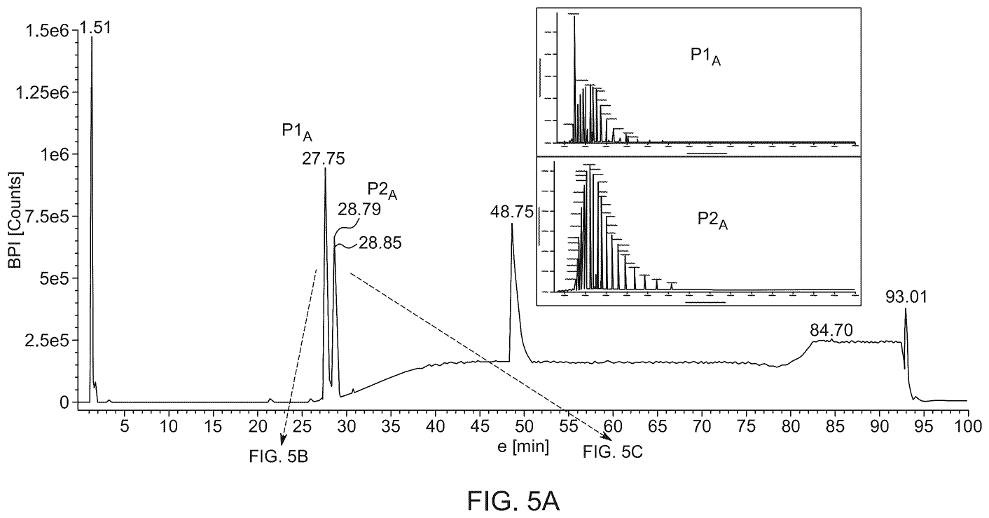

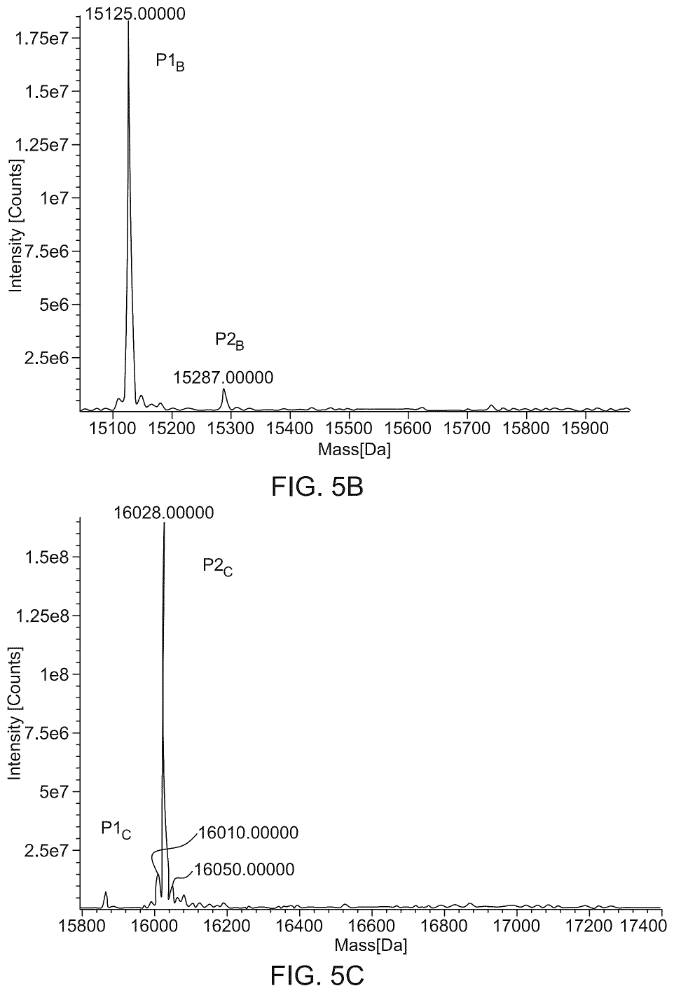

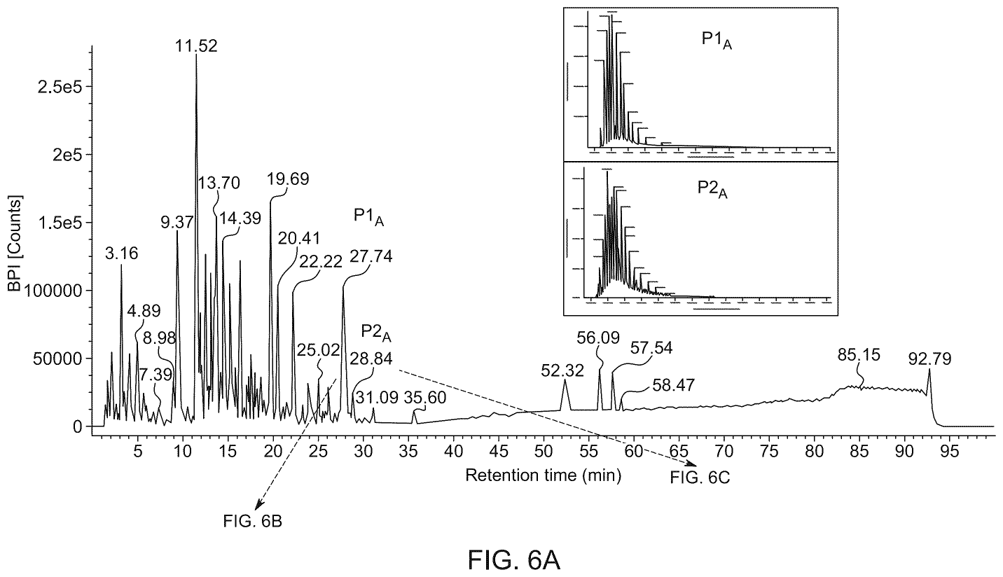

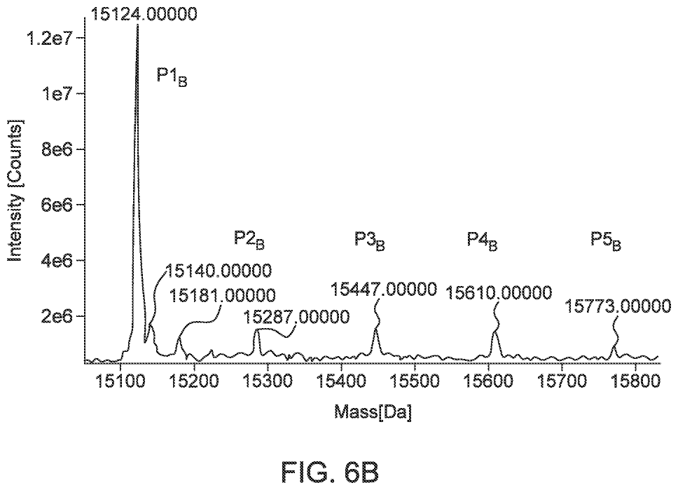

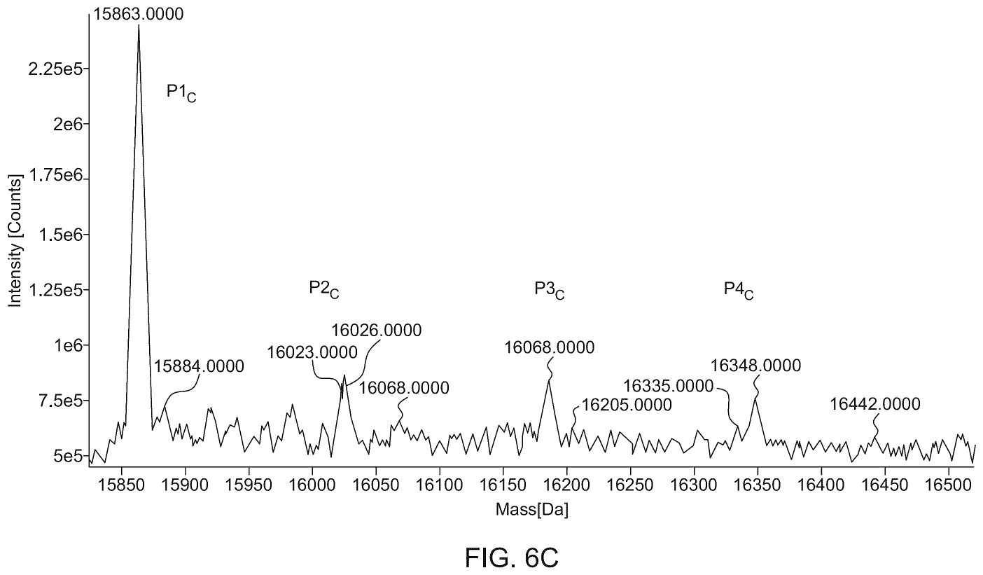

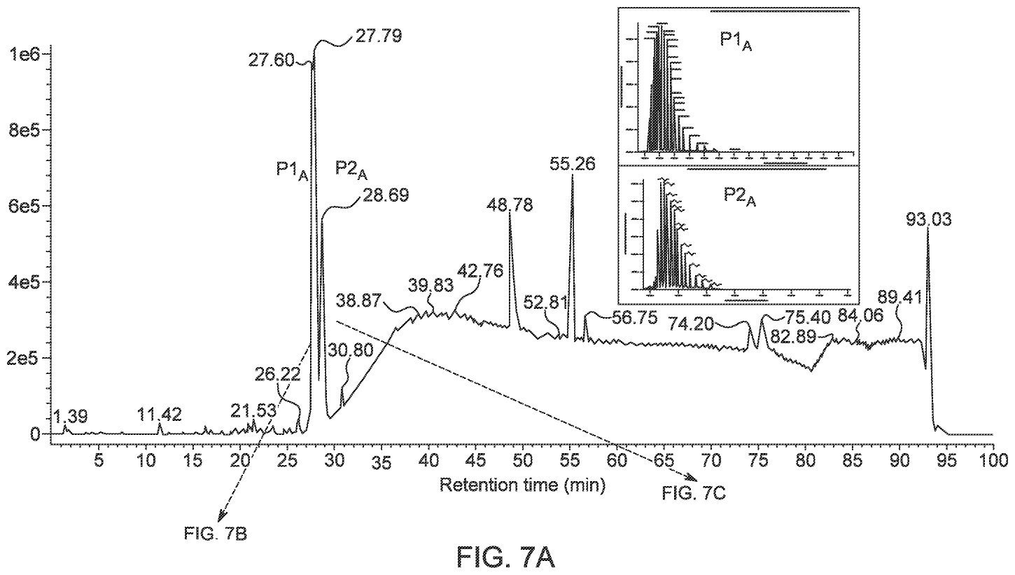

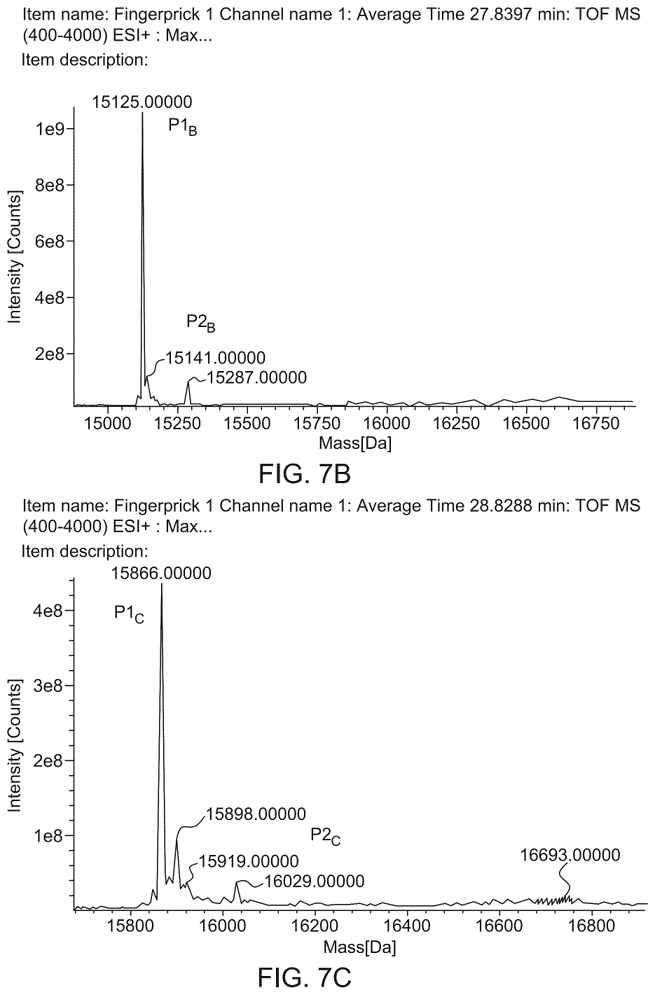

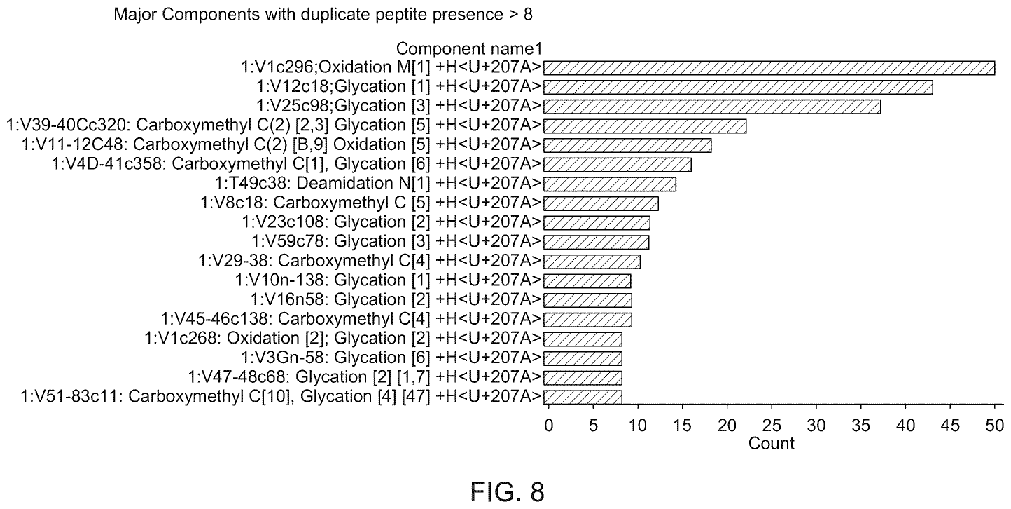

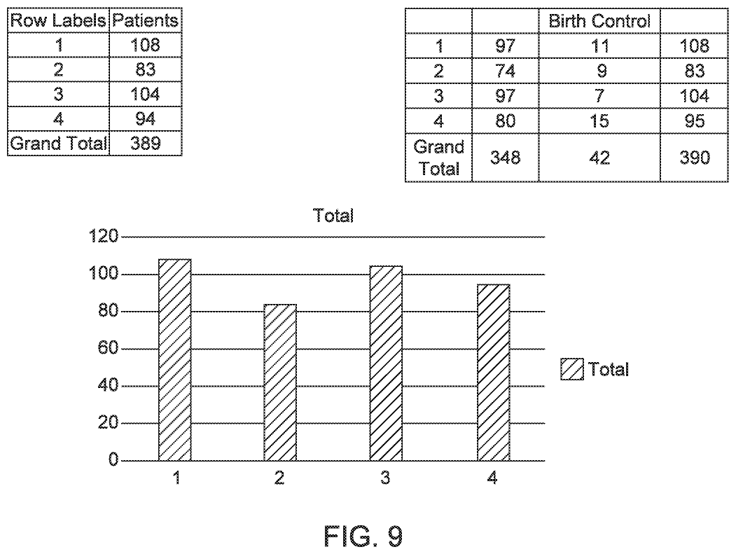

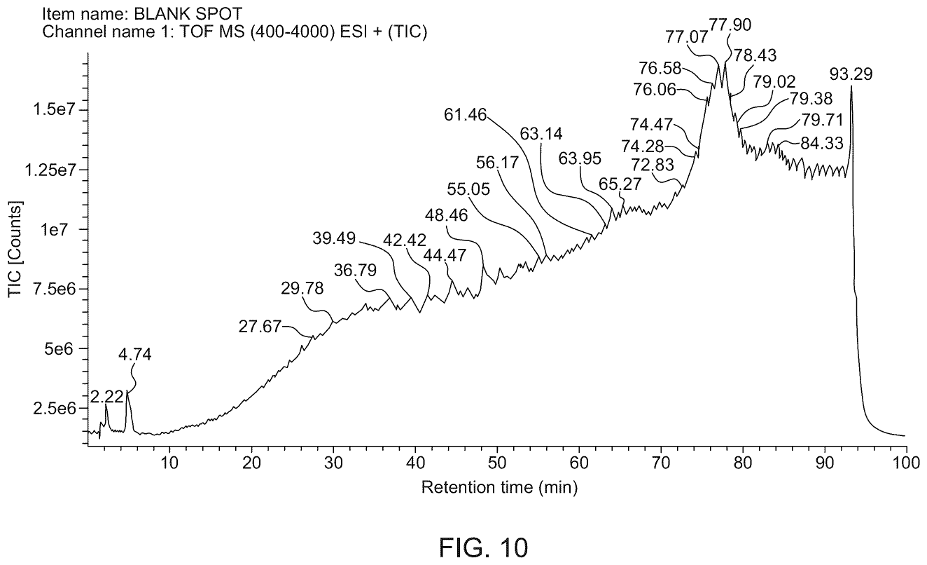

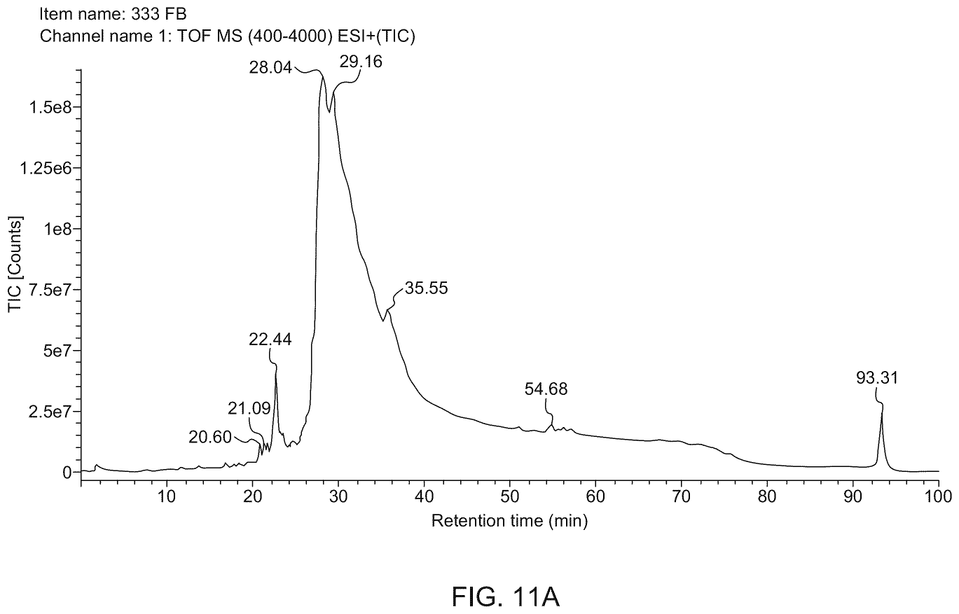

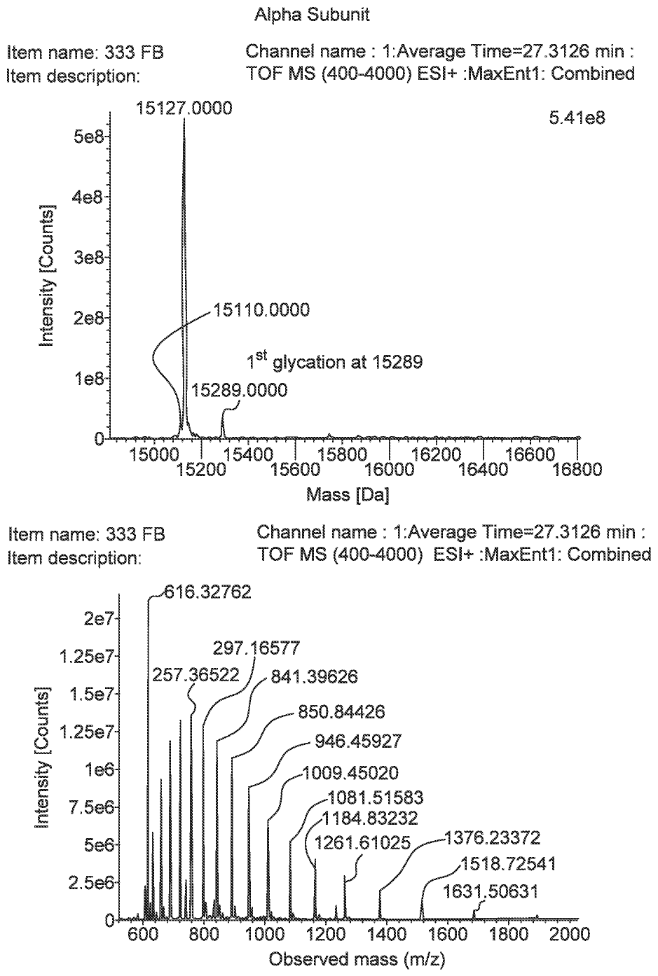

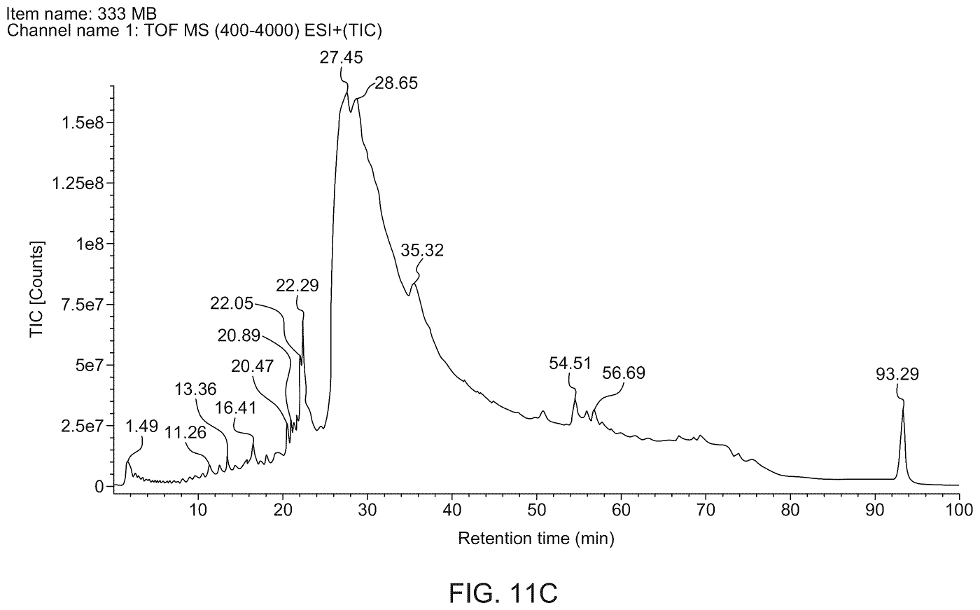

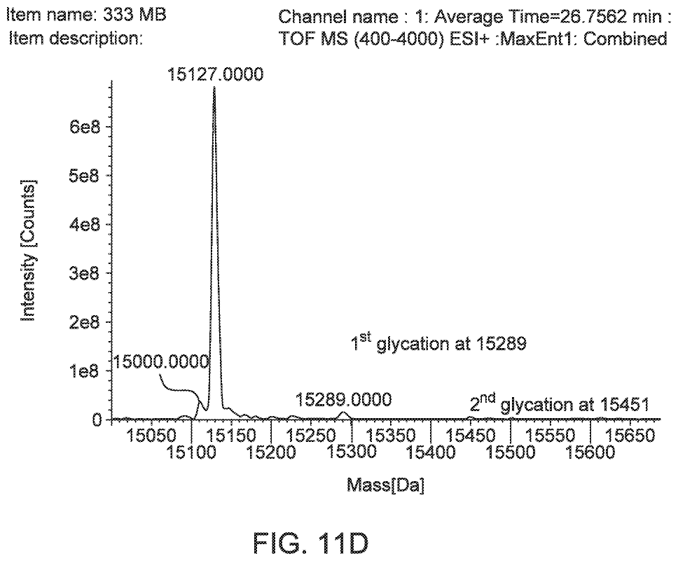

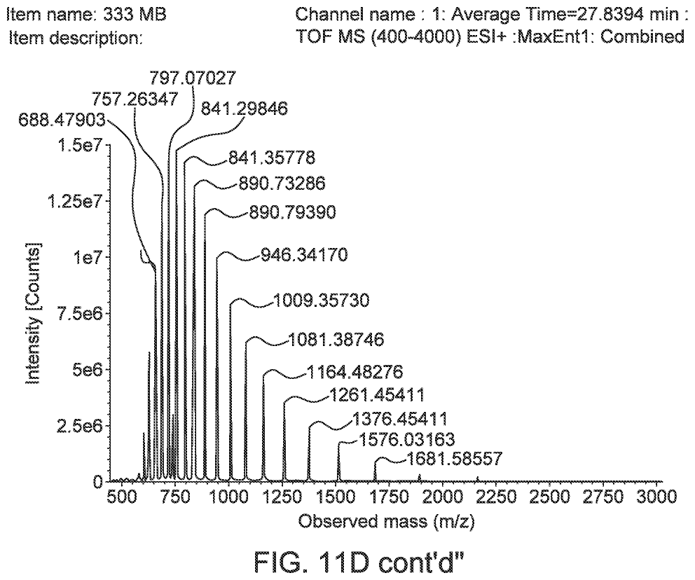

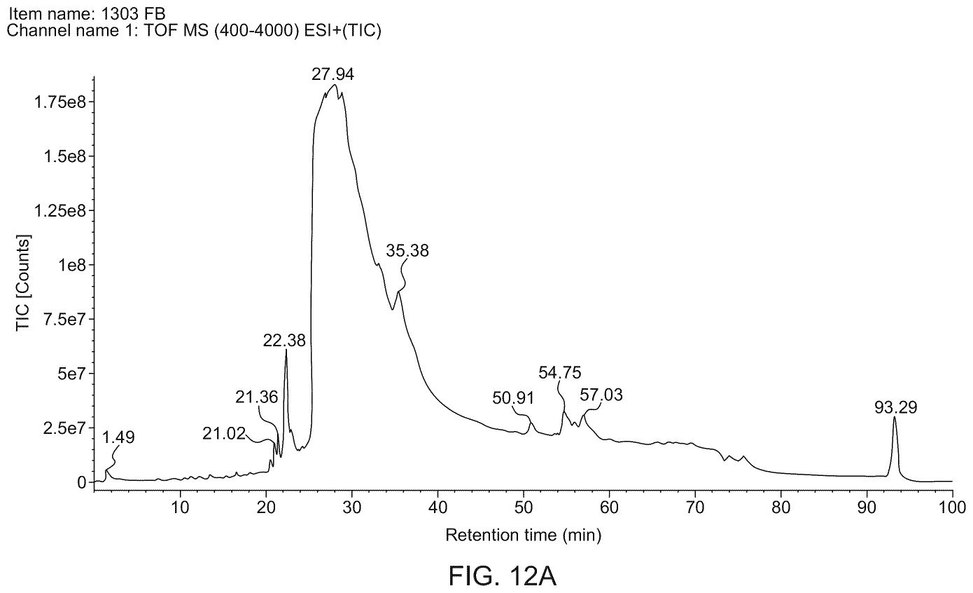

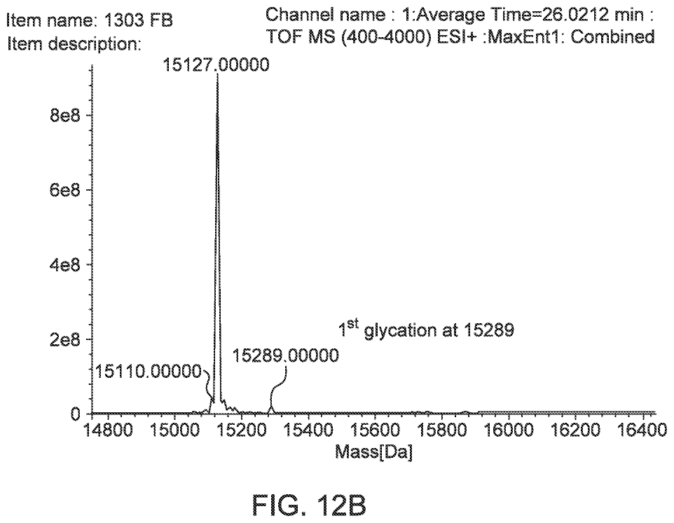

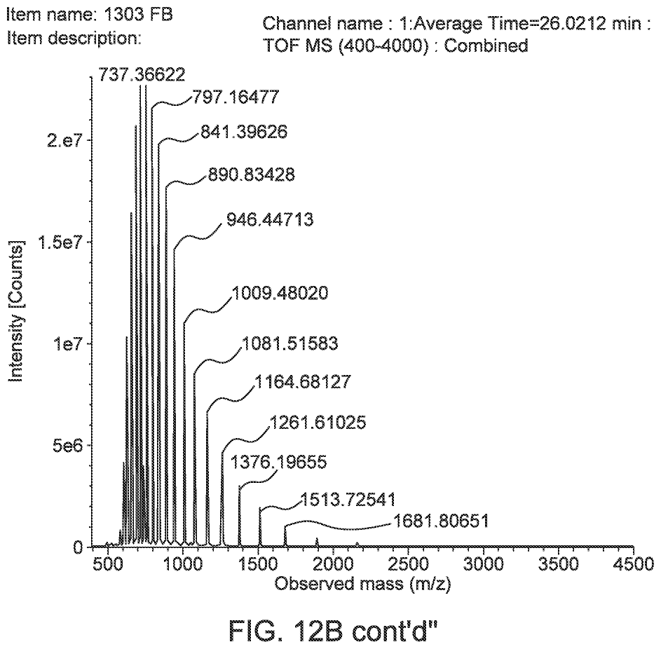

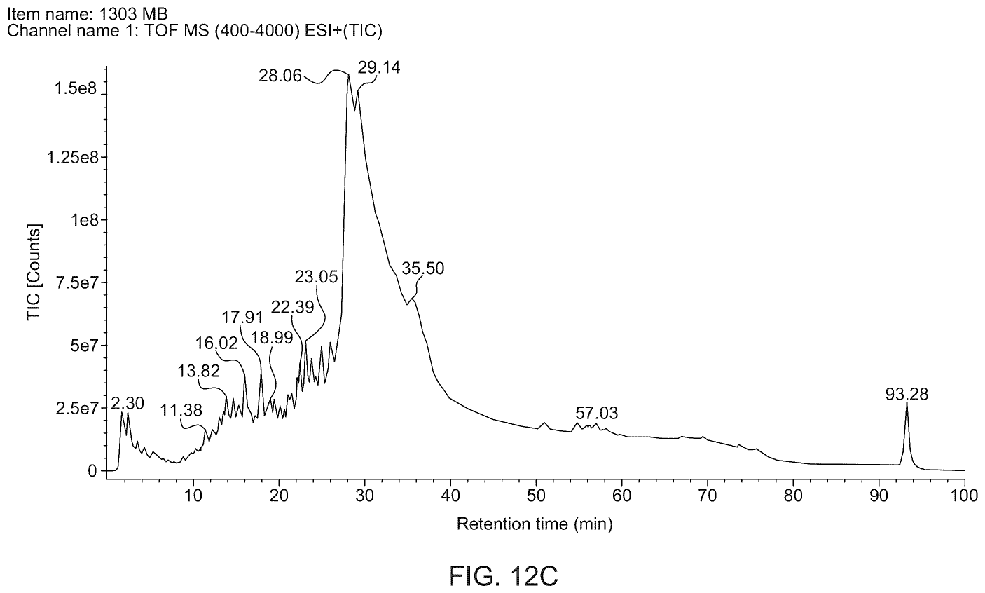

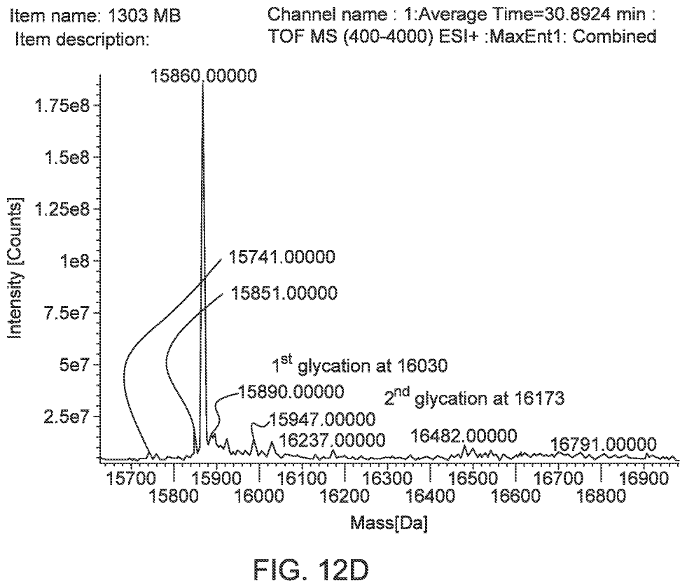

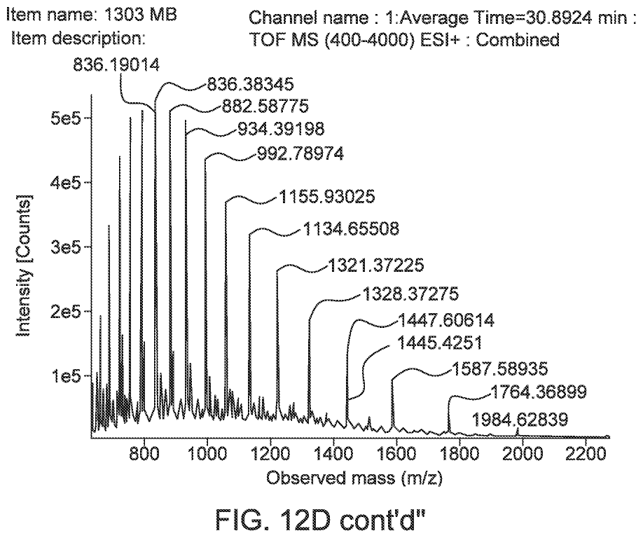

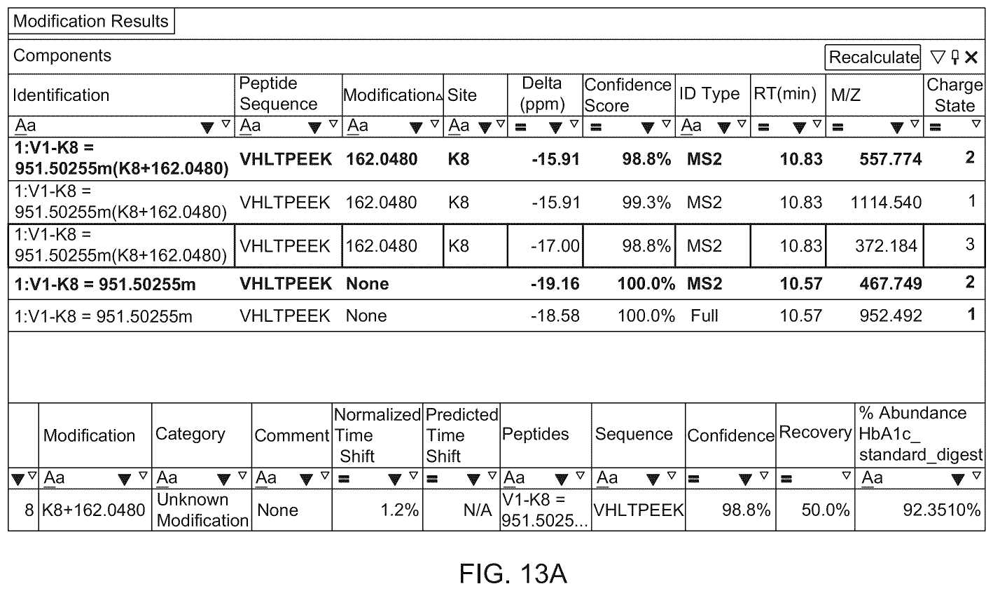

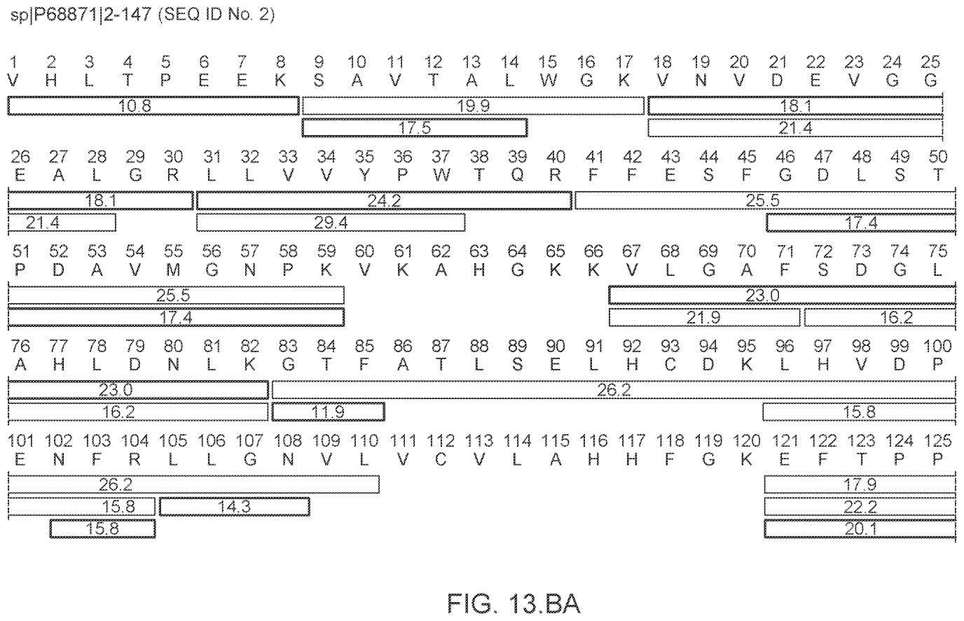

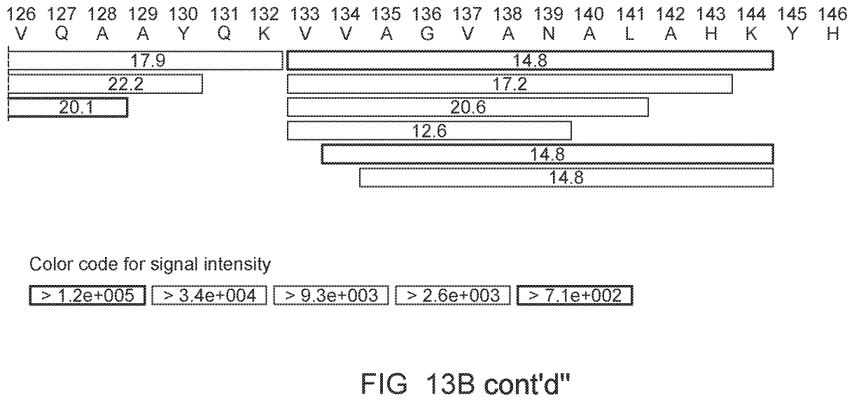

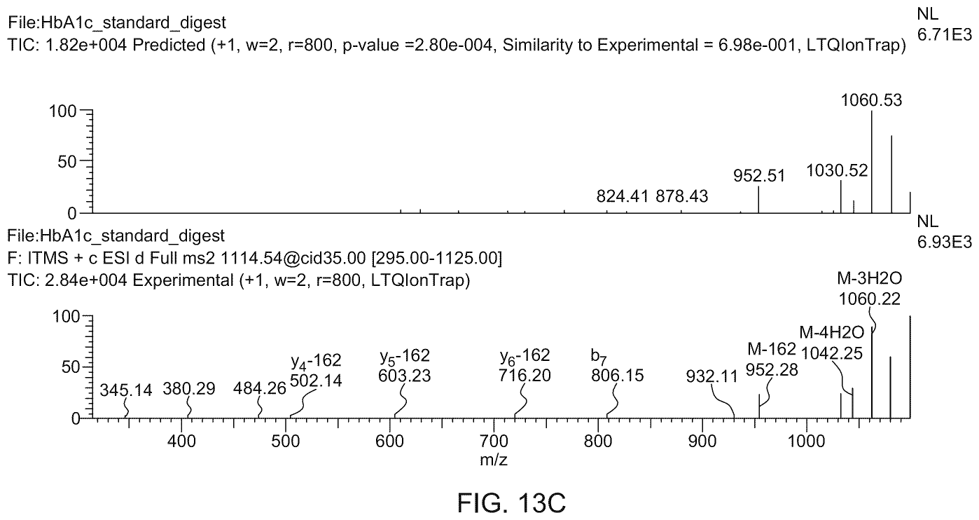

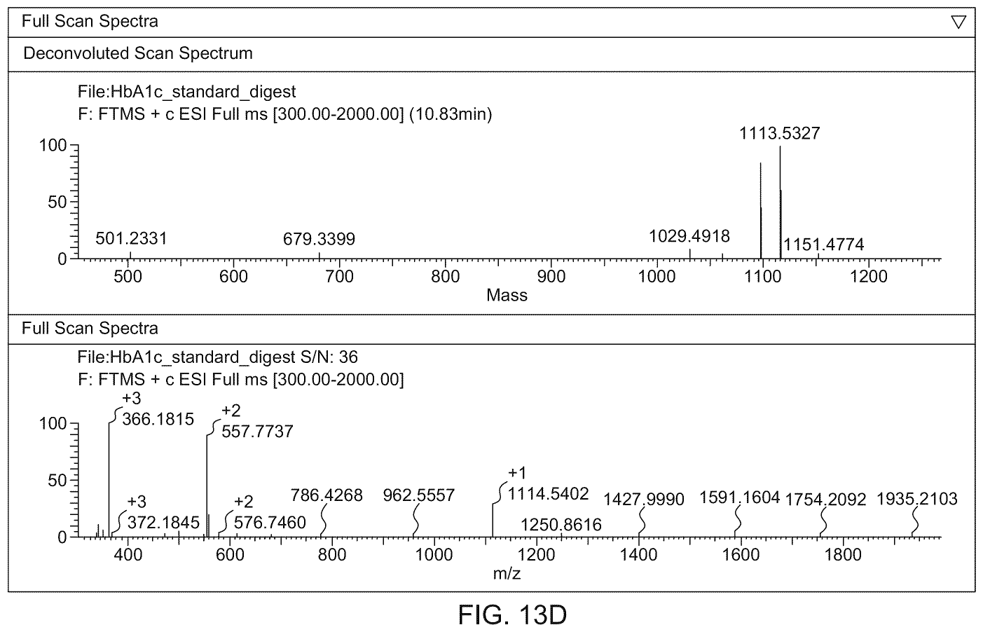

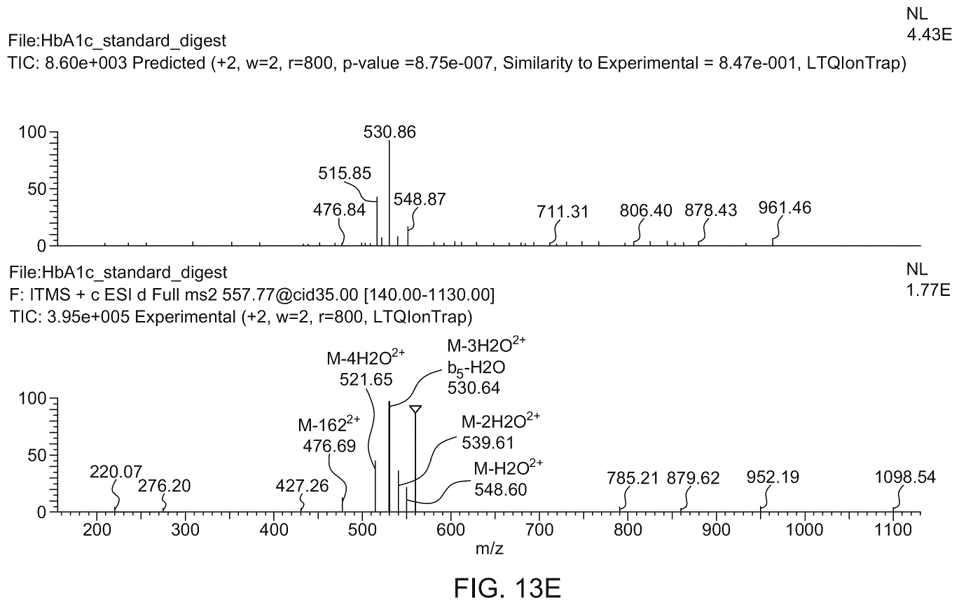

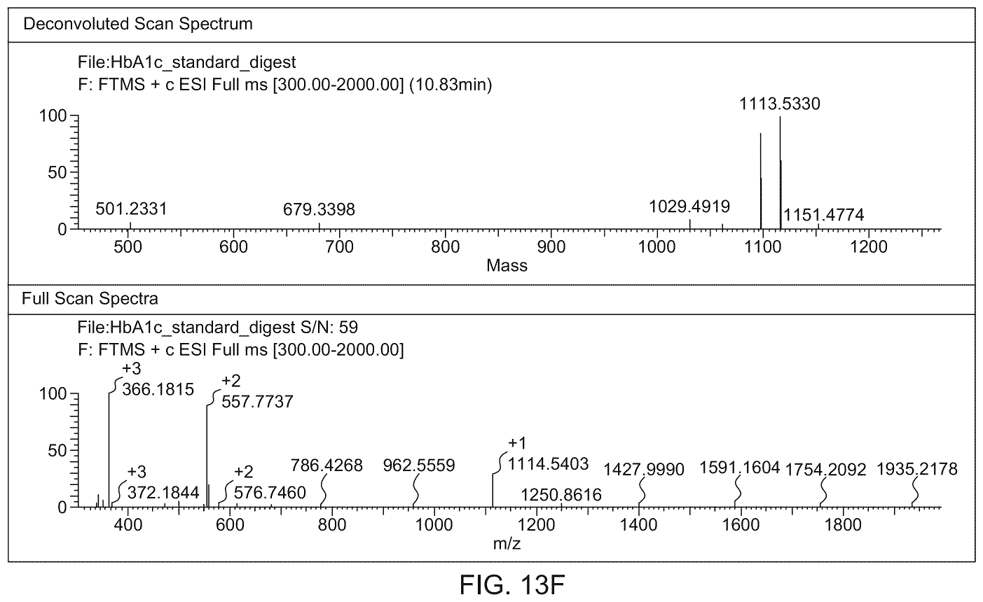

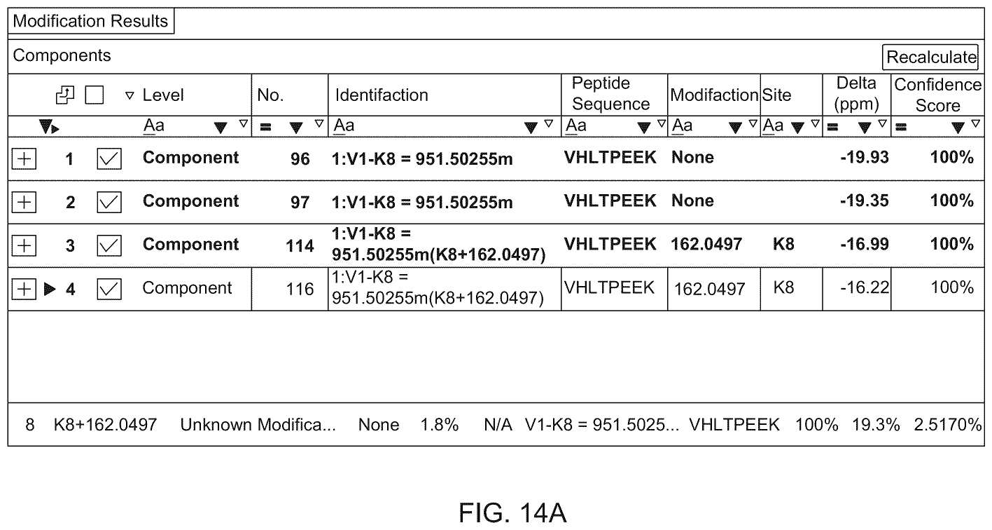

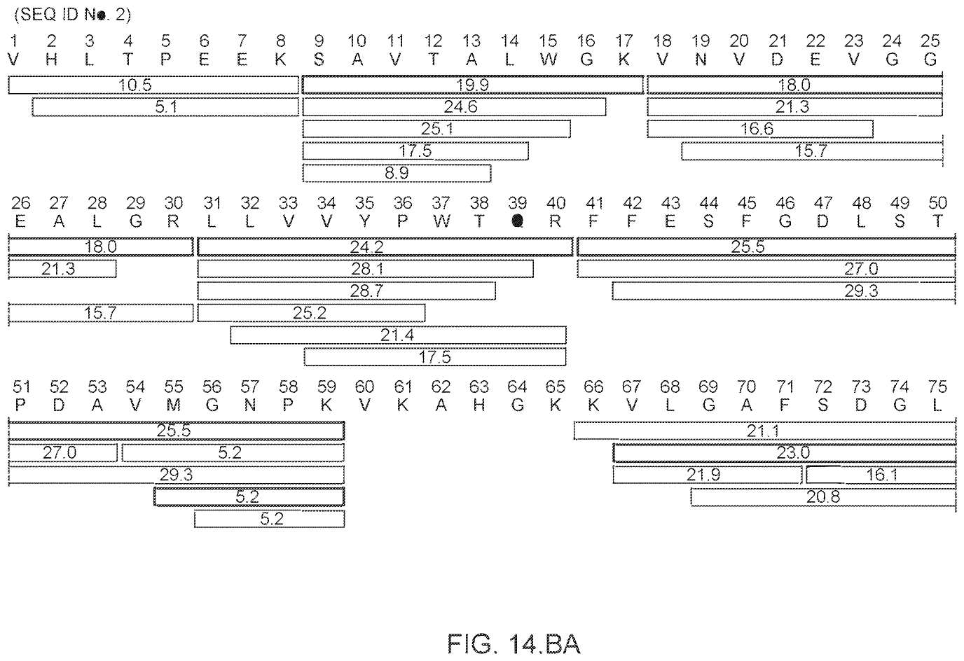

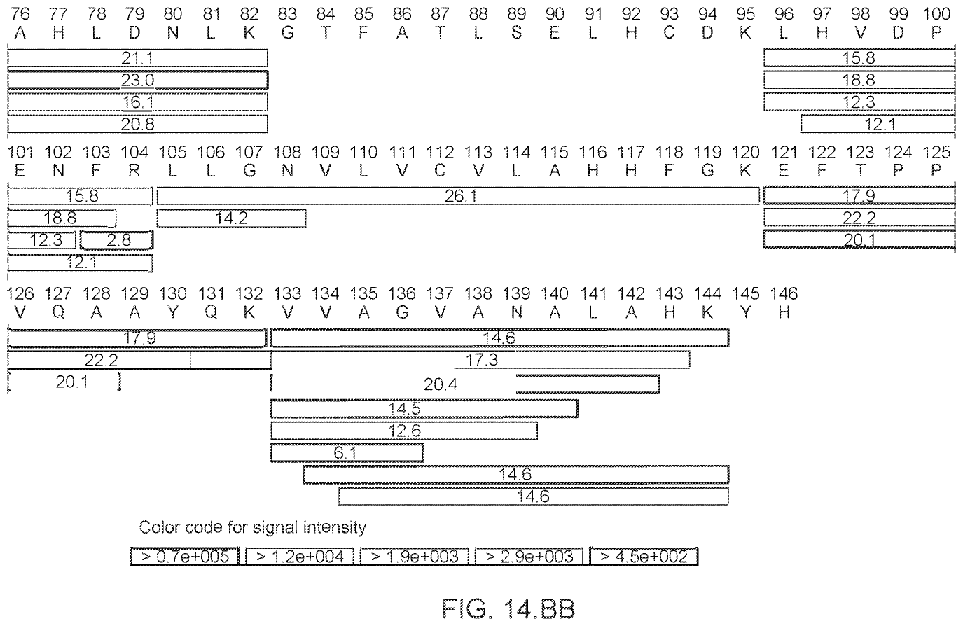

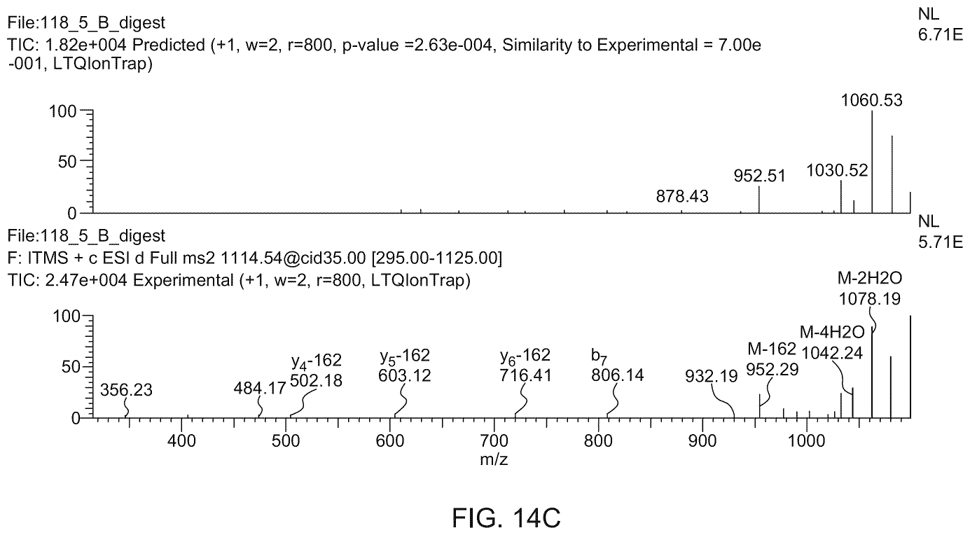

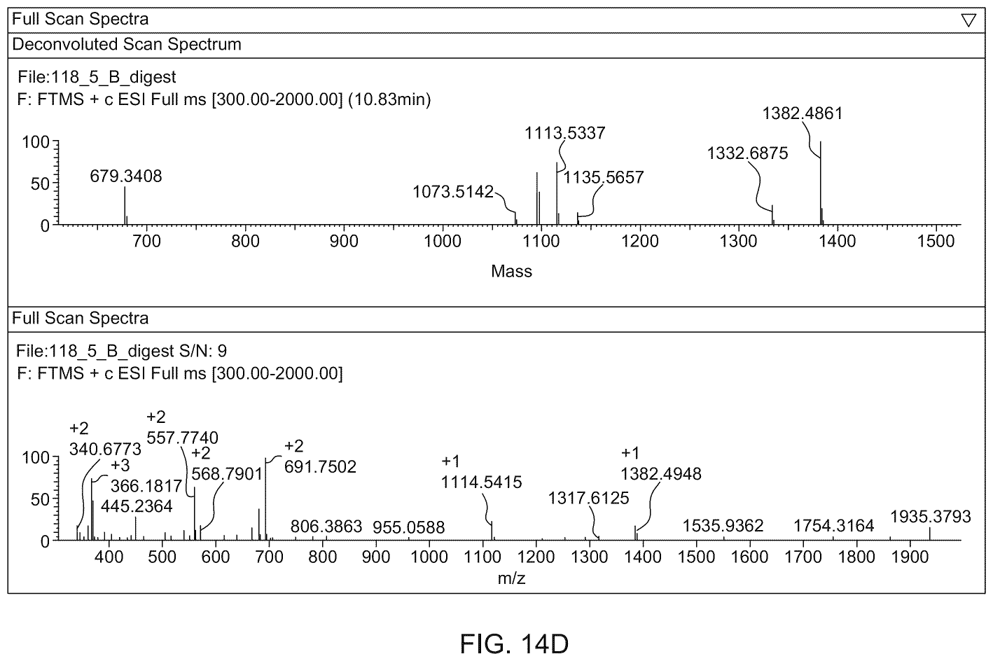

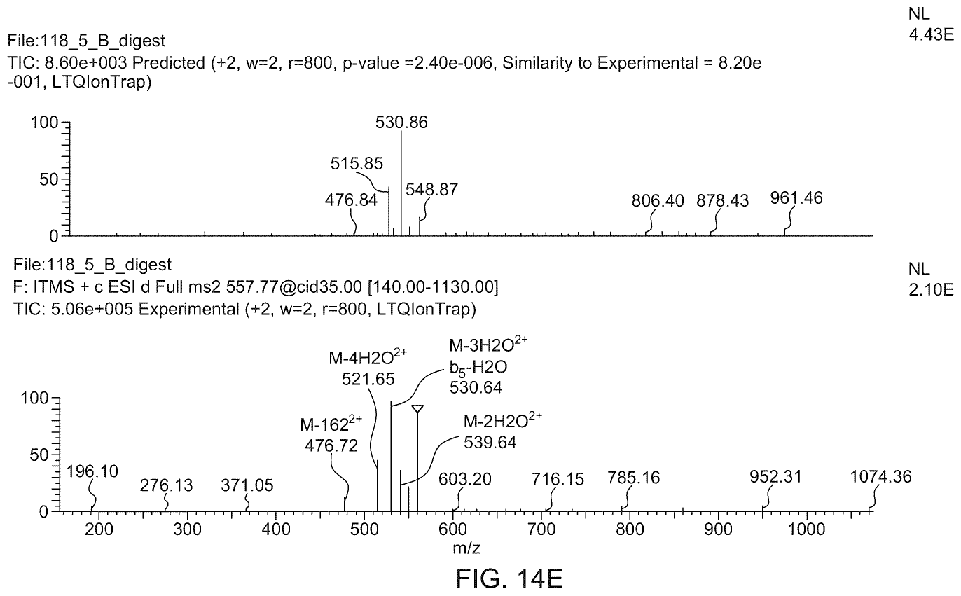

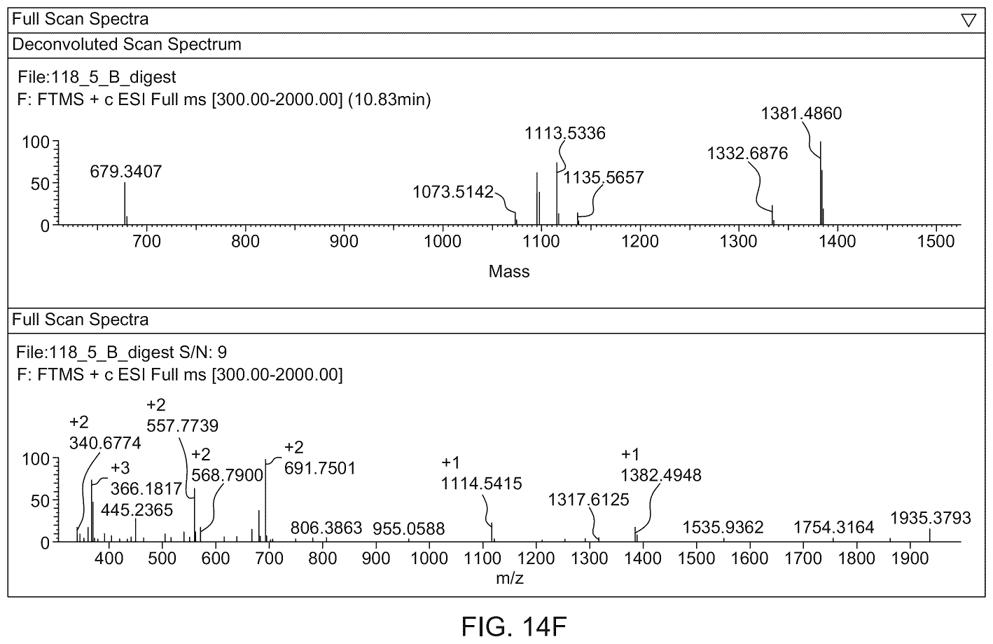

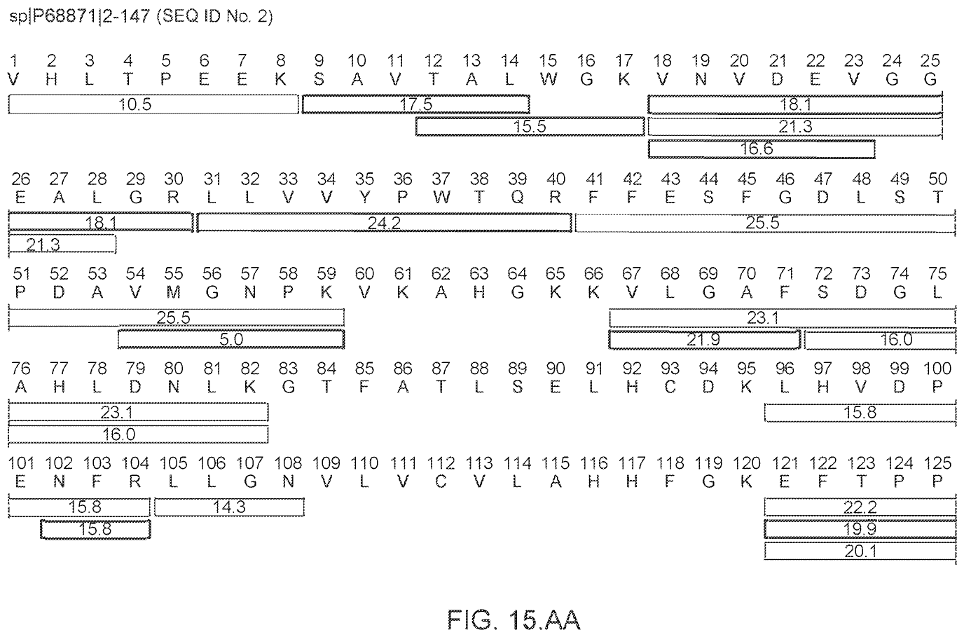

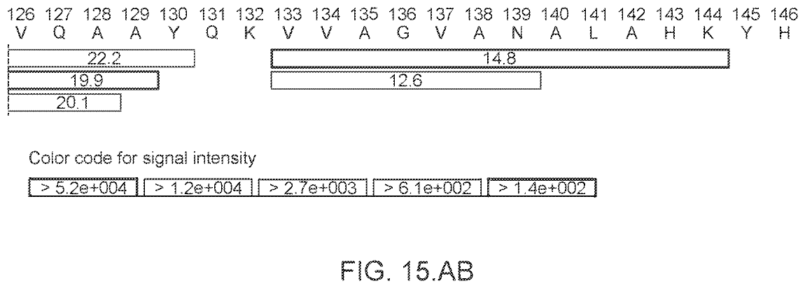

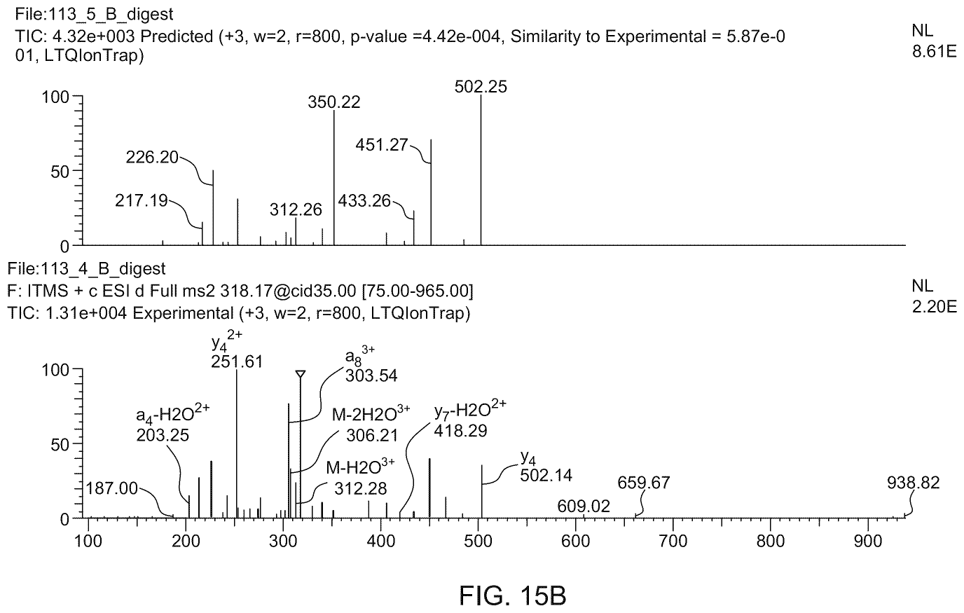

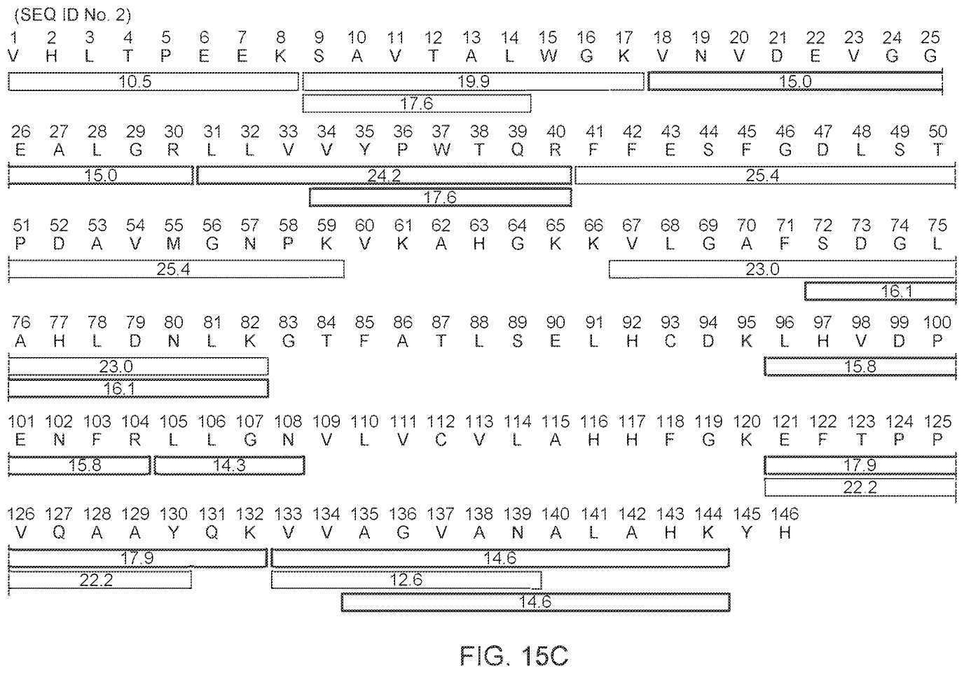

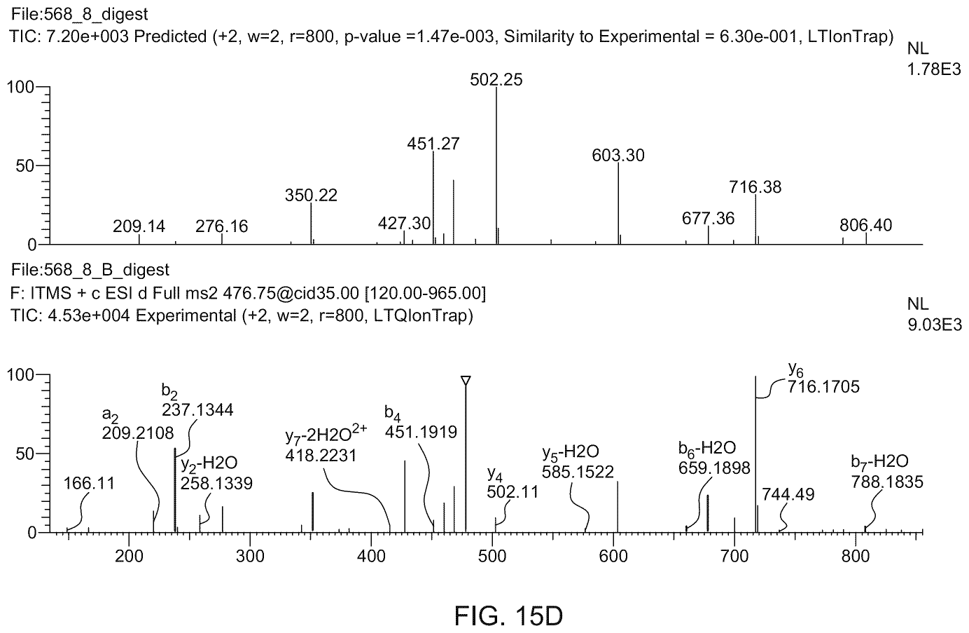

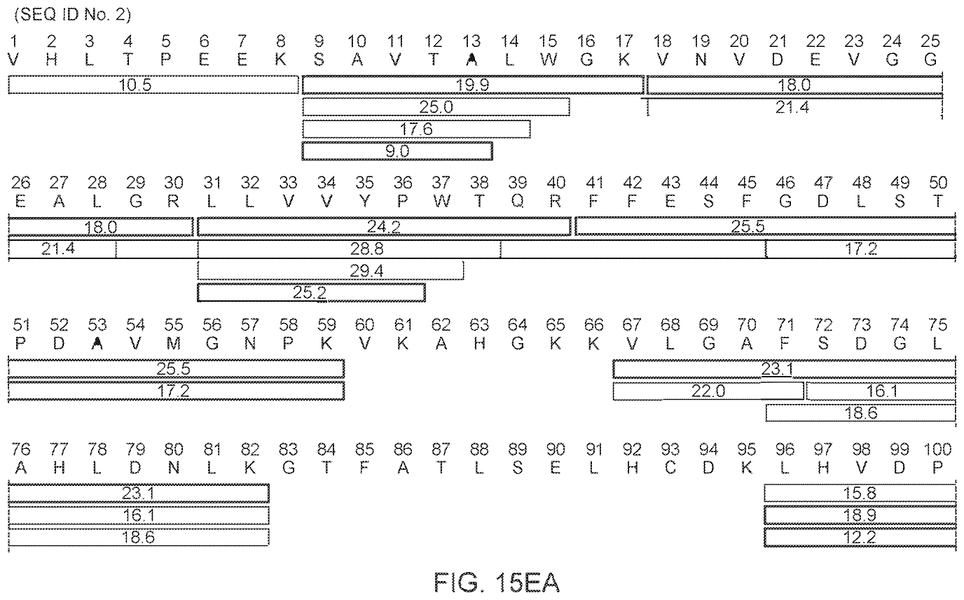

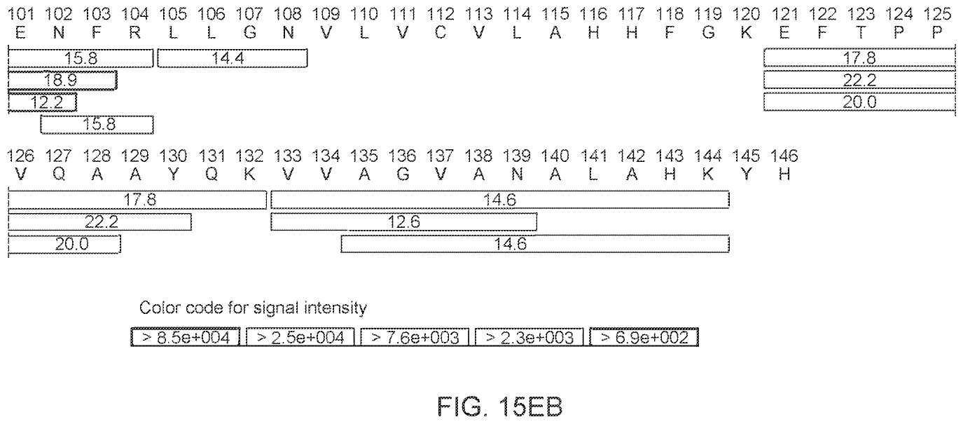

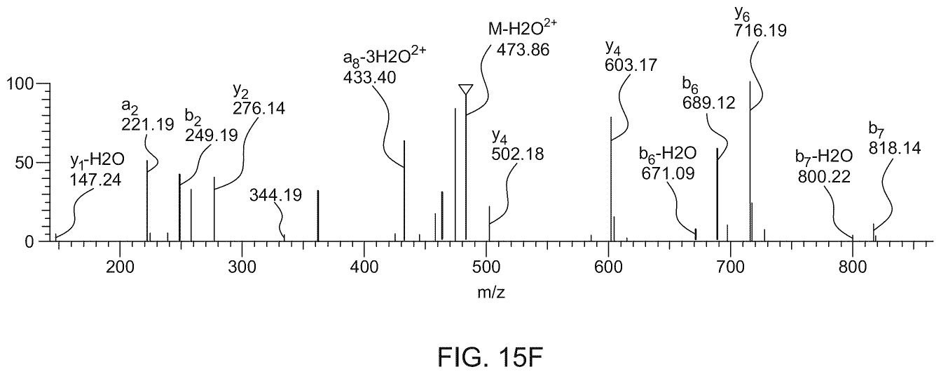

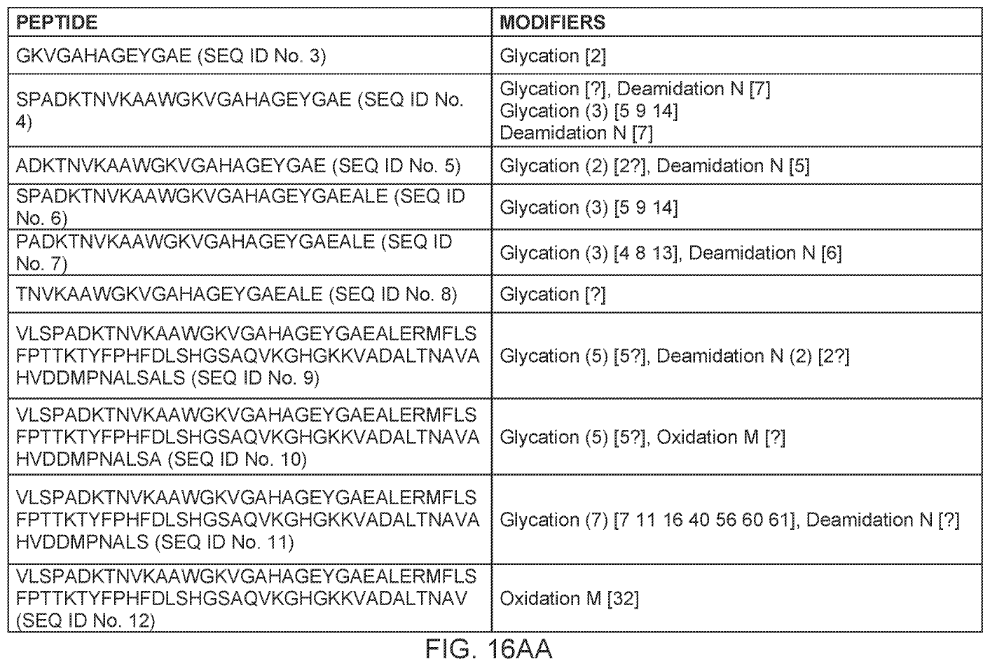

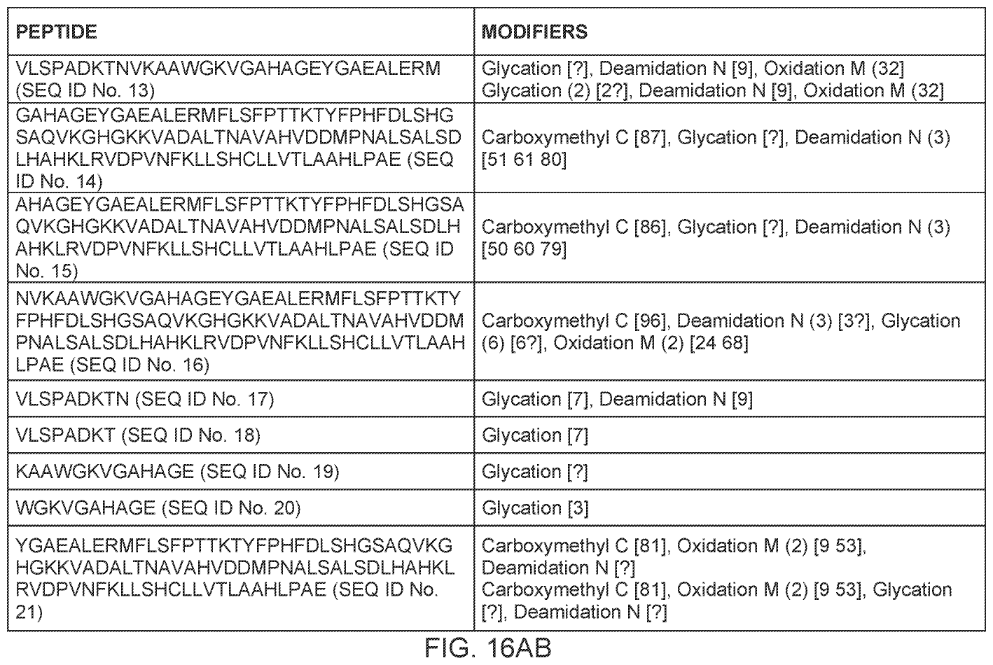

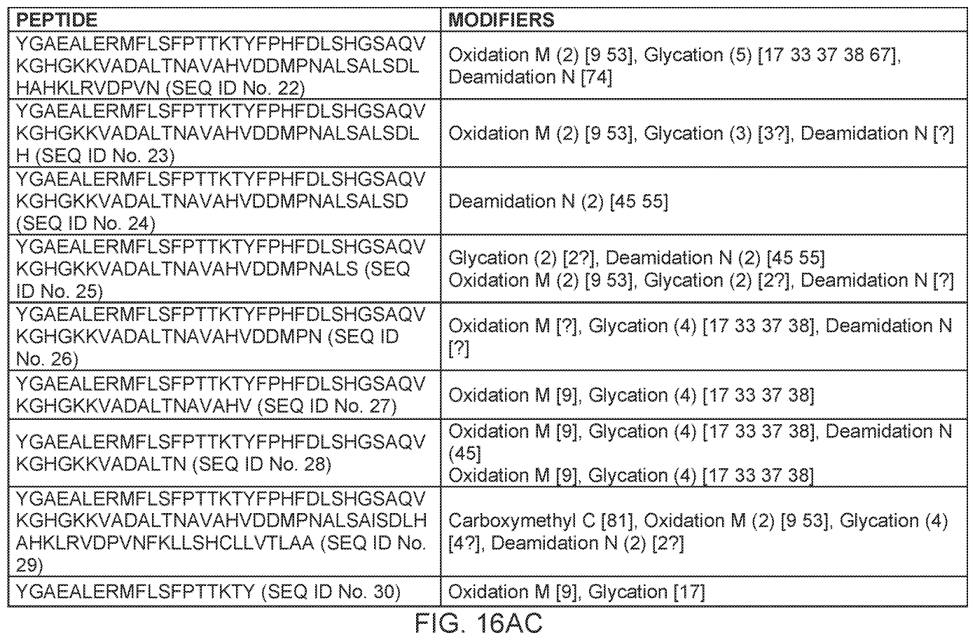

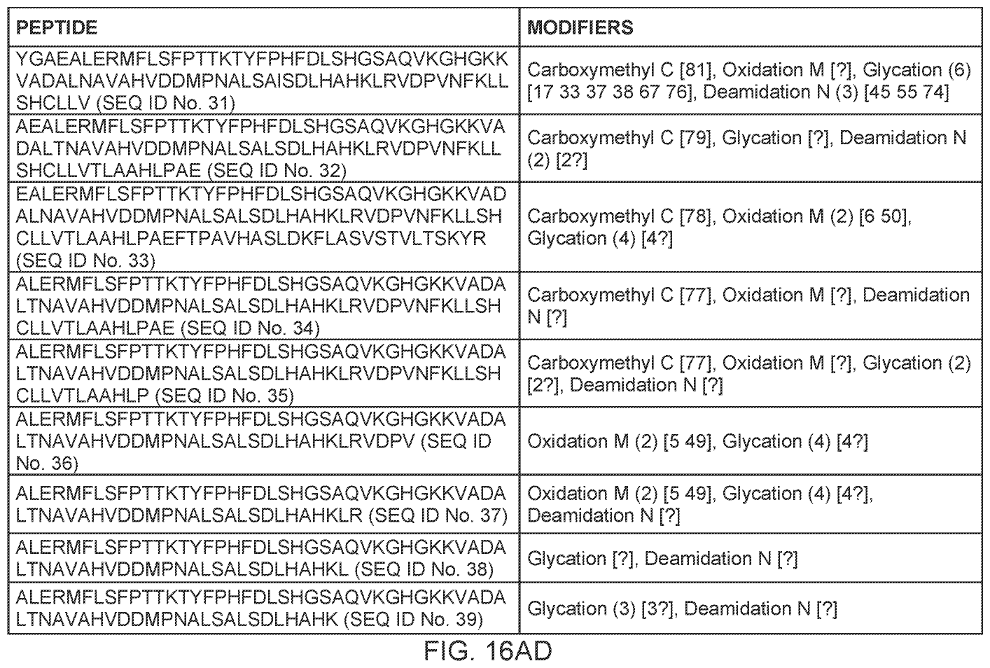

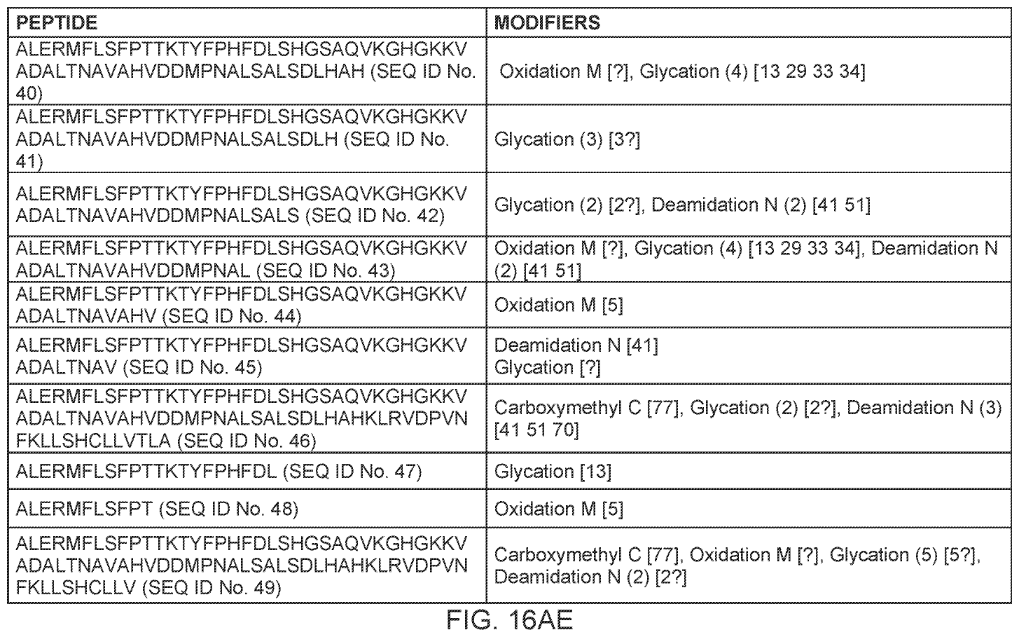

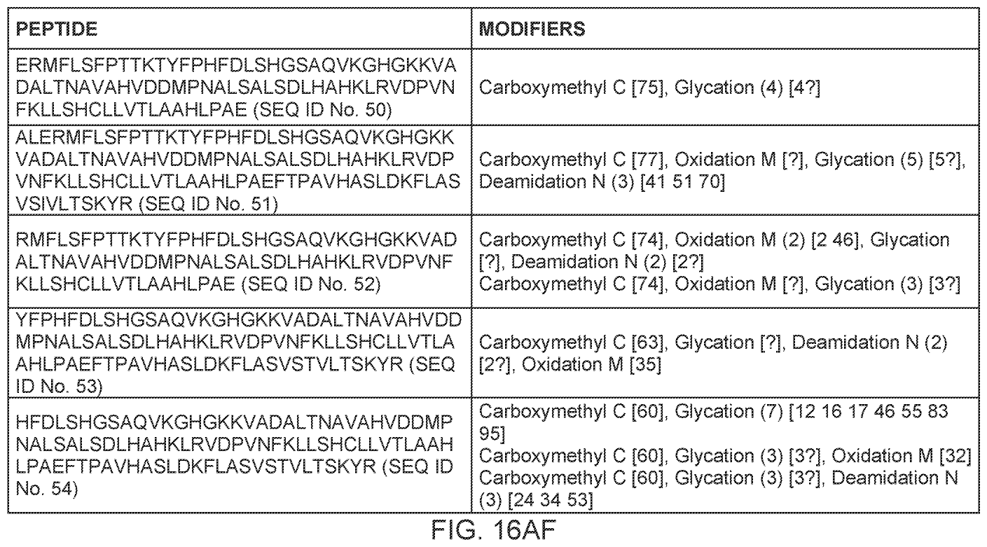

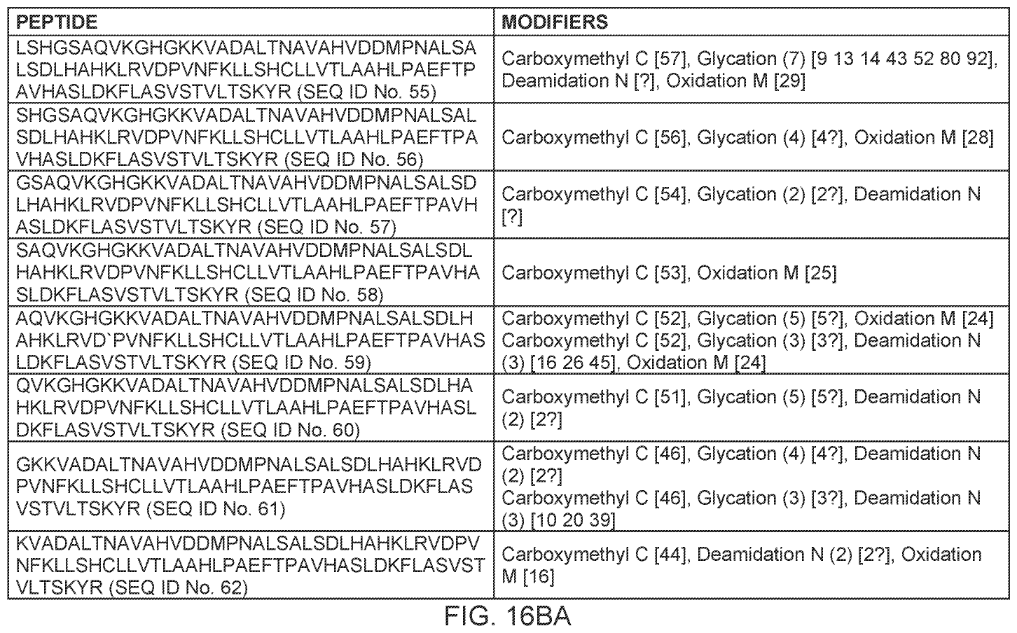

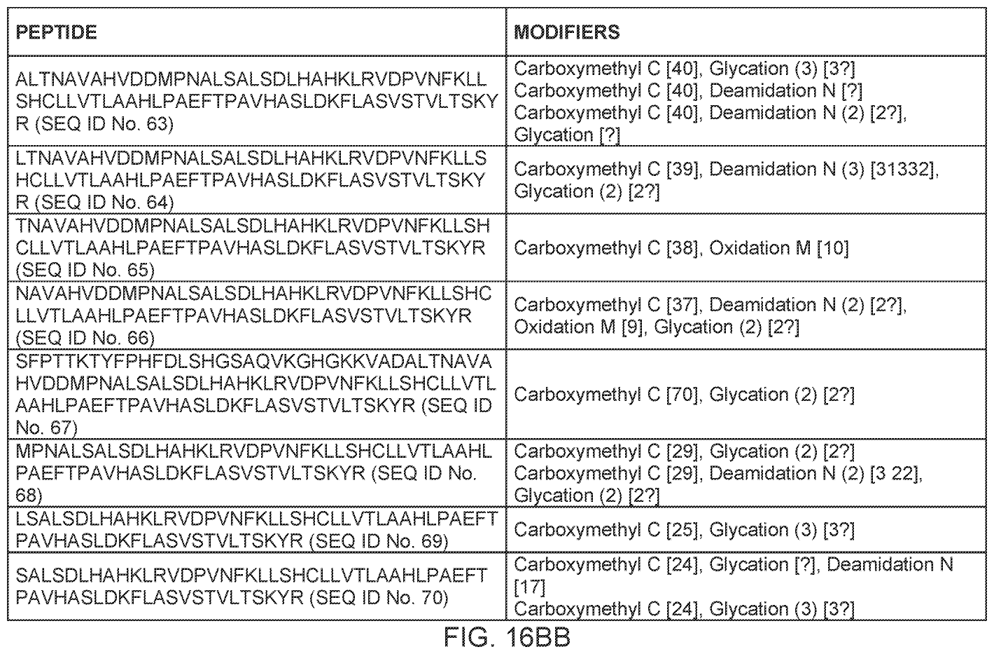

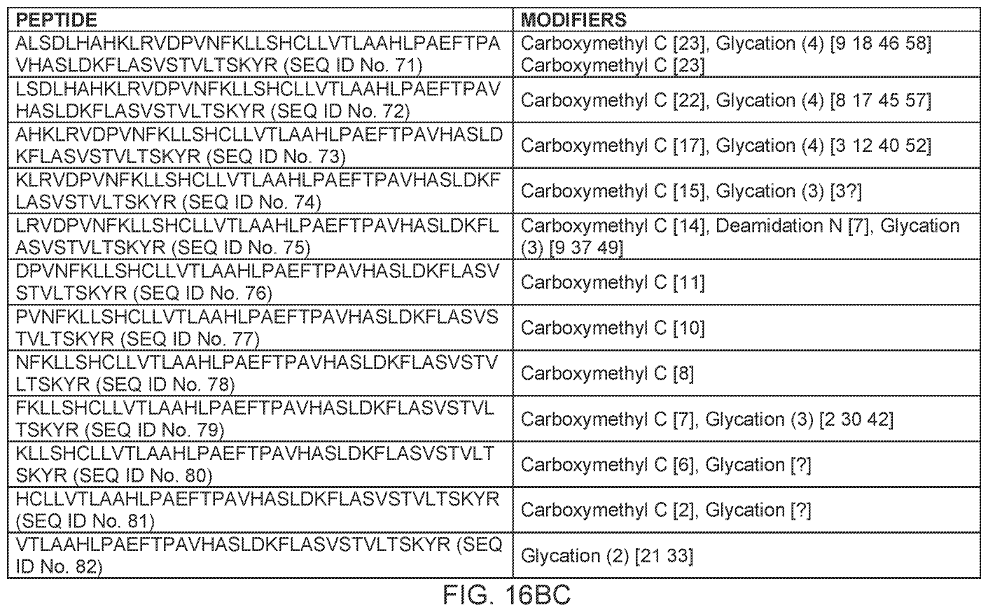

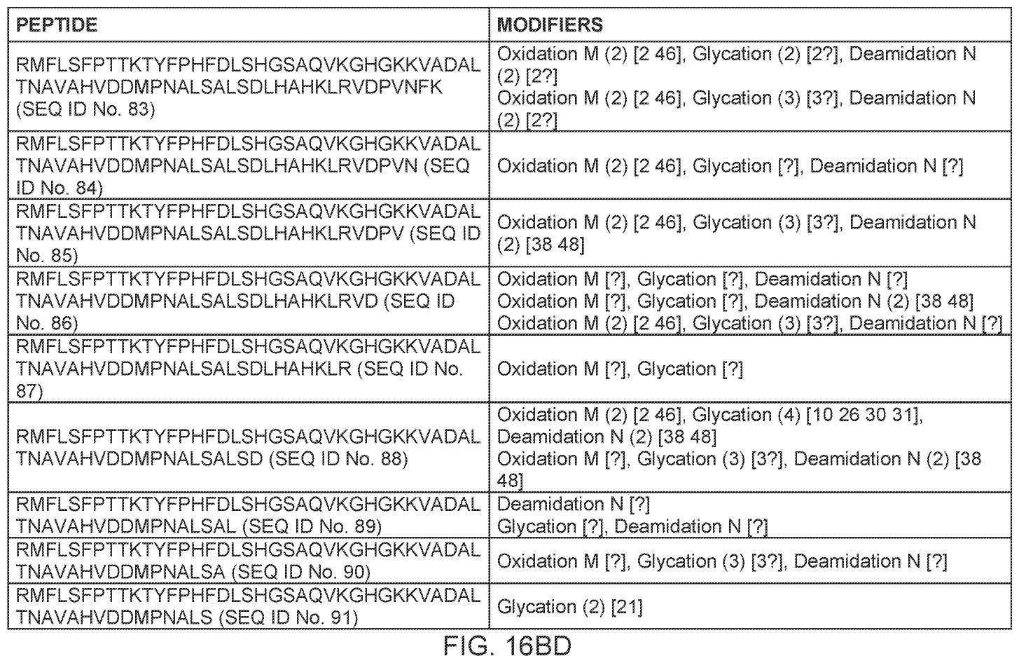

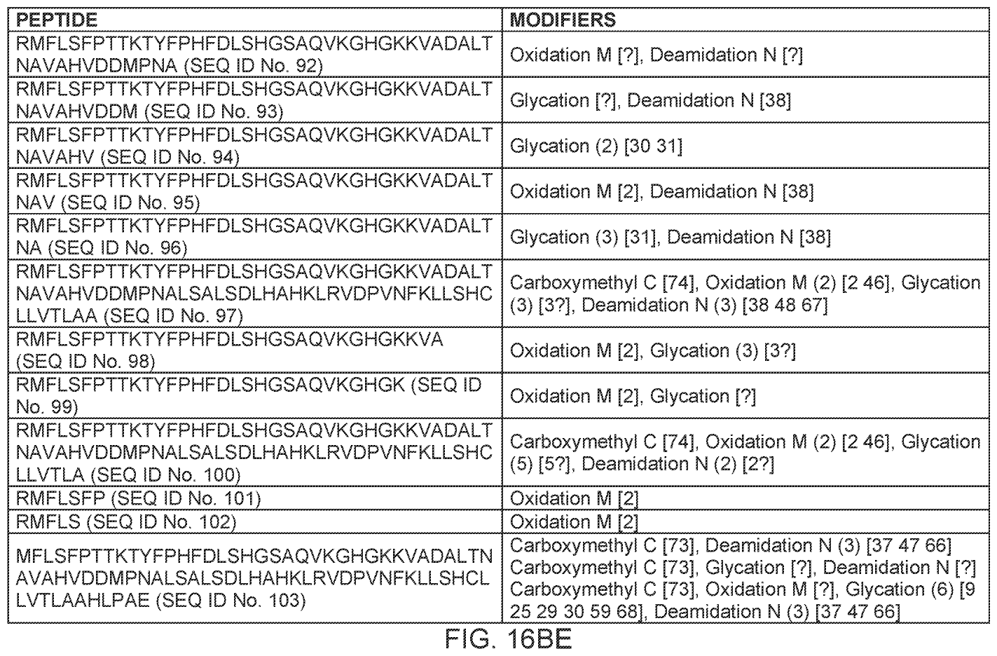

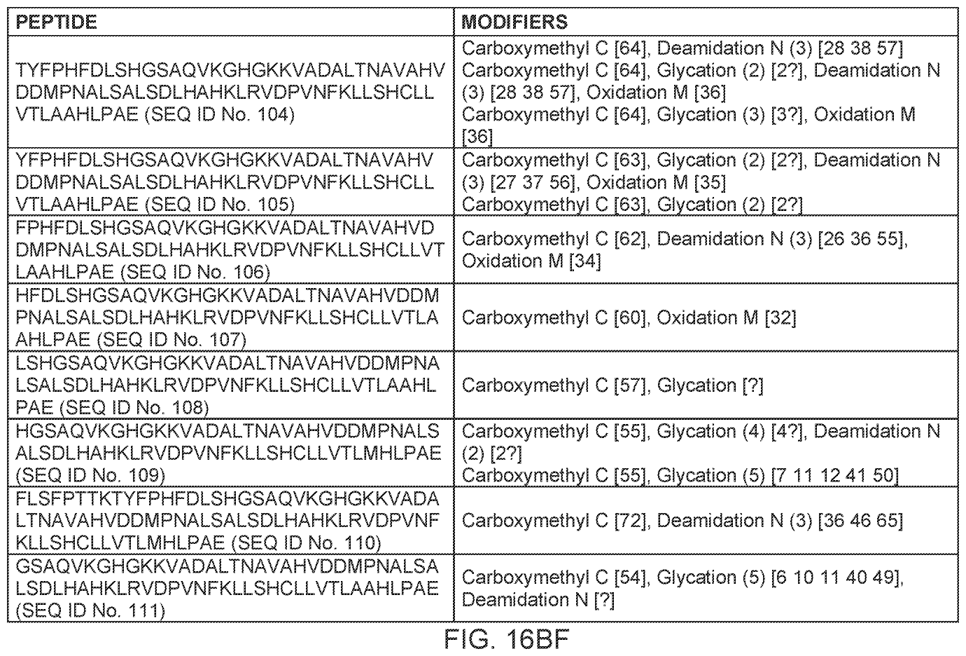

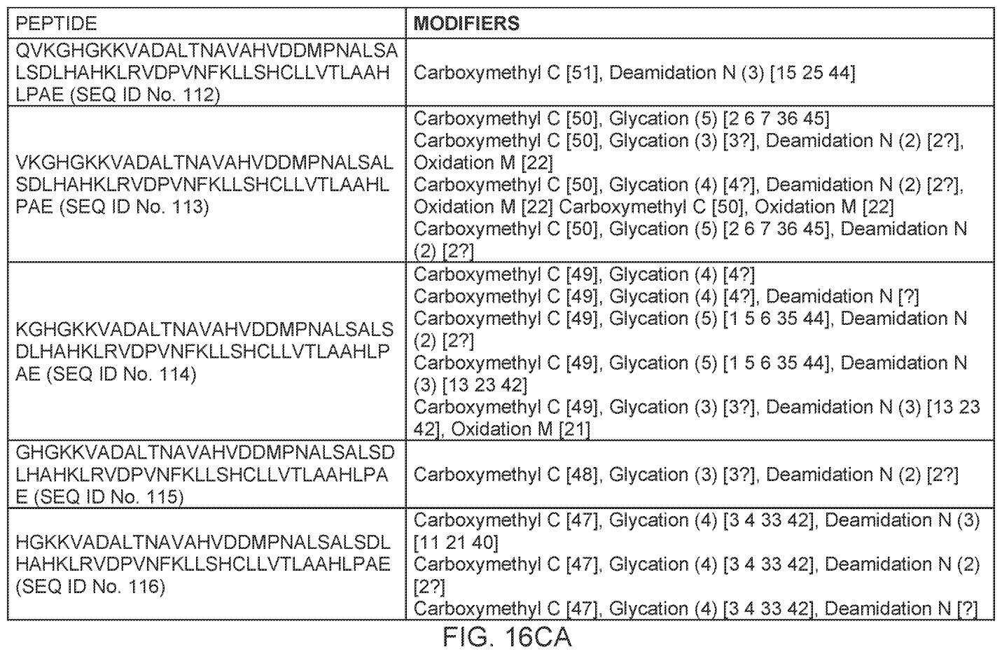

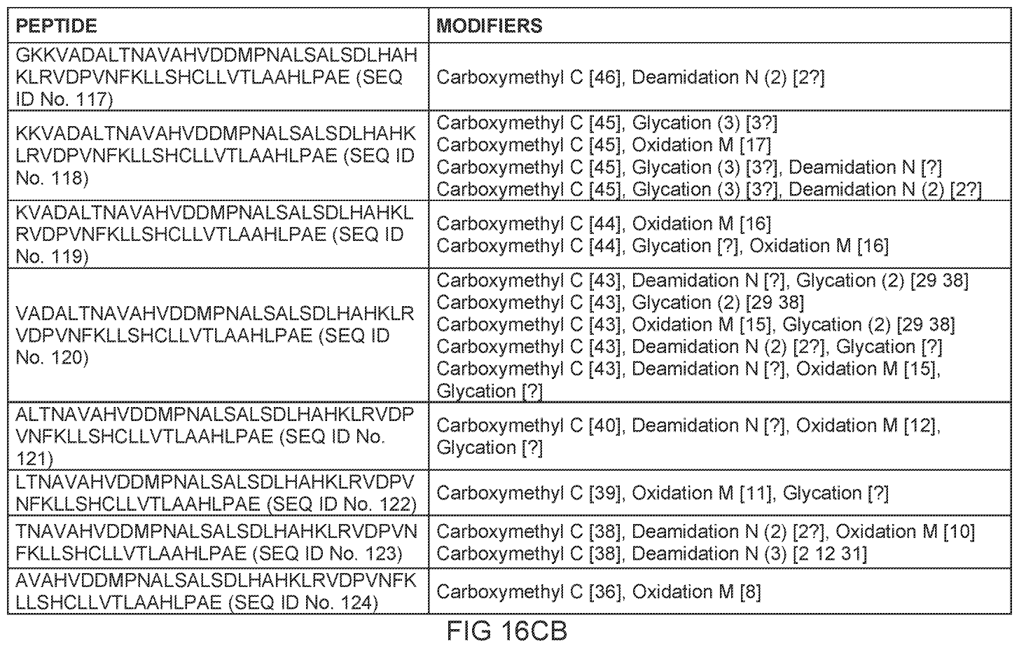

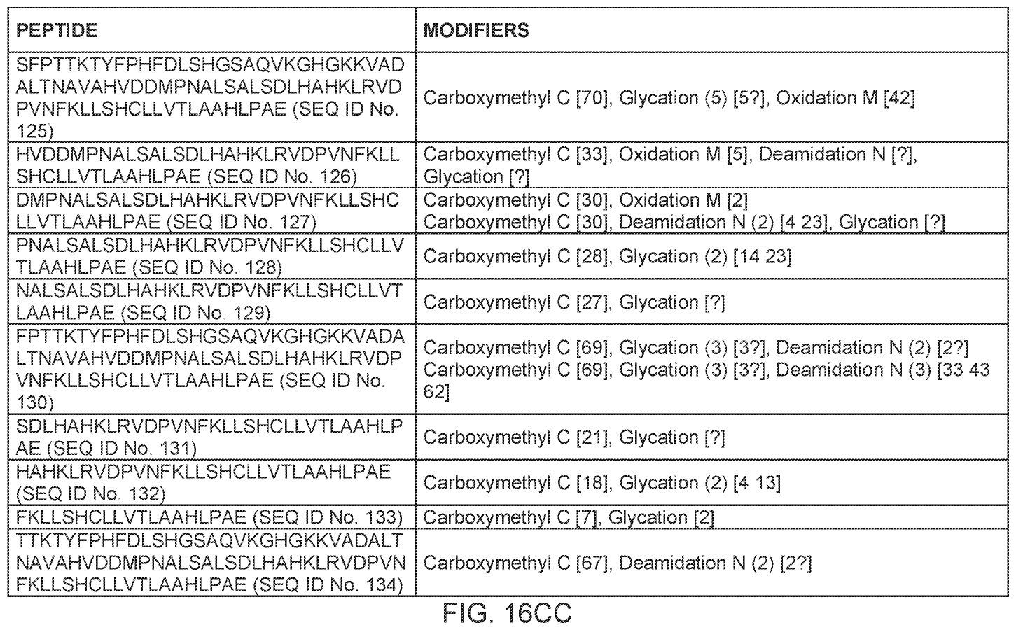

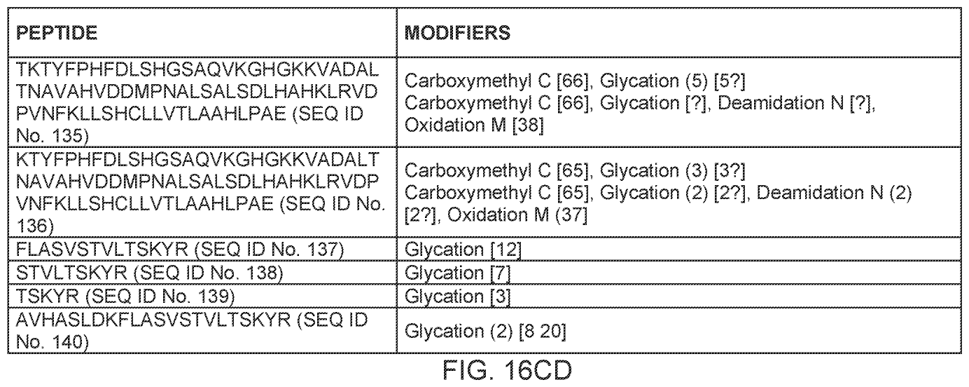

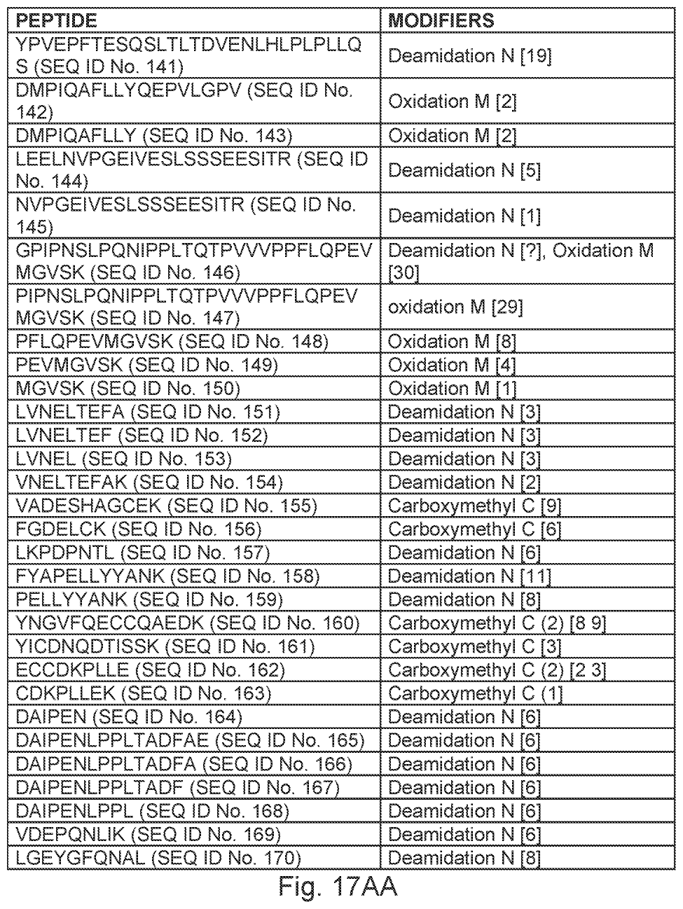

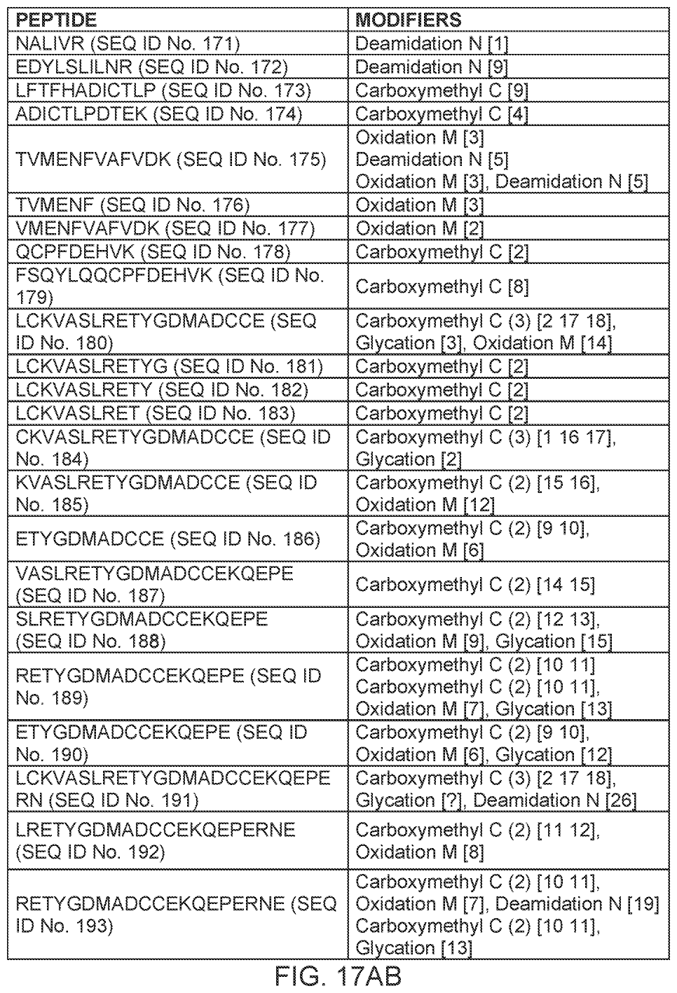

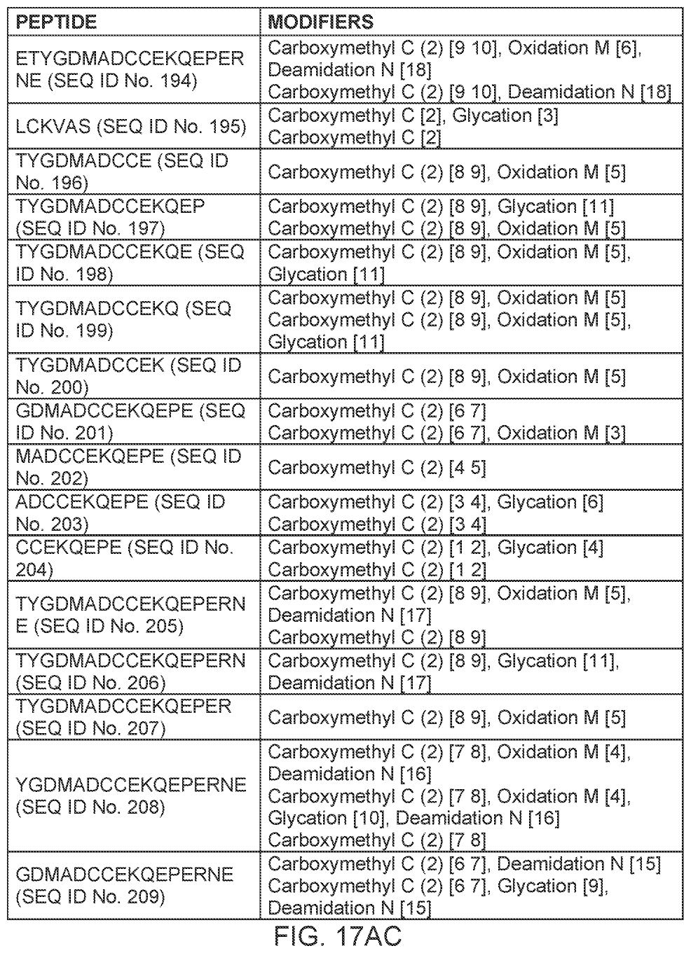

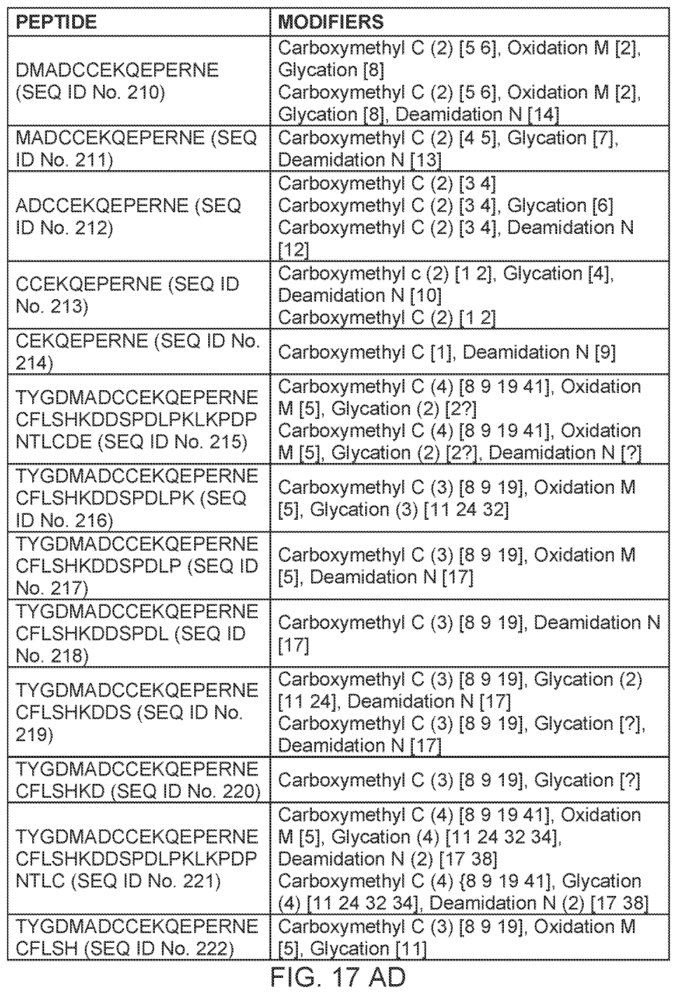

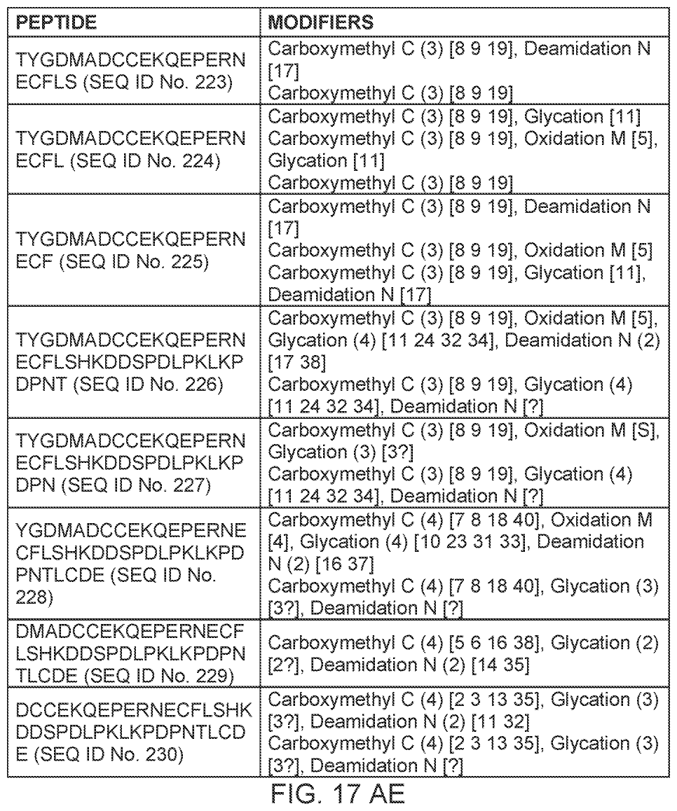

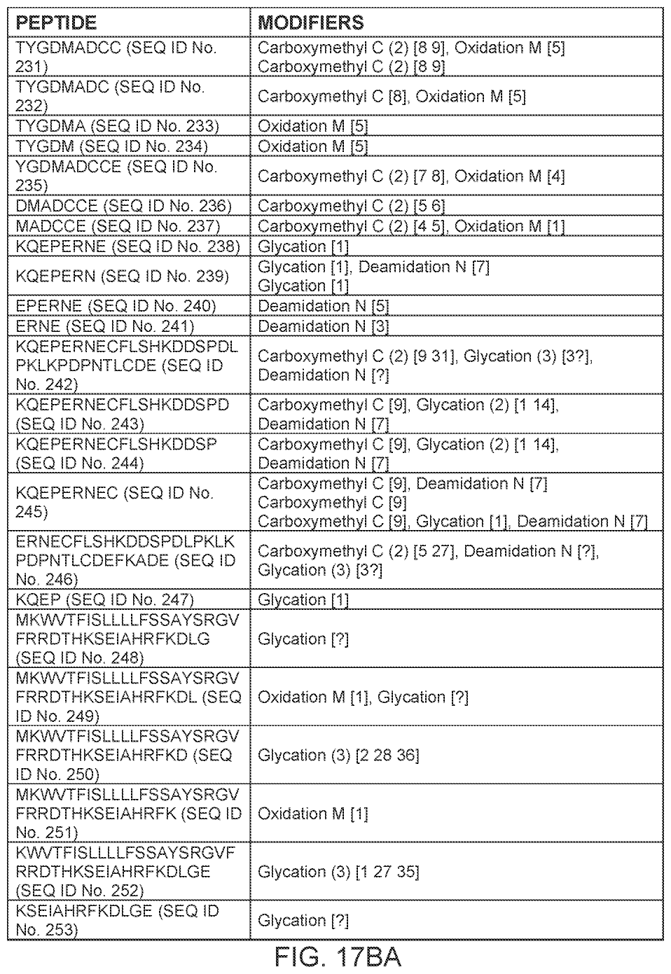

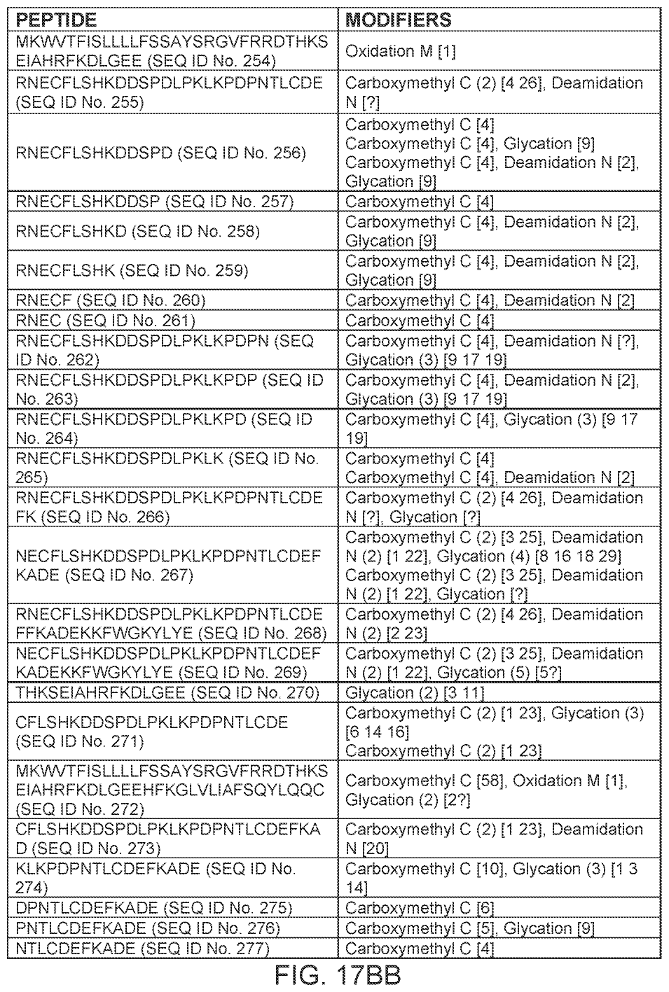

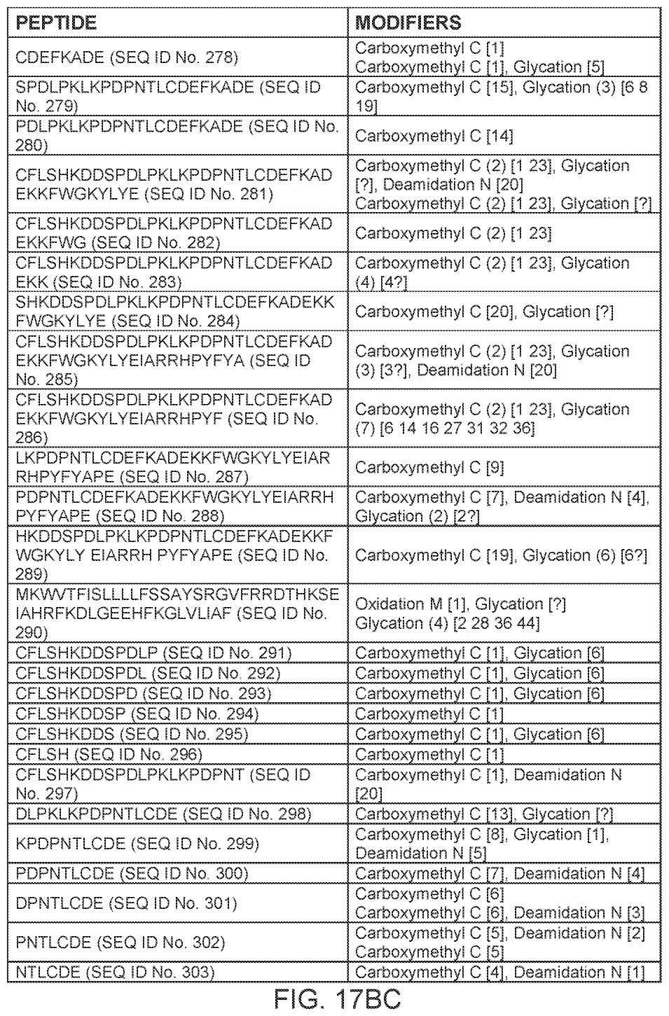

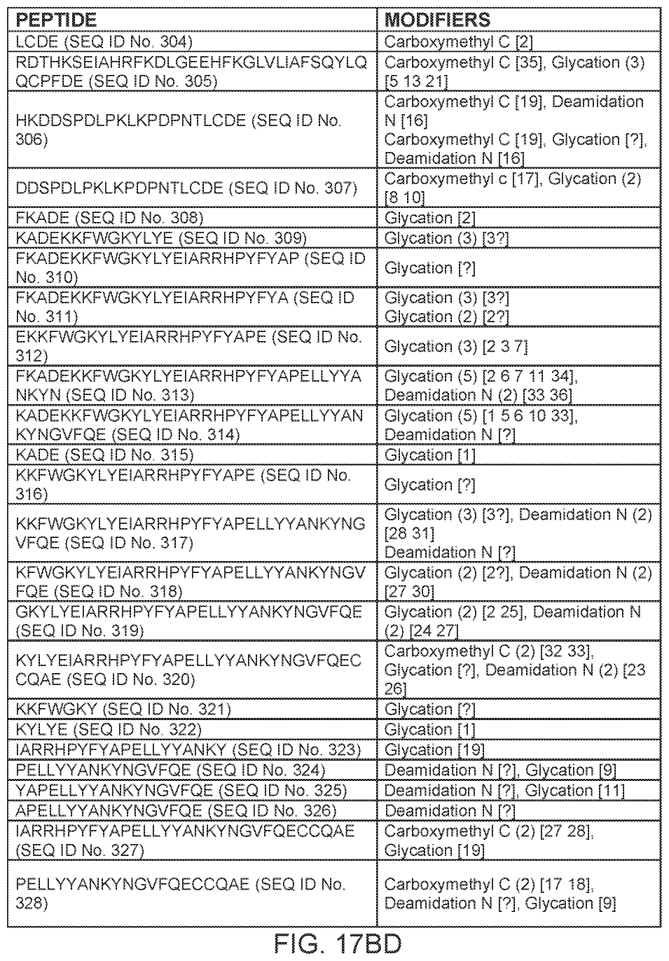

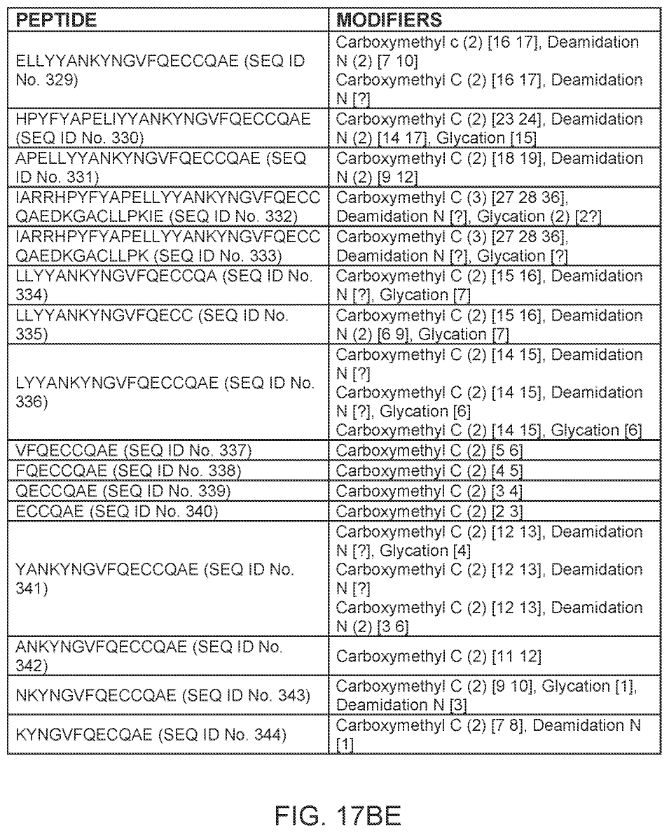

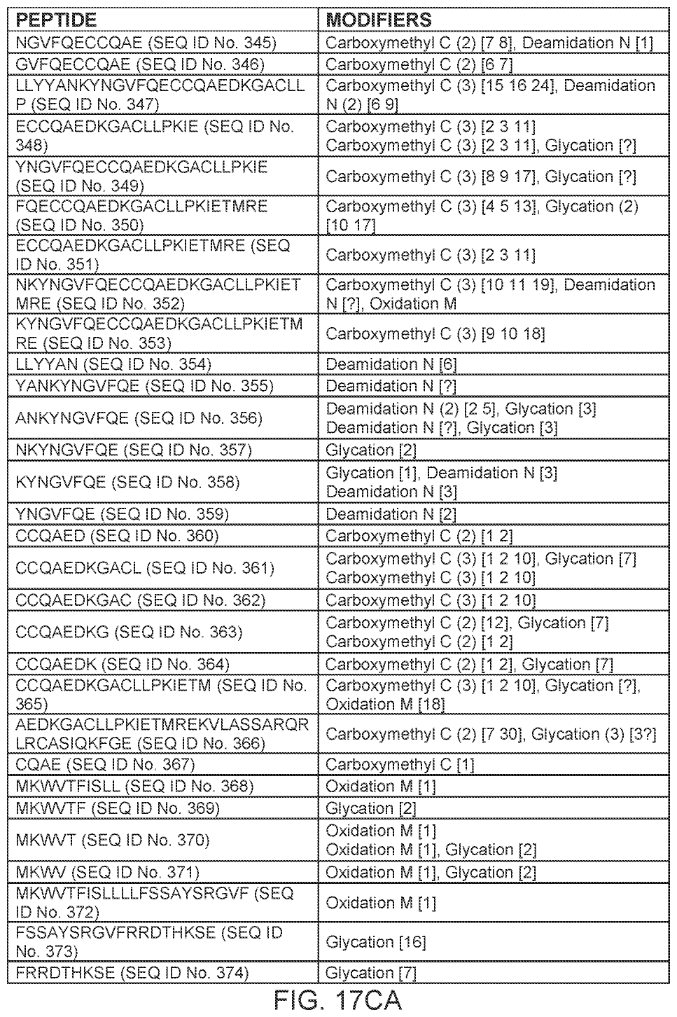

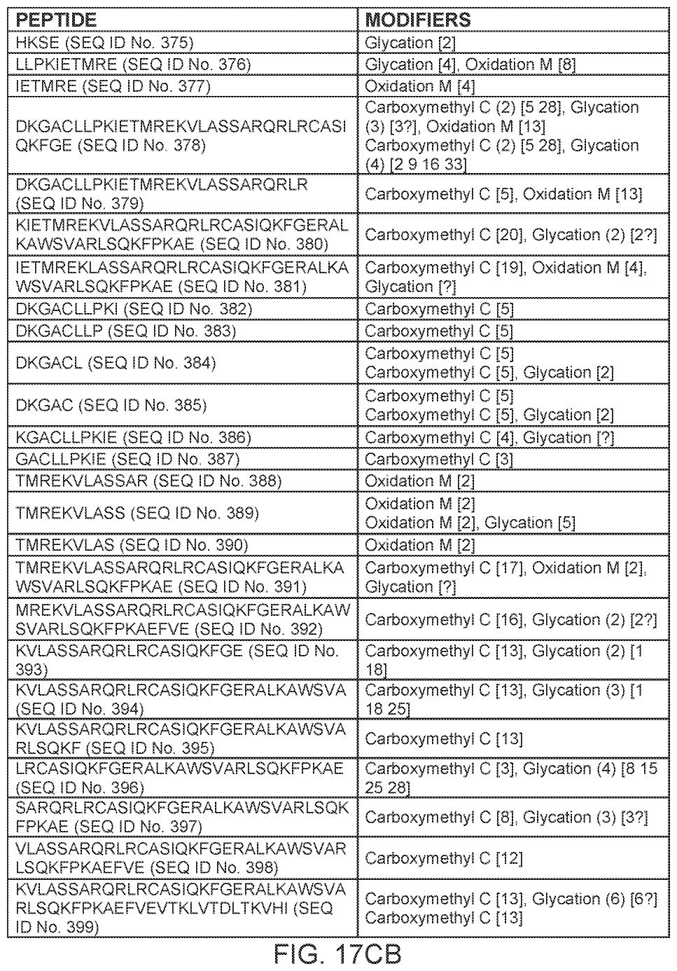

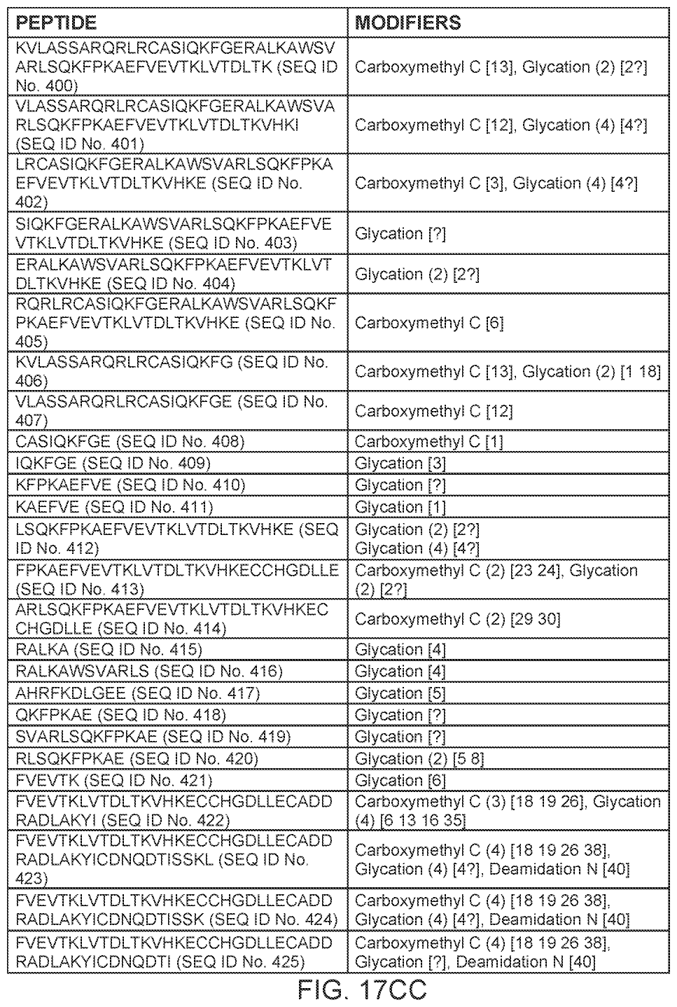

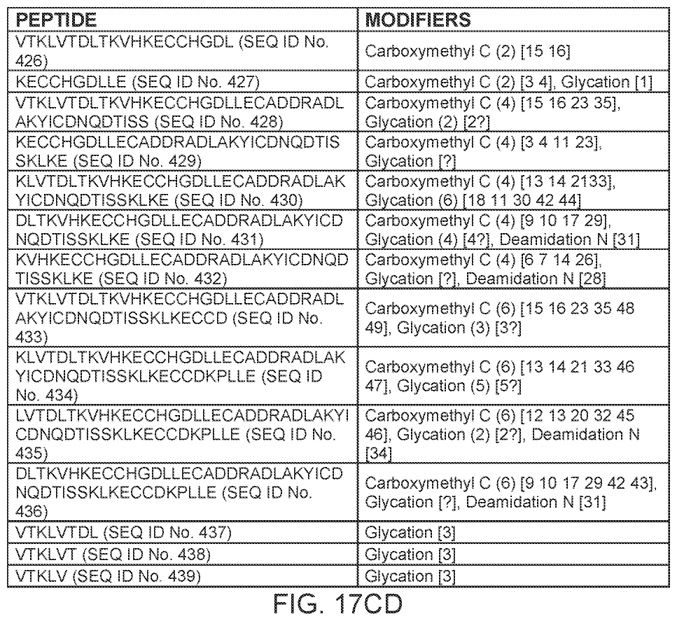

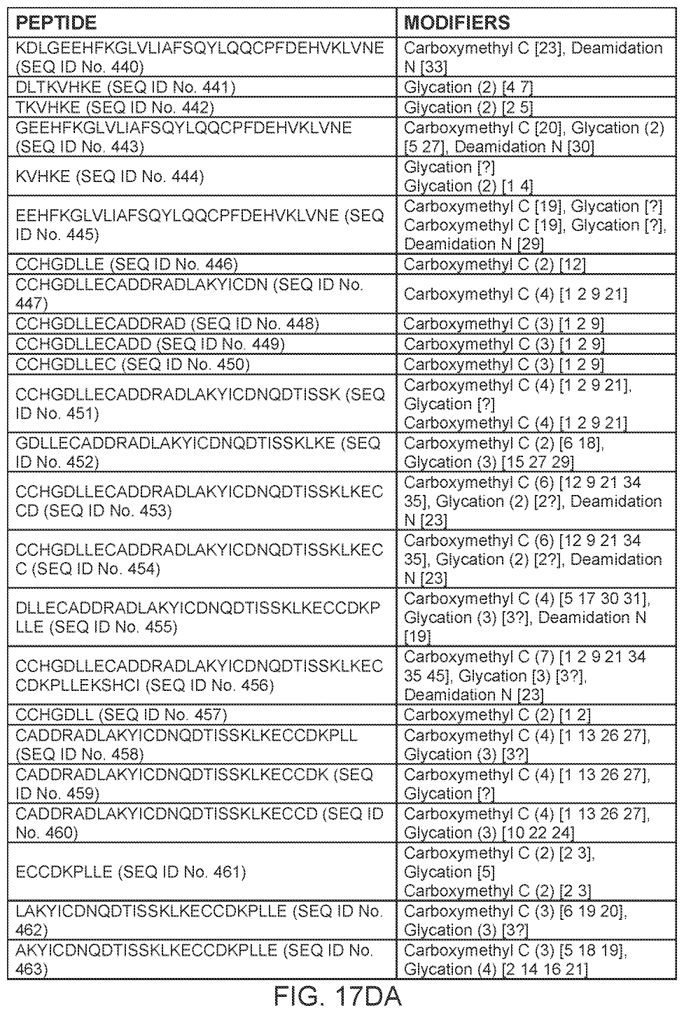

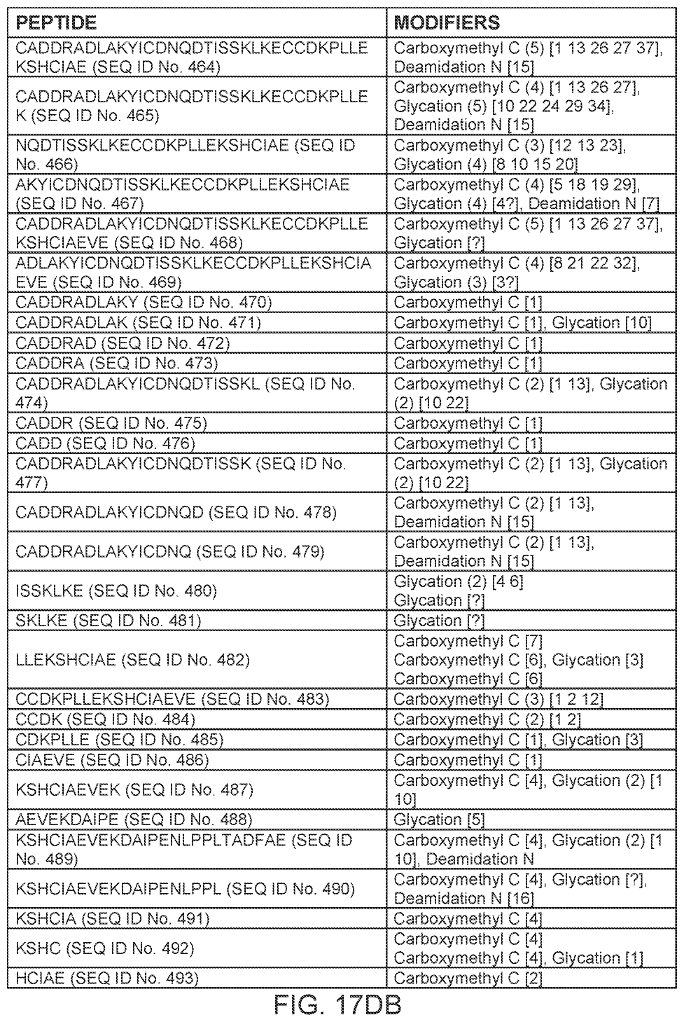

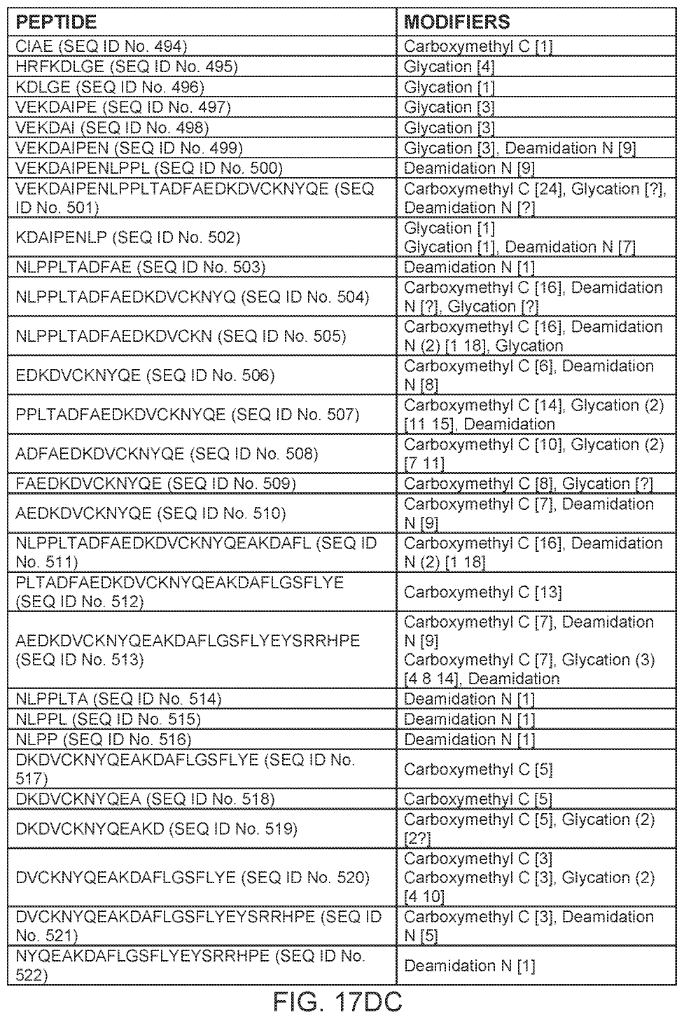

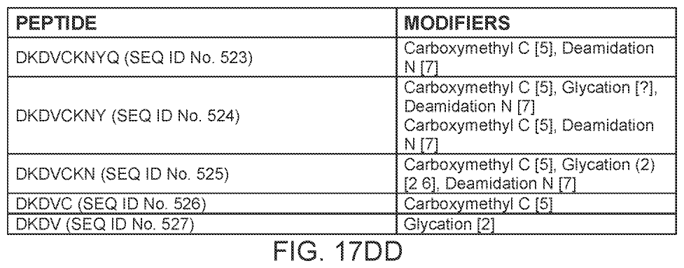

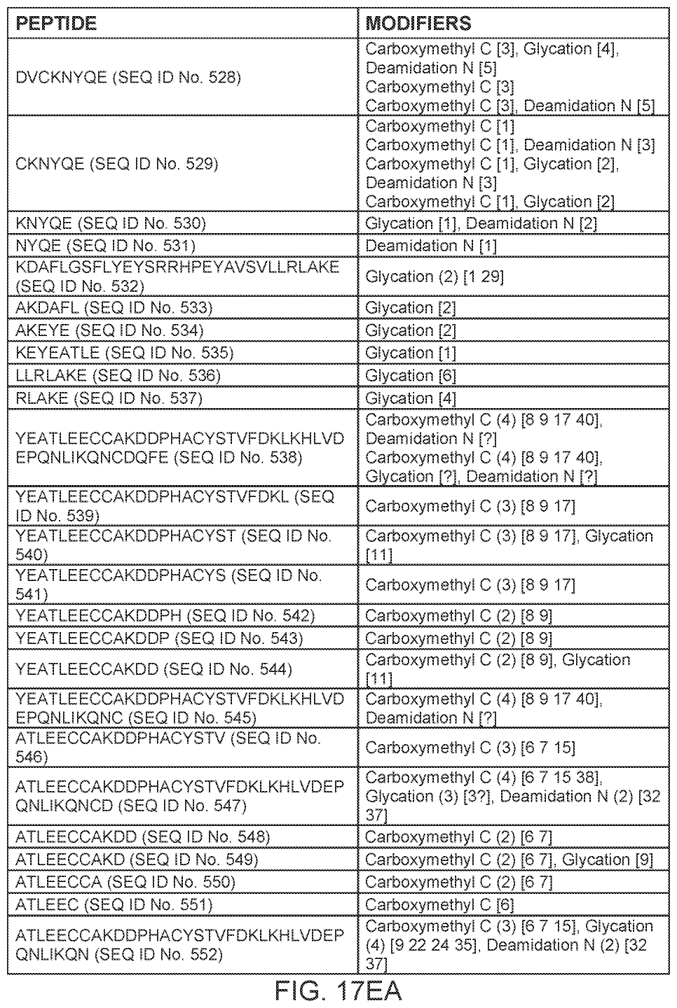

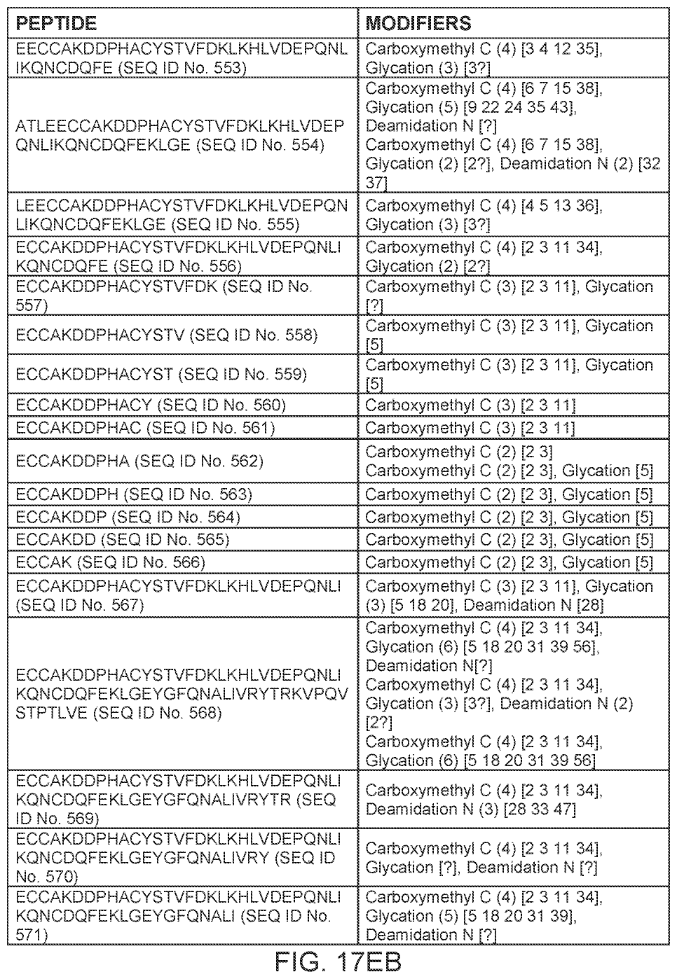

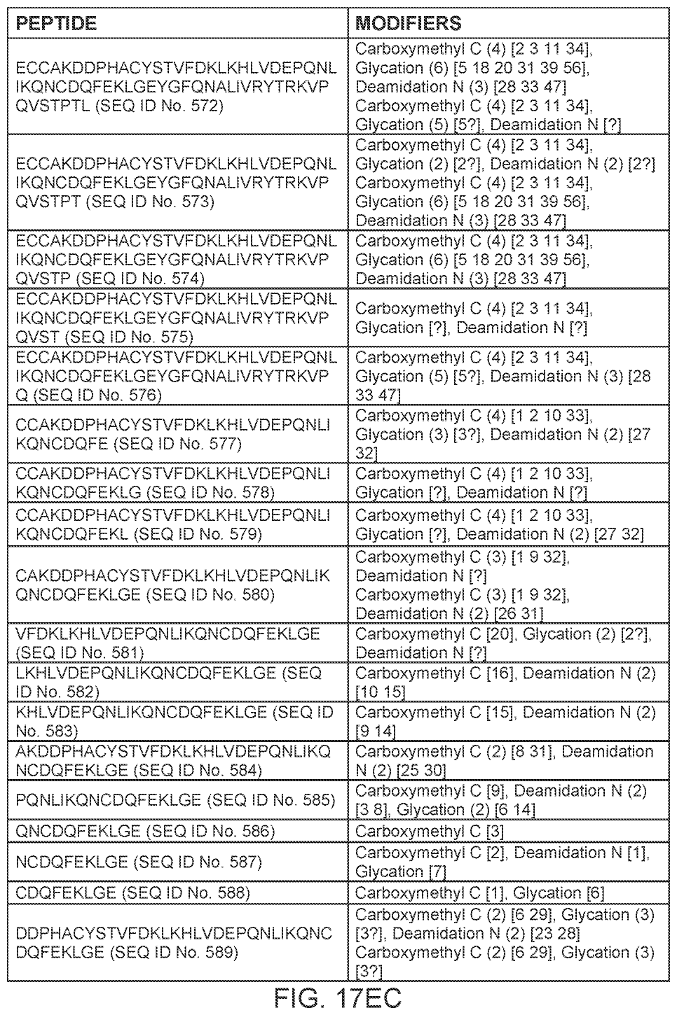

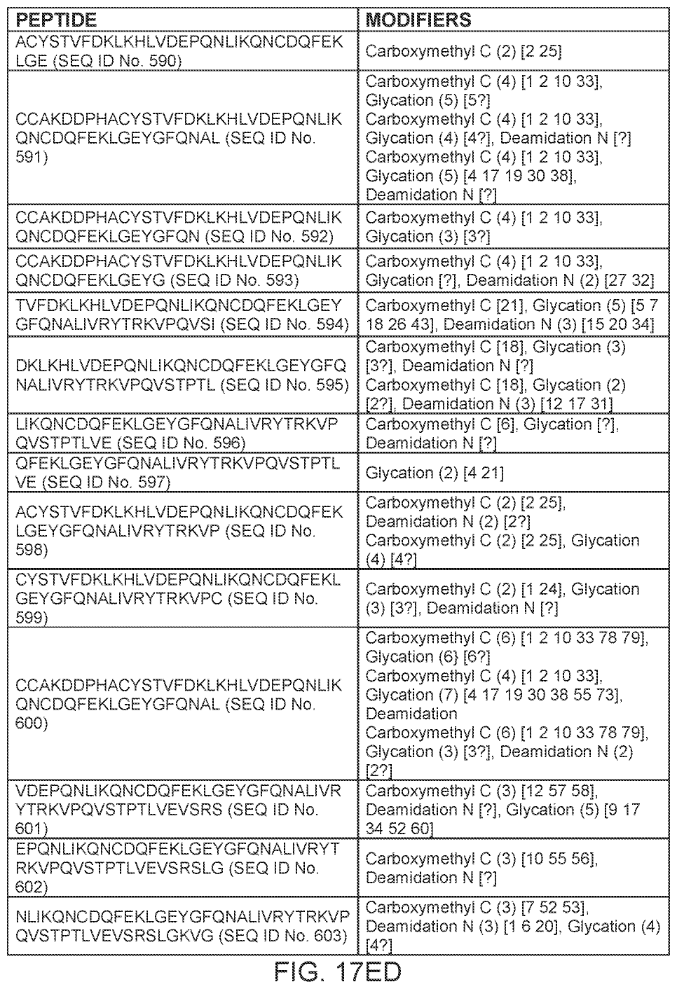

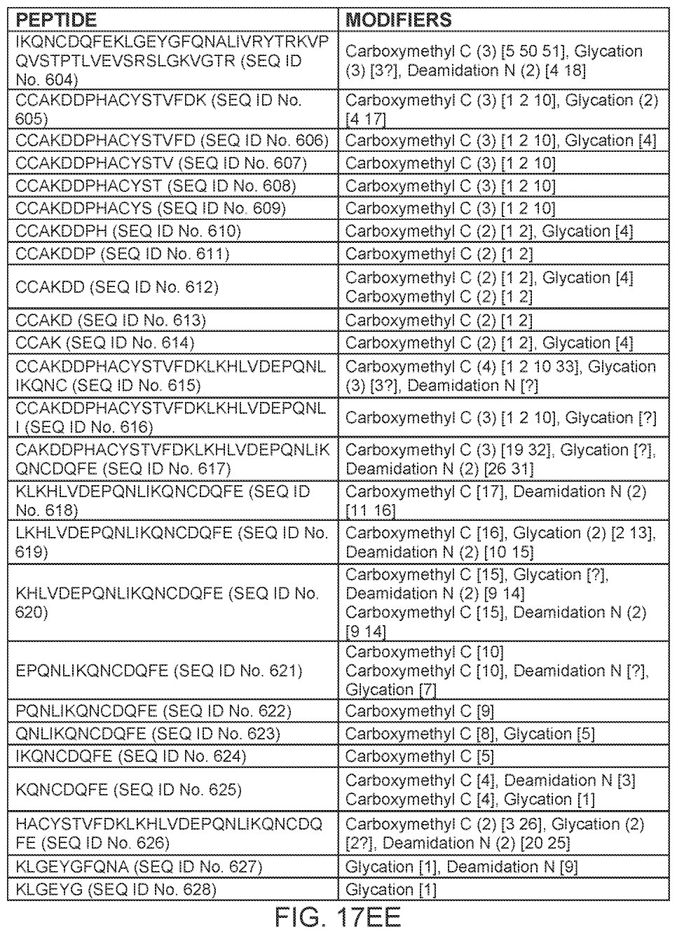

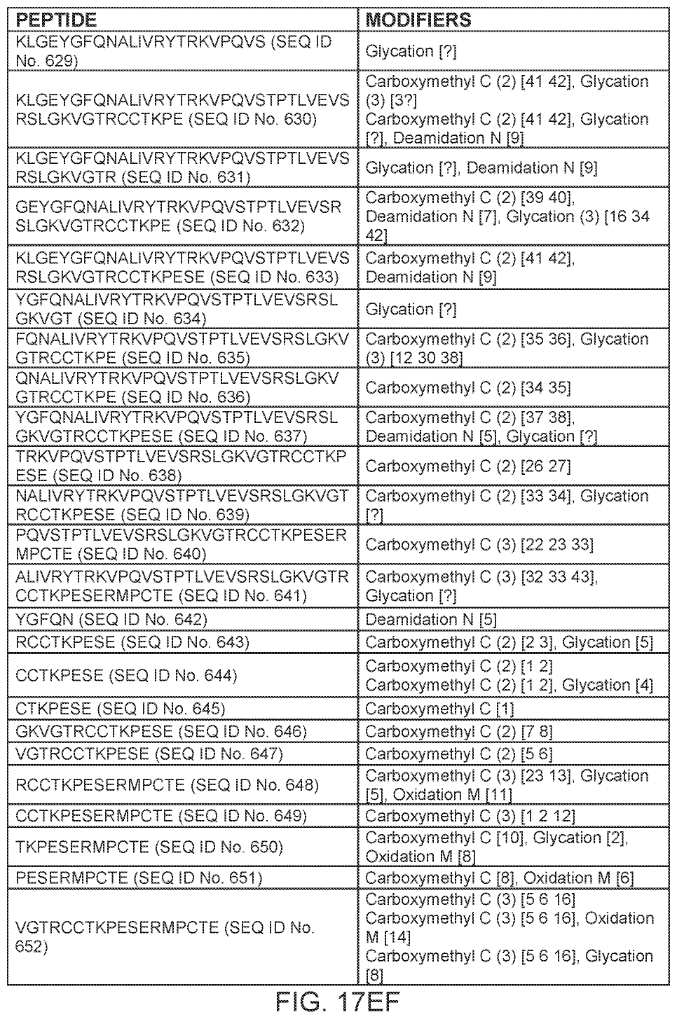

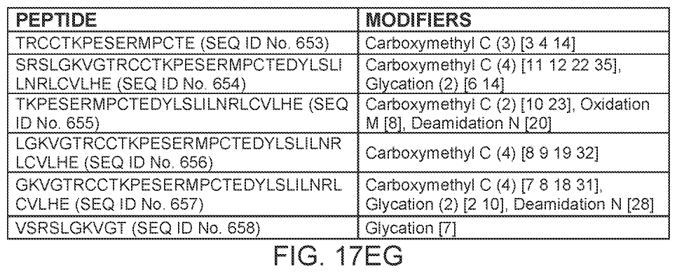

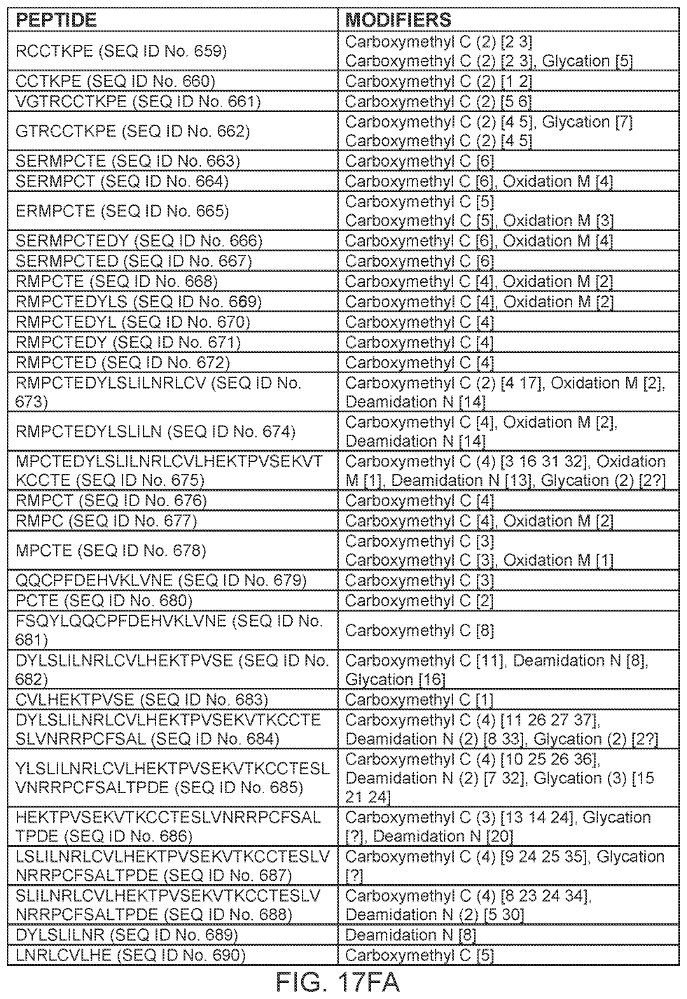

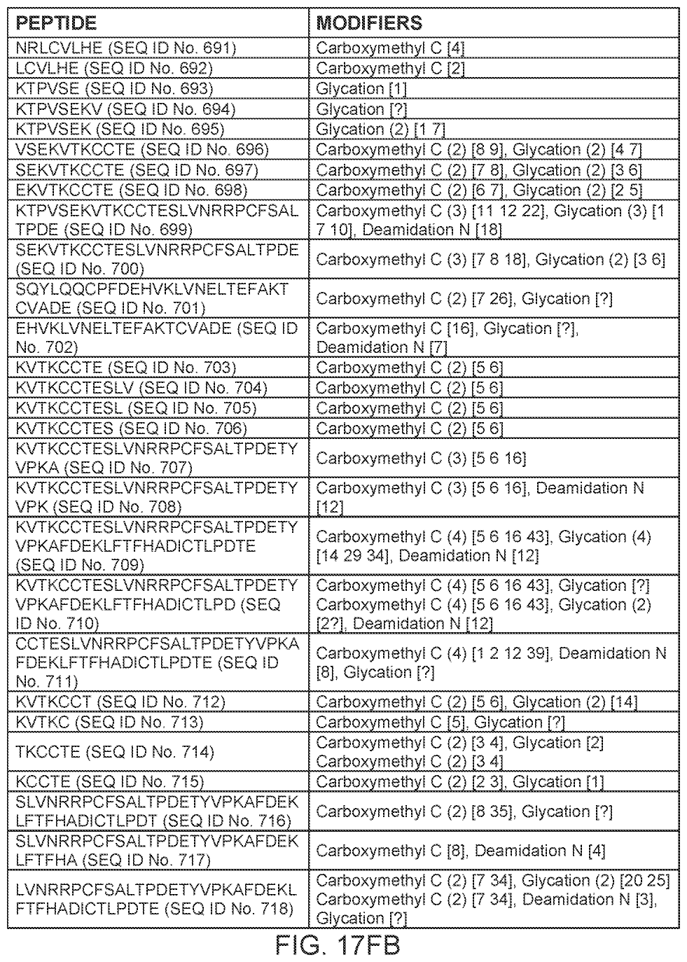

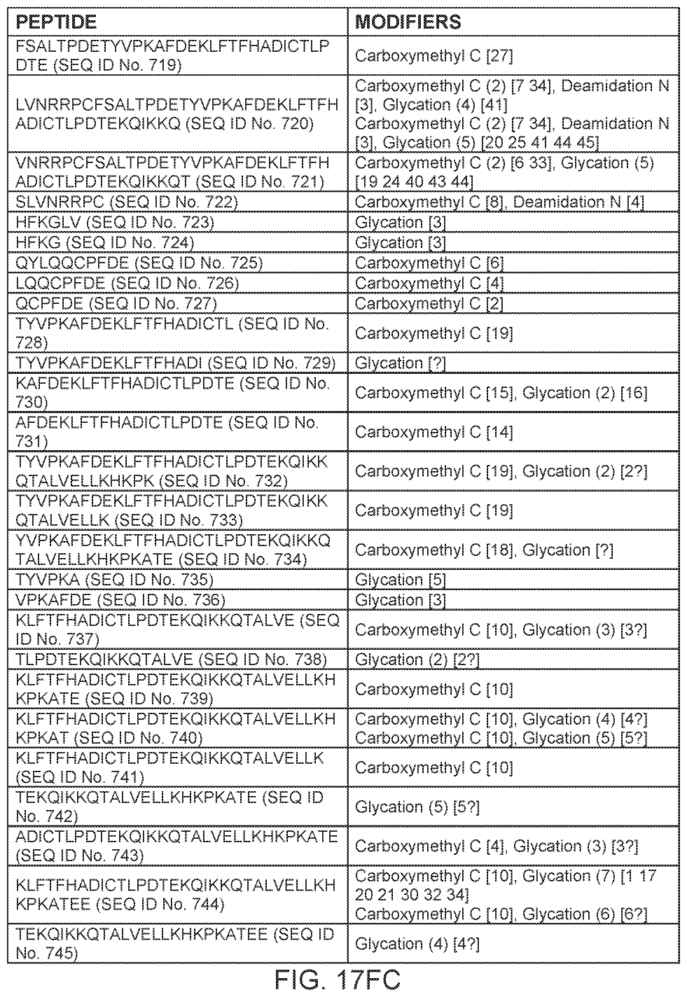

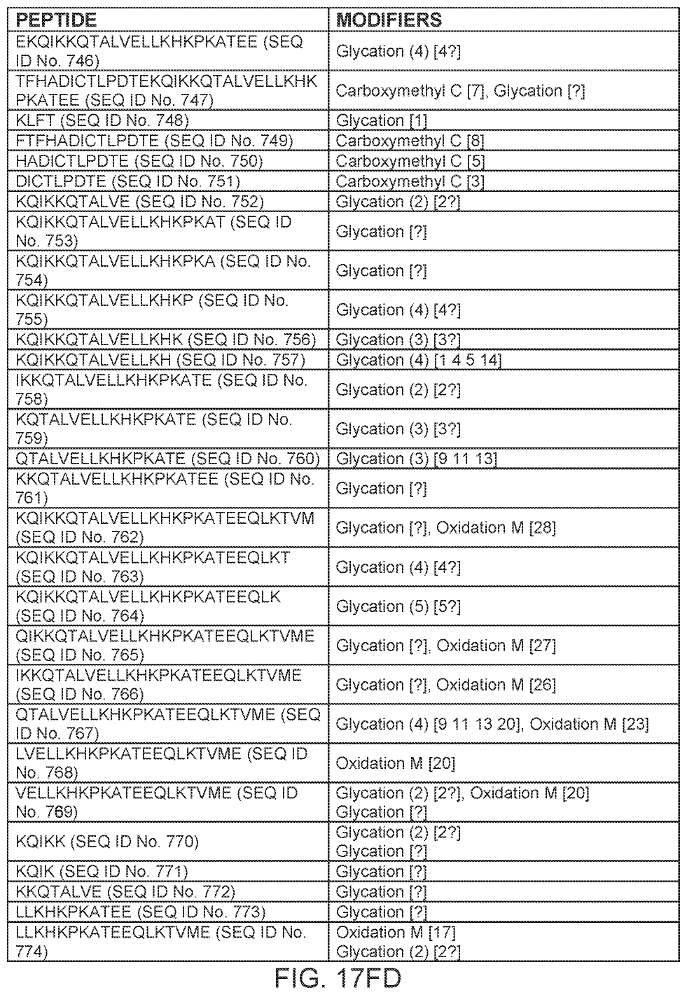

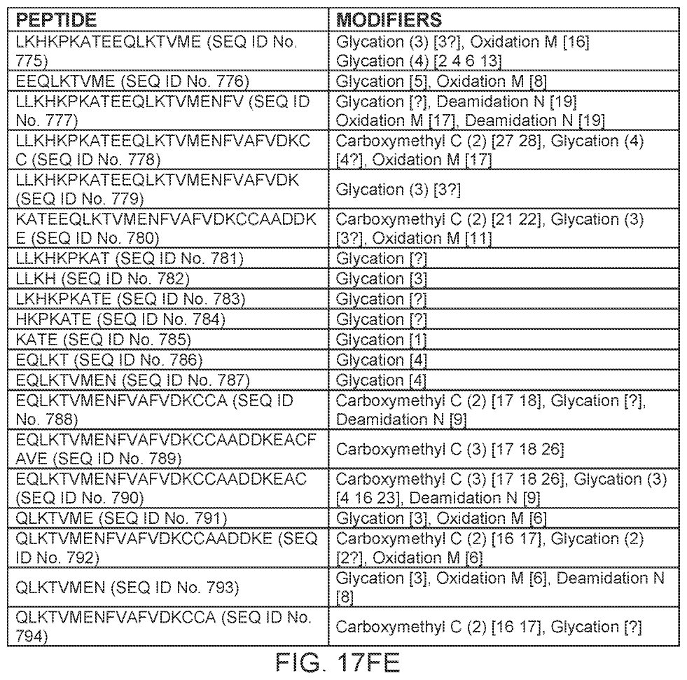

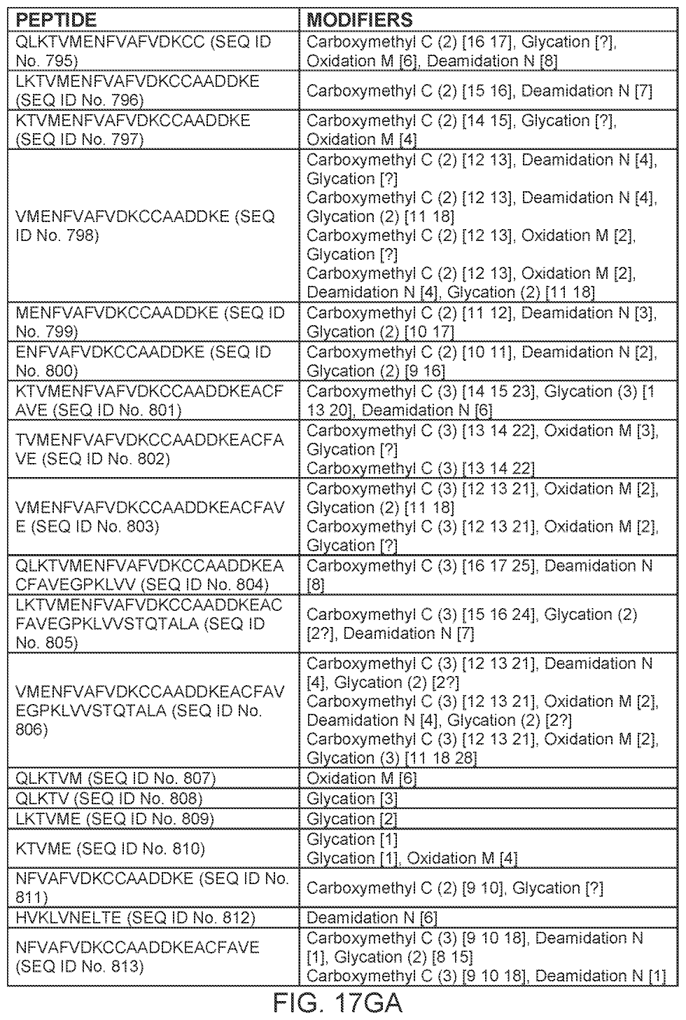

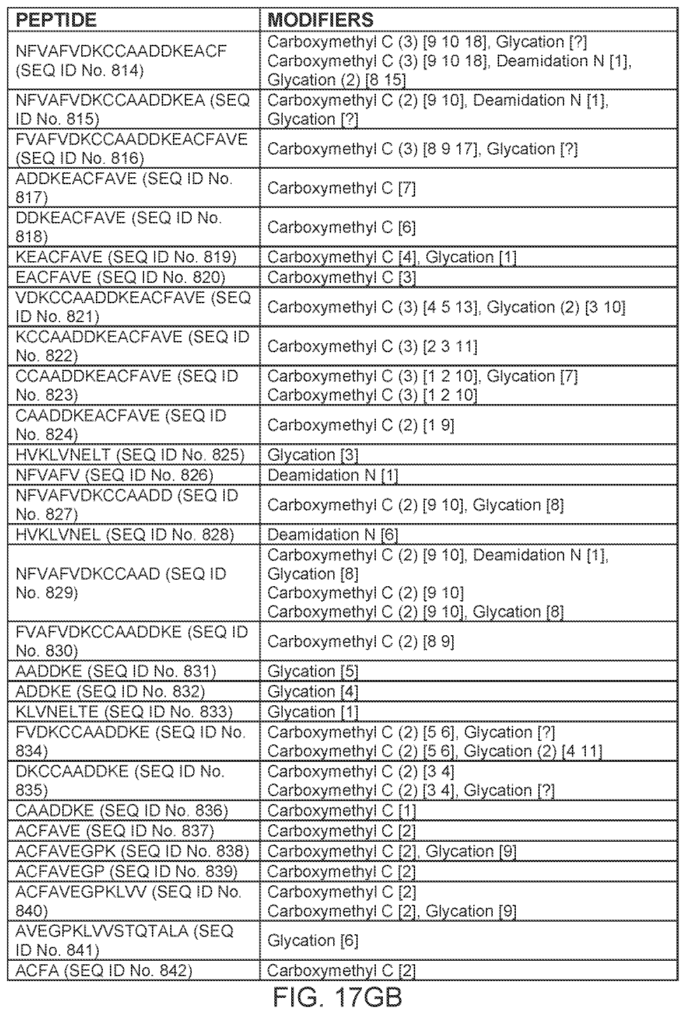

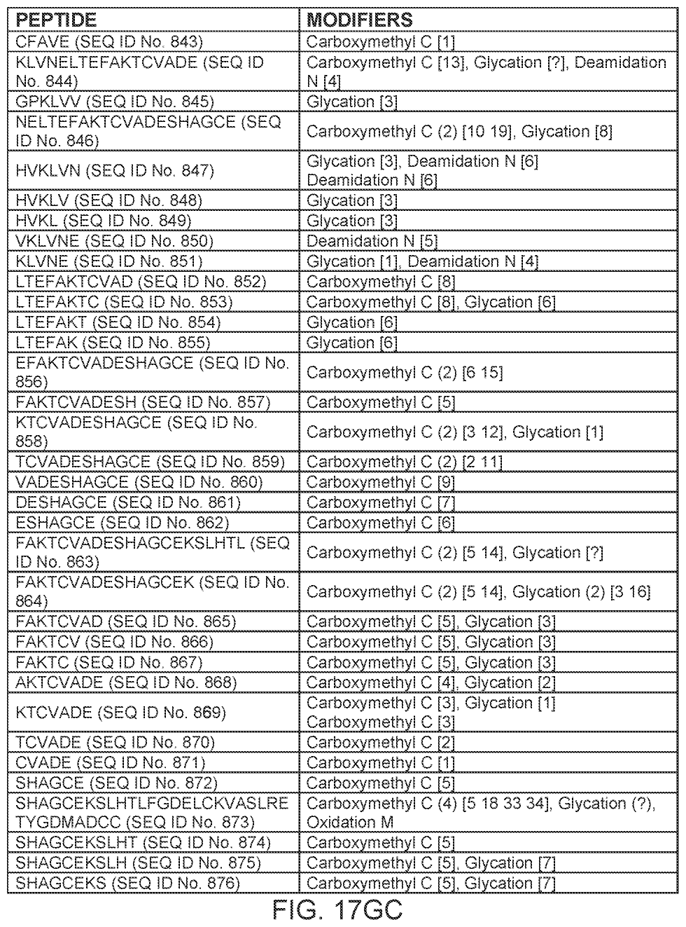

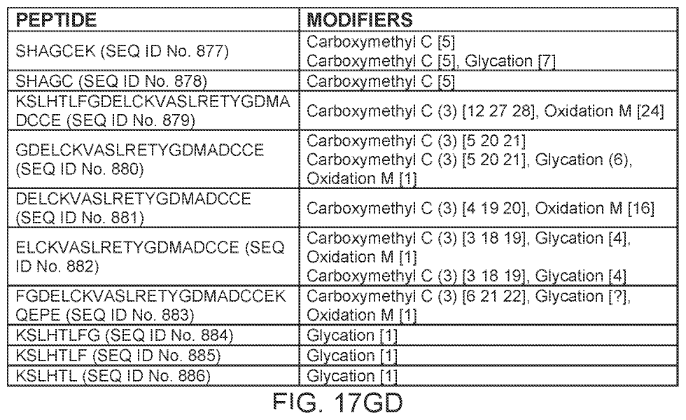

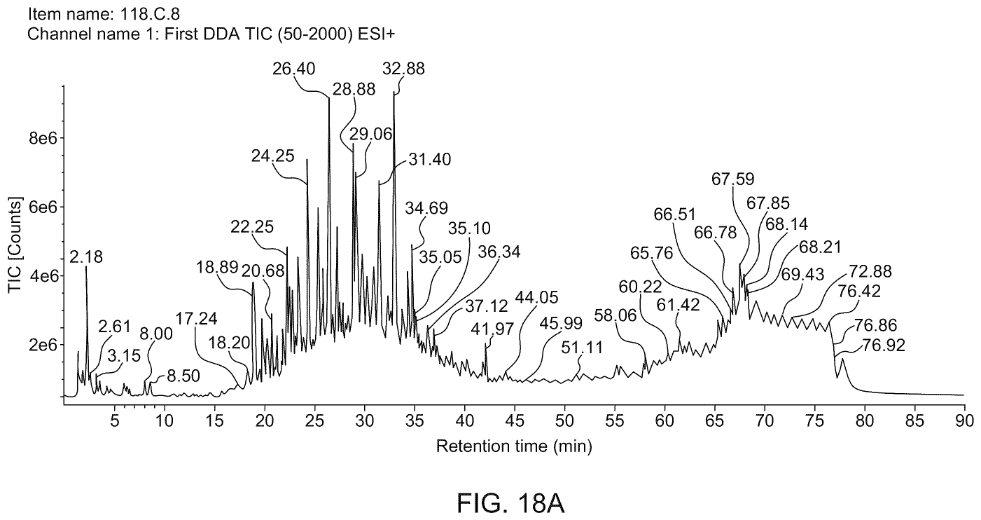

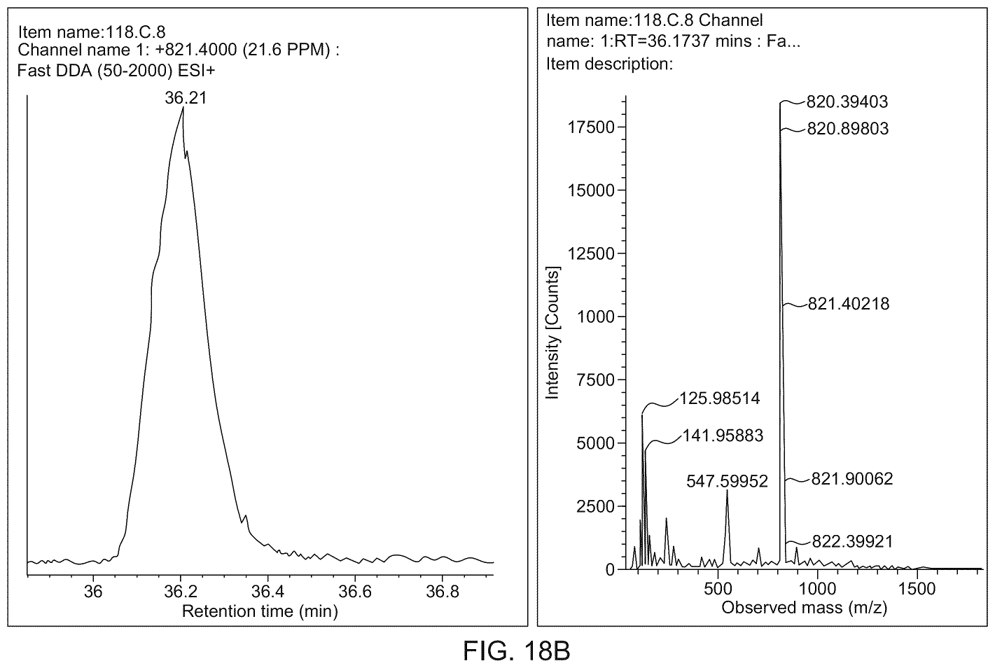

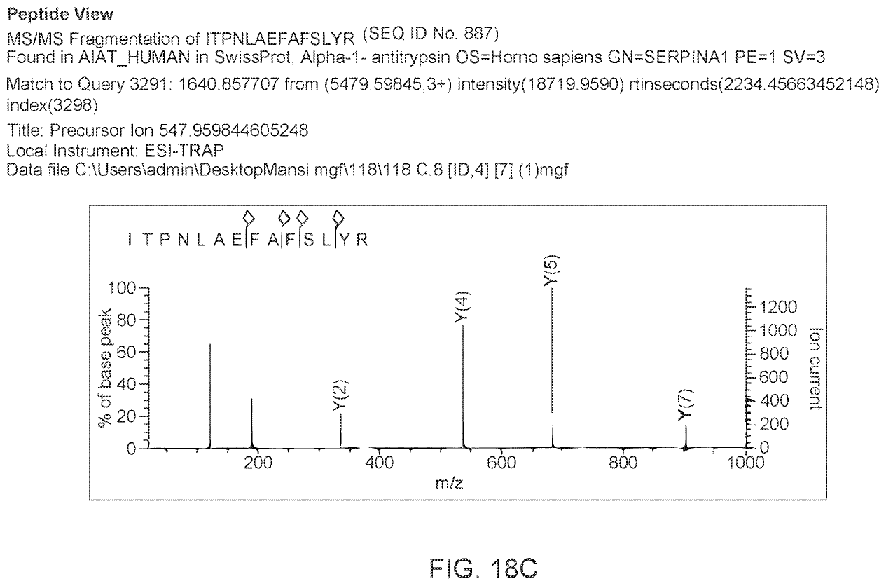

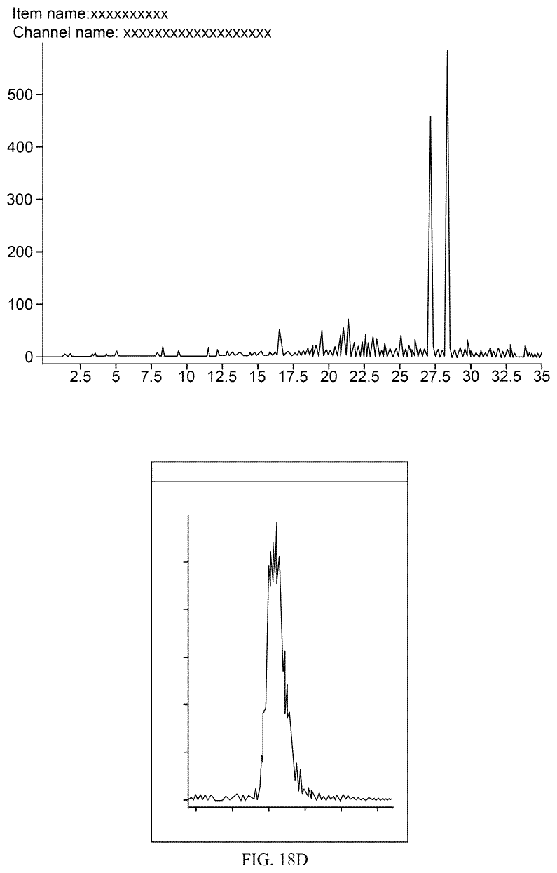

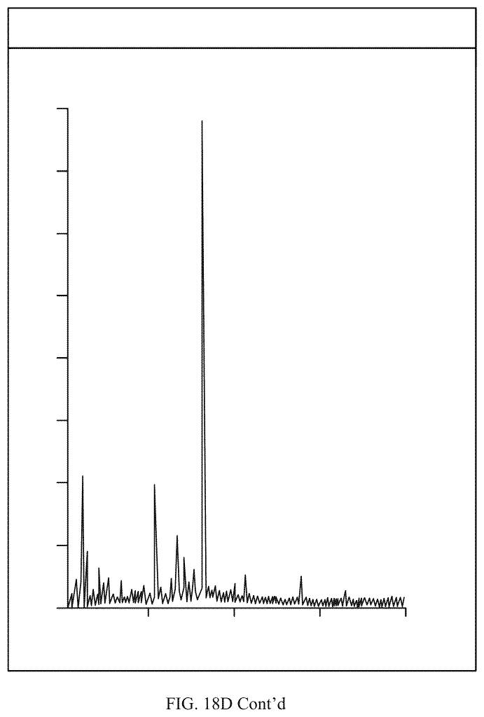

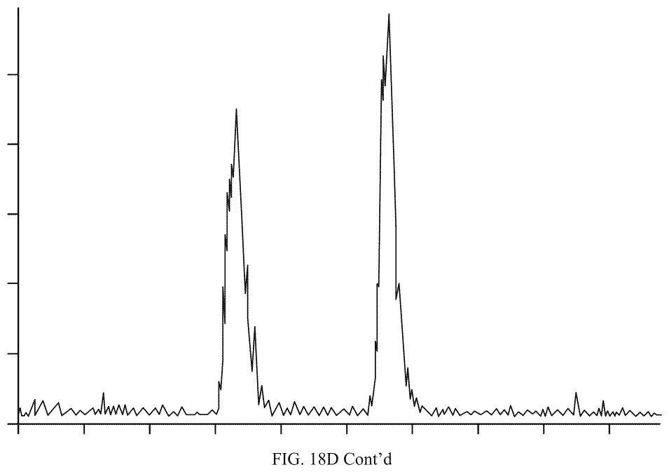

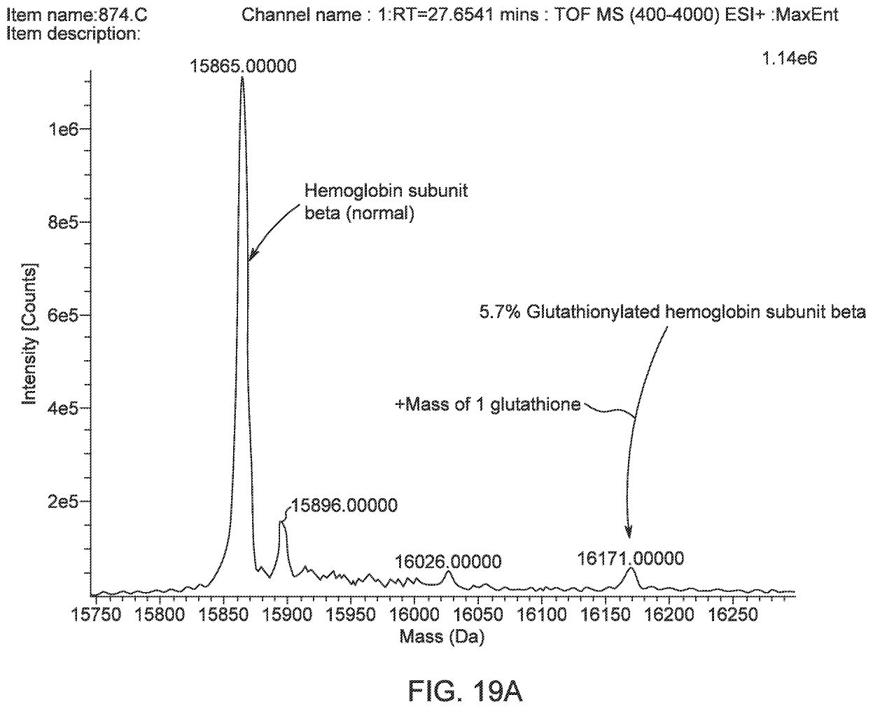

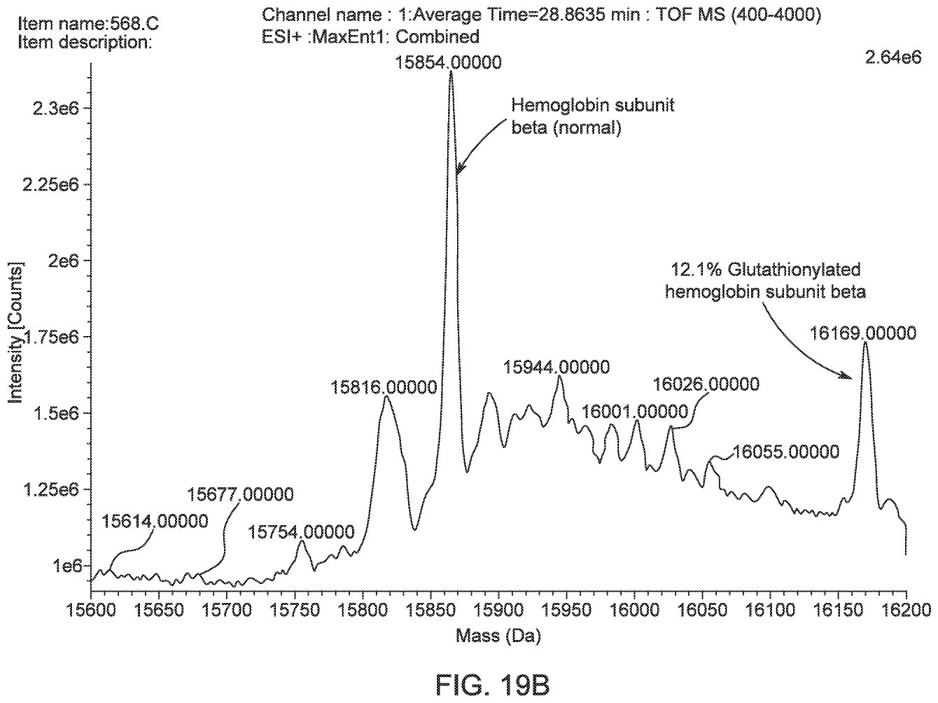

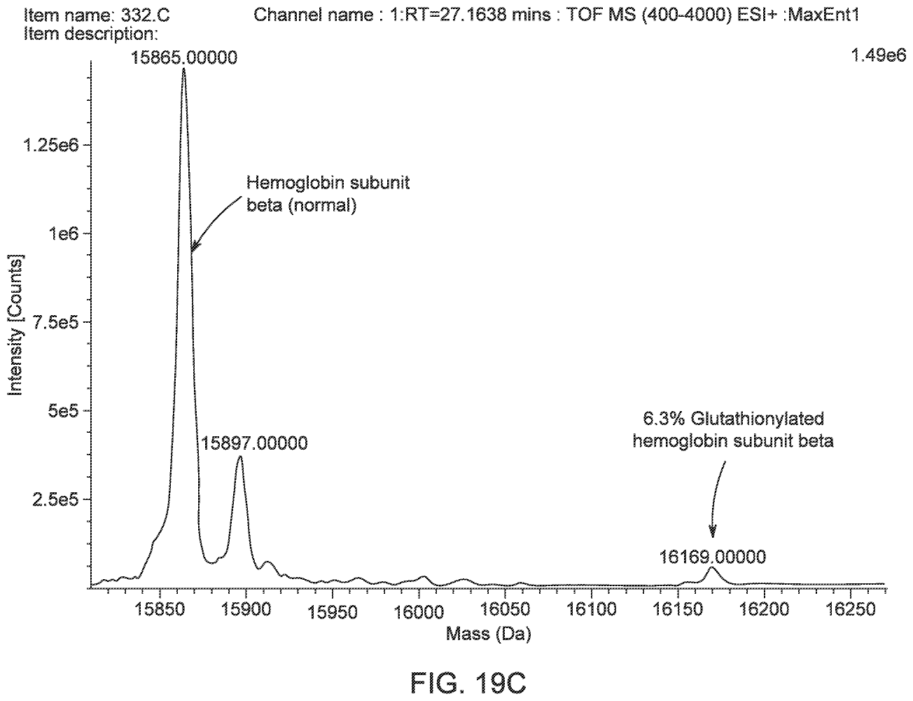

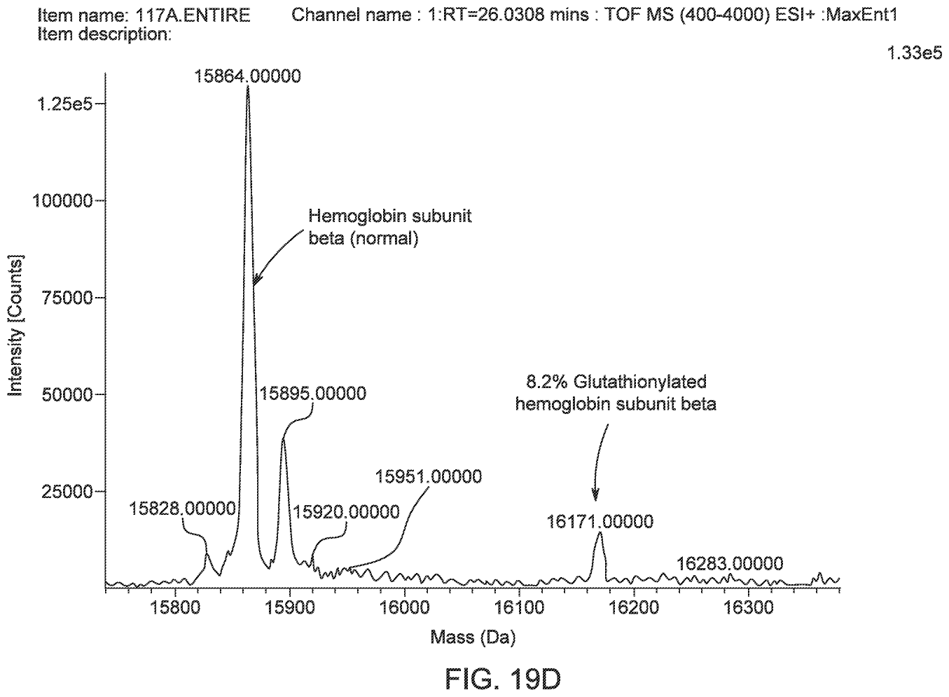

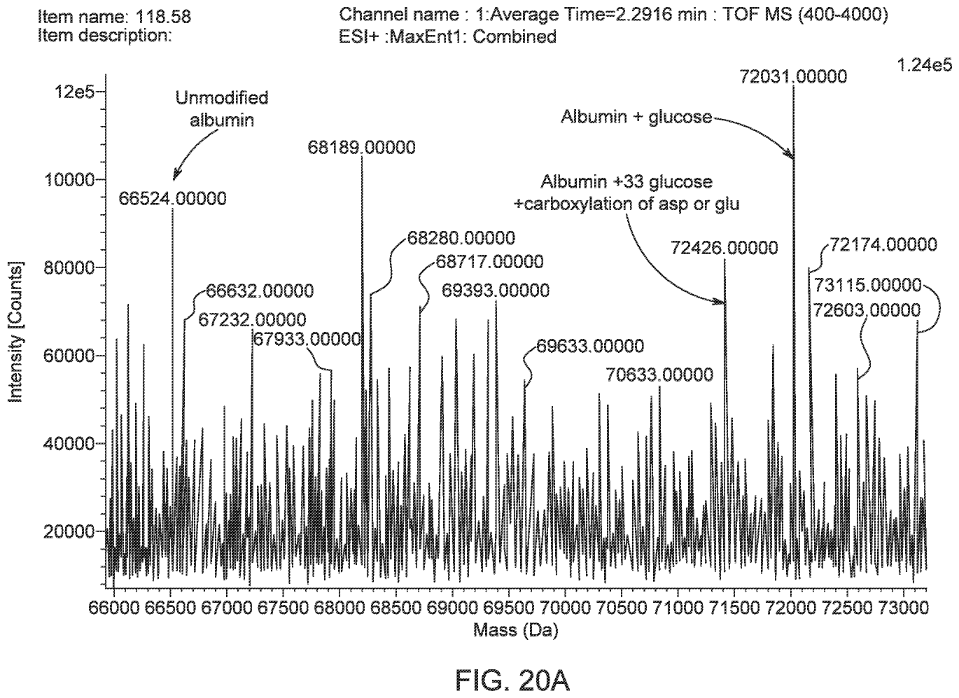

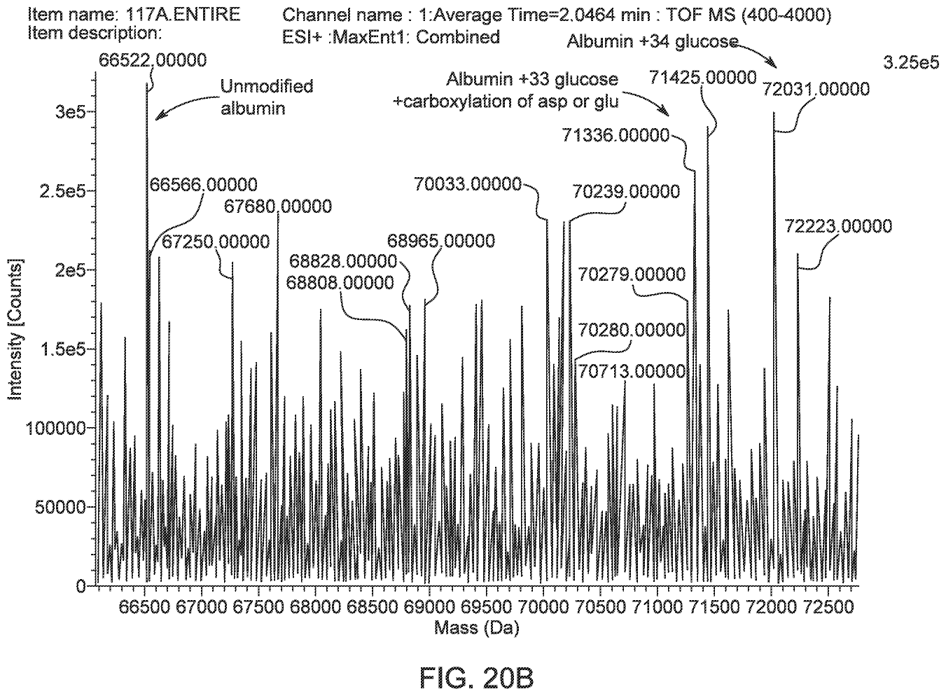

Intact protein analysis is shown in FIGS. 5A, 6A, and 7A. While the hemoglobin subunits from fingerpick blood appeared to be glycated in a similar quantitative pattern as the HbAlc standard (1 glycation event per subunit), the menstrual fluid sample exhibited multiple glycation most clearly on the beta subunit. To investigate this phenomenon further, several menstrual fluid samples were subjected to alternative digestion (Glu-C and trypsin) and analyzed via QTOF MS/MS for the elucidation of the position of glycation. Fragment masses were detected that matched specific amino acid sequences in the hemoglobin subunit plus the mass of a glucose attached to a lysine.

Several of the sites of glycation on menstrual fluid hemoglobin subunit alpha I reported here were not detected as glycated in circulating blood. Thus, we can conclude that the additional sites are specific to menstrual blood hemoglobin subunit alpha, or at least they are the product of particularly high glucose gradients that might be present in the menstrual fluid milieu.

As suggested from the intact analysis, the predominate glycation on menstrual fluid hemoglobin was on the alpha subunit and thus the shear amount of glycated proteoforms allowed for positive identification of sites of glycation on that subunit.

1. A method of stratifying human female subject, comprising
a. obtaining an initial sample of the female subject's menstrual fluid;
b. separating proteins of the menstrual fluid;
c. measuring the level of glycation, and, optionally, glutathionylation, of the hemoglobin subunit alpha in the sample and determining the number of glycation events, and, optionally, glutathionylation events on the hemoglobin alpha subunit;
d. obtaining a subsequent sample of the female subject's menstrual fluid and performing the separating and measuring steps to determine the number of glycation events, and, optionally, glutathionylation events, on the hemoglobin alpha subunit in the subsequent sample; and
e. assigning female subject to a category based on the number of glycation events, and, optionally glutathionylation events on the hemoglobin alpha subunit in the initial and subsequent sample of the female subject's menstrual fluid and using said category for clinical trial enrollment and/or medication dosing.

2. The method of claim 1, wherein the hemoglobin subunit alpha is positively identified from the sample through MS analysis.

3. The method of claim 1 wherein a post-translation modification of glycation and glutathionylation on the hemoglobin subunit alpha is detected.

4. The method of claim 1, wherein the initial and subsequent samples of menstrual fluid is collected using a 903 dried blood spot collection card.

5. The method of claim 1, wherein the hemoglobin subunit alpha is detected using LC-MS/MS peptide sequencing.

6. The method of claim 1, wherein multiple glycation and glutathionylation events are observed on the hemoglobin subunit alpha and assigning female subject to a category based on the number of glycation and glutathionylation events on the hemoglobin alpha subunit in the initial and subsequent sample of the female subject's menstrual fluid and using said category for clinical trial enrollment and medication dosing.

7. The method of claim 1, wherein the initial and subsequent samples of the female subject's menstrual fluid is obtained using a device comprising a disposable cartridge which is insertable into a wireless enabled device.

8. The method of claim 1, wherein the continued use of subsequent menstrual blood collection and analysis for about 3 months, or about 6 months, or about 9 months, or about 1 year, or about 2 years, or about 3 years, or about 4 years, or about 10 years, or about 20 years, or about 30 years, or about 40 years, or about 50 years is used to evaluate the increase, decrease or trend of the glycation, and, optionally, glutathionylation of the hemoglobin alpha subunit.

9. A method of detecting glycated, and, optionally, glutathionylated hemoglobin on the alpha subunit in a female patient, said method comprising:
a. obtaining an initial menstrual fluid sample from a human female patient;
b. detecting whether glycation, and, optionally, glutathionylation of hemoglobin alpha subunit is present in the initial menstrual fluid sample by using LC-MS/MS peptide sequencing;
c. obtaining a subsequent menstrual fluid sample from a human female patient;
d. detecting whether glycation, and, optionally, glutathionylation of hemoglobin alpha subunit is present in the subsequent menstrual fluid sample from a human female patient; and
e. evaluating the increase. decrease or trend of the number of glycation events, and, optionally, glutathionylation events, on the hemoglobin alpha subunit in the initial and subsequent menstrual fluid samples to determine the diabetes and/or prediabetes status of the female patient.

10. The method of claim 9 for detecting modifications of any additional proteins on the hemoglobin alpha subunit in a female patient, said method further comprising:
obtaining initial and subsequent menstrual fluid samples from the female patient; and
detecting whether modification on one or more additional proteins is present in the initial and subsequent menstrual fluid sample samples by using LC-MS/MS peptide sequencing.

11. The method of claim 9 for diagnosing pre-diabetes or diabetes in a female patient, said method further comprising:
a. obtaining initial and subsequent menstrual fluid samples from the female patient;
b. detecting whether glycation, and, optionally, glutathionylation, of hemoglobin alpha subunit is present in the initial and subsequent menstrual fluid samples by using LC-MS/MS peptide sequencing; and
c. diagnosing the female patient with pre-diabetes or diabetes when the presence of one or more glycation events, and, optionally, glutathionylation events, in the initial and subsequent menstrual fluid samples is detected.

12. A method of medication dosing for diabetes or prediabetes in a female patient, said method comprising:
a. obtaining an initial and subsequent menstrual fluid sample from the female patient;
b. measuring the amount of glycation, and, optionally, glutathionylation of hemoglobin subunit alpha in the initial and subsequent menstrual fluid samples;
c. assigning the female patient to a category based on the amount of glycation, and, optionally, glutathionylation of hemoglobin subunit alpha in the initial and subsequent menstrual fluid samples and based on the increase or decrease in the amount of glycation, and, optionally, glutathionylation of hemoglobin subunit alpha in the initial and subsequent menstrual fluid samples; and
d. administering an effective amount of medication for treating diabetes or prediabetes to the patient based on the category assigned to the patient.


SEQUENCE LISTING (888 sequences)

SEQ ID NO: 1 - protein, 8 aa, Homo sapiens
VHLTPEEK

SEQ ID NO: 2 - protein, 146 aa, Homo sapiens
VHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 3 - protein, 13 aa, Homo sapiens
  MOD_RES 2: note = Glycation [2]
GKVGAHAGEYGAE

SEQ ID NO: 4 - protein, 25 aa, Homo sapiens
  MOD_RES 1..25: note = Glycation [?]
  MOD_RES 5..14: note = Glycation (3) [5, 9, 14]
  MOD_RES 7: note = Deamidation N [5]
SPADKTNVKAAWGKVGAHAGEYGAE

SEQ ID NO: 5 - protein, 23 aa, Homo sapiens
  MOD_RES 1..23: note = Glycation [2 ?]
  MOD_RES 5: note = Deamidation N [5]
ADKTNVKAAWGKVGAHAGEYGAE

SEQ ID NO: 6 - protein, 28 aa, Homo sapiens
  MOD_RES 5..14: note = Glycation (3) [5, 9, and/or 14]
SPADKTNVKAAWGKVGAHAGEYGAEALE

SEQ ID NO: 7 - protein, 27 aa, Homo sapiens
  MOD_RES 4..13: note = Glycation (3) [4, 8, and/or 13]
  MOD_RES 6: note = Deamidation N [6]
PADKTNVKAAWGKVGAHAGEYGAEALE

SEQ ID NO: 8 - protein, 23 aa, Homo sapiens
  MOD_RES 1..23: note = Glycation [?]
TNVKAAWGKVGAHAGEYGAEALE

SEQ ID NO: 9 - protein, 84 aa, Homo sapiens
  MOD_RES 1..84: note = Glycation (5) [5?]
  MOD_RES 1..84: note = Deamidation N (2) [2?]
VLSPADKTNVKAAWGKVGAHAGEYGAEALERMFLSFPTTKTYFPHFDLSHGSAQVKGHGKKVADALTNAVAHVDDMPNALSALS

SEQ ID NO: 10 - protein, 82 aa, Homo sapiens
  MOD_RES 1..82: note = Glycation (5) [5?]
  MOD_RES 1..82: note = Oxidation M [?]
VLSPADKTNVKAAWGKVGAHAGEYGAEALERMFLSFPTTKTYFPHFDLSHGSAQVKGHGKKVADALTNAVAHVDDMPNALSA

SEQ ID NO: 11 - protein, 81 aa, Homo sapiens
  MOD_RES 1..81: note = Deamidation N [?]
  MOD_RES 7..61: note = Glycation (7) [7, 11, 16, 40, 56, 60, and/or 61]
VLSPADKTNVKAAWGKVGAHAGEYGAEALERMFLSFPTTKTYFPHFDLSHGSAQVKGHGKKVADALTNAVAHVDDMPNALS

SEQ ID NO: 12 - protein, 70 aa, Homo sapiens
  MOD_RES 32: note = Oxidation M [32]
VLSPADKTNVKAAWGKVGAHAGEYGAEALERMFLSFPTTKTYFPHFDLSHGSAQVKGHGKKVADALTNAV

SEQ ID NO: 13 - protein, 32 aa, Homo sapiens
  MOD_RES 1..32: note = Glycation [?]
  MOD_RES 1..32: note = Glycation (2) [2?]
  MOD_RES 9: note = Deamidation N [9]
  MOD_RES 32: note = Oxidation M [32]
VLSPADKTNVKAAWGKVGAHAGEYGAEALERM

SEQ ID NO: 14 - protein, 99 aa, Homo sapiens
  MOD_RES 1..99: note = Glycation [?]
  MOD_RES 51..80: note = Deamidation N (3) [51, 61, and/or 80]
  MOD_RES 87: note = Carboxymethyl C [87]
GAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAE

SEQ ID NO: 15 - protein, 98 aa, Homo sapiens
  MOD_RES 1..98: note = Glycation [?]
  MOD_RES 50..79: note = Deamidation N (3) [50, 60, and/or 79]
  MOD_RES 86: note = Carboxymethyl C [86]
AHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAE

SEQ ID NO: 16 - protein, 108 aa, Homo sapiens
  MOD_RES 1..108: note = Glycation (6) [6?]
  MOD_RES 1..108: note = Deamidation N (3) [3?]
  MOD_RES 24..68: note = Oxidation M (2) [24 and/or 68]
  MOD_RES 96: note = Carboxymethyl C [96]
NVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAE

SEQ ID NO: 17 - protein, 9 aa, Homo sapiens
  MOD_RES 7: note = Glycation [7]
  MOD_RES 9: note = Deamidation N [9]
VLSPADKTN

SEQ ID NO: 18 - protein, 8 aa, Homo sapiens
  MOD_RES 7: note = Glycation [7]
VLSPADKT

SEQ ID NO: 19 - protein, 13 aa, Homo sapiens
  MOD_RES 1..13: note = Glycation [?]
KAAWGKVGAHAGE

SEQ ID NO: 20 - protein, 10 aa, Homo sapiens
  MOD_RES 3: note = Glycation [3]
WGKVGAHAGE

SEQ ID NO: 21 - protein, 93 aa, Homo sapiens
  MOD_RES 1..93: note = Glycation [?]
  MOD_RES 1..93: note = Deamidation N [?]
  MOD_RES 9..53: note = Oxidation M (2) [9 and/or 53]
  MOD_RES 81: note = Carboxymethyl C [81]
YGAEALERMFLSFPTTKTYFPHFDLSHGSAQVKGHGKKVADALTNAVAHVDDMPNALSALSDLHAHKLRVDPVNFKLLSHCLLVTLAAHLPAE

SEQ ID NO: 22 - protein, 74 aa, Homo sapiens
  MOD_RES 9..53: note = Oxidation M (2) [9 and/or 53]
  MOD_RES 17..67: note = Glycation (5) [17, 33, 37, 38, and/or 67]
  MOD_RES 74: note = Deamidation N [74]
YGAEALERMFLSFPTTKTYFPHFDLSHGSAQVKGHGKKVADALTNAVAHVDDMPNALSALSDLHAHKLRVDPVN

SEQ ID NO: 23 - protein, 64 aa, Homo sapiens
  MOD_RES 1..64: note = Deamidation N [?]
  MOD_RES 1..64: note = Glycation (3) [3?]
  MOD_RES 9..53: note = Oxidation M (2) [9 and/or 53]
YGAEALERMFLSFPTTKTYFPHFDLSHGSAQVKGHGKKVADALTNAVAHVDDMPNALSALSDLH

SEQ ID NO: 24 - protein, 62 aa, Homo sapiens
  MOD_RES 45..55: note = Deamidation N (2) [45 and/or 55]
YGAEALERMFLSFPTTKTYFPHFDLSHGSAQVKGHGKKVADALTNAVAHVDDMPNALSALSD

SEQ ID NO: 25 - protein, 58 aa, Homo sapiens
  MOD_RES 1..58: note = Glycation (2) [2?]
  MOD_RES 1..58: note = Deamidation N [?]
  MOD_RES 9..53: note = Oxidation M (2) [9 and/or 53]
  MOD_RES 45..55: note = Deamidation N (2) [45 and/or 55]
YGAEALERMFLSFPTTKTYFPHFDLSHGSAQVKGHGKKVADALTNAVAHVDDMPNALS

SEQ ID NO: 26 - protein, 55 aa, Homo sapiens
  MOD_RES 1..55: note = Oxidation M [?]
  MOD_RES 1..55: note = Deamidation N [?]
  MOD_RES 17..38: note = Glycation (4) [17, 33, 37, and/or 38]
YGAEALERMFLSFPTTKTYFPHFDLSHGSAQVKGHGKKVADALTNAVAHVDDMPN

SEQ ID NO: 27 - protein, 50 aa, Homo sapiens
  MOD_RES 9: note = Oxidation M [9]
  MOD_RES 17..38: note = Glycation (4) [17, 33, 37, and/or 38]
YGAEALERMFLSFPTTKTYFPHFDLSHGSAQVKGHGKKVADALTNAVAHV

SEQ ID NO: 28 - protein, 45 aa, Homo sapiens
  MOD_RES 9: note = Oxidation M [9]
  MOD_RES 17..38: note = Glycation (4) [17, 33, 37, and/or 38]
  MOD_RES 45: note = Deamidation N [45]
YGAEALERMFLSFPTTKTYFPHFDLSHGSAQVKGHGKKVADALTN

SEQ ID NO: 29 - protein, 88 aa, Homo sapiens
  MOD_RES 1..88: note = Deamidation N (2) [2?]
  MOD_RES 1..88: note = Glycation (4) [4?]
  MOD_RES 9..53: note = Oxidation M (2) [9 and/or 53]
  MOD_RES 81: note = Carboxymethyl C [81]
YGAEALERMFLSFPTTKTYFPHFDLSHGSAQVKGHGKKVADALTNAVAHVDDMPNALSALSDLHAHKLRVDPVNFKLLSHCLLVTLAA

SEQ ID NO: 30 - protein, 19 aa, Homo sapiens
  MOD_RES 9: note = Oxidation M [9]
  MOD_RES 17: note = Glycation [17]
YGAEALERMFLSFPTTKTY

SEQ ID NO: 31 - protein, 83 aa, Homo sapiens
  MOD_RES 1..83: note = Oxidation M [?]
  MOD_RES 17..76: note = Glycation (6) [17, 33, 37, 38, 67, and/or 76]
  MOD_RES 45..74: note = Deamidation N (3) [45, 55, and/or 74]
  MOD_RES 81: note = Carboxymethyl C [81]
YGAEALERMFLSFPTTKTYFPHFDLSHGSAQVKGHGKKVADALNAVAHVDDMPNALSALSDLHAHKLRVDPVNFKLLSHCLLV

SEQ ID NO: 32 - protein, 91 aa, Homo sapiens
  MOD_RES 1..91: note = Deamidation N (2) [2?]
  MOD_RES 1..91: note = Glycation [?]
  MOD_RES 79: note = Carboxymethyl C [79]
AEALERMFLSFPTTKTYFPHFDLSHGSAQVKGHGKKVADALTNAVAHVDDMPNALSALSDLHAHKLRVDPVNFKLLSHCLLVTLAAHLPAE

SEQ ID NO: 33 - protein, 114 aa, Homo sapiens
  MOD_RES 1..114: note = Glycation (4) [4?]
  MOD_RES 6..50: note = Oxidation M (2) [6 and/or 50]
  MOD_RES 78: note = Carboxymethyl C [78]
EALERMFLSFPTTKTYFPHFDLSHGSAQVKGHGKKVADALNAVAHVDDMPNALSALSDLHAHKLRVDPVNFKLLSHCLLVTLAAHLPAEFTPAVHASLDKFLASVSTVLTSKYR

SEQ ID NO: 34 - protein, 89 aa, Homo sapiens
  MOD_RES 1..89: note = Oxidation M [?]
  MOD_RES 1..89: note = Deamidation N [?]
  MOD_RES 77: note = Carboxymethyl C [77]
ALERMFLSFPTTKTYFPHFDLSHGSAQVKGHGKKVADALTNAVAHVDDMPNALSALSDLHAHKLRVDPVNFKLLSHCLLVTLAAHLPAE

SEQ ID NO: 35 - protein, 87 aa, Homo sapiens
  MOD_RES 1..87: note = Oxidation M [?]
  MOD_RES 1..87: note = Glycation (2) [2?]
  MOD_RES 1..87: note = Deamidation N [?]
  MOD_RES 77: note = Carboxymethyl C [77]
ALERMFLSFPTTKTYFPHFDLSHGSAQVKGHGKKVADALTNAVAHVDDMPNALSALSDLHAHKLRVDPVNFKLLSHCLLVTLAAHLP

SEQ ID NO: 36 - protein, 69 aa, Homo sapiens
  MOD_RES 1..69: note = Glycation (4) [4?]
  MOD_RES 5..49: note = Oxidation M (2) [5 and/or 49]
ALERMFLSFPTTKTYFPHFDLSHGSAQVKGHGKKVADALTNAVAHVDDMPNALSALSDLHAHKLRVDPV

SEQ ID NO: 37 - protein, 65 aa, Homo sapiens
  MOD_RES 1..65: note = Glycation (4) [4?]
  MOD_RES 1..65: note = Deamidation N [?]
  MOD_RES 5..49: note = Oxidation M (2) [5 and/or 49]
ALERMFLSFPTTKTYFPHFDLSHGSAQVKGHGKKVADALTNAVAHVDDMPNALSALSDLHAHKLR

SEQ ID NO: 38 - protein, 64 aa, Homo sapiens
  MOD_RES 1..64: note = Glycation [?]
  MOD_RES 1..64: note = Deamidation N [?]
ALERMFLSFPTTKTYFPHFDLSHGSAQVKGHGKKVADALTNAVAHVDDMPNALSALSDLHAHKL

SEQ ID NO: 39 - protein, 63 aa, Homo sapiens
  MOD_RES 1..63: note = Glycation (3) [3?]
  MOD_RES 1..63: note = Deamidation N [?]
ALERMFLSFPTTKTYFPHFDLSHGSAQVKGHGKKVADALTNAVAHVDDMPNALSALSDLHAHK

SEQ ID NO: 40 - protein, 62 aa, Homo sapiens
  MOD_RES 1..62: note = Oxidation M [?]
  MOD_RES 13..34: note = Glycation (4) [13, 29, 33, and/or 34]
ALERMFLSFPTTKTYFPHFDLSHGSAQVKGHGKKVADALTNAVAHVDDMPNALSALSDLHAH

SEQ ID NO: 41 - protein, 60 aa, Homo sapiens
  MOD_RES 1..60: note = Glycation (3) [3?]
ALERMFLSFPTTKTYFPHFDLSHGSAQVKGHGKKVADALTNAVAHVDDMPNALSALSDLH

SEQ ID NO: 42 - protein, 57 aa, Homo sapiens
  MOD_RES 1..57: note = Glycation (2) [2?]
  MOD_RES 41..51: note = Deamidation N (2) [41 and/or 51]
ALERMFLSFPTTKTYFPHFDLSHGSAQVKGHGKKVADALTNAVAHVDDMPNALSALS

SEQ ID NO: 43 - protein, 53 aa, Homo sapiens
  MOD_RES 1..53: note = Oxidation M [?]
  MOD_RES 13..34: note = Glycation (4) [13, 29, 33, and/or 34]
  MOD_RES 41..51: note = Deamidation N (2) [41 and/or 51]
ALERMFLSFPTTKTYFPHFDLSHGSAQVKGHGKKVADALTNAVAHVDDMPNAL

SEQ ID NO: 44 - protein, 46 aa, Homo sapiens
  MOD_RES 5: note = Oxidation M [5]
ALERMFLSFPTTKTYFPHFDLSHGSAQVKGHGKKVADALTNAVAHV

SEQ ID NO: 45 - protein, 43 aa, Homo sapiens
  MOD_RES 1..43: note = Glycation [?]
  MOD_RES 41: note = Deamidation N [41]
ALERMFLSFPTTKTYFPHFDLSHGSAQVKGHGKKVADALTNAV

SEQ ID NO: 46 - protein, 83 aa, Homo sapiens
  MOD_RES 1..83: note = Glycation (2) [2?]
  MOD_RES 41..70: note = Deamidation N (3) [41, 51, and/or 70]
  MOD_RES 77: note = Carboxymethyl C [77]
ALERMFLSFPTTKTYFPHFDLSHGSAQVKGHGKKVADALTNAVAHVDDMPNALSALSDLHAHKLRVDPVNFKLLSHCLLVTLA

SEQ ID NO: 47 - protein, 21 aa, Homo sapiens
  MOD_RES 13: note = Glycation [13]
ALERMFLSFPTTKTYFPHFDL

SEQ ID NO: 48 - protein, 11 aa, Homo sapiens
  MOD_RES 5: note = Oxidation M [5]
ALERMFLSFPT

SEQ ID NO: 49 - protein, 80 aa, Homo sapiens
  MOD_RES 1..80: note = Deamidation N (2) [2?]
  MOD_RES 1..80: note = Glycation (5) [5?]
  MOD_RES 1..80: note = Oxidation M [?]
  MOD_RES 77: note = Carboxymethyl C [77]
ALERMFLSFPTTKTYFPHFDLSHGSAQVKGHGKKVADALTNAVAHVDDMPNALSALSDLHAHKLRVDPVNFKLLSHCLLV

SEQ ID NO: 50 - protein, 87 aa, Homo sapiens
  MOD_RES 1..87: note = Glycation (4) [4?]
  MOD_RES 75: note = Carboxymethyl C [75]
ERMFLSFPTTKTYFPHFDLSHGSAQVKGHGKKVADALTNAVAHVDDMPNALSALSDLHAHKLRVDPVNFKLLSHCLLVTLAAHLPAE

SEQ ID NO: 51 - protein, 114 aa, Homo sapiens
  MOD_RES 1..114: note = Oxidation M [?]
  MOD_RES 1..114: note = Glycation (5) [5?]
  MOD_RES 41..70: note = Deamidation N (3) [41, 51, and/or 70]
  MOD_RES 77: note = Carboxymethyl C [77]
ALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSLVLTSKYR

SEQ ID NO: 52 - protein, 86 aa, Homo sapiens
  MOD_RES 1..86: note = Glycation (3) [3?]
  MOD_RES 1..86: note = Oxidation M [?]
  MOD_RES 1..86: note = Deamidation N (2) [2?]
  MOD_RES 1..86: note = Glycation [?]
  MOD_RES 2..46: note = Oxidation M (2) [2 and/or 46]
  MOD_RES 74: note = Carboxymethyl C [74]
RMFLSFPTTKTYFPHFDLSHGSAQVKGHGKKVADALTNAVAHVDDMPNALSALSDLHAHKLRVDPVNFKLLSHCLLVTLAAHLPAE

SEQ ID NO: 53 - protein, 100 aa, Homo sapiens
  MOD_RES 1..100: note = Glycation [?]
  MOD_RES 1..100: note = Deamidation N (2) [2?]
  MOD_RES 35: note = Oxidation M [35]
  MOD_RES 63: note = Carboxymethyl C [63]
YFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 54 - protein, 97 aa, Homo sapiens
  MOD_RES 1..97: note = Glycation (3) [3?]
  MOD_RES 12..95: note = Glycation (7) [12, 16, 17, 46, 55, 83, and/or 95]
  MOD_RES 32: note = Oxidation M [32]
  MOD_RES 60: note = Carboxymethyl C [60]
HFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 55 - protein, 94 aa, Homo sapiens
  MOD_RES 1..94: note = Deamidation N [?]
  MOD_RES 9..92: note = Glycation (7) [9, 13, 14, 43, 52, 80, and/or 92]
  MOD_RES 29: note = Oxidation M [29]
  MOD_RES 57: note = Carboxymethyl C [57]
LSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 56 - protein, 93 aa, Homo sapiens
  MOD_RES 1..93: note = Glycation (4) [4?]
  MOD_RES 28: note = Oxidation M [28]
  MOD_RES 56: note = Carboxymethyl C [56]
SHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 57 - protein, 91 aa, Homo sapiens
  MOD_RES 1..91: note = Deamidation N [?]
  MOD_RES 1..91: note = Glycation (2) [2 ?]
  MOD_RES 54: note = Carboxymethyl C [54]
GSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 58 - protein, 90 aa, Homo sapiens
  MOD_RES 25: note = Oxidation M [25]
  MOD_RES 53: note = Carboxymethyl C [53]
SAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 59 - protein, 89 aa, Homo sapiens
  MOD_RES 1..89: note = Glycation (5) [5?]
  MOD_RES 1..89: note = Glycation (3) [3?]
  MOD_RES 16..45: note = Deamidation N (3) [16, 26, and/or 45]
  MOD_RES 24: note = Oxidation M [24]
  MOD_RES 52: note = Carboxymethyl C [52]
AQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 60 - protein, 88 aa, Homo sapiens
  MOD_RES 1..88: note = Deamidation N (2) [2?]
  MOD_RES 1..88: note = Glycation (5) [5?]
  MOD_RES 51: note = Carboxymethyl C [51]
QVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 61 - protein, 83 aa, Homo sapiens
  MOD_RES 1..83: note = Glycation (4) [4?]
  MOD_RES 1..83: note = Glycation (3) [3?]
  MOD_RES 1..83: note = Deamidation N (2) [2?]
  MOD_RES 10..39: note = Deamidation N (3) [10, 20, and/or 39]
  MOD_RES 46: note = Carboxymethyl C [46]
GKKVADALTNAVAHVDDMPNALSALSDLHAHKLRVDPVNFKLLSHCLLVTLAAHLPAEFTPAVHASLDKFLASVSTVLTSKYR

SEQ ID NO: 62 - protein, 81 aa, Homo sapiens
  MOD_RES 1..81: note = Deamidation N (2) [2?]
  MOD_RES 16: note = Oxidation M [16]
  MOD_RES 44: note = Carboxymethyl C [44]
KVADALTNAVAHVDDMPNALSALSDLHAHKLRVDPVNFKLLSHCLLVTLAAHLPAEFTPAVHASLDKFLASVSTVLTSKYR

SEQ ID NO: 63 - protein, 77 aa, Homo sapiens
  MOD_RES 1..77: note = Glycation [?]
  MOD_RES 1..77: note = Deamidation N (2) [2?]
  MOD_RES 1..77: note = Glycation (3) [3?]
  MOD_RES 1..77: note = Deamidation N [?]
  MOD_RES 40: note = Carboxymethyl C [40]
ALTNAVAHVDDMPNALSALSDLHAHKLRVDPVNFKLLSHCLLVTLAAHLPAEFTPAVHASLDKFLASVSTVLTSKYR

SEQ ID NO: 64 - protein, 76 aa, Homo sapiens
  MOD_RES 1..76: note = Glycation (2) [2?]
  MOD_RES 3..32: note = Deamidation N (3) [3, 13, and/or 32]
  MOD_RES 39: note = Carboxymethyl C [39]
LTNAVAHVDDMPNALSALSDLHAHKLRVDPVNFKLLSHCLLVTLAAHLPAEFTPAVHASLDKFLASVSTVLTSKYR

SEQ ID NO: 65 - protein, 75 aa, Homo sapiens
  MOD_RES 10: note = Oxidation M [10]
  MOD_RES 38: note = Carboxymethyl C [38]
TNAVAHVDDMPNALSALSDLHAHKLRVDPVNFKLLSHCLLVTLAAHLPAEFTPAVHASLDKFLASVSTVLTSKYR

SEQ ID NO: 66 - protein, 74 aa, Homo sapiens
  MOD_RES 1..74: note = Deamidation N (2) [2?]
  MOD_RES 1..74: note = Glycation (2) [2?]
  MOD_RES 9: note = Oxidation M [9]
  MOD_RES 37: note = Carboxymethyl C [37]
NAVAHVDDMPNALSALSDLHAHKLRVDPVNFKLLSHCLLVTLAAHLPAEFTPAVHASLDKFLASVSTVLTSKYR

SEQ ID NO: 67 - protein, 107 aa, Homo sapiens
  MOD_RES 1..107: note = Glycation (2) [2?]
  MOD_RES 70: note = Carboxymethyl C [70]
SFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 68 - protein, 66 aa, Homo sapiens
  MOD_RES 1..66: note = Glycation (2) [2?]
  MOD_RES 3..22: note = Deamidation N (2) [3 and/or 22]
  MOD_RES 29: note = Carboxymethyl C [29]
MPNALSALSDLHAHKLRVDPVNFKLLSHCLLVTLAAHLPAEFTPAVHASLDKFLASVSTVLTSKYR

SEQ ID NO: 69 - protein, 62 aa, Homo sapiens
  MOD_RES 1..62: note = Glycation (3) [3?]
  MOD_RES 25: note = Carboxymethyl C [25]
LSALSDLHAHKLRVDPVNFKLLSHCLLVTLAAHLPAEFTPAVHASLDKFLASVSTVLTSKYR

SEQ ID NO: 70 - protein, 61 aa, Homo sapiens
  MOD_RES 1..61: note = Glycation (3) [3?]
  MOD_RES 1..61: note = Glycation [?]
  MOD_RES 17: note = Deamidation N [17]
  MOD_RES 24: note = Carboxymethyl C [24]
SALSDLHAHKLRVDPVNFKLLSHCLLVTLAAHLPAEFTPAVHASLDKFLASVSTVLTSKYR

SEQ ID NO: 71 - protein, 60 aa, Homo sapiens
  MOD_RES 9..58: note = Glycation (4) [9, 18, 46, and/or 58]
  MOD_RES 23: note = Carboxymethyl C [23]
ALSDLHAHKLRVDPVNFKLLSHCLLVTLAAHLPAEFTPAVHASLDKFLASVSTVLTSKYR

SEQ ID NO: 72 - protein, 59 aa, Homo sapiens
  MOD_RES 8..57: note = Glycation (4) [8, 17, 45, and/or 57]
  MOD_RES 22: note = Carboxymethyl C [22]
LSDLHAHKLRVDPVNFKLLSHCLLVTLAAHLPAEFTPAVHASLDKFLASVSTVLTSKYR

SEQ ID NO: 73 - protein, 54 aa, Homo sapiens
  MOD_RES 3..52: note = Glycation (4) [3, 12, 40, and/or 52]
  MOD_RES 17: note = Carboxymethyl C [17]
AHKLRVDPVNFKLLSHCLLVTLAAHLPAEFTPAVHASLDKFLASVSTVLTSKYR

SEQ ID NO: 74 - protein, 52 aa, Homo sapiens
  MOD_RES 1..52: note = Glycation (3) [3?]
  MOD_RES 15: note = Carboxymethyl C [15]
KLRVDPVNFKLLSHCLLVTLAAHLPAEFTPAVHASLDKFLASVSTVLTSKYR

SEQ ID NO: 75 - protein, 51 aa, Homo sapiens
  MOD_RES 7: note = Deamidation N [7]
  MOD_RES 9..49: note = Glycation (3) [9, 37, and/or 49]
  MOD_RES 14: note = Carboxymethyl C [14]
LRVDPVNFKLLSHCLLVTLAAHLPAEFTPAVHASLDKFLASVSTVLTSKYR

SEQ ID NO: 76 - protein, 48 aa, Homo sapiens
  MOD_RES 11: note = Carboxymethyl C [11]
DPVNFKLLSHCLLVTLAAHLPAEFTPAVHASLDKFLASVSTVLTSKYR

SEQ ID NO: 77 - protein, 47 aa, Homo sapiens
  MOD_RES 10: note = Carboxymethyl C [10]
PVNFKLLSHCLLVTLAAHLPAEFTPAVHASLDKFLASVSTVLTSKYR

SEQ ID NO: 78 - protein, 45 aa, Homo sapiens
  MOD_RES 8: note = Carboxymethyl C [8]
NFKLLSHCLLVTLAAHLPAEFTPAVHASLDKFLASVSTVLTSKYR

SEQ ID NO: 79 - protein, 44 aa, Homo sapiens
  MOD_RES 2..42: note = Glycation (3) [2, 30, and/or 42]
  MOD_RES 7: note = Carboxymethyl C [7]
FKLLSHCLLVTLAAHLPAEFTPAVHASLDKFLASVSTVLTSKYR

SEQ ID NO: 80 - protein, 43 aa, Homo sapiens
  MOD_RES 1..43: note = Glycation [?]
  MOD_RES 6: note = Carboxymethyl C [6]
KLLSHCLLVTLAAHLPAEFTPAVHASLDKFLASVSTVLTSKYR

SEQ ID NO: 81 - protein, 39 aa, Homo sapiens
  MOD_RES 1..39: note = Glycation [?]
  MOD_RES 2: note = Carboxymethyl C [2]
HCLLVTLAAHLPAEFTPAVHASLDKFLASVSTVLTSKYR

SEQ ID NO: 82 - protein, 35 aa, Homo sapiens
  MOD_RES 21..33: note = Glycation (2) [21 and/or 33]
VTLAAHLPAEFTPAVHASLDKFLASVSTVLTSKYR

SEQ ID NO: 83 - protein, 69 aa, Homo sapiens
  MOD_RES 1..69: note = Deamidation N (2) [2?]
  MOD_RES 1..69: note = Glycation (2) [2?]
  MOD_RES 1..69: note = Glycation (3) [3?]
  MOD_RES 2..46: note = Oxidation M (2) [2 and/or 46]
RMFLSFPTTKTYFPHFDLSHGSAQVKGHGKKVADALTNAVAHVDDMPNALSALSDLHAHKLRVDPVNFK

SEQ ID NO: 84 - protein, 67 aa, Homo sapiens
  MOD_RES 1..67: note = Glycation [?]
  MOD_RES 1..67: note = Deamidation N [?]
  MOD_RES 2..46: note = Oxidation M (2) [2 and/or 46]
RMFLSFPTTKTYFPHFDLSHGSAQVKGHGKKVADALTNAVAHVDDMPNALSALSDLHAHKLRVDPVN

SEQ ID NO: 85 - protein, 66 aa, Homo sapiens
  MOD_RES 1..66: note = Glycation (3) [3?]
  MOD_RES 2..46: note = Oxidation M (2) [2 and/or 46]
  MOD_RES 38..48: note = Deamidation N (2) [38 and/or 48]
RMFLSFPTTKTYFPHFDLSHGSAQVKGHGKKVADALTNAVAHVDDMPNALSALSDLHAHKLRVDPV

SEQ ID NO: 86 - protein, 64 aa, Homo sapiens
  MOD_RES 1..64: note = Oxidation M [?]
  MOD_RES 1..64: note = Glycation (3) [3?]
  MOD_RES 1..64: note = Deamidation N [?]
  MOD_RES 1..64: note = Glycation [?]
  MOD_RES 2..46: note = Oxidation M (2) [2 and/or 46]
  MOD_RES 38..48: note = Deamidation N (2) [38 and/or 48]
RMFLSFPTTKTYFPHFDLSHGSAQVKGHGKKVADALTNAVAHVDDMPNALSALSDLHAHKLRVD

SEQ ID NO: 87 - protein, 62 aa, Homo sapiens
  MOD_RES 1..62: note = Oxidation M [?]
  MOD_RES 1..62: note = Glycation [?]
RMFLSFPTTKTYFPHFDLSHGSAQVKGHGKKVADALTNAVAHVDDMPNALSALSDLHAHKLR

SEQ ID NO: 88 - protein, 55 aa, Homo sapiens
  MOD_RES 1..55: note = Oxidation M [?]
  MOD_RES 1..55: note = Glycation (3) [3?]
  MOD_RES 2..46: note = Oxidation M (2) [2 and/or 46]
  MOD_RES 10..31: note = Glycation (4) [10, 26, 30, and/or 31]
  MOD_RES 38..48: note = Deamidation N (2) [38 and/or 48]
RMFLSFPTTKTYFPHFDLSHGSAQVKGHGKKVADALTNAVAHVDDMPNALSALSD

SEQ ID NO: 89 - protein, 53 aa, Homo sapiens
  MOD_RES 1..53: note = Deamidation N [?]
  MOD_RES 1..53: note = Glycation [?]
RMFLSFPTTKTYFPHFDLSHGSAQVKGHGKKVADALTNAVAHVDDMPNALSAL

SEQ ID NO: 90 - protein, 52 aa, Homo sapiens
  MOD_RES 1..52: note = Oxidation M [?]
  MOD_RES 1..52: note = Deamidation N [?]
  MOD_RES 1..52: note = Glycation (3) [3?]
RMFLSFPTTKTYFPHFDLSHGSAQVKGHGKKVADALTNAVAHVDDMPNALSA

SEQ ID NO: 91 - protein, 51 aa, Homo sapiens
  MOD_RES 1..51: note = Glycation (2) [2?]
RMFLSFPTTKTYFPHFDLSHGSAQVKGHGKKVADALTNAVAHVDDMPNALS

SEQ ID NO: 92 - protein, 49 aa, Homo sapiens
  MOD_RES 1..49: note = Oxidation M [?]
  MOD_RES 1..49: note = Deamidation N [?]
RMFLSFPTTKTYFPHFDLSHGSAQVKGHGKKVADALTNAVAHVDDMPNA

SEQ ID NO: 93 - protein, 46 aa, Homo sapiens
  MOD_RES 1..46: note = Glycation [?]
  MOD_RES 38: note = Deamidation N (38]
RMFLSFPTTKTYFPHFDLSHGSAQVKGHGKKVADALTNAVAHVDDM

SEQ ID NO: 94 - protein, 43 aa, Homo sapiens
  MOD_RES 30..31: note = Glycation (2) [30 and/or 31]
RMFLSFPTTKTYFPHFDLSHGSAQVKGHGKKVADALTNAVAHV

SEQ ID NO: 95 - protein, 40 aa, Homo sapiens
  MOD_RES 2: note = Oxidation M [2]
  MOD_RES 38: note = Deamidation N [38]
RMFLSFPTTKTYFPHFDLSHGSAQVKGHGKKVADALTNAV

SEQ ID NO: 96 - protein, 39 aa, Homo sapiens
  MOD_RES 1..39: note = Glycation (3) [3?]
  MOD_RES 38: note = Deamidation N [38]
RMFLSFPTTKTYFPHFDLSHGSAQVKGHGKKVADALTNA

SEQ ID NO: 97 - protein, 81 aa, Homo sapiens
  MOD_RES 1..81: note = Glycation (3) [3?]
  MOD_RES 2..46: note = Oxidation M (2) [2 and/or 46]
  MOD_RES 38..67: note = Deamidation N (3) [38, 48, and/or 67]
  MOD_RES 74: note = Carboxymethyl C [74]
RMFLSFPTTKTYFPHFDLSHGSAQVKGHGKKVADALTNAVAHVDDMPNALSALSDLHAHKLRVDPVNFKLLSHCLLVTLAA

SEQ ID NO: 98 - protein, 33 aa, Homo sapiens
  MOD_RES 1..33: note = Glycation (3) [3?]
  MOD_RES 2: note = Oxidation M [2]
RMFLSFPTTKTYFPHFDLSHGSAQVKGHGKKVA

SEQ ID NO: 99 - protein, 30 aa, Homo sapiens
  MOD_RES 1..30: note = Glycation [?]
  MOD_RES 2: note = Oxidation M [2]
RMFLSFPTTKTYFPHFDLSHGSAQVKGHGK

SEQ ID NO: 100 - protein, 80 aa, Homo sapiens
  MOD_RES 1..80: note = Deamidation N (2) [2?]
  MOD_RES 1..80: note = Glycation (5) [5?]
  MOD_RES 2..46: note = Oxidation M (2) [2 and/or 46]
  MOD_RES 74: note = Carboxymethyl C [74]
RMFLSFPTTKTYFPHFDLSHGSAQVKGHGKKVADALTNAVAHVDDMPNALSALSDLHAHKLRVDPVNFKLLSHCLLVTLA

SEQ ID NO: 101 - protein, 7 aa, Homo sapiens
  MOD_RES 2: note = Oxidation M [2]
RMFLSFP

SEQ ID NO: 102 - protein, 5 aa, Homo sapiens
  MOD_RES 2: note = Oxidation M [2]
RMFLS

SEQ ID NO: 103 - protein, 85 aa, Homo sapiens
  MOD_RES 1..85: note = Oxidation M [?]
  MOD_RES 1..85: note = Deamidation N [?]
  MOD_RES 1..85: note = Glycation [?]
  MOD_RES 9..68: note = Glycation (6) [9, 25, 29, 30, 59, and/or 68]
  MOD_RES 37..66: note = Deamidation N (3) [37, 47, and/or 66]
  MOD_RES 73: note = Carboxymethyl C [73]
MFLSFPTTKTYFPHFDLSHGSAQVKGHGKKVADALTNAVAHVDDMPNALSALSDLHAHKLRVDPVNFKLLSHCLLVTLAAHLPAE

SEQ ID NO: 104 - protein, 76 aa, Homo sapiens
  MOD_RES 1..76: note = Glycation (2) [2?]
  MOD_RES 1..76: note = Glycation (3) [3?]
  MOD_RES 28..57: note = Deamidation N (3) [28, 38, and/or 57]
  MOD_RES 36: note = Oxidation M [36]
  MOD_RES 64: note = Carboxymethyl C [64]
TYFPHFDLSHGSAQVKGHGKKVADALTNAVAHVDDMPNALSALSDLHAHKLRVDPVNFKLLSHCLLVTLAAHLPAE

SEQ ID NO: 105 - protein, 75 aa, Homo sapiens
  MOD_RES 1..75: note = Glycation (2) [2?]
  MOD_RES 27..56: note = Deamidation N (3) [27, 37, and/or 56]
  MOD_RES 35: note = Oxidation M [35]
  MOD_RES 63: note = Carboxymethyl C [63]
YFPHFDLSHGSAQVKGHGKKVADALTNAVAHVDDMPNALSALSDLHAHKLRVDPVNFKLLSHCLLVTLAAHLPAE

SEQ ID NO: 106 - protein, 74 aa, Homo sapiens
  MOD_RES 26..55: note = Deamidation N (3) [26, 36, and/or 55]
  MOD_RES 34: note = Oxidation M [34]
  MOD_RES 62: note = Carboxymethyl C [62]
FPHFDLSHGSAQVKGHGKKVADALTNAVAHVDDMPNALSALSDLHAHKLRVDPVNFKLLSHCLLVTLAAHLPAE

SEQ ID NO: 107 - protein, 72 aa, Homo sapiens
  MOD_RES 32: note = Oxidation M [32]
  MOD_RES 60: note = Carboxymethyl C [60]
HFDLSHGSAQVKGHGKKVADALTNAVAHVDDMPNALSALSDLHAHKLRVDPVNFKLLSHCLLVTLAAHLPAE

SEQ ID NO: 108 - protein, 69 aa, Homo sapiens
  MOD_RES 1..69: note = Glycation [?]
  MOD_RES 57: note = Carboxymethyl C [57]
LSHGSAQVKGHGKKVADALTNAVAHVDDMPNALSALSDLHAHKLRVDPVNFKLLSHCLLVTLAAHLPAE

SEQ ID NO: 109 - protein, 67 aa, Homo sapiens
  MOD_RES 1..67: note = Glycation (4) [4?]
  MOD_RES 1..67: note = Deamidation N (2) [2?]
  MOD_RES 7..50: note = Glycation (5) [7, 11, 12, 41, and/or 50]
  MOD_RES 55: note = Carboxymethyl C [55]
HGSAQVKGHGKKVADALTNAVAHVDDMPNALSALSDLHAHKLRVDPVNFKLLSHCLLVTLAAHLPAE

SEQ ID NO: 110 - protein, 84 aa, Homo sapiens
  MOD_RES 36..65: note = Deamidation N (3) [36, 46, and/or 65]
  MOD_RES 72: note = Carboxymethyl C [72]
FLSFPTTKTYFPHFDLSHGSAQVKGHGKKVADALTNAVAHVDDMPNALSALSDLHAHKLRVDPVNFKLLSHCLLVTLAAHLPAE

SEQ ID NO: 111 - protein, 66 aa, Homo sapiens
  MOD_RES 1..66: note = Deamidation N [?]
  MOD_RES 6..49: note = Glycation (5) [6, 10, 11, 40, and/or 49]
  MOD_RES 54: note = Carboxymethyl C [54]
GSAQVKGHGKKVADALTNAVAHVDDMPNALSALSDLHAHKLRVDPVNFKLLSHCLLVTLAAHLPAE

SEQ ID NO: 112 - protein, 63 aa, Homo sapiens
  MOD_RES 15..44: note = Deamidation N (3) [15, 25, and/or 44]
  MOD_RES 51: note = Carboxymethyl C [51]
QVKGHGKKVADALTNAVAHVDDMPNALSALSDLHAHKLRVDPVNFKLLSHCLLVTLAAHLPAE

SEQ ID NO: 113 - protein, 62 aa, Homo sapiens
  MOD_RES 1..62: note = Glycation (3) [3?]
  MOD_RES 1..62: note = Glycation (4) [4?]
  MOD_RES 1..62: note = Deamidation N (2) [2?]
  MOD_RES 2..45: note = Glycation (5) [2, 6, 7, 36, and/or 45]
  MOD_RES 22: note = Oxidation M [22]
  MOD_RES 50: note = Carboxymethyl C [50]
VKGHGKKVADALTNAVAHVDDMPNALSALSDLHAHKLRVDPVNFKLLSHCLLVTLAAHLPAE

SEQ ID NO: 114 - protein, 61 aa, Homo sapiens
  MOD_RES 1..61: note = Glycation (3) [3?]
  MOD_RES 1..44: note = Glycation (5) [1, 5, 6, 35, and/or 44]
  MOD_RES 1..61: note = Deamidation N [?]
  MOD_RES 1..61: note = Glycation (4) [4?]
  MOD_RES 1..61: note = Deamidation N (2) [2?]
  MOD_RES 13..42: note = Deamidation N (3) [13, 23, and/or 42]
  MOD_RES 21: note = Oxidation M [21]
  MOD_RES 49: note = Carboxymethyl C [49]
KGHGKKVADALTNAVAHVDDMPNALSALSDLHAHKLRVDPVNFKLLSHCLLVTLAAHLPAE

SEQ ID NO: 115 - protein, 60 aa, Homo sapiens
  MOD_RES 1..60: note = Deamidation N (2) [2?]
  MOD_RES 1..60: note = Glycation (3) [3?]
  MOD_RES 48: note = Carboxymethyl C [48]
GHGKKVADALTNAVAHVDDMPNALSALSDLHAHKLRVDPVNFKLLSHCLLVTLAAHLPAE

SEQ ID NO: 116 - protein, 59 aa, Homo sapiens
  MOD_RES 1..59: note = Deamidation N (2) [2?]
  MOD_RES 1..59: note = Deamidation N [?]
  MOD_RES 3..42: note = Glycation (4) [3, 4, 33, and/or 42]
  MOD_RES 11..40: note = Deamidation N (3) [11, 21, and/or 40]
  MOD_RES 47: note = Carboxymethyl C [47]
HGKKVADALTNAVAHVDDMPNALSALSDLHAHKLRVDPVNFKLLSHCLLVTLAAHLPAE

SEQ ID NO: 117 - protein, 58 aa, Homo sapiens
  MOD_RES 1..58: note = Deamidation N (2) [2?]
  MOD_RES 46: note = Carboxymethyl C [46]
GKKVADALTNAVAHVDDMPNALSALSDLHAHKLRVDPVNFKLLSHCLLVTLAAHLPAE

SEQ ID NO: 118 - protein, 57 aa, Homo sapiens
  MOD_RES 1..57: note = Deamidation N (2) [2?]
  MOD_RES 1..57: note = Glycation (3) [3?]
  MOD_RES 1..57: note = Deamidation N [?]
  MOD_RES 17: note = Oxidation M [17]
  MOD_RES 45: note = Carboxymethyl C [45]
KKVADALTNAVAHVDDMPNALSALSDLHAHKLRVDPVNFKLLSHCLLVTLAAHLPAE

SEQ ID NO: 119 - protein, 55 aa, Homo sapiens
  MOD_RES 1..55: note = Glycation [?]
  MOD_RES 16: note = Oxidation M [16]
  MOD_RES 44: note = Carboxymethyl C [44]
KVADALTNAVAHVDDMPNALSALSDLHAHKLRVDPVNFKLLSHCLLVTLMHLPAE

SEQ ID NO: 120 - protein, 55 aa, Homo sapiens
  MOD_RES 1..54: note = Glycation [?]
  MOD_RES 1..54: note = Glycation [?]
  MOD_RES 1..54: note = Deamidation N (2) [2?]
  MOD_RES 1..54: note = Deamidation N [?]
  MOD_RES 15: note = Oxidation M [15]
  MOD_RES 29..38: note = Glycation (2) [29 and/or 38]
  MOD_RES 43: note = Carboxymethyl C [43]
VADALTNAVAHVDDMPNALSALSDLHAHKLRVDPVNFKLLSHCLLVTLAAHLPAE

SEQ ID NO: 121 - protein, 52 aa, Homo sapiens
  MOD_RES 1..52: note = Glycation [?]
  MOD_RES 1..52: note = Deamidation N [?]
  MOD_RES 12: note = Oxidation M [12]
  MOD_RES 40: note = Carboxymethyl C [40]
ALTNAVAHVDDMPNALSALSDLHAHKLRVDPVNFKLLSHCLLVTLAAHLPAE

SEQ ID NO: 122 - protein, 51 aa, Homo sapiens
  MOD_RES 1..51: note = Glycation [?]
  MOD_RES 11: note = Oxidation M [11]
  MOD_RES 39: note = Carboxymethyl C [39]
LTNAVAHVDDMPNALSALSDLHAHKLRVDPVNFKLLSHCLLVTLAAHLPAE

SEQ ID NO: 123 - protein, 50 aa, Homo sapiens
  MOD_RES 1..50: note = Deamidation N (2) [2?]
  MOD_RES 2..31: note = Deamidation N (3) [2, 12, and/or 31]
  MOD_RES 10: note = Oxidation M [10]
  MOD_RES 38: note = Carboxymethyl C [38]
TNAVAHVDDMPNALSALSDLHAHKLRVDPVNFKLLSHCLLVTLAAHLPAE

SEQ ID NO: 124 - protein, 48 aa, Homo sapiens
  MOD_RES 8: note = Oxidation M [8]
  MOD_RES 36: note = Carboxymethyl C [36]
AVAHVDDMPNALSALSDLHAHKLRVDPVNFKLLSHCLLVTLAAHLPAE

SEQ ID NO: 125 - protein, 82 aa, Homo sapiens
  MOD_RES 1..82: note = Glycation (5) [5?]
  MOD_RES 42: note = Oxidation M [42]
  MOD_RES 70: note = Carboxymethyl C [70]
SFPTTKTYFPHFDLSHGSAQVKGHGKKVADALTNAVAHVDDMPNALSALSDLHAHKLRVDPVNFKLLSHCLLVTLAAHLPAE

SEQ ID NO: 126 - protein, 45 aa, Homo sapiens
  MOD_RES 1..45: note = Glycation [?]
  MOD_RES 1..45: note = Deamidation N [?]
  MOD_RES 5: note = Oxidation M [5]
  MOD_RES 33: note = Carboxymethyl C [33]
HVDDMPNALSALSDLHAHKLRVDPVNFKLLSHCLLVTLAAHLPAE

SEQ ID NO: 127 - protein, 42 aa, Homo sapiens
  MOD_RES 1..42: note = Glycation [?]
  MOD_RES 2: note = Oxidation M [2]
  MOD_RES 4..23: note = Deamidation N (2) [4 and/or 23]
  MOD_RES 30: note = Carboxymethyl C [30]
DMPNALSALSDLHAHKLRVDPVNFKLLSHCLLVTLAAHLPAE

SEQ ID NO: 128 - protein, 40 aa, Homo sapiens
  MOD_RES 14..23: note = Glycation (2) [14 and/or 23]
  MOD_RES 28: note = Carboxymethyl C [28]
PNALSALSDLHAHKLRVDPVNFKLLSHCLLVTLAAHLPAE

SEQ ID NO: 129 - protein, 39 aa, Homo sapiens
  MOD_RES 1..39: note = Glycation [?]
  MOD_RES 27: note = Carboxymethyl C [27]
NALSALSDLHAHKLRVDPVNFKLLSHCLLVTLAAHLPAE

SEQ ID NO: 130 - protein, 81 aa, Homo sapiens
  MOD_RES 1..81: note = Glycation (3) [3?]
  MOD_RES 1..81: note = Deamidation N (2) [2?]
  MOD_RES 33..62: note = Deamidation N (3) [33, 43, and/or 62]
  MOD_RES 69: note = Carboxymethyl C [69]
FPTTKTYFPHFDLSHGSAQVKGHGKKVADALTNAVAHVDDMPNALSALSDLHAHKLRVDPVNFKLLSHCLLVTLAAHLPAE

SEQ ID NO: 131 - protein, 33 aa, Homo sapiens
  MOD_RES 1..33: note = Glycation [?]
  MOD_RES 21: note = Carboxymethyl C [21]
SDLHAHKLRVDPVNFKLLSHCLLVTLAAHLPAE

SEQ ID NO: 132 - protein, 30 aa, Homo sapiens
  MOD_RES 4..13: note = Glycation (2) [4 and/or 13]
  MOD_RES 18: note = Carboxymethyl C [18]
HAHKLRVDPVNFKLLSHCLLVTLAAHLPAE

SEQ ID NO: 133 - protein, 19 aa, Homo sapiens
  MOD_RES 2: note = Glycation [2]
  MOD_RES 7: note = Carboxymethyl C [7]
FKLLSHCLLVTLAAHLPAE

SEQ ID NO: 134 - protein, 79 aa, Homo sapiens
  MOD_RES 1..79: note = Deamidation N (2) [2?]
  MOD_RES 67: note = Carboxymethyl C [67]
TTKTYFPHFDLSHGSAQVKGHGKKVADALTNAVAHVDDMPNALSALSDLHAHKLRVDPVNFKLLSHCLLVTLAAHLPAE

SEQ ID NO: 135 - protein, 78 aa, Homo sapiens
  MOD_RES 1..78: note = Deamidation N [?]
  MOD_RES 1..78: note = Glycation [?]
  MOD_RES 1..78: note = Glycation (5) [5?]
  MOD_RES 38: note = Oxidation M [38]
  MOD_RES 66: note = Carboxymethyl C [66]
TKTYFPHFDLSHGSAQVKGHGKKVADALTNAVAHVDDMPNALSALSDLHAHKLRVDPVNFKLLSHCLLVTLAAHLPAE

SEQ ID NO: 136 - protein, 76 aa, Homo sapiens
  MOD_RES 1..76: note = Deamidation N (2) [2?]
  MOD_RES 1..76: note = Glycation (2) [2?]
  MOD_RES 1..76: note = Glycation (3) [3?]
  MOD_RES 37: note = Oxidation M [37]
  MOD_RES 65: note = Carboxymethyl C [65]
KTYFPHFDLSHGSAQVKGHGKKVADALTNAVAHVDDMPNALSALSDLHAHKLRVDPVNFKLLSHCLLVTLMHLPAE

SEQ ID NO: 137 - protein, 14 aa, Homo sapiens
  MOD_RES 12: note = Glycation [12]
  MOD_RES 12: note = Glycation
FLASVSTVLTSKYR

SEQ ID NO: 138 - protein, 9 aa, Homo sapiens
  MOD_RES 7: note = Glycation [7]
STVLTSKYR

SEQ ID NO: 139 - protein, 5 aa, Homo sapiens
  MOD_RES 3: note = Glycation [3]
TSKYR

SEQ ID NO: 140 - protein, 22 aa, Homo sapiens
  MOD_RES 8..20: note = Glycation (2) [8 and/or 20]
AVHASLDKFLASVSTVLTSKYR

SEQ ID NO: 141 - protein, 29 aa, Homo sapiens
  MOD_RES 19: note = Deamidation N [19]
YPVEPFTESQSLTLTDVENLHLPLPLLQS

SEQ ID NO: 142 - protein, 18 aa, Homo sapiens
  MOD_RES 2: note = Oxidation M [2]
DMPIQAFLLYQEPVLGPV

SEQ ID NO: 143 - protein, 10 aa, Homo sapiens
  MOD_RES 2: note = Oxidation M [2]
DMPIQAFLLY

SEQ ID NO: 144 - protein, 23 aa, Homo sapiens
  MOD_RES 5: note = Deamidation N [5]
LEELNVPGEIVESLSSSEESITR

SEQ ID NO: 145 - protein, 19 aa, Homo sapiens
  MOD_RES 1: note = Deamidation N [1]
NVPGEIVESLSSSEESITR

SEQ ID NO: 146 - protein, 34 aa, Homo sapiens
  MOD_RES 1..34: note = Deamidation N I?]
  MOD_RES 30: note = Oxidation M [30]
GPIPNSLPQNIPPLTQTPVVVPPFLQPEVMGVSK

SEQ ID NO: 147 - protein, 33 aa, Homo sapiens
  MOD_RES 29: note = Oxidation M [29]
PIPNSLPQNIPPLTQTPVVVPPFLQPEVMGVSK

SEQ ID NO: 148 - protein, 12 aa, Homo sapiens
  MOD_RES 8: note = Oxidation M [8]
PFLQPEVMGVSK

SEQ ID NO: 149 - protein, 8 aa, Homo sapiens
  MOD_RES 4: note = Oxidation M [4]
PEVMGVSK

SEQ ID NO: 150 - protein, 5 aa, Homo sapiens
  MOD_RES 1: note = Oxidation M [1]
MGVSK

SEQ ID NO: 151 - protein, 9 aa, Homo sapiens
  MOD_RES 3: note = Deamidation N [3]
LVNELTEFA

SEQ ID NO: 152 - protein, 8 aa, Homo sapiens
  MOD_RES 3: note = Deamidation N [3]
LVNELTEF

SEQ ID NO: 153 - protein, 5 aa, Homo sapiens
  MOD_RES 3: note = Deamidation N [3]
LVNEL

SEQ ID NO: 154 - protein, 9 aa, Homo sapiens
  MOD_RES 2: note = Deamidation N [2]
VNELTEFAK

SEQ ID NO: 155 - protein, 11 aa, Homo sapiens
  MOD_RES 9: note = Carboxymethyl C [9]
VADESHAGCEK

SEQ ID NO: 156 - protein, 7 aa, Homo sapiens
  MOD_RES 5: note = Carboxymethyl C [5]
FGDELCK

SEQ ID NO: 157 - protein, 8 aa, Homo sapiens
  MOD_RES 6: note = Deamidation N [6]
LKPDPNTL

SEQ ID NO: 158 - protein, 12 aa, Homo sapiens
  MOD_RES 11: note = Deamidation N [11]
FYAPELLYYANK

SEQ ID NO: 159 - protein, 9 aa, Homo sapiens
  MOD_RES 8: note = Deamidation N [8]
PELLYYANK

SEQ ID NO: 160 - protein, 14 aa, Homo sapiens
  MOD_RES 8..9: note = Carboxymethyl C (2) [8 and/or 9]
YNGVFQECCQAEDK

SEQ ID NO: 161 - protein, 12 aa, Homo sapiens
  MOD_RES 3: note = Carboxymethyl C [3]
YICDNQDTISSK

SEQ ID NO: 162 - protein, 9 aa, Homo sapiens
  MOD_RES 2..3: note = Carboxymethyl C (2) [2 and/or 3]
ECCDKPLLE

SEQ ID NO: 163 - protein, 8 aa, Homo sapiens
  MOD_RES 1: note = Carboxymethyl C [1]
CDKPLLEK

SEQ ID NO: 164 - protein, 6 aa, Homo sapiens
  MOD_RES 6: note = Deamidation N [6]
DAIPEN

SEQ ID NO: 165 - protein, 16 aa, Homo sapiens
  MOD_RES 6: note = Deamidation N [6]
DAIPENLPPLTADFAE

SEQ ID NO: 166 - protein, 15 aa, Homo sapiens
  MOD_RES 6: note = Deamidation N [6]
DAIPENLPPLTADFA

SEQ ID NO: 167 - protein, 14 aa, Homo sapiens
  MOD_RES 6: note = Deamidation N [6]
DAIPENLPPLTADF

SEQ ID NO: 168 - protein, 10 aa, Homo sapiens
  MOD_RES 6: note = Deamidation N [6]
DAIPENLPPL

SEQ ID NO: 169 - protein, 9 aa, Homo sapiens
  MOD_RES 6: note = Deamidation N [6]
VDEPQNLIK

SEQ ID NO: 170 - protein, 10 aa, Homo sapiens
  MOD_RES 8: note = Deamidation N [8]
LGEYGFQNAL

SEQ ID NO: 171 - protein, 6 aa, Homo sapiens
  MOD_RES 1: note = Deamidation N [1]
NALIVR

SEQ ID NO: 172 - protein, 10 aa, Homo sapiens
  MOD_RES 9: note = Deamidation N [9]
EDYLSLILNR

SEQ ID NO: 173 - protein, 12 aa, Homo sapiens
  MOD_RES 9: note = Carboxymethyl C [9]
LFTFHADICTLP

SEQ ID NO: 174 - protein, 11 aa, Homo sapiens
  MOD_RES 4: note = Carboxymethyl C [4]
ADICTLPDTEK

SEQ ID NO: 175 - protein, 12 aa, Homo sapiens
  MOD_RES 3: note = Oxidation M [3]
  MOD_RES 5: note = Deamidation N [5]
TVMENFVAFVDK

SEQ ID NO: 176 - protein, 6 aa, Homo sapiens
  MOD_RES 3: note = Oxidation M [3]
TVMENF

SEQ ID NO: 177 - protein, 11 aa, Homo sapiens
  MOD_RES 2: note = Oxidation M [2]
VMENFVAFVDK

SEQ ID NO: 178 - protein, 9 aa, Homo sapiens
  MOD_RES 2: note = Carboxymethyl C [2]
QCPFDEHVK

SEQ ID NO: 179 - protein, 15 aa, Homo sapiens
  MOD_RES 8: note = Carboxymethyl C [8]
FSQYLQQCPFDEHVK

SEQ ID NO: 180 - protein, 19 aa, Homo sapiens
  MOD_RES 2..18: note = Carboxymethyl C (3) [2, 17, and/or 18]
  MOD_RES 3: note = Glycation [3]
  MOD_RES 14: note = Oxidation M [14]
LCKVASLRETYGDMADCCE

SEQ ID NO: 181 - protein, 12 aa, Homo sapiens
  MOD_RES 2: note = Carboxymethyl C [2]
LCKVASLRETYG

SEQ ID NO: 182 - protein, 11 aa, Homo sapiens
  MOD_RES 2: note = Carboxymethyl C [2]
LCKVASLRETY

SEQ ID NO: 183 - protein, 10 aa, Homo sapiens
  MOD_RES 2: note = Carboxymethyl C [2]
LCKVASLRET

SEQ ID NO: 184 - protein, 18 aa, Homo sapiens
  MOD_RES 1..17: note = Carboxymethyl C (3) [1, 16, and/or 17]
  MOD_RES 2: note = Glycation I2]
CKVASLRETYGDMADCCE

SEQ ID NO: 185 - protein, 17 aa, Homo sapiens
  MOD_RES 12: note = Oxidation M [12]
  MOD_RES 15..16: note = Carboxymethyl C (2) [15 and/or 16]
KVASLRETYGDMADCCE

SEQ ID NO: 186 - protein, 11 aa, Homo sapiens
  MOD_RES 6: note = Oxidation M [6]
  MOD_RES 9..10: note = Carboxymethyl C (2) [9 and/or 10]
ETYGDMADCCE

SEQ ID NO: 187 - protein, 21 aa, Homo sapiens
  MOD_RES 14..15: note = Carboxymethyl C (2) [14 and/or 15]
VASLRETYGDMADCCEKQEPE

SEQ ID NO: 188 - protein, 19 aa, Homo sapiens
  MOD_RES 9: note = Oxidation M [9]
  MOD_RES 12..13: note = Carboxyrnethyl C (2) [12 and/or 13]
  MOD_RES 15: note = Glycation [15]
SLRETYGDMADCCEKQEPE

SEQ ID NO: 189 - protein, 17 aa, Homo sapiens
  MOD_RES 7: note = Oxidation M [7]
  MOD_RES 10..11: note = Carboxymethyl C (2) [10 and/or 11]
  MOD_RES 13: note = Glycation [13]
RETYGDMADCCEKQEPE

SEQ ID NO: 190 - protein, 16 aa, Homo sapiens
  MOD_RES 5: note = Oxidation M [5]
  MOD_RES 9..10: note = Carboxymethyl C (2) [9 and/or 10]
  MOD_RES 12: note = Glycation [12]
ETYGDMADCCEKQEPE

SEQ ID NO: 191 - protein, 26 aa, Homo sapiens
  MOD_RES 1..26: note = Glycation [?]
  MOD_RES 2..18: note = Carboxymethyl C (3) [2, 17, and/or 18]
  MOD_RES 26: note = Deamidation N [26]
LCKVASLRETYGDMADCCEKQEPERN

SEQ ID NO: 192 - protein, 21 aa, Homo sapiens
  MOD_RES 8: note = Oxidation M [8]
  MOD_RES 11..12: note = Carboxymethyl C (2) [11 and/or 12]
LRETYGDMADCCEKQEPERNE

SEQ ID NO: 193 - protein, 20 aa, Homo sapiens
  MOD_RES 7: note = Oxidation M [7]
  MOD_RES 10..11: note = Carboxymethyl C (2) [10 and/or 11]
  MOD_RES 13: note = Glycation [13]
  MOD_RES 19: note = Deamidation N [19]
RETYGDMADCCEKQEPERNE

SEQ ID NO: 194 - protein, 19 aa, Homo sapiens
  MOD_RES 6: note = Oxidation M [6]
  MOD_RES 9..10: note = Carboxymethyl C (2) [9 and/or 10]
  MOD_RES 18: note = Deamidation N [18]
ETYGDMADCCEKQEPERNE

SEQ ID NO: 195 - protein, 6 aa, Homo sapiens
  MOD_RES 2: note = Carboxymethyl C [2]
  MOD_RES 3: note = Glycation [3]
LCKVAS

SEQ ID NO: 196 - protein, 10 aa, Homo sapiens
  MOD_RES 5: note = Oxidation M [5]
  MOD_RES 8..9: note = Carboxyrnethyl C (2) [8 and/or 9]
TYGDMADCCE

SEQ ID NO: 197 - protein, 14 aa, Homo sapiens
  MOD_RES 5: note = Oxidation M [5]
  MOD_RES 8..9: note = Carboxymethyl C (2) [8 and/or 9]
  MOD_RES 11: note = Glycation [11]
TYGDMADCCEKQEP

SEQ ID NO: 198 - protein, 13 aa, Homo sapiens
  MOD_RES 5: note = Oxidation M [5]
  MOD_RES 8..9: note = Carboxymethyl C (2) [8 and/or 9]
  MOD_RES 11: note = Glycation [11]
TYGDMADCCEKQE

SEQ ID NO: 199 - protein, 12 aa, Homo sapiens
  MOD_RES 5: note = Oxidation M [5]
  MOD_RES 8..9: note = Carboxymethyl C (2) [8 and/or 9]
  MOD_RES 11: note = Glycation [11]
TYGDMADCCEKQ

SEQ ID NO: 200 - protein, 11 aa, Homo sapiens
  MOD_RES 5: note = Oxidation M [5]
  MOD_RES 8..9: note = Carboxymethyl C (2) [8 and/or 9]
TYGDMADCCEK

SEQ ID NO: 201 - protein, 13 aa, Homo sapiens
  MOD_RES 3: note = Oxidation M [3]
  MOD_RES 6..7: note = Carboxymethyl C (2) [6 and/or 7]
GDMADCCEKQEPE

SEQ ID NO: 202 - protein, 11 aa, Homo sapiens
  MOD_RES 4..5: note = Carboxymethyl C (2) [4 and/or 5]
MADCCEKQEPE

SEQ ID NO: 203 - protein, 10 aa, Homo sapiens
  MOD_RES 3..4: note = Carboxymethyl C (2) [3 and/or 4]
  MOD_RES 6: note = Glycation [6]
ADCCEKQEPE

SEQ ID NO: 204 - protein, 8 aa, Homo sapiens
  MOD_RES 1..2: note = Carboxymethyl C (2) [1 and/or 2]
  MOD_RES 4: note = Glycation [4]
CCEKQEPE

SEQ ID NO: 205 - protein, 18 aa, Homo sapiens
  MOD_RES 5: note = Oxidation M [5]
  MOD_RES 8..9: note = Carboxymethyl C (2) [8 and/or 9]
  MOD_RES 17: note = Deamidation N [17]
TYGDMADCCEKQEPERNE

SEQ ID NO: 206 - protein, 17 aa, Homo sapiens
  MOD_RES 8..9: note = Carboxymethyl C (2) [8 and/or 9]
  MOD_RES 11: note = Glycation [11]
  MOD_RES 17: note = Deamidation N [17]
TYGDMADCCEKQEPERN

SEQ ID NO: 207 - protein, 16 aa, Homo sapiens
  MOD_RES 5: note = Oxidation M [5]
  MOD_RES 8..9: note = Carboxymethyl C (2) [8 and/or 9]
TYGDMADCCEKQEPER

SEQ ID NO: 208 - protein, 17 aa, Homo sapiens
  MOD_RES 4: note = Oxidation M [4]
  MOD_RES 7..8: note = Carboxymethyl C (2) [7 and/or 8]
  MOD_RES 10: note = Glycation [10]
  MOD_RES 16: note = Deamidation N [16]
YGDMADCCEKQEPERNE

SEQ ID NO: 209 - protein, 16 aa, Homo sapiens
  MOD_RES 6..7: note = Carboxymethyl C (2) [6 and/or 7]
  MOD_RES 9: note = Glycation [9]
  MOD_RES 15: note = Deamidation N [15]
GDMADCCEKQEPERNE

SEQ ID NO: 210 - protein, 15 aa, Homo sapiens
  MOD_RES 2: note = Oxidation M [2]
  MOD_RES 5..6: note = Carboxymethyl C (2) [5 and/or 6]
  MOD_RES 8: note = Glycation [8]
  MOD_RES 14: note = Deamidation N [14]
DMADCCEKQEPERNE

SEQ ID NO: 211 - protein, 14 aa, Homo sapiens
  MOD_RES 4..5: note = Carboxymethyl C (2) [4 and/or 5]
  MOD_RES 7: note = Glycation [7]
  MOD_RES 13: note = Deamidation N [13]
MADCCEKQEPERNE

SEQ ID NO: 212 - protein, 13 aa, Homo sapiens
  MOD_RES 3..4: note = Carboxymethyl C (2) [3 and/or 4]
  MOD_RES 6: note = Glycation [6]
  MOD_RES 12: note = Deamidation N [12]
ADCCEKQEPERNE

SEQ ID NO: 213 - protein, 11 aa, Homo sapiens
  MOD_RES 1..2: note = Carboxymethyl C (2) [1 and/or 2]
  MOD_RES 4: note = Glycation [4]
  MOD_RES 10: note = Deamidation N [10]
CCEKQEPERNE

SEQ ID NO: 214 - protein, 10 aa, Homo sapiens
  MOD_RES 1: note = Carboxymethyl C [1]
  MOD_RES 9: note = Deamidation N [9]
CEKQEPERNE

SEQ ID NO: 215 - protein, 43 aa, Homo sapiens
  MOD_RES 1..43: note = Glycation (2) [2 ?]
  MOD_RES 1..43: note = Deamidation N [?]
  MOD_RES 5: note = Oxidation M [5]
  MOD_RES 8..41: note = Carboxymethyl C (4) [8, 9, 19, and/or 41]
TYGDMADCCEKQEPERNECFLSHKDDSPDLPKLKPDPNTLCDE

SEQ ID NO: 216 - protein, 32 aa, Homo sapiens
  MOD_RES 5: note = Oxidation M [5]
  MOD_RES 8..19: note = Carboxymethyl C (3) [8, 9, and/or 19]
  MOD_RES 11..32: note = Glycation (3) [11, 24, and/or 32]
TYGDMADCCEKQEPERNECFLSHKDDSPDLPK

SEQ ID NO: 217 - protein, 31 aa, Homo sapiens
  MOD_RES 5: note = Oxidation M [5]
  MOD_RES 8..19: note = Carboxyrnethyl C (3) [8, 9, and/or 19]
  MOD_RES 17: note = Deamidation N [17]
TYGDMADCCEKQEPERNECFLSHKDDSPDLP

SEQ ID NO: 218 - protein, 30 aa, Homo sapiens
  MOD_RES 8..19: note = Carboxymethyl C (3) [8, 9, and/or 19]
  MOD_RES 17: note = Deamidation N [17]
TYGDMADCCEKQEPERNECFLSHKDDSPDL

SEQ ID NO: 219 - protein, 27 aa, Homo sapiens
  MOD_RES 1..27: note = Glycation [?]
  MOD_RES 8..19: note = Carboxymethyl C (3) [8, 9, and/or 19]
  MOD_RES 11..24: note = Glycation (2) [11 and/or 24]
  MOD_RES 17: note = Deamidation N [17]
TYGDMADCCEKQEPERNECFLSHKDDS

SEQ ID NO: 220 - protein, 25 aa, Homo sapiens
  MOD_RES 1..25: note = Glycation [?]
  MOD_RES 8..19: note = Carboxymethyl C (3) [8, 9, and/or 19]
TYGDMADCCEKQEPERNECFLSHKD

SEQ ID NO: 221 - protein, 41 aa, Homo sapiens
  MOD_RES 5: note = Oxidation M [5]
  MOD_RES 8..41: note = Carboxymethyl C [8, 9, 19, and/or 41]
  MOD_RES 11..34: note = Glycation (4) [11, 24, 32, and/or 34]
  MOD_RES 17..38: note = Deamidation N (2) [17 and/or 38]
TYGDMADCCEKQEPERNECFLSHKDDSPDLPKLKPDPNTLC

SEQ ID NO: 222 - protein, 23 aa, Homo sapiens
  MOD_RES 5: note = Oxidation M [5]
  MOD_RES 8..19: note = Carboxymethyl C (3) [8, 9, and/or 19]
  MOD_RES 11: note = Glycation [11]
TYGDMADCCEKQEPERNECFLSH

SEQ ID NO: 223 - protein, 22 aa, Homo sapiens
  MOD_RES 8..19: note = Carboxymethyl C (3) [8, 9, and/or 19]
  MOD_RES 17: note = Deamidation N [17]
TYGDMADCCEKQEPERNECFLS

SEQ ID NO: 224 - protein, 21 aa, Homo sapiens
  MOD_RES 5: note = Oxidation M [5]
  MOD_RES 8..19: note = Carboxymethyl C (3) [8, 9, and/or 19]
  MOD_RES 11: note = Glycation [11]
TYGDMADCCEKQEPERNECFL

SEQ ID NO: 225 - protein, 20 aa, Homo sapiens
  MOD_RES 5: note = Oxidation M [5]
  MOD_RES 8..19: note = Carboxymethyl C (3) [8, 9, and/or 19]
  MOD_RES 11: note = Glycation [11]
  MOD_RES 17: note = Deamidation N [17]
TYGDMADCCEKQEPERNECF

SEQ ID NO: 226 - protein, 39 aa, Homo sapiens
  MOD_RES 1..39: note = Deamidation N [?]
  MOD_RES 5: note = Oxidation M [5]
  MOD_RES 8..19: note = Carboxymethyl C (3) [8, 9, and/or 19]
  MOD_RES 11..34: note = Glycation (4) [11, 24, 32, and/or 34]
  MOD_RES 17..38: note = Deamidation N (2) [17 and/or 38]
TYGDMADCCEKQEPERNECFLSHKDDSPDLPKLKPDPNT

SEQ ID NO: 227 - protein, 38 aa, Homo sapiens
  MOD_RES 1..38: note = Glycation (3) [3?]
  MOD_RES 1..38: note = Deamidation N [?]
  MOD_RES 5: note = Oxidation M [5]
  MOD_RES 8..19: note = Carboxymethyl C (3) [8, 9, and/or 19]
  MOD_RES 11..34: note = Glycation (4) [11, 24, 32, and/or 34]
TYGDMADCCEKQEPERNECFLSHKDDSPDLPKLKPDPN

SEQ ID NO: 228 - protein, 42 aa, Homo sapiens
  MOD_RES 1..42: note = Glycation (3) [3?]
  MOD_RES 1..42: note = Dearnidation N [?]
  MOD_RES 4: note = Oxidation M [4]
  MOD_RES 7..40: note = Carboxymethyl C (4) [7, 8, 18, and/or 40]
  MOD_RES 10..33: note = Glycation (4) [10, 23, 31, and/or 33]
  MOD_RES 16..37: note = Deamidation N (2) [16 and/or 37]
YGDMADCCEKQEPERNECFLSHKDDSPDLPKLKPDPNTLCDE

SEQ ID NO: 229 - protein, 40 aa, Homo sapiens
  MOD_RES 1..40: note = Glycation (2) [2?]
  MOD_RES 5..38: note = Carboxymethyl C [5, 6, 16, and/or 38]
  MOD_RES 14..35: note = Deamidation N (2) [14 and/or 35]
DMADCCEKQEPERNECFLSHKDDSPDLPKLKPDPNTLCDE

SEQ ID NO: 230 - protein, 37 aa, Homo sapiens
  MOD_RES 1..37: note = Glycation (3) [3 ?]
  MOD_RES 1..37: note = Deamidation N [?]
  MOD_RES 2..35: note = Carboxymethyl C (4) [2, 3, 13, and/or 35]
  MOD_RES 11..32: note = Deamidation N (2) [11 and/or 32]
DCCEKQEPERNECFLSHKDDSPDLPKLKPDPNTLCDE

SEQ ID NO: 231 - protein, 9 aa, Homo sapiens
  MOD_RES 5: note = Oxidation M [5]
  MOD_RES 8..9: note = Carboxymethyl C (2) [8 and/or 9]
TYGDMADCC

SEQ ID NO: 232 - protein, 8 aa, Homo sapiens
  MOD_RES 5: note = Oxidation M [5]
  MOD_RES 8: note = Carboxymethyl C [8]
TYGDMADC

SEQ ID NO: 233 - protein, 6 aa, Homo sapiens
  MOD_RES 5: note = Oxidation M[5]
TYGDMA

SEQ ID NO: 234 - protein, 5 aa, Homo sapiens
  MOD_RES 5: note = Oxidation M[5]
TYGDM

SEQ ID NO: 235 - protein, 9 aa, Homo sapiens
  MOD_RES 4: note = Oxidation M [4]
  MOD_RES 7..8: note = Carboxymethyl C (2) [7 and/or 8]
YGDMADCCE

SEQ ID NO: 236 - protein, 7 aa, Homo sapiens
  MOD_RES 5..6: note = Carboxymethyl C (2) [5 and/or 6]
DMADCCE

SEQ ID NO: 237 - protein, 6 aa, Homo sapiens
  MOD_RES 1: note = Oxidation M [1]
  MOD_RES 4..5: note = Carboxymethyl C (2) [4 and/or 5]
MADCCE

SEQ ID NO: 238 - protein, 8 aa, Homo sapiens
  MOD_RES 1: note = Glycation [1]
KQEPERNE

SEQ ID NO: 239 - protein, 7 aa, Homo sapiens
  MOD_RES 1: note = GlycatIon [1]
  MOD_RES 7: note = Deamidation N [7]
KQEPERN

SEQ ID NO: 240 - protein, 6 aa, Homo sapiens
  MOD_RES 5: note = Deamidation N [5]
EPERNE

SEQ ID NO: 241 - protein, 4 aa, Homo sapiens
  MOD_RES 3: note = Deamidation N [3]
ERNE

SEQ ID NO: 242 - protein, 33 aa, Homo sapiens
  MOD_RES 1..33: note = Glycation (3) [3 ?]
  MOD_RES 1..33: note = Deamidation N [?]
  MOD_RES 9..31: note = Carboxymethyl C (2) [9 and/or 31]
KQEPERNECFLSHKDDSPDLPKLKPDPNTLCDE

SEQ ID NO: 243 - protein, 19 aa, Homo sapiens
  MOD_RES 1..14: note = Glycation (2) [1 and/or 14]
  MOD_RES 7: note = Deamidation N [7]
  MOD_RES 9: note = Carboxymethyl C [9]
KQEPERNECFLSHKDDSPD

SEQ ID NO: 244 - protein, 18 aa, Homo sapiens
  MOD_RES 1..14: note = Glycation (2) [1 and/or 14]
  MOD_RES 7: note = Deamidation N [7]
  MOD_RES 9: note = Carboxymethyl C [9]
KQEPERNECFLSHKDDSP

SEQ ID NO: 245 - protein, 9 aa, Homo sapiens
  MOD_RES 1: note = Glycation [1]
  MOD_RES 7: note = Deamidation N [7]
  MOD_RES 9: note = Carboxymethyl C [9]
KQEPERNEC

SEQ ID NO: 246 - protein, 34 aa, Homo sapiens
  MOD_RES 1..34: note = Deamidation N [?]
  MOD_RES 1..34: note = Glycation (3) [3 ?]
  MOD_RES 5..27: note = Carboxymethyl C (2) [5 and/or 27]
ERNECFLSHKDDSPDLPKLKPDPNTLCDEFKADE

SEQ ID NO: 247 - protein, 4 aa, Homo sapiens
  MOD_RES 1: note = Glycation [1]
KQEP

SEQ ID NO: 248 - protein, 39 aa, Homo sapiens
  MOD_RES 1..39: note = Glycation [?]
MKWVTFISLLLLFSSAYSRGVFRRDTHKSEIAHRFKDLG

SEQ ID NO: 249 - protein, 38 aa, Homo sapiens
  MOD_RES 1: note = Oxidation M [1]
  MOD_RES 1..38: note = Glycation [?]
MKWVTFISLLLLFSSAYSRGVFRRDTHKSEIAHRFKDL

SEQ ID NO: 250 - protein, 37 aa, Homo sapiens
  MOD_RES 2..35: note = Glycation (3) [2, 28, and/or 35]
MKWVTFISLLLLFSSAYSRGVFRRDTHKSEIAHRFKD

SEQ ID NO: 251 - protein, 36 aa, Homo sapiens
  MOD_RES 1: note = Oxidation M [1]
MKWVTFISLLLLFSSAYSRGVFRRDTHKSEIAHRFK

SEQ ID NO: 252 - protein, 39 aa, Homo sapiens
  MOD_RES 1..35: note = Glycation (3) [1, 27, and/or 35]
KWVTFISLLLLFSSAYSRGVFRRDTHKSEIAHRFKDLGE

SEQ ID NO: 253 - protein, 13 aa, Homo sapiens
  MOD_RES 1..13: note = Glycation [?]
KSEIAHRFKDLGE

SEQ ID NO: 254 - protein, 41 aa, Homo sapiens
  MOD_RES 1: note = Oxidation M [1]
MKWVTFISLLLLFSSAYSRGVFRRDTHKSEIAHRFKDLGEE

SEQ ID NO: 255 - protein, 28 aa, Homo sapiens
  MOD_RES 1..28: note = Deamidation N [?]
  MOD_RES 4..26: note = Carboxymethyl C (2) [4 and/or 26]
RNECFLSHKDDSPDLPKLKPDPNTLCDE

SEQ ID NO: 256 - protein, 14 aa, Homo sapiens
  MOD_RES 2: note = Deamidation N [2]
  MOD_RES 4: note = Carboxymethyl C [4]
  MOD_RES 9: note = Glycation [9]
RNECFLSHKDDSPD

SEQ ID NO: 257 - protein, 13 aa, Homo sapiens
  MOD_RES 4: note = Carboxymethyl C [4]
RNECFLSHKDDSP

SEQ ID NO: 258 - protein, 10 aa, Homo sapiens
  MOD_RES 2: note = Deamidation N [2]
  MOD_RES 4: note = Carboxymethyl C [4]
  MOD_RES 9: note = Glycation [9]
RNECFLSHKD

SEQ ID NO: 259 - protein, 9 aa, Homo sapiens
  MOD_RES 2: note = Deamidation N [2]
  MOD_RES 4: note = Carboxymethyl C [4]
  MOD_RES 9: note = Glycation [9]
RNECFLSHK

SEQ ID NO: 260 - protein, 5 aa, Homo sapiens
  MOD_RES 2: note = Deamidation N [2]
  MOD_RES 4: note = Carboxymethyl C [4]
RNECF

SEQ ID NO: 261 - protein, 4 aa, Homo sapiens
  MOD_RES 4: note = Carboxymethyl C [4]
RNEC

SEQ ID NO: 262 - protein, 23 aa, Homo sapiens
  MOD_RES 1..23: note = Deamidation N [?]
  MOD_RES 4: note = Carboxymethyl C [4]
  MOD_RES 9..19: note = Glycation (3) [9, 17, and/or 19]
RNECFLSHKDDSPDLPKLKPDPN

SEQ ID NO: 263 - protein, 22 aa, Homo sapiens
  MOD_RES 2: note = Deamidation N [2]
  MOD_RES 4: note = Carboxymethyl C [4]
  MOD_RES 9..19: note = Glycation (3) [9, 17, and/or 19]
RNECFLSHKDDSPDLPKLKPDP

SEQ ID NO: 264 - protein, 21 aa, Homo sapiens
  MOD_RES 4: note = Carboxymethyl C [4]
  MOD_RES 9..19: note = Glycation (3) [9, 17, and/or 19]
RNECFLSHKDDSPDLPKLKPD

SEQ ID NO: 265 - protein, 19 aa, Homo sapiens
  MOD_RES 2: note = Deamidation N [2]
  MOD_RES 4: note = Carboxymethyl C [4]
RNECFLSHKDDSPDLPKLK

SEQ ID NO: 266 - protein, 30 aa, Homo sapiens
  MOD_RES 1..30: note = Deamidation N[?]
  MOD_RES 1..30: note = Glycation [?]
  MOD_RES 4..26: note = Carboxymethyl C (2) [4 and/or 26]
RNECFLSHKDDSPDLPKLKPDPNTLCDEFK

SEQ ID NO: 267 - protein, 32 aa, Homo sapiens
  MOD_RES 1..22: note = Deamidation N (2) [1 and/or 22]
  MOD_RES 1..32: note = Glycation [?]
  MOD_RES 3..25: note = Carboxymethyl C (2) [3 and/or 25]
  MOD_RES 8..29: note = Glycation (4) [8 16 18 29]
NECFLSHKDDSPDLPKLKPDPNTLCDEFKADE

SEQ ID NO: 268 - protein, 44 aa, Homo sapiens
  MOD_RES 2..23: note = Deamidation N (2) [2 and/or 23]
  MOD_RES 4..26: note = Carboxymethyl C (2) [4 and/or 26]
RNECFLSHKDDSPDLPKLKPDPNTLCDEFFKADEKKFWGKYLYE

SEQ ID NO: 269 - protein, 42 aa, Homo sapiens
  MOD_RES 1..22: note = Deamidation N (2) [1 and/or 22]
  MOD_RES 1..42: note = Glycation (5) [5?]
  MOD_RES 3..25: note = Carboxymethyl C (2) [3 and/or 25]
NECFLSHKDDSPDLPKLKPDPNTLCDEFKADEKKFWGKYLYE

SEQ ID NO: 270 - protein, 16 aa, Homo sapiens
  MOD_RES 3..11: note = Glycation (2) [3 and/or 11]
THKSEIAHRFKDLGEE

SEQ ID NO: 271 - protein, 25 aa, Homo sapiens
  MOD_RES 1..23: note = Carboxymethyl C (2) [1 and/or 23]
  MOD_RES 6..16: note = Glycation (3) [6, 14, and/or 16]
CFLSHKDDSPDLPKLKPDPNTLCDE

SEQ ID NO: 272 - protein, 58 aa, Homo sapiens
  MOD_RES 1: note = Oxidation M [1]
  MOD_RES 1..58: note = Glycation (2) [2?]
  MOD_RES 58: note = Carboxymethyl C [58]
MKWVTFISLLLLFSSAYSRGVFRRDTHKSEIAHRFKDLGEEHFKGLVLIAFSQYLQQC

SEQ ID NO: 273 - protein, 29 aa, Homo sapiens
  MOD_RES 1..23: note = Carboxymethyl C (2) [1 and/or 23]
  MOD_RES 20: note = Deamidation N [20]
CFLSHKDDSPDLPKLKPDPNTLCDEFKAD

SEQ ID NO: 274 - protein, 17 aa, Homo sapiens
  MOD_RES 1..14: note = Glycation (3) [1, 3, and/or 14]
  MOD_RES 10: note = Carboxymethyl C [10]
KLKPDPNTLCDEFKADE

SEQ ID NO: 275 - protein, 13 aa, Homo sapiens
  MOD_RES 6: note = Carboxymethyl C [6]
DPNTLCDEFKADE

SEQ ID NO: 276 - protein, 12 aa, Homo sapiens
  MOD_RES 5: note = Carboxymethyl C [5]
  MOD_RES 9: note = Glycation [9]
PNTLCDEFKADE

SEQ ID NO: 277 - protein, 11 aa, Homo sapiens
  MOD_RES 4: note = Carboxymethyl C [4]
NTLCDEFKADE

SEQ ID NO: 278 - protein, 8 aa, Homo sapiens
  MOD_RES 1: note = Carboxymethyl C [1]
  MOD_RES 5: note = Glycation [5]
CDEFKADE

SEQ ID NO: 279 - protein, 22 aa, Homo sapiens
  MOD_RES 6..19: note = Glycation (3) [6, 8, and/or 19]
  MOD_RES 15: note = Carboxymethyl C [15]
SPDLPKLKPDPNTLCDEFKADE

SEQ ID NO: 280 - protein, 21 aa, Homo sapiens
  MOD_RES 14: note = Carboxymethyl C [14]
PDLPKLKPDPNTLCDEFKADE

SEQ ID NO: 281 - protein, 40 aa, Homo sapiens
  MOD_RES 1..23: note = Carboxymethyl C (2) [1 and/or 23]
  MOD_RES 1..40: note = Glycation [?]
  MOD_RES 20: note = Deamidation N [20]
CFLSHKDDSPDLPKLKPDPNTLCDEFKADEKKFWGKYLYE

SEQ ID NO: 282 - protein, 35 aa, Homo sapiens
  MOD_RES 1..23: note = Carboxymethyl C (2) [1 and/or 23]
CFLSHKDDSPDLPKLKPDPNTLCDEFKADEKKFWG

SEQ ID NO: 283 - protein, 32 aa, Homo sapiens
  MOD_RES 1..23: note = Carboxymethyl C (2) [1 and/or 23]
  MOD_RES 1..32: note = Glycation (4) [4?]
CFLSHKDDSPDLPKLKPDPNTLCDEFKADEKK

SEQ ID NO: 284 - protein, 37 aa, Homo sapiens
  MOD_RES 1..37: note = Glycation [?]
  MOD_RES 20: note = Carboxymethyl C [20]
SHKDDSPDLPKLKPDPNTLCDEFKADEKKFWGKYLYE

SEQ ID NO: 285 - protein, 50 aa, Homo sapiens
  MOD_RES 1..23: note = Carboxymethyl C (2) [1 and/or 23]
  MOD_RES 1..50: note = Glycation (3) [3 ?]
  MOD_RES 20: note = Deamidation N [20]
CFLSHKDDSPDLPKLKPDPNTLCDEFKADEKKFWGKYLYEIARRHPYFYA

SEQ ID NO: 286 - protein, 48 aa, Homo sapiens
  MOD_RES 1..23: note = Carboxymethyl C (2) [1 and/or 23]
  MOD_RES 6..36: note = Glycation (7) [6, 14, 16, 27, 31, 32, and/or 36]
  MOD_RES 6..36: note = Glycation (7) [6, 14, 16, 27, 31, 32, and/or 36]
CFLSHKDDSPDLPKLKPDPNTLCDEFKADEKKFWGKYLYEIARRHPYF

SEQ ID NO: 287 - protein, 38 aa, Homo sapiens
  MOD_RES 9: note = Carboxymethyl C [9]
LKPDPNTLCDEFKADEKKFWGKYLYEIARRHPYFYAPE

SEQ ID NO: 288 - protein, 36 aa, Homo sapiens
  MOD_RES 1..36: note = Glycation (2) [2 ?]
  MOD_RES 4: note = Deamidation N [4]
  MOD_RES 7: note = Carboxymethyl C [7]
PDPNTLCDEFKADEKKFWGKYLYEIARRHPYFYAPE

SEQ ID NO: 289 - protein, 48 aa, Homo sapiens
  MOD_RES 1..48: note = Glycation (6) [6 ?]
  MOD_RES 19: note = Carboxymethyl C [19]
HKDDSPDLPKLKPDPNTLCDEFKADEKKFWGKYLYEIARRHPYFYAPE

SEQ ID NO: 290 - protein, 51 aa, Homo sapiens
  MOD_RES 1: note = Oxidation M [1]
  MOD_RES 1..51: note = Glycation [?]
  MOD_RES 2..44: note = Glycation (4) [2, 28, 36, and/or 44]
MKWVTFISLLLLFSSAYSRGVFRRDTHKSEIAHRFKDLGEEHFKGLVLIAF

SEQ ID NO: 291 - protein, 13 aa, Homo sapiens
  MOD_RES 1: note = Carboxymethyl C [1]
  MOD_RES 6: note = Glycation [6]
CFLSHKDDSPDLP

SEQ ID NO: 292 - protein, 12 aa, Homo sapiens
  MOD_RES 1: note = Carboxymethyl C [1]
  MOD_RES 6: note = Glycation [6]
CFLSHKDDSPDL

SEQ ID NO: 293 - protein, 11 aa, Homo sapiens
  MOD_RES 1: note = Carboxymethyl C [1]
  MOD_RES 6: note = Glycation [6]
CFLSHKDDSPD

SEQ ID NO: 294 - protein, 10 aa, Homo sapiens
  MOD_RES 1: note = Carboxymethyl C [1]
CFLSHKDDSP

SEQ ID NO: 295 - protein, 9 aa, Homo sapiens
  MOD_RES 1: note = Carboxymethyl C [1]
  MOD_RES 6: note = Glycation [6]
CFLSHKDDS

SEQ ID NO: 296 - protein, 5 aa, Homo sapiens
  MOD_RES 1: note = Carboxymethyl C [1]
CFLSH

SEQ ID NO: 297 - protein, 21 aa, Homo sapiens
  MOD_RES 1: note = Carboxymethyl C [1]
  MOD_RES 20: note = Deamidation N [20]
CFLSHKDDSPDLPKLKPDPNT

SEQ ID NO: 298 - protein, 15 aa, Homo sapiens
  MOD_RES 1..15: note = Glycation [?]
  MOD_RES 13: note = Carboxymethyl C [13]
DLPKLKPDPNTLCDE

SEQ ID NO: 299 - protein, 10 aa, Homo sapiens
  MOD_RES 1: note = Glycation [1]
  MOD_RES 5: note = Deamidation N [5]
  MOD_RES 8: note = Carboxymethyl C [8]
KPDPNTLCDE

SEQ ID NO: 300 - protein, 9 aa, Homo sapiens
  MOD_RES 4: note = Deamidation N [4]
  MOD_RES 7: note = Carboxymethyl C [7]
PDPNTLCDE

SEQ ID NO: 301 - protein, 8 aa, Homo sapiens
  MOD_RES 3: note = Deamidation N [3]
  MOD_RES 3: note = Deamidation N [3]
  MOD_RES 6: note = Carboxymethyl C [6]
DPNTLCDE

SEQ ID NO: 302 - protein, 7 aa, Homo sapiens
  MOD_RES 2: note = Deamidation N [2]
  MOD_RES 2: note = Deamidation N [2]
  MOD_RES 5: note = Carboxymethyl C [5]
PNTLCDE

SEQ ID NO: 303 - protein, 6 aa, Homo sapiens
  MOD_RES 1: note = Deamidation N [1]
  MOD_RES 4: note = Carboxymethyl C [4]
NTLCDE

SEQ ID NO: 304 - protein, 4 aa, Homo sapiens
  MOD_RES 2: note = Carboxymethyl C [2]
LCDE

SEQ ID NO: 305 - protein, 39 aa, Homo sapiens
  MOD_RES 5..21: note = Glycation (3) [5, 13, and/or 21]
  MOD_RES 35: note = Carboxymethyl C [35]
RDTHKSEIAHRFKDLGEEHFKGLVLIAFSQYLQQCPFDE

SEQ ID NO: 306 - protein, 21 aa, Homo sapiens
  MOD_RES 1..21: note = Glycation [?]
  MOD_RES 16: note = Deamidation N [16]
  MOD_RES 19: note = Carboxymethyl C [19]
HKDDSPDLPKLKPDPNTLCDE

SEQ ID NO: 307 - protein, 19 aa, Homo sapiens
  MOD_RES 8..10: note = Glycation (2) [8 and/or 10]
  MOD_RES 17: note = Carboxymethyl C [17]
DDSPDLPKLKPDPNTLCDE

SEQ ID NO: 308 - protein, 5 aa, Homo sapiens
  MOD_RES 2: note = Glycation [2]
FKADE

SEQ ID NO: 309 - protein, 14 aa, Homo sapiens
  MOD_RES 1..14: note = Glycation (3) [3 ?]
KADEKKFWGKYLYE

SEQ ID NO: 310 - protein, 26 aa, Homo sapiens
  MOD_RES 1..26: note = Glycation [?]
FKADEKKFWGKYLYEIARRHPYFYAP

SEQ ID NO: 311 - protein, 25 aa, Homo sapiens
  MOD_RES 1..25: note = Glycation (3) [3 ?]
  MOD_RES 1..25: note = Glycation (2) [2 ?]
FKADEKKFWGKYLYEIARRHPYFYA

SEQ ID NO: 312 - protein, 23 aa, Homo sapiens
  MOD_RES 2..7: note = Glycation (3) [2, 3, and/or 7]
EKKFWGKYLYEIARRHPYFYAPE

SEQ ID NO: 313 - protein, 36 aa, Homo sapiens
  MOD_RES 2..34: note = Glycation (5) [2, 6, 7, 11, and/or 34]
  MOD_RES 33..36: note = Deamidation N (2) [33 and/or 36]
FKADEKKFWGKYLYEIARRHPYFYAPELLYYANKYN

SEQ ID NO: 314 - protein, 40 aa, Homo sapiens
  MOD_RES 1..33: note = Glycation (5) [1, 5, 6, 10, and/or 33]
  MOD_RES 1..40: note = Deamidation N [?]
KADEKKFWGKYLYEIARRHPYFYAPELLYYANKYNGVFQE

SEQ ID NO: 315 - protein, 4 aa, Homo sapiens
  MOD_RES 1: note = Glycation [1]
KADE

SEQ ID NO: 316 - protein, 22 aa, Homo sapiens
  MOD_RES 1..22: note = Glycation [?]
KKFWGKYLYEIARRHPYFYAPE

SEQ ID NO: 317 - protein, 36 aa, Homo sapiens
  MOD_RES 1..36: note = Glycation (3) [3 ?]
  MOD_RES 1..36: note = Deamidation N [?]
  MOD_RES 28..31: note = Deamidation N (2) [28 and/or 31]
KKFWGKYLYEIARRHPYFYAPELLYYANKYNGVFQE

SEQ ID NO: 318 - protein, 35 aa, Homo sapiens
  MOD_RES 1..35: note = Glycation (2) [2 ?]
  MOD_RES 27..30: note = Deamidation N (2) [27 and/or 30]
KFWGKYLYEIARRHPYFYAPELLYYANKYNGVFQE

SEQ ID NO: 319 - protein, 32 aa, Homo sapiens
  MOD_RES 2..25: note = Glycation (2) [2 and/or 25]
  MOD_RES 24..27: note = Deamidation N (2) [24 and/or 27]
GKYLYEIARRHPYFYAPELLYYANKYNGVFQE

SEQ ID NO: 320 - protein, 36 aa, Homo sapiens
  MOD_RES 1..36: note = Glycation [?]
  MOD_RES 23..26: note = Deamidation N (2) [23 and/or 26]
  MOD_RES 32..33: note = Carboxymethyl C (2) [32 and/or 33]
KYLYEIARRHPYFYAPELLYYANKYNGVFQECCQAE

SEQ ID NO: 321 - protein, 7 aa, Homo sapiens
  MOD_RES 1..7: note = Glycation [?]
KKFWGKY

SEQ ID NO: 322 - protein, 5 aa, Homo sapiens
  MOD_RES 1: note = Glycation [1]
KYLYE

SEQ ID NO: 323 - protein, 20 aa, Homo sapiens
  MOD_RES 19: note = Glycation [19]
IARRHPYFYAPELLYYANKY

SEQ ID NO: 324 - protein, 16 aa, Homo sapiens
  MOD_RES 1..16: note = Deamidation N [?]
  MOD_RES 9: note = Glycation [9]
PELLYYANKYNGVFQE

SEQ ID NO: 325 - protein, 18 aa, Homo sapiens
  MOD_RES 1..18: note = Deamidation N [?]
  MOD_RES 11: note = Glycation [11]
YAPELLYYANKYNGVFQE

SEQ ID NO: 326 - protein, 17 aa, Homo sapiens
  MOD_RES 1..17: note = Deamidation N [?]
APELLYYANKYNGVFQE

SEQ ID NO: 327 - protein, 31 aa, Homo sapiens
  MOD_RES 19: note = Glycation [19]
  MOD_RES 27..28: note = Carboxymethyl C (2) [27 and/or 28]
IARRHPYFYAPELLYYANKYNGVFQECCQAE

SEQ ID NO: 328 - protein, 21 aa, Homo sapiens
  MOD_RES 1..21: note = Deamidation N [?]
  MOD_RES 9: note = Glycation [9]
  MOD_RES 17..18: note = Carboxymethyl C (2) [17 and/or 18]
PELLYYANKYNGVFQECCQAE

SEQ ID NO: 329 - protein, 20 aa, Homo sapiens
  MOD_RES 1..20: note = Deamidation N [?]
  MOD_RES 7..10: note = Deamidation N (2) [7 and/or 10]
  MOD_RES 16..17: note = Carboxymethyl C (2) [16 and/or 17]
ELLYYANKYNGVFQECCQAE

SEQ ID NO: 330 - protein, 27 aa, Homo sapiens
  MOD_RES 14..17: note = Deamidation N (2) [14 and/or 17]
  MOD_RES 15: note = Glycation [15]
  MOD_RES 23..24: note = Carboxymethyl C (2) [23 and/or 24]
HPYFYAPELIYYANKYNGVFQECCQAE

SEQ ID NO: 331 - protein, 22 aa, Homo sapiens
  MOD_RES 9..12: note = Deamidation N (2) [9 and/or 12]
  MOD_RES 18..19: note = Carboxymethyl C (2) [18 and/or 19]
APELLYYANKYNGVFQECCQAE

SEQ ID NO: 332 - protein, 42 aa, Homo sapiens
  MOD_RES 1..42: note = Deamidation N [?]
  MOD_RES 1..42: note = Glycation (2) [2 ?]
  MOD_RES 27..36: note = Carboxymethyl C (3] [27, 28, and/or 36]
IARRHPYFYAPELLYYANKYNGVFQECCQAEDKGACLLPKIE

SEQ ID NO: 333 - protein, 40 aa, Homo sapiens
  MOD_RES 1..40: note = Deamidation N [?]
  MOD_RES 1..40: note = Glycation [?]
  MOD_RES 27..36: note = Carboxymethyl C (3) [27, 28, and/or 36]
IARRHPYFYAPELLYYANKYNGVFQECCQAEDKGACLLPK

SEQ ID NO: 334 - protein, 18 aa, Homo sapiens
  MOD_RES 1..18: note = Deamidation N [?]
  MOD_RES 7: note = Glycation [7]
  MOD_RES 15..16: note = Carboxymethyl C (2) [15 and/or 16]
LLYYANKYNGVFQECCQA

SEQ ID NO: 335 - protein, 16 aa, Homo sapiens
  MOD_RES 6..9: note = Deamidation N (2) [6 and/or 9]
  MOD_RES 7: note = Glycation [7]
  MOD_RES 15..16: note = Carboxymethyl C (2) [15 and/or 16]
LLYYANKYNGVFQECC

SEQ ID NO: 336 - protein, 18 aa, Homo sapiens
  MOD_RES 1..18: note = Deamidation N [?]
  MOD_RES 6: note = Glycation [6]
  MOD_RES 14..15: note = Carboxymethyl C (2) [14 and/or 15]
LYYANKYNGVFQECCQAE

SEQ ID NO: 337 - protein, 9 aa, Homo sapiens
  MOD_RES 5..6: note = Carboxymethyl C (2) [5 and/or 6]
VFQECCQAE

SEQ ID NO: 338 - protein, 8 aa, Homo sapiens
  MOD_RES 4..5: note = Carboxymethyl C (2) [4 and/or 5]
FQECCQAE

SEQ ID NO: 339 - protein, 7 aa, Homo sapiens
  MOD_RES 3..4: note = Carboxymethyl C (2) [3 and/or 4]
QECCQAE

SEQ ID NO: 340 - protein, 6 aa, Homo sapiens
  MOD_RES 2..3: note = Carboxymethyl C (2) [2 and/or 3]
ECCQAE

SEQ ID NO: 341 - protein, 16 aa, Homo sapiens
  MOD_RES 1..16: note = Deamidation N [?]
  MOD_RES 3..6: note = Deamidation N (2) [3 and/or 6]
  MOD_RES 4: note = Glycation [4]
  MOD_RES 12..13: note = Carboxymethyl C (2) [12 and/or 13]
YANKYNGVFQECCQAE

SEQ ID NO: 342 - protein, 15 aa, Homo sapiens
  MOD_RES 11..12: note = Carboxymethyl C (2) [11 and/or 12]
ANKYNGVFQECCQAE

SEQ ID NO: 343 - protein, 14 aa, Homo sapiens
  MOD_RES 1..14: note = Deamidation N [?]
  MOD_RES 1..4: note = Deamidation N (2) [1 and/or 4]
  MOD_RES 2: note = Glycation [2]
  MOD_RES 10..11: note = Carboxymethyl C (2) [10 and/or 11]
NKYNGVFQECCQAE

SEQ ID NO: 344 - protein, 12 aa, Homo sapiens
  MOD_RES 1: note = Glycation [1]
  MOD_RES 3: note = Deamidation N [3]
  MOD_RES 9..10: note = Carboxymethyl C (2) [9 and/or 10]
KYNGVFQECQAE

SEQ ID NO: 345 - protein, 11 aa, Homo sapiens
  MOD_RES 1: note = Deamidation N [1]
  MOD_RES 7..8: note = Carboxymethyl C (2) [7 and/or 8]
NGVFQECCQAE

SEQ ID NO: 346 - protein, 10 aa, Homo sapiens
  MOD_RES 6..7: note = Carboxymethyl C (2)[6 and/or 7]
GVFQECCQAE

SEQ ID NO: 347 - protein, 27 aa, Homo sapiens
  MOD_RES 6..9: note = Deamidation N (2) [6 and/or 9]
  MOD_RES 15..24: note = Carboxymethyl C (3) [15, 16, and/or 24]
LLYYANKYNGVFQECCQAEDKGACLLP

SEQ ID NO: 348 - protein, 17 aa, Homo sapiens
  MOD_RES 1..17: note = Glycation [?]
  MOD_RES 2..11: note = Carboxymethyl C (3) [2, 3, and/or 11]
ECCQAEDKGACLLPKIE

SEQ ID NO: 349 - protein, 23 aa, Homo sapiens
  MOD_RES 1..23: note = Glycation [?]
  MOD_RES 8..17: note = Carboxymethyl C (3) [8, 9, and/or 17]
YNGVFQECCQAEDKGACLLPKIE

SEQ ID NO: 350 - protein, 23 aa, Homo sapiens
  MOD_RES 4..13: note = Carboxymethyl C (3) [4, 5, and/or 13]
  MOD_RES 10..17: note = Glycation (2) [10 17]
FQECCQAEDKGACLLPKIETMRE

SEQ ID NO: 351 - protein, 21 aa, Homo sapiens
  MOD_RES 2..11: note = Carboxymethyl C (3) [2, 3, and/or 11]
ECCQAEDKGACLLPKIETMRE

SEQ ID NO: 352 - protein, 29 aa, Homo sapiens
  MOD_RES 1..29: note = Deamidation N [?]
  MOD_RES 1..29: note = Oxidation M [?]
  MOD_RES 10..19: note = Carboxymethyl C (3) [10, 11, and/or 19]
NKYNGVFQECCQAEDKGACLLPKIETMRE

SEQ ID NO: 353 - protein, 28 aa, Homo sapiens
  MOD_RES 9..18: note = Carboxymethyl C (3) [9, 10, and/or 18]
KYNGVFQECCQAEDKGACLLPKIETMRE

SEQ ID NO: 354 - protein, 6 aa, Homo sapiens
  MOD_RES 6: note = Deamidation N [6]
LLYYAN

SEQ ID NO: 355 - protein, 11 aa, Homo sapiens
  MOD_RES 1..11: note = Deamidation N [?]
YANKYNGVFQE

SEQ ID NO: 356 - protein, 10 aa, Homo sapiens
  MOD_RES 1..10: note = Deamidation [?]
  MOD_RES 2..5: note = Deamidation N (2) [2 and/or 5]
  MOD_RES 3: note = Glycation [3]
ANKYNGVFQE

SEQ ID NO: 357 - protein, 9 aa, Homo sapiens
  MOD_RES 2: note = Glycation [2]
NKYNGVFQE

SEQ ID NO: 358 - protein, 8 aa, Homo sapiens
  MOD_RES 1: note = Glycation [1]
  MOD_RES 3: note = Deamidation N [3]
KYNGVFQE

SEQ ID NO: 359 - protein, 7 aa, Homo sapiens
  MOD_RES 2: note = Deamidation N [2]
YNGVFQE

SEQ ID NO: 360 - protein, 6 aa, Homo sapiens
  MOD_RES 1..2: note = Carboxymethyl C (2) [1 and/or 2]
CCQAED

SEQ ID NO: 361 - protein, 11 aa, Homo sapiens
  MOD_RES 1..10: note = Carboxymethyl C (3) [1, 2, and/or 10]
  MOD_RES 7: note = Glycation [7]
CCQAEDKGACL

SEQ ID NO: 362 - protein, 10 aa, Homo sapiens
  MOD_RES 1..10: note = Carboxymethyl C (3) [1, 2, and/or 10]
CCQAEDKGAC

SEQ ID NO: 363 - protein, 8 aa, Homo sapiens
  MOD_RES 1..2: note = Carboxymethyl C (2) [1 and/or 2]
  MOD_RES 7: note = Glycation [7]
CCQAEDKG

SEQ ID NO: 364 - protein, 7 aa, Homo sapiens
  MOD_RES 1..2: note = Carboxymethyl C (2) [1 and/or 2]
  MOD_RES 7: note = Glycation [7]
CCQAEDK

SEQ ID NO: 365 - protein, 18 aa, Homo sapiens
  MOD_RES 1..10: note = Carboxymethyl C (3) [1, 2, and/or 10]
  MOD_RES 1..18: note = Glycation [?]
  MOD_RES 18: note = Oxidation M [18]
CCQAEDKGACLLPKIETM

SEQ ID NO: 366 - protein, 38 aa, Homo sapiens
  MOD_RES 1..38: note = Glycation (3) [3 ?]
  MOD_RES 7..30: note = Carboxymethyl C (2) [7 and/or 30]
AEDKGACLLPKIETMREKVLASSARQRLRCASIQKFGE

SEQ ID NO: 367 - protein, 4 aa, Homo sapiens
  MOD_RES 1: note = Carboxymethyl C [1]
CQAE

SEQ ID NO: 368 - protein, 10 aa, Homo sapiens
  MOD_RES 1: note = Oxidation M [1]
MKWVTFISLL

SEQ ID NO: 369 - protein, 6 aa, Homo sapiens
  MOD_RES 2: note = Glycation [2]
MKWVTF

SEQ ID NO: 370 - protein, 5 aa, Homo sapiens
  MOD_RES 1: note = Oxidation M [1]
  MOD_RES 2: note = Glycation [2]
MKWVT

SEQ ID NO: 371 - protein, 4 aa, Homo sapiens
  MOD_RES 1: note = Oxidation M [1]
  MOD_RES 2: note = Glycation [2]
MKWV

SEQ ID NO: 372 - protein, 22 aa, Homo sapiens
  MOD_RES 1: note = Oxidation M [1]
MKWVTFISLLLLFSSAYSRGVF

SEQ ID NO: 373 - protein, 18 aa, Homo sapiens
  MOD_RES 16: note = Glycation [16]
FSSAYSRGVFRRDTHKSE

SEQ ID NO: 374 - protein, 9 aa, Homo sapiens
  MOD_RES 7: note = Glycation [7]
FRRDTHKSE

SEQ ID NO: 375 - protein, 4 aa, Homo sapiens
  MOD_RES 2: note = Glycation [2]
HKSE

SEQ ID NO: 376 - protein, 10 aa, Homo sapiens
  MOD_RES 4: note = Glycation [4]
  MOD_RES 8: note = Oxidation M [8]
LLPKIETMRE

SEQ ID NO: 377 - protein, 6 aa, Homo sapiens
  MOD_RES 4: note = Oxidation M [4]
IETMRE

SEQ ID NO: 378 - protein, 36 aa, Homo sapiens
  MOD_RES 1..36: note = Glycation (3) [3 ?]
  MOD_RES 2..33: note = Glycation (4) [2, 9, 16, and/or 33]
  MOD_RES 5..28: note = Carboxymethyl C (2) [5 and/or 28]
  MOD_RES 13: note = Oxidation M [13]
DKGACLLPKIETMREKVLASSARQRLRCASIQKFGE

SEQ ID NO: 379 - protein, 27 aa, Homo sapiens
  MOD_RES 5: note = Carboxymethyl C [5]
  MOD_RES 13: note = Oxidation M [13]
DKGACLLPKIETMREKVLASSARQRLR

SEQ ID NO: 380 - protein, 47 aa, Homo sapiens
  MOD_RES 1..47: note = Glycation (2) [2 ?]
  MOD_RES 20: note = Carboxymethyl C [20]
KIETMREKVLASSARQRLRCASIQKFGERALKAWSVARLSQKFPKAE

SEQ ID NO: 381 - protein, 45 aa, Homo sapiens
  MOD_RES 1..45: note = Glycation [?]
  MOD_RES 4: note = Oxidation M [4]
  MOD_RES 19: note = Carboxymethyl C [19]
IETMREKLASSARQRLRCASIQKFGERALKAWSVARLSQKFPKAE

SEQ ID NO: 382 - protein, 10 aa, Homo sapiens
  MOD_RES 5: note = Carboxymethyl C [5]
DKGACLLPKI

SEQ ID NO: 383 - protein, 8 aa, Homo sapiens
  MOD_RES 5: note = Carboxymethyl C [5]
DKGACLLP

SEQ ID NO: 384 - protein, 6 aa, Homo sapiens
  MOD_RES 2: note = Glycation [2]
  MOD_RES 5: note = Carboxymethyl C [5]
DKGACL

SEQ ID NO: 385 - protein, 5 aa, Homo sapiens
  MOD_RES 2: note = Glycation [2]
  MOD_RES 5: note = Carboxymethyl C [5]
DKGAC

SEQ ID NO: 386 - protein, 10 aa, Homo sapiens
  MOD_RES 1..10: note = Glycation [?]
  MOD_RES 4: note = Carboxymethyl C [4]
KGACLLPKIE

SEQ ID NO: 387 - protein, 9 aa, Homo sapiens
  MOD_RES 3: note = Carboxymethyl C [3]
GACLLPKIE

SEQ ID NO: 388 - protein, 12 aa, Homo sapiens
  MOD_RES 2: note = Oxidation M [2]
TMREKVLASSAR

SEQ ID NO: 389 - protein, 10 aa, Homo sapiens
  MOD_RES 2: note = Oxidation M [2]
  MOD_RES 5: note = Glycation [5]
TMREKVLASS

SEQ ID NO: 390 - protein, 9 aa, Homo sapiens
  MOD_RES 2: note = Oxidation M [2]
TMREKVLAS

SEQ ID NO: 391 - protein, 44 aa, Homo sapiens
  MOD_RES 1..44: note = Glycation [?]
  MOD_RES 2: note = Oxidation M [2]
  MOD_RES 17: note = Carboxymethyl C [17]
TMREKVLASSARQRLRCASIQKFGERALKAWSVARLSQKFPKAE

SEQ ID NO: 392 - protein, 46 aa, Homo sapiens
  MOD_RES 1..46: note = Glycation (2) [2 ?]
  MOD_RES 16: note = Carboxymethyl C [16]
MREKVLASSARQRLRCASIQKFGERALKAWSVARLSQKFPKAEFVE

SEQ ID NO: 393 - protein, 21 aa, Homo sapiens
  MOD_RES 1..18: note = Glycation (2) [1 and/or 18]
  MOD_RES 13: note = Carboxymethyl C [13]
KVLASSARQRLRCASIQKFGE

SEQ ID NO: 394 - protein, 30 aa, Homo sapiens
  MOD_RES 1..25: note = Glycation (3) [1, 18, and/or 25]
  MOD_RES 13: note = Carboxymethyl C [13]
KVLASSARQRLRCASIQKFGERALKAWSVA

SEQ ID NO: 395 - protein, 36 aa, Homo sapiens
  MOD_RES 13: note = Carboxymethyl C [13]
KVLASSARQRLRCASIQKFGERALKAWSVARLSQKF

SEQ ID NO: 396 - protein, 30 aa, Homo sapiens
  MOD_RES 3: note = Carboxymethyl C [3]
  MOD_RES 8..28: note = Glycation (4) [8, 15, 25, and/or 28]
LRCASIQKFGERALKAWSVARLSQKFPKAE

SEQ ID NO: 397 - protein, 35 aa, Homo sapiens
  MOD_RES 1..35: note = Glycation (3) [3 ?]
  MOD_RES 8: note = Carboxymethyl C [8]
SARQRLRCASIQKFGERALKAWSVARLSQKFPKAE

SEQ ID NO: 398 - protein, 42 aa, Homo sapiens
  MOD_RES 12: note = Carboxymethyl C [12]
VLASSARQRLRCASIQKFGERALKAWSVARLSQKFPKAEFVE

SEQ ID NO: 399 - protein, 56 aa, Homo sapiens
  MOD_RES 1..56: note = Glycation (6) [6 ?]
  MOD_RES 13: note = Carboxymethyl C [13]
KVLASSARQRLRCASIQKFGERALKAWSVARLSQKFPKAEFVEVTKLVTDLTKVHI

SEQ ID NO: 400 - protein, 53 aa, Homo sapiens
  MOD_RES 1..53: note = Glycation (2) [2 ?]
  MOD_RES 13: note = Carboxymethyl C [13]
KVLASSARQRLRCASIQKFGERALKAWSVARLSQKFPKAEFVEVTKLVTDLTK

SEQ ID NO: 401 - protein, 56 aa, Homo sapiens
  MOD_RES 1..56: note = Glycation (4) [4 ?]
  MOD_RES 12: note = Carboxymethyl C [12]
VLASSARQRLRCASIQKFGERALKAWSVARLSQKFPKAEFVEVTKLVTDLTKVHKI

SEQ ID NO: 402 - protein, 47 aa, Homo sapiens
  MOD_RES 1..47: note = Glycation (4) [4 ?]
  MOD_RES 3: note = Carboxymethyl C [3]
LRCASIQKFGERALKAWSVARLSQKFPKAEFVEVTKLVTDLTKVHKE

SEQ ID NO: 403 - protein, 43 aa, Homo sapiens
  MOD_RES 1..43: note = Glycation [?]
SIQKFGERALKAWSVARLSQKFPKAEFVEVTKLVTDLTKVHKE

SEQ ID NO: 404 - protein, 37 aa, Homo sapiens
  MOD_RES 1..37: note = Glycation (2) [2 ?]
ERALKAWSVARLSQKFPKAEFVEVTKLVTDLTKVHKE

SEQ ID NO: 405 - protein, 50 aa, Homo sapiens
  MOD_RES 6: note = Carboxymethyl C [6]
RQRLRCASIQKFGERALKAWSVARLSQKFPKAEFVEVTKLVTDLTKVHKE

SEQ ID NO: 406 - protein, 20 aa, Homo sapiens
  MOD_RES 1..18: note = Glycation (2) [1 and/or 18]
  MOD_RES 13: note = Carboxymethyl C [13]
KVLASSARQRLRCASIQKFG

SEQ ID NO: 407 - protein, 20 aa, Homo sapiens
  MOD_RES 12: note = Carboxymethyl C [12]
VLASSARQRLRCASIQKFGE

SEQ ID NO: 408 - protein, 9 aa, Homo sapiens
  MOD_RES 1: note = Carboxymethyl C [1]
CASIQKFGE

SEQ ID NO: 409 - protein, 6 aa, Homo sapiens
  MOD_RES 3: note = Glycation [3]
IQKFGE

SEQ ID NO: 410 - protein, 9 aa, Homo sapiens
  MOD_RES 1..9: note = Glycation [?]
KFPKAEFVE

SEQ ID NO: 411 - protein, 6 aa, Homo sapiens
  MOD_RES 1: note = Glycation [1]
KAEFVE

SEQ ID NO: 412 - protein, 26 aa, Homo sapiens
  MOD_RES 1..26: note = Glycation (2) [2 ?]
  MOD_RES 1..26: note = Glycation (4) [4 ?]
LSQKFPKAEFVEVTKLVTDLTKVHKE

SEQ ID NO: 413 - protein, 30 aa, Homo sapiens
  MOD_RES 1..30: note = Glycation (2) [2 ?]
  MOD_RES 23..24: note = Carboxymethyl C (2) [23 and/or 24]
FPKAEFVEVTKLVTDLTKVHKECCHGDLLE

SEQ ID NO: 414 - protein, 36 aa, Homo sapiens
  MOD_RES 29..30: note = Carboxymethyl C (2) [29 and/or 30]
ARLSQKFPKAEFVEVTKLVTDLTKVHKECCHGDLLE

SEQ ID NO: 415 - protein, 5 aa, Homo sapiens
  MOD_RES 4: note = Glycation [4]
RALKA

SEQ ID NO: 416 - protein, 12 aa, Homo sapiens
  MOD_RES 4: note = Glycation [4]
RALKAWSVARLS

SEQ ID NO: 417 - protein, 10 aa, Homo sapiens
  MOD_RES 5: note = Glycation [5]
AHRFKDLGEE

SEQ ID NO: 418 - protein, 7 aa, Homo sapiens
  MOD_RES 1..7: note = Glycation [?]
QKFPKAE

SEQ ID NO: 419 - protein, 13 aa, Homo sapiens
  MOD_RES 1..13: note = Glycation [?]
SVARLSQKFPKAE

SEQ ID NO: 420 - protein, 10 aa, Homo sapiens
  MOD_RES 5..8: note = Glycation (2) [5 and/or 8]
RLSQKFPKAE

SEQ ID NO: 421 - protein, 6 aa, Homo sapiens
  MOD_RES 6: note = Glycation [6]
FVEVTK

SEQ ID NO: 422 - protein, 37 aa, Homo sapiens
  MOD_RES 6..35: note = Glycation (4) [6, 13, 16, and/or 35]
  MOD_RES 18..26: note = Carboxymethyl C (3) [18, 19, and/or 26]
FVEVTKLVTDLTKVHKECCHGDLLECADDRADLAKYI

SEQ ID NO: 423 - protein, 48 aa, Homo sapiens
  MOD_RES 1..48: note = Glycation (4) [4?]
  MOD_RES 18..38: note = Carboxymethyl C (4) [18, 19, 26, and/or 38]
  MOD_RES 40: note = Deamidation N [40]
FVEVTKLVTDLTKVHKECCHGDLLECADDRADLAKYICDNQDTISSKL

SEQ ID NO: 424 - protein, 47 aa, Homo sapiens
  MOD_RES 1..47: note = Glycation (4) [4 ?]
  MOD_RES 18..38: note = Carboxymethyl C (4) [18, 19, 26, and/or 38]
  MOD_RES 40: note = Deamidation N [40]
FVEVTKLVTDLTKVHKECCHGDLLECADDRADLAKYICDNQDTISSK

SEQ ID NO: 425 - protein, 44 aa, Homo sapiens
  MOD_RES 1..44: note = Glycation [?]
  MOD_RES 18..38: note = Carboxymethyl C(4) [18, 19, 26, and/or 38]
  MOD_RES 40: note = Deamidation N [40]
FVEVTKLVTDLTKVHKECCHGDLLECADDRADLAKYICDNQDTI

SEQ ID NO: 426 - protein, 20 aa, Homo sapiens
  MOD_RES 15..16: note = Carboxymethyl C (2) [15 and/or 16]
VTKLVTDLTKVHKECCHGDL

SEQ ID NO: 427 - protein, 10 aa, Homo sapiens
  MOD_RES 1: note = Glycation [1]
  MOD_RES 3..4: note = Carboxymethyl C (2) [3 and/or 4]
KECCHGDLLE

SEQ ID NO: 428 - protein, 43 aa, Homo sapiens
  MOD_RES 1..43: note = Glycation (2) [2 ?]
  MOD_RES 15..35: note = Carboxymethyl C (4) [15, 16, ,23, and/or 35]
VTKLVTDLTKVHKECCHGDLLECADDRADLAKYICDNQDTISS

SEQ ID NO: 429 - protein, 35 aa, Homo sapiens
  MOD_RES 1..35: note = Glycation [?]
  MOD_RES 3..23: note = Carboxymethyl C (4) [3, 4, 11, and/or 23]
KECCHGDLLECADDRADLAKYICDNQDTISSKLKE

SEQ ID NO: 430 - protein, 45 aa, Homo sapiens
  MOD_RES 1..44: note = Glycation (6) [1, 8, 11, 30, 42, and/or 44]
  MOD_RES 13..33: note = Carboxymethyl C (4) [13, 14, 21, and/or 33]
KLVTDLTKVHKECCHGDLLECADDRADLAKYICDNQDTISSKLKE

SEQ ID NO: 431 - protein, 41 aa, Homo sapiens
  MOD_RES 1..41: note = Glycation (4) [4 ?]
  MOD_RES 9..29: note = Carboxymethyl C (4) [9, 10, 17, and/or 29]
  MOD_RES 31: note = Deamidation N [31]
DLTKVHKECCHGDLLECADDRADLAKYICDNQDTISSKLKE

SEQ ID NO: 432 - protein, 38 aa, Homo sapiens
  MOD_RES 1..38: note = Glycation [?]
  MOD_RES 6..26: note = Carboxymethyl C (4) [6, 7, 14, and/or 26]
  MOD_RES 28: note = Deamidation N [28]
KVHKECCHGDLLECADDRADLAKYICDNQDTISSKLKE

SEQ ID NO: 433 - protein, 50 aa, Homo sapiens
  MOD_RES 1..50: note = Glycation (3) [3?]
  MOD_RES 15..49: note = Carboxymethyl C (6) [15, 16, 23, 35, 48, and/or 49]
VTKLVTDLTKVHKECCHGDLLECADDRADLAKYICDNQDTISSKLKECCD

SEQ ID NO: 434 - protein, 53 aa, Homo sapiens
  MOD_RES 1..53: note = Glycation (5) [5 ?]
  MOD_RES 13..47: note = Carboxymethyl C (6) [13, 14, 21, 33, 46, and/or 47]
KLVTDLTKVHKECCHGDLLECADDRADLAKYICDNQDTISSKLKECCDKPLLE

SEQ ID NO: 435 - protein, 52 aa, Homo sapiens
  MOD_RES 1..52: note = Glycation (2) [2 ?]
  MOD_RES 12..46: note = Carboxymethyl C (6) [12, 13, 20, 32, 45, and/or 46]
  MOD_RES 34: note = Deamidation N [34]
LVTDLTKVHKECCHGDLLECADDRADLAKYICDNQDTISSKLKECCDKPLLE

SEQ ID NO: 436 - protein, 49 aa, Homo sapiens
  MOD_RES 1..49: note = Glycation [?]
  MOD_RES 9..43: note = Carboxymethyl C (6) [9, 10, 17, 29, 42, and/or 43]
  MOD_RES 31: note = Deamidation N [31]
DLTKVHKECCHGDLLECADDRADLAKYICDNQDTISSKLKECCDKPLLE

SEQ ID NO: 437 - protein, 8 aa, Homo sapiens
  MOD_RES 3: note = Glycation [3]
VTKLVTDL

SEQ ID NO: 438 - protein, 6 aa, Homo sapiens
  MOD_RES 3: note = Glycation [3]
VTKLVT

SEQ ID NO: 439 - protein, 5 aa, Homo sapiens
  MOD_RES 3: note = Glycation [3]
VTKLV

SEQ ID NO: 440 - protein, 34 aa, Homo sapiens
  MOD_RES 23: note = Carboxymethyl C [23]
  MOD_RES 33: note = Deamidation N [33]
KDLGEEHFKGLVLIAFSQYLQQCPFDEHVKLVNE

SEQ ID NO: 441 - protein, 8 aa, Homo sapiens
  MOD_RES 4..7: note = Glycation (2) [4 and/or 7]
DLTKVHKE

SEQ ID NO: 442 - protein, 6 aa, Homo sapiens
  MOD_RES 2..5: note = Glycation (2) [2 and/or 5]
TKVHKE

SEQ ID NO: 443 - protein, 31 aa, Homo sapiens
  MOD_RES 5..27: note = Glycation (2) [5 and/or 27]
  MOD_RES 20: note = Carboxymethyl C [20]
  MOD_RES 30: note = Deamidation N [30]
GEEHFKGLVLIAFSQYLQQCPFDEHVKLVNE

SEQ ID NO: 444 - protein, 5 aa, Homo sapiens
  MOD_RES 1..5: note = Glycation [?]
  MOD_RES 1..4: note = Glycation (2) [1 and/or 4]
KVHKE

SEQ ID NO: 445 - protein, 30 aa, Homo sapiens
  MOD_RES 1..30: note = Glycation [?]
  MOD_RES 19: note = Carboxymethyl C [19]
  MOD_RES 29: note = Deamidation N [29]
EEHFKGLVLIAFSQYLQQCPFDEHVKLVNE

SEQ ID NO: 446 - protein, 8 aa, Homo sapiens
  MOD_RES 1..2: note = Carboxymethyl C (2) [1 and/or 2]
CCHGDLLE

SEQ ID NO: 447 - protein, 23 aa, Homo sapiens
  MOD_RES 1..21: note = Carboxymethyl C (4) [1, 2, 9, and/or 21]
CCHGDLLECADDRADLAKYICDN

SEQ ID NO: 448 - protein, 15 aa, Homo sapiens
  MOD_RES 1..9: note = Carboxymethyl C (3) [1, 2, and/or 9]
CCHGDLLECADDRAD

SEQ ID NO: 449 - protein, 12 aa, Homo sapiens
  MOD_RES 1..9: note = Carboxymethyl C (3) [1, 2, and/or 9]
CCHGDLLECADD

SEQ ID NO: 450 - protein, 9 aa, Homo sapiens
  MOD_RES 1..9: note = Carboxymethyl C (3) [1, 2, and/or 9]
CCHGDLLEC

SEQ ID NO: 451 - protein, 30 aa, Homo sapiens
  MOD_RES 1..21: note = Carboxymethyl C (4) [1, 2, 9, and/or 21]
  MOD_RES 1..30: note = Glycation [?]
CCHGDLLECADDRADLAKYICDNQDTISSK

SEQ ID NO: 452 - protein, 30 aa, Homo sapiens
  MOD_RES 6..18: note = Carboxymethyl C (2) [6 and/or 18]
  MOD_RES 15..29: note = Glycation (3) [15, 27, and/or 29]
GDLLECADDRADLAKYICDNQDTISSKLKE

SEQ ID NO: 453 - protein, 36 aa, Homo sapiens
  MOD_RES 1..35: note = Carboxymethyl C (5) [1, 2, 9, 21, 34, and/or 35]
  MOD_RES 1..36: note = Glycation (2) [2 ?]
  MOD_RES 23: note = Deamidation N [23]
CCHGDLLECADDRADLAKYICDNQDTISSKLKECCD

SEQ ID NO: 454 - protein, 35 aa, Homo sapiens
  MOD_RES 1..35: note = Carboxymethyl C (5) [1, 2, 9, 21, 34, and/or 35]
  MOD_RES 1..35: note = Glycation (2) [2 ?]
  MOD_RES 23: note = Deamidation N [23]
CCHGDLLECADDRADLAKYICDNQDTISSKLKECC

SEQ ID NO: 455 - protein, 37 aa, Homo sapiens
  MOD_RES 1..37: note = Glycation (3) [3 ?]
  MOD_RES 5..31: note = Carboxymethyl C (4) [5, 17, 30, and/or 31]
  MOD_RES 19: note = Deamidation N [19]
DLLECADDRADLAKYICDNQDTISSKLKECCDKPLLE

SEQ ID NO: 456 - protein, 46 aa, Homo sapiens
  MOD_RES 1..45: note = Carboxymethyl C (7) [1, 2, 9, 21, 34, 35, and/or 45]
  MOD_RES 1..46: note = Glycation (3) [3 ?]
  MOD_RES 23: note = Deamidation N [23]
CCHGDLLECADDRADLAKYICDNQDTISSKLKECCDKPLLEKSHCI

SEQ ID NO: 457 - protein, 7 aa, Homo sapiens
  MOD_RES 1..2: note = Carboxymethyl C (2) [1 and/or 2]
CCHGDLL

SEQ ID NO: 458 - protein, 32 aa, Homo sapiens
  MOD_RES 1..27: note = Carboxymethyl C (4) [1, 13, 26, and/or 27]
  MOD_RES 1..32: note = Glycation (3) [3 ?]
CADDRADLAKYICDNQDTISSKLKECCDKPLL

SEQ ID NO: 459 - protein, 29 aa, Homo sapiens
  MOD_RES 1..27: note = Carboxymethyl C (4) [1, 13, 26, and/or 27]
  MOD_RES 1..29: note = Glycation [?]
CADDRADLAKYICDNQDTISSKLKECCDK

SEQ ID NO: 460 - protein, 28 aa, Homo sapiens
  MOD_RES 1..27: note = Carboxymethyl C (4) [1, 13, 26, and/or 27]
  MOD_RES 10..24: note = Glycation (3) [10, 22, and/or 24]
CADDRADLAKYICDNQDTISSKLKECCD

SEQ ID NO: 461 - protein, 9 aa, Homo sapiens
  MOD_RES 2..3: note = Carboxymethyl C (2) [2 and/or 3]
  MOD_RES 5: note = Glycation [5]
ECCDKPLLE

SEQ ID NO: 462 - protein, 26 aa, Homo sapiens
  MOD_RES 1..26: note = Glycation (3) [3 ?]
  MOD_RES 6..20: note = Carboxymethyl C (3) [6, 19, and/or 20]
LAKYICDNQDTISSKLKECCDKPLLE

SEQ ID NO: 463 - protein, 25 aa, Homo sapiens
  MOD_RES 2..21: note = Glycation (4) [2, 14, 16, and/or 21]
  MOD_RES 5..19: note = Carboxymethyl C (3) [5, 18, and/or 19]
AKYICDNQDTISSKLKECCDKPLLE

SEQ ID NO: 464 - protein, 40 aa, Homo sapiens
  MOD_RES 1..37: note = Carboxymethyl C (5) [1, 13, 26, 27, and/or 37]
  MOD_RES 15: note = DeamIdatIon N [15]
CADDRADLAKYICDNQDTISSKLKECCDKPLLEKSHCIAE

SEQ ID NO: 465 - protein, 34 aa, Homo sapiens
  MOD_RES 1..27: note = Carboxymethyl C (4) [1, 13, 26, and/or 27]
  MOD_RES 10..34: note = Glycation (5) [10, 22, 24, 29, and/or 34]
  MOD_RES 15: note = Deamidation N [15]
CADDRADLAKYICDNQDTISSKLKECCDKPLLEK

SEQ ID NO: 466 - protein, 26 aa, Homo sapiens
  MOD_RES 8..20: note = Glycation (4) [8, 10, 15, and/or 20]
  MOD_RES 12..23: note = Carboxymethyl C (3) [12, 13, and/or 23]
NQDTISSKLKECCDKPLLEKSHCIAE

SEQ ID NO: 467 - protein, 32 aa, Homo sapiens
  MOD_RES 1..32: note = Glycation (4) [4 ?]
  MOD_RES 5..29: note = Carboxymethyl C (4) [5, 18, 19, and/or 29]
  MOD_RES 7: note = Deamidation N [7]
AKYICDNQDTISSKLKECCDKPLLEKSHCIAE

SEQ ID NO: 468 - protein, 42 aa, Homo sapiens
  MOD_RES 1..37: note = Carboxymethyl C (5) [1, 13, 26, 27, and/or 37]
  MOD_RES 1..42: note = Glycation [?]
CADDRADLAKYICDNQDTISSKLKECCDKPLLEKSHCIAEVE

SEQ ID NO: 469 - protein, 37 aa, Homo sapiens
  MOD_RES 1..37: note = Glycation (3) [3 ?]
  MOD_RES 8..32: note = Carboxymethyl C (4) [8, 21, 22, and/or 32]
ADLAKYICDNQDTISSKLKECCDKPLLEKSHCIAEVE

SEQ ID NO: 470 - protein, 11 aa, Homo sapiens
  MOD_RES 1: note = Carboxymethyl C [1]
CADDRADLAKY

SEQ ID NO: 471 - protein, 10 aa, Homo sapiens
  MOD_RES 1: note = Carboxymethyl C [1]
  MOD_RES 10: note = Glycation [10]
CADDRADLAK

SEQ ID NO: 472 - protein, 7 aa, Homo sapiens
  MOD_RES 1: note = Carboxymethyl C [1]
CADDRAD

SEQ ID NO: 473 - protein, 6 aa, Homo sapiens
  MOD_RES 1: note = Carboxymethyl C [1]
CADDRA

SEQ ID NO: 474 - protein, 23 aa, Homo sapiens
  MOD_RES 1..13: note = Carboxymethyl C (2) [1 and/or 13]
  MOD_RES 10..22: note = Glycation (2) [10 and/or 22]
CADDRADLAKYICDNQDTISSKL

SEQ ID NO: 475 - protein, 5 aa, Homo sapiens
  MOD_RES 1: note = Carboxymethyl C [1]
CADDR

SEQ ID NO: 476 - protein, 4 aa, Homo sapiens
  MOD_RES 1: note = Carboxymethyl C [1]
CADD

SEQ ID NO: 477 - protein, 22 aa, Homo sapiens
  MOD_RES 1..13: note = Carboxymethyl C (2) [1 and/or 13]
  MOD_RES 10..22: note = Glycation (2) [10 22]
CADDRADLAKYICDNQDTISSK

SEQ ID NO: 478 - protein, 17 aa, Homo sapiens
  MOD_RES 1..13: note = Carboxymethyl C (2) [1 and/or 13]
  MOD_RES 15: note = Deamidation N [15]
CADDRADLAKYICDNQD

SEQ ID NO: 479 - protein, 16 aa, Homo sapiens
  MOD_RES 1..13: note = Carboxymethyl C (2) [1 and/or 13]
  MOD_RES 15: note = Deamidation N [15]
CADDRADLAKYICDNQ

SEQ ID NO: 480 - protein, 7 aa, Homo sapiens
  MOD_RES 4..6: note = Glycation (2) [4 and/or 6]
ISSKLKE

SEQ ID NO: 481 - protein, 5 aa, Homo sapiens
  MOD_RES 1..5: note = Glycation [?]
SKLKE

SEQ ID NO: 482 - protein, 10 aa, Homo sapiens
  MOD_RES 7: note = Carboxymethyl C [7]
LLEKSHCIAE

SEQ ID NO: 483 - protein, 17 aa, Homo sapiens
  MOD_RES 1..12: note = Carboxymethyl C (3) [1, 2, and/or 12]
CCDKPLLEKSHCIAEVE

SEQ ID NO: 484 - protein, 4 aa, Homo sapiens
  MOD_RES 1..2: note = Carboxymethyl C (2) [1 and/or 2]
CCDK

SEQ ID NO: 485 - protein, 7 aa, Homo sapiens
  MOD_RES 1: note = Carboxymethyl C [1]
  MOD_RES 3: note = Glycation [3]
CDKPLLE

SEQ ID NO: 486 - protein, 6 aa, Homo sapiens
  MOD_RES 1: note = Carboxymethyl C [1]
CIAEVE

SEQ ID NO: 487 - protein, 10 aa, Homo sapiens
  MOD_RES 1..10: note = Glycation (2) [1 and/or 10]
  MOD_RES 4: note = Carboxymethyl C [4]
KSHCIAEVEK

SEQ ID NO: 488 - protein, 10 aa, Homo sapiens
  MOD_RES 5: note = Glycation [5]
AEVEKDAIPE

SEQ ID NO: 489 - protein, 26 aa, Homo sapiens
  MOD_RES 1..10: note = Glycation (2) [1 and/or 10]
  MOD_RES 1: note = Deamidation N [1]
  MOD_RES 4: note = Carboxymethyl C [4]
KSHCIAEVEKDAIPENLPPLTADFAE

SEQ ID NO: 490 - protein, 20 aa, Homo sapiens
  MOD_RES 1..20: note = Glycation [?]
  MOD_RES 4: note = Carboxymethyl C [4]
  MOD_RES 16: note = Deamidation N [16]
KSHCIAEVEKDAIPENLPPL

SEQ ID NO: 491 - protein, 6 aa, Homo sapiens
  MOD_RES 4: note = Carboxymethyl C [4]
KSHCIA

SEQ ID NO: 492 - protein, 4 aa, Homo sapiens
  MOD_RES 1: note = Glycation [1]
  MOD_RES 4: note = Carboxymethyl C [4]
KSHC

SEQ ID NO: 493 - protein, 5 aa, Homo sapiens
  MOD_RES 2: note = Carboxymethyl C [2]
HCIAE

SEQ ID NO: 494 - protein, 4 aa, Homo sapiens
  MOD_RES 1: note = Carboxymethyl C [1]
CIAE

SEQ ID NO: 495 - protein, 8 aa, Homo sapiens
  MOD_RES 4: note = Glycation [4]
HRFKDLGE

SEQ ID NO: 496 - protein, 5 aa, Homo sapiens
  MOD_RES 1: note = Glycation [1]
KDLGE

SEQ ID NO: 497 - protein, 8 aa, Homo sapiens
  MOD_RES 3: note = Glycation [3]
VEKDAIPE

SEQ ID NO: 498 - protein, 6 aa, Homo sapiens
  MOD_RES 3: note = Glycation [3]
VEKDAI

SEQ ID NO: 499 - protein, 9 aa, Homo sapiens
  MOD_RES 3: note = Glycation [3]
  MOD_RES 9: note = DearnIdatIon N [9]
VEKDAIPEN

SEQ ID NO: 500 - protein, 13 aa, Homo sapiens
  MOD_RES 9: note = Deamidation N [9]
VEKDAIPENLPPL

SEQ ID NO: 501 - protein, 29 aa, Homo sapiens
  MOD_RES 1..29: note = Glycation [?]
  MOD_RES 7: note = Deamidation N [7]
  MOD_RES 24: note = Carboxymethyl C [24]
VEKDAIPENLPPLTADFAEDKDVCKNYQE

SEQ ID NO: 502 - protein, 9 aa, Homo sapiens
  MOD_RES 1: note = Glycation [1]
KDAIPENLP

SEQ ID NO: 503 - protein, 11 aa, Homo sapiens
  MOD_RES 1: note = Glycation [1]
  MOD_RES 7: note = Deamidation N [7]
NLPPLTADFAE

SEQ ID NO: 504 - protein, 20 aa, Homo sapiens
  MOD_RES 1..20: note = Deamidation N [?]
  MOD_RES 1..20: note = Glycation [?]
  MOD_RES 16: note = Carboxymethyl C [16]
NLPPLTADFAEDKDVCKNYQ

SEQ ID NO: 505 - protein, 18 aa, Homo sapiens
  MOD_RES 1..18: note = Deamidation N (2) [1 and/or 18]
  MOD_RES 1: note = Glycation [1]
  MOD_RES 16: note = Carboxymethyl C [16]
NLPPLTADFAEDKDVCKN

SEQ ID NO: 506 - protein, 11 aa, Homo sapiens
  MOD_RES 6: note = Carboxymethyl C [6]
  MOD_RES 8: note = Deamidation N [8]
EDKDVCKNYQE

SEQ ID NO: 507 - protein, 19 aa, Homo sapiens
  MOD_RES 1: note = Deamidation [1]
  MOD_RES 11..15: note = Glycation (2) [11 and/or 15]
  MOD_RES 14: note = Carboxymethyl C [14]
PPLTADFAEDKDVCKNYQE

SEQ ID NO: 508 - protein, 15 aa, Homo sapiens
  MOD_RES 7..11: note = Glycation (2) [7 and/or 11]
  MOD_RES 10: note = Carboxymethyl C [10]
ADFAEDKDVCKNYQE

SEQ ID NO: 509 - protein, 13 aa, Homo sapiens
  MOD_RES 1..13: note = Glycation [?]
  MOD_RES 8: note = Carboxymethyl C [8]
FAEDKDVCKNYQE

SEQ ID NO: 510 - protein, 12 aa, Homo sapiens
  MOD_RES 7: note = Carboxymethyl C [7]
  MOD_RES 9: note = Deamidation N [9]
AEDKDVCKNYQE

SEQ ID NO: 511 - protein, 27 aa, Homo sapiens
  MOD_RES 1..18: note = Deamidation N (2) [1 and/or 18]
  MOD_RES 15: note = Carboxymethyl C [15]
NLPPLTADFAEDKDVCKNYQEAKDAFL

SEQ ID NO: 512 - protein, 30 aa, Homo sapiens
  MOD_RES 13: note = Carboxymethyl C [13]
PLTADFAEDKDVCKNYQEAKDAFLGSFLYE

SEQ ID NO: 513 - protein, 31 aa, Homo sapiens
  MOD_RES 4..18: note = Glycation (3) [4, 8, and/or 14]
  MOD_RES 7: note = Carboxymethyl C [7]
  MOD_RES 9: note = Deamidation N [9]
AEDKDVCKNYQEAKDAFLGSFLYEYSRRHPE

SEQ ID NO: 514 - protein, 7 aa, Homo sapiens
  MOD_RES 1: note = Deamidation N [1]
NLPPLTA

SEQ ID NO: 515 - protein, 5 aa, Homo sapiens
  MOD_RES 1: note = Deamidation N [1]
NLPPL

SEQ ID NO: 516 - protein, 4 aa, Homo sapiens
  MOD_RES 1: note = Deamidation N [1]
NLPP

SEQ ID NO: 517 - protein, 22 aa, Homo sapiens
  MOD_RES 5: note = Carboxymethyl C [5]
DKDVCKNYQEAKDAFLGSFLYE

SEQ ID NO: 518 - protein, 11 aa, Homo sapiens
  MOD_RES 5: note = Carboxymethyl C [5]
DKDVCKNYQEA

SEQ ID NO: 519 - protein, 13 aa, Homo sapiens
  MOD_RES 1..13: note = Glycation (2) [2 ?]
  MOD_RES 5: note = Carboxymethyl C [5]
DKDVCKNYQEAKD

SEQ ID NO: 520 - protein, 20 aa, Homo sapiens
  MOD_RES 3: note = Carboxymethyl C [3]
  MOD_RES 4..10: note = Glycation (2) [4 and/or 10]
  MOD_RES 4..10: note = Glycation (2) [4 and/or 10]
DVCKNYQEAKDAFLGSFLYE

SEQ ID NO: 521 - protein, 27 aa, Homo sapiens
  MOD_RES 3: note = Carboxymethyl C [3]
  MOD_RES 5: note = Deamidation N [5]
DVCKNYQEAKDAFLGSFLYEYSRRHPE

SEQ ID NO: 522 - protein, 23 aa, Homo sapiens
  MOD_RES 1: note = Deamidation N [1]
NYQEAKDAFLGSFLYEYSRRHPE

SEQ ID NO: 523 - protein, 9 aa, Homo sapiens
  MOD_RES 5: note = Carboxymethyl C [5]
  MOD_RES 7: note = Deamidation N [7]
DKDVCKNYQ

SEQ ID NO: 524 - protein, 8 aa, Homo sapiens
  MOD_RES 1..8: note = Glycation [?]
  MOD_RES 5: note = Carboxymethyl C [5]
  MOD_RES 7: note = Deamidation N [7]
DKDVCKNY

SEQ ID NO: 525 - protein, 7 aa, Homo sapiens
  MOD_RES 1..7: note = Glycation [?]
  MOD_RES 5: note = Carboxymethyl C [5]
  MOD_RES 7: note = Deamidation N [7]
DKDVCKN

SEQ ID NO: 526 - protein, 5 aa, Homo sapiens
  MOD_RES 5: note = Carboxymethyl C [5]
DKDVC

SEQ ID NO: 527 - protein, 4 aa, Homo sapiens
  MOD_RES 2: note = Glycation [2]
  MOD_RES 2: note = Glycation [2]
DKDV

SEQ ID NO: 528 - protein, 8 aa, Homo sapiens
  MOD_RES 3: note = Carboxymethyl C [3]
  MOD_RES 4: note = Glycation [4]
  MOD_RES 5: note = Deamidation N [5]
DVCKNYQE

SEQ ID NO: 529 - protein, 6 aa, Homo sapiens
  MOD_RES 1: note = Carboxymethyl C [1]
  MOD_RES 2: note = Glycation [2]
  MOD_RES 3: note = Deamidation N [3]
CKNYQE

SEQ ID NO: 530 - protein, 5 aa, Homo sapiens
  MOD_RES 1: note = Glycation [1]
  MOD_RES 2: note = Deamidation N [2]
KNYQE

SEQ ID NO: 531 - protein, 4 aa, Homo sapiens
  MOD_RES 1: note = Deamidation N [1]
NYQE

SEQ ID NO: 532 - protein, 30 aa, Homo sapiens
  MOD_RES 1..29: note = Glycation (2) [1 and/or 29]
KDAFLGSFLYEYSRRHPEYAVSVLLRLAKE

SEQ ID NO: 533 - protein, 6 aa, Homo sapiens
  MOD_RES 2: note = Glycation [2]
AKDAFL

SEQ ID NO: 534 - protein, 5 aa, Homo sapiens
  MOD_RES 2: note = Glycation [2]
AKEYE

SEQ ID NO: 535 - protein, 8 aa, Homo sapiens
  MOD_RES 1: note = Glycation [1]
KEYEATLE

SEQ ID NO: 536 - protein, 7 aa, Homo sapiens
  MOD_RES 6: note = Glycation [6]
LLRLAKE

SEQ ID NO: 537 - protein, 5 aa, Homo sapiens
  MOD_RES 4: note = Glycation [4]
RLAKE

SEQ ID NO: 538 - protein, 44 aa, Homo sapiens
  MOD_RES 7: note = Glycation [7]
  MOD_RES 7: note = Deamidation N [7]
  MOD_RES 8..40: note = Carboxymethyl C (4) [8, 9, 17, and/or 40]
YEATLEECCAKDDPHACYSTVFDKLKHLVDEPQNLIKQNCDQFE

SEQ ID NO: 539 - protein, 25 aa, Homo sapiens
  MOD_RES 8..17: note = Carboxymethyl C (3) [8, 9 and/or 17]
YEATLEECCAKDDPHACYSTVFDKL

SEQ ID NO: 540 - protein, 20 aa, Homo sapiens
  MOD_RES 8..17: note = Carboxymethyl C (3) [8, 9, and/or 17]
  MOD_RES 11: note = Glycation [11]
YEATLEECCAKDDPHACYST

SEQ ID NO: 541 - protein, 19 aa, Homo sapiens
  MOD_RES 8..17: note = Carboxymethyl C (3) [8, 9, and/or 17]
YEATLEECCAKDDPHACYS

SEQ ID NO: 542 - protein, 15 aa, Homo sapiens
  MOD_RES 8..9: note = Carboxymethyl C (2) [8 and/or 9]
YEATLEECCAKDDPH

SEQ ID NO: 543 - protein, 14 aa, Homo sapiens
  MOD_RES 8..9: note = Carboxymethyl C (2) [8 and/or 9]
YEATLEECCAKDDP

SEQ ID NO: 544 - protein, 13 aa, Homo sapiens
  MOD_RES 8..9: note = Carboxymethyl C (2) [8 and/or 9]
  MOD_RES 11: note = Glycation [11]
YEATLEECCAKDD

SEQ ID NO: 545 - protein, 40 aa, Homo sapiens
  MOD_RES 1..40: note = Deamidation N [?]
  MOD_RES 8..40: note = Carboxymethyl C (4) [8, 9, 17, and/or 40]
YEATLEECCAKDDPHACYSTVFDKLKHLVDEPQNLIKQNC

SEQ ID NO: 546 - protein, 19 aa, Homo sapiens
  MOD_RES 6..15: note = Carboxymethyl C (3) [6, 7, and/or 15]
ATLEECCAKDDPHACYSTV

SEQ ID NO: 547 - protein, 39 aa, Homo sapiens
  MOD_RES 1..39: note = Glycation (3) [3?]
  MOD_RES 6..38: note = Carboxymethyl C (4) [6, 7, 15, and/or 38]
  MOD_RES 32..37: note = Deamidation N (2) [32 and/or 37]
ATLEECCAKDDPHACYSTVFDKLKHLVDEPQNLIKQNCD

SEQ ID NO: 548 - protein, 11 aa, Homo sapiens
  MOD_RES 6..7: note = Carboxymethyl C (2) [6 and/or 7]
ATLEECCAKDD

SEQ ID NO: 549 - protein, 10 aa, Homo sapiens
  MOD_RES 6..7: note = Carboxymethyl C (2) [6 and/or 7]
  MOD_RES 9: note = Glycation [9]
ATLEECCAKD

SEQ ID NO: 550 - protein, 8 aa, Homo sapiens
  MOD_RES 6..7: note = Carboxymethyl C (2) [6 and/or 7]
ATLEECCA

SEQ ID NO: 551 - protein, 6 aa, Homo sapiens
  MOD_RES 6: note = Carboxymethyl C [6]
ATLEEC

SEQ ID NO: 552 - protein, 37 aa, Homo sapiens
  MOD_RES 6..15: note = Carboxymethyl C (3) [6, 7, and/or 15]
  MOD_RES 9..35: note = Glycation (4) [9, 22, 24, and/or 35]
  MOD_RES 32..37: note = Deamidation N (2) [32 and/or 37]
ATLEECCAKDDPHACYSTVFDKLKHLVDEPQNLIKQN

SEQ ID NO: 553 - protein, 39 aa, Homo sapiens
  MOD_RES 1..39: note = Glycation (3) [3?]
  MOD_RES 3..35: note = Carboxymethyl C (4) [3, 4, 12, and/or 35]
EECCAKDDPHACYSTVFDKLKHLVDEPQNLIKQNCDQFE

SEQ ID NO: 554 - protein, 46 aa, Homo sapiens
  MOD_RES 1..46: note = Deamidation N [?]
  MOD_RES 1..46: note = Glycation (2) [2?]
  MOD_RES 6..38: note = Carboxymethyl C (4) [6, 7, 15, and/or 38]
  MOD_RES 9..43: note = Glycation (5) [9, 22, 24, 35, and/or 43]
  MOD_RES 32..37: note = Deamidation N (2) [32 and/or 37]
ATLEECCAKDDPHACYSTVFDKLKHLVDEPQNLIKQNCDQFEKLGE

SEQ ID NO: 555 - protein, 44 aa, Homo sapiens
  MOD_RES 1..44: note = Glycation (3) [3?]
  MOD_RES 4..36: note = Carboxymethyl C (4) [4, 5, 13, and/or 36]
LEECCAKDDPHACYSTVFDKLKHLVDEPQNLIKQNCDQFEKLGE

SEQ ID NO: 556 - protein, 38 aa, Homo sapiens
  MOD_RES 1..38: note = Glycation (2) [2?]
  MOD_RES 2..34: note = Carboxymethyl C (4) [2, 3, 11, and/or 34]
ECCAKDDPHACYSTVFDKLKHLVDEPQNLIKQNCDQFE

SEQ ID NO: 557 - protein, 18 aa, Homo sapiens
  MOD_RES 1..18: note = Glycation [?]
  MOD_RES 2..11: note = Carboxymethyl C (3) [2, 3, and/or 11]
ECCAKDDPHACYSTVFDK

SEQ ID NO: 558 - protein, 15 aa, Homo sapiens
  MOD_RES 2..11: note = Carboxymethyl C (3) [2, 3, and/or 11]
  MOD_RES 5: note = Glycation [5]
ECCAKDDPHACYSTV

SEQ ID NO: 559 - protein, 14 aa, Homo sapiens
  MOD_RES 2..11: note = Carboxymethyl C (3) [2, 3, and/or 11]
  MOD_RES 5: note = Glycation [5]
ECCAKDDPHACYST

SEQ ID NO: 560 - protein, 12 aa, Homo sapiens
  MOD_RES 2..11: note = Carboxymethyl C (3) [2, 3, and/or 11]
ECCAKDDPHACY

SEQ ID NO: 561 - protein, 11 aa, Homo sapiens
  MOD_RES 2..11: note = Carboxymethyl C (3) [2, 3, and/or 11]
ECCAKDDPHAC

SEQ ID NO: 562 - protein, 10 aa, Homo sapiens
  MOD_RES 2..3: note = Carboxymethyl C (2) [2 and/or 3]
  MOD_RES 5: note = Glycation [5]
ECCAKDDPHA

SEQ ID NO: 563 - protein, 9 aa, Homo sapiens
  MOD_RES 2..3: note = Carboxymethyl C (2) [2 and/or 3]
  MOD_RES 5: note = Glycation [5]
ECCAKDDPH

SEQ ID NO: 564 - protein, 8 aa, Homo sapiens
  MOD_RES 2..3: note = Carboxymethyl C (2) [2 and/or 3]
  MOD_RES 5: note = Glycation [5]
ECCAKDDP

SEQ ID NO: 565 - protein, 7 aa, Homo sapiens
  MOD_RES 2..3: note = Carboxymethyl C (2) [2 and/or 3]
  MOD_RES 5: note = Glycation [5]
ECCAKDD

SEQ ID NO: 566 - protein, 5 aa, Homo sapiens
  MOD_RES 2..3: note = Carboxymethyl C (2) [2 and/or 3]
  MOD_RES 5: note = Glycation [5]
ECCAK

SEQ ID NO: 567 - protein, 30 aa, Homo sapiens
  MOD_RES 2..11: note = Carboxymethyl C (3) [2, 3, and/or 11]
  MOD_RES 5..20: note = Glycation (3) [5, 18, and/or 20]
  MOD_RES 28: note = Deamidation N [28]
ECCAKDDPHACYSTVFDKLKHLVDEPQNLI

SEQ ID NO: 568 - protein, 67 aa, Homo sapiens
  MOD_RES 1..67: note = Deamidation N [?]
  MOD_RES 1..67: note = Deamidation N (2) [2?]
  MOD_RES 1..67: note = Glycation (3) [3?]
  MOD_RES 2..34: note = Carboxymethyl C (4) [2, 3, 11, and/or 34]
  MOD_RES 5..56: note = Glycation (6) [5, 18, 20, 31, 39, and/or 56]
ECCAKDDPHACYSTVFDKLKHLVDEPQNLIKQNCDQFEKLGEYGFQNALIVRYTRKVPQVSTPTLVE

SEQ ID NO: 569 - protein, 55 aa, Homo sapiens
  MOD_RES 2..34: note = Carboxymethyl C (4) [2, 3, 11, and/or 34]
  MOD_RES 28..47: note = Deamidation N (3) [28, 33, and/or 47]
ECCAKDDPHACYSTVFDKLKHLVDEPQNLIKQNCDQFEKLGEYGFQNALIVRYTR

SEQ ID NO: 570 - protein, 53 aa, Homo sapiens
  MOD_RES 1..53: note = Glycation [?]
  MOD_RES 1..53: note = Deamidation N [?]
  MOD_RES 2..34: note = Carboxymethyl C (4) [2, 3, 11, and/or 34]
ECCAKDDPHACYSTVFDKLKHLVDEPQNLIKQNCDQFEKLGEYGFQNALIVRY

SEQ ID NO: 571 - protein, 50 aa, Homo sapiens
  MOD_RES 1..50: note = Deamidation N [?]
  MOD_RES 2..34: note = Carboxymethyl C (4) [2, 3, 11, and/or 34]
  MOD_RES 5..39: note = Glycation (5) [5, 18, 20, 31, and/or 39]
ECCAKDDPHACYSTVFDKLKHLVDEPQNLIKQNCDQFEKLGEYGFQNALI

SEQ ID NO: 572 - protein, 65 aa, Homo sapiens
  MOD_RES 1..65: note = Deamidation N [?]
  MOD_RES 1..65: note = Glycation (5) [5?]
  MOD_RES 2..34: note = Carboxymethyl C (4) [2, 3, 11, and/or 34]
  MOD_RES 5..56: note = Glycation (6) [5, 18, 20, 31, 39, and/or 56]
  MOD_RES 28..47: note = Deamidation N (3) [28, 33, and/or 47]
ECCAKDDPHACYSTVFDKLKHLVDEPQNLIKQNCDQFEKLGEYGFQNALIVRYTRKVPQVSTPTL

SEQ ID NO: 573 - protein, 64 aa, Homo sapiens
  MOD_RES 1..64: note = Deamidation N (2) [2?]
  MOD_RES 1..64: note = Glycation (2) [2?]
  MOD_RES 2..34: note = Carboxymethyl C (4) [2, 3, 11, and/or 34]
  MOD_RES 5..56: note = Glycation (6) [5, 18, 20, 31, 39, and/or 56]
  MOD_RES 28..47: note = Deamidation N (3) [28, 33, and/or 47]
ECCAKDDPHACYSTVFDKLKHLVDEPQNLIKQNCDQFEKLGEYGFQNALIVRYTRKVPQVSTPT

SEQ ID NO: 574 - protein, 63 aa, Homo sapiens
  MOD_RES 2..34: note = Carboxymethyl C (4) [2, 3, 11, and/or 34]
  MOD_RES 5..56: note = Glycation (6) [5, 18, 20, 31, 39, and/or 56]
  MOD_RES 28..47: note = Deamidation N (3) [28, 33, and/or 47]
ECCAKDDPHACYSTVFDKLKHLVDEPQNLIKQNCDQFEKLGEYGFQNALIVRYTRKVPQVSTP

SEQ ID NO: 575 - protein, 62 aa, Homo sapiens
  MOD_RES 1..62: note = Glycation [?]
  MOD_RES 1..62: note = Deamidation N [?]
  MOD_RES 2..34: note = Carboxymethyl C (4) [2, 3, 11, and/or 34]
ECCAKDDPHACYSTVFDKLKHLVDEPQNLIKQNCDQFEKLGEYGFQNALIVRYTRKVPQVST

SEQ ID NO: 576 - protein, 59 aa, Homo sapiens
  MOD_RES 1..59: note = Glycation (5) [5?]
  MOD_RES 2..34: note = Carboxymethyl C (4) [2, 3, 11, and/or 34]
  MOD_RES 28..47: note = Deamidation N (3) [28, 33, and/or 47]
ECCAKDDPHACYSTVFDKLKHLVDEPQNLIKQNCDQFEKLGEYGFQNALIVRYTRKVPQ

SEQ ID NO: 577 - protein, 37 aa, Homo sapiens
  MOD_RES 1..33: note = Carboxymethyl C (4) [1, 2, 10, and/or 33]
  MOD_RES 1..37: note = Glycation (3) [3?]
  MOD_RES 27..32: note = Deamidation N (2) [27 and/or 32]
CCAKDDPHACYSTVFDKLKHLVDEPQNLIKQNCDQFE

SEQ ID NO: 578 - protein, 40 aa, Homo sapiens
  MOD_RES 1..33: note = Carboxymethyl C (4) [1, 2, 10, and/or 33]
  MOD_RES 1..40: note = Glycation [?]
  MOD_RES 1..40: note = Deamidation N [?]
CCAKDDPHACYSTVFDKLKHLVDEPQNLIKQNCDQFEKLG

SEQ ID NO: 579 - protein, 39 aa, Homo sapiens
  MOD_RES 1..33: note = Carboxymethyl C (4) [1, 2, 10, and/or 33]
  MOD_RES 1..39: note = Glycation [?]
  MOD_RES 27..32: note = Deamidation N (2) [27 and/or 32]
CCAKDDPHACYSTVFDKLKHLVDEPQNLIKQNCDQFEKL

SEQ ID NO: 580 - protein, 40 aa, Homo sapiens
  MOD_RES 1..32: note = Carboxymethyl C (3) [1, 9, and/or 32]
  MOD_RES 1..40: note = Deamidation N [?]
  MOD_RES 26..31: note = Deamidation N (2) [26 and/or 31]
CAKDDPHACYSTVFDKLKHLVDEPQNLIKQNCDQFEKLGE

SEQ ID NO: 581 - protein, 28 aa, Homo sapiens
  MOD_RES 1..28: note = Glycation (2) [2?]
  MOD_RES 1..28: note = Deamidation N [?]
  MOD_RES 20: note = Carboxymethyl C [20]
VFDKLKHLVDEPQNLIKQNCDQFEKLGE

SEQ ID NO: 582 - protein, 24 aa, Homo sapiens
  MOD_RES 10..15: note = Deamidation N (2) [10 and/or 15]
  MOD_RES 16: note = Carboxymethyl C [16]
LKHLVDEPQNLIKQNCDQFEKLGE

SEQ ID NO: 583 - protein, 23 aa, Homo sapiens
  MOD_RES 2..14: note = Deamidation N (2) [9 and/or 14]
  MOD_RES 15: note = Carboxymethyl C [15]
KHLVDEPQNLIKQNCDQFEKLGE

SEQ ID NO: 584 - protein, 39 aa, Homo sapiens
  MOD_RES 8..31: note = Carboxymethyl C (2) [8 and/or 31]
  MOD_RES 25..30: note = Deamidation N (2) [25 and/or 30]
AKDDPHACYSTVFDKLKHLVDEPQNLIKQNCDQFEKLGE

SEQ ID NO: 585 - protein, 17 aa, Homo sapiens
  MOD_RES 3..8: note = Deamidation N (2) [3 and/or 8]
  MOD_RES 6..14: note = Glycation (2) [6 and/or 14]
  MOD_RES 9: note = Carboxymethyl C [9]
PQNLIKQNCDQFEKLGE

SEQ ID NO: 586 - protein, 11 aa, Homo sapiens
  MOD_RES 3: note = Carboxymethyl C [3]
QNCDQFEKLGE

SEQ ID NO: 587 - protein, 10 aa, Homo sapiens
  MOD_RES 1: note = Deamidation N [1]
  MOD_RES 2: note = Carboxymethyl C [2]
  MOD_RES 7: note = Glycation [7]
NCDQFEKLGE

SEQ ID NO: 588 - protein, 9 aa, Homo sapiens
  MOD_RES 1: note = Carboxymethyl C [1]
  MOD_RES 6: note = Glycation [6]
CDQFEKLGE

SEQ ID NO: 589 - protein, 37 aa, Homo sapiens
  MOD_RES 1..37: note = Glycation (3) [3?]
  MOD_RES 6..29: note = Carboxymethyl C (2) [6 and/or 29]
  MOD_RES 23..28: note = Deamidation N (2) [23 and/or 28]
DDPHACYSTVFDKLKHLVDEPQNLIKQNCDQFEKLGE

SEQ ID NO: 590 - protein, 33 aa, Homo sapiens
  MOD_RES 2..25: note = Carboxymethyl C (2) [2 and/or 25]
ACYSTVFDKLKHLVDEPQNLIKQNCDQFEKLGE

SEQ ID NO: 591 - protein, 48 aa, Homo sapiens
  MOD_RES 1..33: note = Carboxymethyl C (4) [1, 2, 10, and/or 33]
  MOD_RES 1..48: note = Carboxymethyl C (4) [1, 2, 10, 33, and/or 48]
  MOD_RES 1..48: note = Glycation (5) [5?]
  MOD_RES 1..48: note = Glycation (6) [6?]
  MOD_RES 1..48: note = Glycation (4) [4?]
  MOD_RES 1..48: note = Glycation (3) [3?]
  MOD_RES 1..48: note = Deamidation N [?]
  MOD_RES 1..48: note = Deamidation N (2) [2?]
  MOD_RES 1..48: note = Glycation [?]
  MOD_RES 1..48: note = Deamidation N (2) [2?]
  MOD_RES 4..48: note = Glycation (6) [4, 17, 10, 30, 38, and/or 48]
  MOD_RES 4..38: note = Glycation (5) [4, 17, 19, 30, and/or 38)
  MOD_RES 27..32: note = Deamidation N (2) [27 and/or 32]
  MOD_RES 27..46: note = Deamidation N (3) [27, 32, and/or 46]
CCAKDDPHACYSTVFDKLKHLVDEPQNLIKQNCDQFEKLGEYGFQNAL

SEQ ID NO: 592 - protein, 46 aa, Homo sapiens
  MOD_RES 1..33: note = Carboxymethyl C (4) [1, 2, 10, and/or 33]
  MOD_RES 1..46: note = Glycation (3) [3?]
CCAKDDPHACYSTVFDKLKHLVDEPQNLIKQNCDQFEKLGEYGFQN

SEQ ID NO: 593 - protein, 43 aa, Homo sapiens
  MOD_RES 1..33: note = Carboxymethyl C (4) [1, 2, 10, and/or 33]
  MOD_RES 1..43: note = Glycation [?]
  MOD_RES 27..32: note = Deamidation N (2) [27 and/or 32]
CCAKDDPHACYSTVFDKLKHLVDEPQNLIKQNCDQFEKLGEYG

SEQ ID NO: 594 - protein, 49 aa, Homo sapiens
  MOD_RES 5..43: note = Glycation (5) [5, 7, 18, 26, and/or 43]
  MOD_RES 15..34: note = Deamidation N (3) [15, 20, and/or 34]
  MOD_RES 21: note = Carboxymethyl C [21]
TVFDKLKHLVDEPQNLIKQNCDQFEKLGEYGFQNALIVRYTRKVPQVST

SEQ ID NO: 595 - protein, 49 aa, Homo sapiens
  MOD_RES 1..49: note = Deamidation N [?]
  MOD_RES 1..49: note = Glycation (3) [3?]
  MOD_RES 1..49: note = Glycation (2) [2?]
  MOD_RES 12..31: note = Deamidation N (3) [12, 17, and/or 31]
  MOD_RES 18: note = Carboxymethyl C [18]
DKLKHLVDEPQNLIKQNCDQFEKLGEYGFQNALIVRYTRKVPQVSTPTL

SEQ ID NO: 596 - protein, 39 aa, Homo sapiens
  MOD_RES 1..39: note = Glycation [?]
  MOD_RES 1..39: note = Deamidation N [?]
  MOD_RES 6: note = Carboxymethyl C [6]
LIKQNCDQFEKLGEYGFQNALIVRYTRKVPQVSTPTLVE

SEQ ID NO: 597 - protein, 32 aa, Homo sapiens
  MOD_RES 4..21: note = Glycation (2) [4 and/or 21]
QFEKLGEYGFQNALIVRYTRKVPQVSTPTLVE

SEQ ID NO: 598 - protein, 49 aa, Homo sapiens
  MOD_RES 1..49: note = Deamidation N (2) [2?]
  MOD_RES 1..49: note = Glycation (4) [4?]
  MOD_RES 2..25: note = Carboxymethyl C (2) [2 and/or 25]
ACYSTVFDKLKHLVDEPQNLIKQNCDQFEKLGEYGFQNALIVRYTRKVP

SEQ ID NO: 599 - protein, 49 aa, Homo sapiens
  MOD_RES 1..24: note = Carboxymethyl C (2) [1 and/or 24]
  MOD_RES 1..49: note = Glycation (3) [3?]
  MOD_RES 1..49: note = Deamidation N [?]
CYSTVFDKLKHLVDEPQNLIKQNCDQFEKLGEYGFQNALIVRYTRKVPC

SEQ ID NO: 600 - protein, 49 aa, Homo sapiens
  MOD_RES 1..49: note = Deamidation N [?]
  MOD_RES 9..49: note = Glycation (4) [9, 17, 34, and/or 49]
  MOD_RES 12..49: note = Carboxymethyl C (2) [12 and/or 49]
VDEPQNLIKQNCDQFEKLGEYGFQNALIVRYTRKVPQVSTPTLVEVSRS

SEQ ID NO: 601 - protein, 49 aa, Homo sapiens
  MOD_RES 1..49: note = Deamidation N [?]
  MOD_RES 10..49: note = Carboxymethyl C (2) [10 and/or 49]
EPQNLIKQNCDQFEKLGEYGFQNALIVRYTRKVPQVSTPTLVEVSRSLG

SEQ ID NO: 602 - protein, 49 aa, Homo sapiens
  MOD_RES 1..49: note = Glycation (4) [4?]
  MOD_RES 1..20: note = Deamidation N (3) [1, 6, and/or 20]
  MOD_RES 7..49: note = Carboxymethyl C (2) [7 and/or 49]
NLIKQNCDQFEKLGEYGFQNALIVRYTRKVPQVSTPTLVEVSRSLGKVG

SEQ ID NO: 603 - protein, 49 aa, Homo sapiens
  MOD_RES 1..49: note = Glycation (3) [3?]
  MOD_RES 4..18: note = Deamidation N (2) [4 and/or 18]
  MOD_RES 5..49: note = Carboxymethyl C (2) [5 and/or 49]
IKQNCDQFEKLGEYGFQNALIVRYTRKVPQVSTPTLVEVSRSLGKVGTR

SEQ ID NO: 604 - protein, 17 aa, Homo sapiens
  MOD_RES 1..10: note = Carboxymethyl C (3) [1, 2, and/or 10]
  MOD_RES 4..17: note = Glycation (2) [4 and/or 17]
CCAKDDPHACYSTVFDK

SEQ ID NO: 605 - protein, 16 aa, Homo sapiens
  MOD_RES 1..10: note = Carboxymethyl C (3) [1, 2, and/or 10]
  MOD_RES 4: note = Glycation [4]
CCAKDDPHACYSTVFD

SEQ ID NO: 606 - protein, 14 aa, Homo sapiens
  MOD_RES 1..10: note = Carboxymethyl C (3) [1, 2, and/or 10]
CCAKDDPHACYSTV

SEQ ID NO: 607 - protein, 13 aa, Homo sapiens
  MOD_RES 1..10: note = Carboxymethyl C (3) [1, 2, and/or 10]
CCAKDDPHACYST

SEQ ID NO: 608 - protein, 12 aa, Homo sapiens
  MOD_RES 1..10: note = Carboxymethyl C (3) [1, 2, and/or 10]
CCAKDDPHACYS

SEQ ID NO: 609 - protein, 8 aa, Homo sapiens
  MOD_RES 1..2: note = Carboxymethyl C (2) [1 and/or 2]
  MOD_RES 4: note = Glycation [4]
CCAKDDPH

SEQ ID NO: 610 - protein, 7 aa, Homo sapiens
  MOD_RES 1..2: note = Carboxymethyl C (2) [1 and/or 2]
CCAKDDP

SEQ ID NO: 611 - protein, 6 aa, Homo sapiens
  MOD_RES 1..2: note = Carboxymethyl C (2) [1 and/or 2]
  MOD_RES 4: note = Glycation [4]
CCAKDD

SEQ ID NO: 612 - protein, 5 aa, Homo sapiens
  MOD_RES 1..2: note = Carboxymethyl C (2) [1 and/or 2]
  MOD_RES 4: note = Glycation [4]
CCAKD

SEQ ID NO: 613 - protein, 4 aa, Homo sapiens
  MOD_RES 1..2: note = Carboxymethyl C (2) [1 and/or 2]
  MOD_RES 4: note = Glycation [4]
CCAK

SEQ ID NO: 614 - protein, 33 aa, Homo sapiens
  MOD_RES 1..33: note = Carboxymethyl C (4) [1, 2, 10, and/or 33]
  MOD_RES 1..33: note = Glycation (3) [3?]
  MOD_RES 1..33: note = Deamidation N [?]
CCAKDDPHACYSTVFDKLKHLVDEPQNLIKQNC

SEQ ID NO: 615 - protein, 29 aa, Homo sapiens
  MOD_RES 1..10: note = Carboxymethyl C (3) [1, 2, and/or 10]
  MOD_RES 1..29: note = Glycation [?]
CCAKDDPHACYSTVFDKLKHLVDEPQNLI

SEQ ID NO: 616 - protein, 36 aa, Homo sapiens
  MOD_RES 1..32: note = Carboxymethyl C (3) [1, 9, and/or 32]
  MOD_RES 1..36: note = Glycation [?]
  MOD_RES 26..31: note = Deamidation N (2) [26 and/or 31]
CAKDDPHACYSTVFDKLKHLVDEPQNLIKQNCDQFE

SEQ ID NO: 617 - protein, 21 aa, Homo sapiens
  MOD_RES 11..16: note = Deamidation N (2) [11 and/or 16]
  MOD_RES 17: note = Carboxymethyl C [17]
KLKHLVDEPQNLIKQNCDQFE

SEQ ID NO: 618 - protein, 20 aa, Homo sapiens
  MOD_RES 2..13: note = Glycation (2) [2 and/or 13]
  MOD_RES 10..15: note = Deamidation N (2) [10 and/or 15]
  MOD_RES 16: note = Carboxymethyl C [16]
LKHLVDEPQNLIKQNCDQFE

SEQ ID NO: 619 - protein, 19 aa, Homo sapiens
  MOD_RES 1..19: note = Glycation [?]
  MOD_RES 9..14: note = Deamidation N (2) [9 and/or 14]
  MOD_RES 15: note = Carboxymethyl C [15]
KHLVDEPQNLIKQNCDQFE

SEQ ID NO: 620 - protein, 14 aa, Homo sapiens
  MOD_RES 1..14: note = Deamidation N [?]
  MOD_RES 7: note = Glycation [7]
  MOD_RES 10: note = Carboxymethyl C [10]
EPQNLIKQNCDQFE

SEQ ID NO: 621 - protein, 13 aa, Homo sapiens
  MOD_RES 9: note = Carboxymethyl C [9]
PQNLIKQNCDQFE

SEQ ID NO: 622 - protein, 12 aa, Homo sapiens
  MOD_RES 5: note = Glycation [5]
  MOD_RES 8: note = Carboxymethyl C [8]
QNLIKQNCDQFE

SEQ ID NO: 623 - protein, 9 aa, Homo sapiens
  MOD_RES 5: note = Carboxymethyl C [5]
IKQNCDQFE

SEQ ID NO: 624 - protein, 8 aa, Homo sapiens
  MOD_RES 1: note = Glycation [1]
  MOD_RES 3: note = Deamidation N [3]
  MOD_RES 4: note = Carboxymethyl C [4]
KQNCDQFE

SEQ ID NO: 625 - protein, 30 aa, Homo sapiens
  MOD_RES 1..30: note = Glycation (2) [2?]
  MOD_RES 3..26: note = Carboxymethyl C (2) [3 and/or 26]
  MOD_RES 20..25: note = Deamidation N (2) [20 and/or 25]
HACYSTVFDKLKHLVDEPQNLIKQNCDQFE

SEQ ID NO: 626 - protein, 10 aa, Homo sapiens
  MOD_RES 1: note = Glycation [1]
  MOD_RES 9: note = Deamidation N [9]
KLGEYGFQNA

SEQ ID NO: 627 - protein, 6 aa, Homo sapiens
  MOD_RES 1: note = Glycation [1]
KLGEYG

SEQ ID NO: 628 - protein, 23 aa, Homo sapiens
KLGEYGFQNALIVRYTRKVPQVS

SEQ ID NO: 629 - protein, 46 aa, Homo sapiens
  MOD_RES 1..46: note = Glycation (3) [3?]
  MOD_RES 1..46: note = Glycation [?]
  MOD_RES 9: note = Deamidation N [9]
  MOD_RES 41..42: note = Carboxymethyl C (2) [41 and/or 42]
KLGEYGFQNALIVRYTRKVPQVSTPTLVEVSRSLGKVGTRCCTKPE

SEQ ID NO: 630 - protein, 40 aa, Homo sapiens
  MOD_RES 1..40: note = Glycation [?]
  MOD_RES 9: note = Deamidation N [9]
KLGEYGFQNALIVRYTRKVPQVSTPTLVEVSRSLGKVGTR

SEQ ID NO: 631 - protein, 44 aa, Homo sapiens
  MOD_RES 7: note = Deamidation N [7]
  MOD_RES 16..42: note = Glycation (3) [16, 34, and/or 42]
  MOD_RES 39..40: note = Carboxymethyl C (2) [39 and/or 40]
GEYGFQNALIVRYTRKVPQVSTPTLVEVSRSLGKVGTRCCTKPE

SEQ ID NO: 632 - protein, 48 aa, Homo sapiens
  MOD_RES 9: note = Deamidation N [9]
  MOD_RES 41..42: note = Carboxymethyl C (2) [41 and/or 42]
KLGEYGFQNALIVRYTRKVPQVSTPTLVEVSRSLGKVGTRCCTKPESE

SEQ ID NO: 633 - protein, 35 aa, Homo sapiens
  MOD_RES 1..35: note = Glycation [?]
YGFQNALIVRYTRKVPQVSTPTLVEVSRSLGKVGT

SEQ ID NO: 634 - protein, 40 aa, Homo sapiens
  MOD_RES 12..38: note = Glycation (3) [12, 30, and/or 38]
  MOD_RES 35..36: note = Carboxymethyl C (2) [35 and/or 36]
FQNALIVRYTRKVPQVSTPTLVEVSRSLGKVGTRCCTKPE

SEQ ID NO: 635 - protein, 39 aa, Homo sapiens
  MOD_RES 34..35: note = Carboxymethyl C (2) [34 and/or 35]
QNALIVRYTRKVPQVSTPTLVEVSRSLGKVGTRCCTKPE

SEQ ID NO: 636 - protein, 44 aa, Homo sapiens
  MOD_RES 1..44: note = Glycation [?]
  MOD_RES 5: note = Deamidation N [5]
  MOD_RES 37..38: note = Carboxymethyl C (2) [37 and/or 38]
YGFQNALIVRYTRKVPQVSTPTLVEVSRSLGKVGTRCCTKPESE

SEQ ID NO: 637 - protein, 33 aa, Homo sapiens
  MOD_RES 26..27: note = Carboxymethyl C (2) [26 and/or 27]
TRKVPQVSTPTLVEVSRSLGKVGTRCCTKPESE

SEQ ID NO: 638 - protein, 40 aa, Homo sapiens
  MOD_RES 1..40: note = Glycation [?]
  MOD_RES 33..34: note = Carboxymethyl C (2) [33 and/or 34]
NALIVRYTRKVPQVSTPTLVEVSRSLGKVGTRCCTKPESE

SEQ ID NO: 639 - protein, 35 aa, Homo sapiens
  MOD_RES 22..33: note = Carboxymethyl C (3) [22, 23, and/or 33]
PQVSTPTLVEVSRSLGKVGTRCCTKPESERMPCTE

SEQ ID NO: 640 - protein, 45 aa, Homo sapiens
  MOD_RES 1..45: note = Glycation [?]
  MOD_RES 32..43: note = Carboxymethyl C (3) [32, 33, and/or 43]
ALIVRYTRKVPQVSTPTLVEVSRSLGKVGTRCCTKPESERMPCTE

SEQ ID NO: 641 - protein, 5 aa, Homo sapiens
  MOD_RES 5: note = Deamidation N [5]
YGFQN

SEQ ID NO: 642 - protein, 9 aa, Homo sapiens
  MOD_RES 2..3: note = Carboxymethyl C (2) [2 and/or 3]
  MOD_RES 5: note = Glycation [5]
RCCTKPESE

SEQ ID NO: 643 - protein, 8 aa, Homo sapiens
  MOD_RES 1..2: note = Carboxymethyl C (2) [1 and/or 2]
  MOD_RES 4: note = Glycation [4]
CCTKPESE

SEQ ID NO: 644 - protein, 7 aa, Homo sapiens
  MOD_RES 1: note = Carboxymethyl C [1]
CTKPESE

SEQ ID NO: 645 - protein, 14 aa, Homo sapiens
  MOD_RES 7..8: note = Carboxymethyl C (2) [7 and/or 8]
GKVGTRCCTKPESE

SEQ ID NO: 646 - protein, 12 aa, Homo sapiens
  MOD_RES 5..6: note = Carboxymethyl C (2) [5 and/or 6]
VGTRCCTKPESE

SEQ ID NO: 647 - protein, 15 aa, Homo sapiens
  MOD_RES 2..13: note = Carboxymethyl C (3) [2, 3, and/or 13]
  MOD_RES 5: note = Glycation [5]
  MOD_RES 11: note = Oxidation M [11]
RCCTKPESERMPCTE

SEQ ID NO: 648 - protein, 14 aa, Homo sapiens
  MOD_RES 1..12: note = Carboxymethyl C (3) [1, 2, and/or 12]
CCTKPESERMPCTE

SEQ ID NO: 649 - protein, 12 aa, Homo sapiens
  MOD_RES 2: note = Glycation [2]
  MOD_RES 8: note = Oxidation M [8]
  MOD_RES 10: note = Carboxymethyl C [10]
TKPESERMPCTE

SEQ ID NO: 650 - protein, 10 aa, Homo sapiens
  MOD_RES 6: note = Oxidation M [6]
  MOD_RES 8: note = Carboxymethyl C [8]
PESERMPCTE

SEQ ID NO: 651 - protein, 18 aa, Homo sapiens
  MOD_RES 5..16: note = Carboxymethyl C (3) [5, 6, and/or 16]
  MOD_RES 8: note = Glycation [8]
  MOD_RES 14: note = Oxidation M [14]
VGTRCCTKPESERMPCTE

SEQ ID NO: 652 - protein, 16 aa, Homo sapiens
  MOD_RES 3..14: note = Carboxymethyl C (3) [3, 4, and/or 14]
TRCCTKPESERMPCTE

SEQ ID NO: 653 - protein, 39 aa, Homo sapiens
  MOD_RES 6..14: note = Glycation (2) [6 and/or 14]
  MOD_RES 11..35: note = Carboxymethyl C (4) [11, 12, 22, and/or 35]
SRSLGKVGTRCCTKPESERMPCTEDYLSLILNRLCVLHE

SEQ ID NO: 654 - protein, 27 aa, Homo sapiens
  MOD_RES 8: note = Oxidation M [8]
  MOD_RES 10..23: note = Carboxymethyl C (2) [10 and/or 23]
  MOD_RES 20: note = Deamidation N [20]
TKPESERMPCTEDYLSLILNRLCVLHE

SEQ ID NO: 655 - protein, 36 aa, Homo sapiens
  MOD_RES 8..32: note = Carboxymethyl C (4) [8, 9, 19, and/or 32]
LGKVGTRCCTKPESERMPCTEDYLSLILNRLCVLHE

SEQ ID NO: 656 - protein, 35 aa, Homo sapiens
  MOD_RES 2..10: note = Glycation (2) [2 and/or 10]
  MOD_RES 7..31: note = Carboxymethyl C (4) [7, 8, 18, and/or 31]
  MOD_RES 28: note = Deamidation N [28]
GKVGTRCCTKPESERMPCTEDYLSLILNRLCVLHE

SEQ ID NO: 657 - protein, 10 aa, Homo sapiens
  MOD_RES 7: note = Glycation [7]
VSRSLGKVGT

SEQ ID NO: 658 - protein, 7 aa, Homo sapiens
  MOD_RES 2..3: note = Carboxymethyl C (2) [2 and/or 3]
  MOD_RES 5: note = Glycation [5]
RCCTKPE

SEQ ID NO: 659 - protein, 6 aa, Homo sapiens
  MOD_RES 1..2: note = Carboxymethyl C (2) [1 and/or 2]
CCTKPE

SEQ ID NO: 660 - protein, 10 aa, Homo sapiens
  MOD_RES 5..6: note = Carboxymethyl C (2) [5 and/or 6]
VGTRCCTKPE

SEQ ID NO: 661 - protein, 9 aa, Homo sapiens
  MOD_RES 4..5: note = Carboxymethyl C (2) [4 and/or 5]
  MOD_RES 7: note = Glycation [7]
GTRCCTKPE

SEQ ID NO: 662 - protein, 8 aa, Homo sapiens
  MOD_RES 6: note = Carboxymethyl C [6]
SERMPCTE

SEQ ID NO: 663 - protein, 7 aa, Homo sapiens
  MOD_RES 4: note = Oxidation M [4]
  MOD_RES 6: note = Carboxymethyl C [6]
SERMPCT

SEQ ID NO: 664 - protein, 7 aa, Homo sapiens
  MOD_RES 3: note = Oxidation M [3]
  MOD_RES 5: note = Carboxymethyl C [5]
ERMPCTE

SEQ ID NO: 665 - protein, 10 aa, Homo sapiens
  MOD_RES 4: note = Oxidation M [4]
  MOD_RES 6: note = Carboxymethyl C [6]
SERMPCTEDY

SEQ ID NO: 666 - protein, 9 aa, Homo sapiens
  MOD_RES 6: note = Carboxymethyl C [6]
SERMPCTED

SEQ ID NO: 667 - protein, 6 aa, Homo sapiens
  MOD_RES 2: note = Oxidation M [2]
  MOD_RES 4: note = Carboxymethyl C [4]
RMPCTE

SEQ ID NO: 668 - protein, 10 aa, Homo sapiens
  MOD_RES 2: note = Oxidation M [2]
  MOD_RES 4: note = Carboxymethyl C [4]
RMPCTEDYLS

SEQ ID NO: 669 - protein, 9 aa, Homo sapiens
  MOD_RES 4: note = Carboxymethyl C [4]
RMPCTEDYL

SEQ ID NO: 670 - protein, 8 aa, Homo sapiens
  MOD_RES 4: note = Carboxymethyl C [4]
RMPCTEDY

SEQ ID NO: 671 - protein, 7 aa, Homo sapiens
  MOD_RES 4: note = Carboxymethyl C [4]
RMPCTED

SEQ ID NO: 672 - protein, 18 aa, Homo sapiens
  MOD_RES 2: note = Oxidation M [2]
  MOD_RES 4..17: note = Carboxymethyl C (2) [4 and/or 17]
  MOD_RES 14: note = Deamidation N [14]
RMPCTEDYLSLILNRLCV

SEQ ID NO: 673 - protein, 14 aa, Homo sapiens
  MOD_RES 2: note = Oxidation M [2]
  MOD_RES 4: note = Carboxymethyl C [4]
  MOD_RES 14: note = Deamidation N [14]
RMPCTEDYLSLILN

SEQ ID NO: 674 - protein, 34 aa, Homo sapiens
  MOD_RES 1: note = Oxidation M [1]
  MOD_RES 1..34: note = Glycation (2) [2 ?]
  MOD_RES 3..32: note = Carboxymethyl C (4) [3, 16, 31, and/or 32]
  MOD_RES 13: note = Deamidation N [13]
MPCTEDYLSLILNRLCVLHEKTPVSEKVTKCCTE

SEQ ID NO: 675 - protein, 5 aa, Homo sapiens
  MOD_RES 4: note = Carboxymethyl C [4]
RMPCT

SEQ ID NO: 676 - protein, 4 aa, Homo sapiens
  MOD_RES 2: note = Oxidation M [2]
  MOD_RES 4: note = Carboxymethyl C [4]
RMPC

SEQ ID NO: 677 - protein, 5 aa, Homo sapiens
  MOD_RES 1: note = Oxidation M [1]
  MOD_RES 3: note = Carboxymethyl C [3]
MPCTE

SEQ ID NO: 678 - protein, 14 aa, Homo sapiens
  MOD_RES 3: note = Carboxymethyl C [3]
QQCPFDEHVKLVNE

SEQ ID NO: 679 - protein, 4 aa, Homo sapiens
  MOD_RES 2: note = Carboxymethyl C [2]
PCTE

SEQ ID NO: 680 - protein, 19 aa, Homo sapiens
  MOD_RES 8: note = Carboxymethyl C [8]
FSQYLQQCPFDEHVKLVNE

SEQ ID NO: 681 - protein, 21 aa, Homo sapiens
  MOD_RES 8: note = Deamidation N [8]
  MOD_RES 11: note = Carboxymethyl C [11]
  MOD_RES 16: note = Glycation [16]
DYLSLILNRLCVLHEKTPVSE

SEQ ID NO: 682 - protein, 41 aa, Homo sapiens
  MOD_RES 1..41: note = Glycation (2) [2 ?]
  MOD_RES 8..33: note = Deamidation N (2) [8 and/or 33]
  MOD_RES 11..37: note = Carboxymethyl C(4) [11, 26, 27, and/or 37]
DYLSLILNRLCVLHEKTPVSEKVTKCCTESLVNRRPCFSAL

SEQ ID NO: 683 - protein, 44 aa, Homo sapiens
  MOD_RES 7..32: note = Deamidation N (2) [7 and/or 32]
  MOD_RES 10..36: note = Carboxymethyl C (4) [10, 25, 26, and/or 36]
  MOD_RES 15..24: note = Glycation (3) [15, 21, and/or 24]
YLSLILNRLCVLHEKTPVSEKVTKCCTESLVNRRPCFSALTPDE

SEQ ID NO: 684 - protein, 32 aa, Homo sapiens
  MOD_RES 1..32: note = Glycation [?]
  MOD_RES 13..24: note = Carboxymethyl C (3) [13, 14, and/or 24]
  MOD_RES 20: note = Deamidation N [20]
HEKTPVSEKVTKCCTESLVNRRPCFSALTPDE

SEQ ID NO: 685 - protein, 43 aa, Homo sapiens
  MOD_RES 1..43: note = Glycation [?]
  MOD_RES 9..35: note = Carboxymethyl C (9) [9, 24, 25, and/or 35]
LSLILNRLCVLHEKTPVSEKVTKCCTESLVNRRPCFSALTPDE

SEQ ID NO: 686 - protein, 42 aa, Homo sapiens
  MOD_RES 5..30: note = Deamidation N (2) [5 and/or 30]
  MOD_RES 8..34: note = Carboxymethyl C (9) [8, 23, 29, and/or 34]
SLILNRLCVLHEKTPVSEKVTKCCTESLVNRRPCFSALTPDE

SEQ ID NO: 687 - protein, 9 aa, Homo sapiens
  MOD_RES 8: note = Deamidation N [8]
DYLSLILNR

SEQ ID NO: 688 - protein, 9 aa, Homo sapiens
  MOD_RES 5: note = Carboxymethyl C [5]
LNRLCVLHE

SEQ ID NO: 689 - protein, 8 aa, Homo sapiens
  MOD_RES 4: note = Carboxymethyl C [4]
NRLCVLHE

SEQ ID NO: 690 - protein, 6 aa, Homo sapiens
  MOD_RES 2: note = Carboxymethyl C [2]
LCVLHE

SEQ ID NO: 691 - protein, 6 aa, Homo sapiens
  MOD_RES 1: note = Glycation [1]
KTPVSE

SEQ ID NO: 692 - protein, 8 aa, Homo sapiens
  MOD_RES 1..8: note = Glycation [?]
  MOD_RES 1..7: note = Glycation (2) [1 and/or 7]
KTPVSEKV

SEQ ID NO: 693 - protein, 7 aa, Homo sapiens
  MOD_RES 1..7: note = Glycation (2) [1 and/or 7]
KTPVSEK

SEQ ID NO: 694 - protein, 11 aa, Homo sapiens
  MOD_RES 4..7: note = Glycation(2) [4 and/or 7]
  MOD_RES 8..9: note = Carboxymethyl C (2) [8 and/or 9]
VSEKVTKCCTE

SEQ ID NO: 695 - protein, 10 aa, Homo sapiens
  MOD_RES 3..6: note = Glycation (2) [3 and/or 6]
  MOD_RES 7..8: note = Carboxymethyl C (2) [7 and/or 8]
SEKVTKCCTE

SEQ ID NO: 696 - protein, 9 aa, Homo sapiens
  MOD_RES 2..5: note = Glycation (2) [2 and/or 5]
  MOD_RES 6..7: note = Carboxymethyl C (2) [6 and/or 7]
EKVTKCCTE

SEQ ID NO: 697 - protein, 30 aa, Homo sapiens
  MOD_RES 1..10: note = Glycation (3) [1, 7, and/or 10]
  MOD_RES 11..22: note = Carboxymethyl C (3) [11, 12, and/or 22]
  MOD_RES 18: note = Deamidation N [18]
KTPVSEKVTKCCTESLVNRRPCFSALTPDE

SEQ ID NO: 698 - protein, 26 aa, Homo sapiens
  MOD_RES 3..6: note = Glycation (2] [3 and/or 6]
  MOD_RES 7..18: note = Carboxymethyl C (3) [7, 8, and/or 18]
SEKVTKCCTESLVNRRPCFSALTPDE

SEQ ID NO: 699 - protein, 30 aa, Homo sapiens
  MOD_RES 1..30: note = Glycation [?]
  MOD_RES 7..26: note = Carboxymethyl C (2) [7 and/or 26]
SQYLQQCPFDEHVKLVNELTEFAKTCVADE

SEQ ID NO: 700 - protein, 20 aa, Homo sapiens
  MOD_RES 1..20: note = Glycation [?]
  MOD_RES 7: note = Deamidation N [7]
  MOD_RES 16: note = Carboxymethyl C [16]
EHVKLVNELTEFAKTCVADE

SEQ ID NO: 701 - protein, 8 aa, Homo sapiens
  MOD_RES 5..6: note = Carboxymethyl C (2) [5 and/or 6]
KVTKCCTE

SEQ ID NO: 702 - protein, 11 aa, Homo sapiens
  MOD_RES 5..6: note = Carboxymethyl C (2) [5 and/or 6]
KVTKCCTESLV

SEQ ID NO: 703 - protein, 10 aa, Homo sapiens
  MOD_RES 5..6: note = Carboxymethyl C (2) [5 and/or 6]
KVTKCCTESL

SEQ ID NO: 704 - protein, 9 aa, Homo sapiens
  MOD_RES 5..6: note = Carboxymethyl C (2) [5 and/or 6]
KVTKCCTES

SEQ ID NO: 705 - protein, 30 aa, Homo sapiens
  MOD_RES 5..16: note = Carboxymethyl C (3) [5, 6, and/or 16]
KVTKCCTESLVNRRPCFSALTPDETYVPKA

SEQ ID NO: 706 - protein, 29 aa, Homo sapiens
  MOD_RES 5..16: note = Carboxymethyl C (3) [5, 6, and/or 16]
  MOD_RES 12: note = Deamidation N [12]
KVTKCCTESLVNRRPCFSALTPDETYVPK

SEQ ID NO: 707 - protein, 49 aa, Homo sapiens
  MOD_RES 1..34: note = Glycation (4) [1, 4, 29, and/or 34]
  MOD_RES 5..43: note = Carboxymethyl C (9) [5, 6, 16, and/or 43]
  MOD_RES 12: note = Deamidation N [12]
KVTKCCTESLVNRRPCFSALTPDETYVPKAFDEKLFTFHADICTLPDTE

SEQ ID NO: 708 - protein, 47 aa, Homo sapiens
  MOD_RES 1..47: note = Glycation [?]
  MOD_RES 1..47: note = Glycation (2) [2 ?]
  MOD_RES 5..43: note = Carboxymethyl C (4) [5, 6, 16, and/or 43]
  MOD_RES 12: note = Deamidation N [12]
KVTKCCTESLVNRRPCFSALTPDETYVPKAFDEKLFTFHADICTLPD

SEQ ID NO: 709 - protein, 45 aa, Homo sapiens
  MOD_RES 1..39: note = Carboxymethyl C (4) [1, 2, 12, and/or 39]
  MOD_RES 1..45: note = Glycation [?]
  MOD_RES 8: note = Deamidation N [8]
CCTESLVNRRPCFSALTPDETYVPKAFDEKLFTFHADICTLPDTE

SEQ ID NO: 710 - protein, 7 aa, Homo sapiens
  MOD_RES 1..4: note = Glycation (2) [1 and/or 4]
  MOD_RES 5..6: note = Carboxymethyl C(2) [5 and/or 6]
KVTKCCT

SEQ ID NO: 711 - protein, 5 aa, Homo sapiens
  MOD_RES 1..5: note = Glycation [?]
  MOD_RES 5: note = Carboxymethyl C [5]
KVTKC

SEQ ID NO: 712 - protein, 6 aa, Homo sapiens
  MOD_RES 2: note = Glycation [2]
  MOD_RES 3..4: note = Carboxymethyl C (2) [3 and/or 4]
TKCCTE

SEQ ID NO: 713 - protein, 5 aa, Homo sapiens
  MOD_RES 1: note = Glycation [1]
  MOD_RES 2..3: note = Carboxymethyl C (2) [2 and/or 3]
KCCTE

SEQ ID NO: 714 - protein, 40 aa, Homo sapiens
  MOD_RES 1..40: note = Glycation [?]
  MOD_RES 8..35: note = Carboxymethyl C (2) [8 and/or 35]
SLVNRRPCFSALTPDETYVPKAFDEKLFTFHADICTLPDT

SEQ ID NO: 715 - protein, 32 aa, Homo sapiens
  MOD_RES 4: note = Deamidation N [4]
  MOD_RES 8: note = Carboxymethyl C [8]
SLVNRRPCFSALTPDETYVPKAFDEKLFTFHA

SEQ ID NO: 716 - protein, 40 aa, Homo sapiens
  MOD_RES 1..40: note = Glycation [?]
  MOD_RES 3: note = Deamidation N[3]
  MOD_RES 7..34: note = Carboxymethyl C (2) [7 and/or 34]
  MOD_RES 20..25: note = Glycation (2) [20 and/or 25]
LVNRRPCFSALTPDETYVPKAFDEKLFTFHADICTLPDTE

SEQ ID NO: 717 - protein, 33 aa, Homo sapiens
  MOD_RES 1..27: note = Carboxymethyl C [27]
FSALTPDETYVPKAFDEKLFTFHADICTLPDTE

SEQ ID NO: 718 - protein, 46 aa, Homo sapiens
  MOD_RES 1..46: note = Glycation (4) [4 ?]
  MOD_RES 3: note = Deamidation N [3]
  MOD_RES 7..34: note = Carboxymethyl C (2) [7 and/or 34]
  MOD_RES 20..45: note = Glycation (5) [20, 25, 41, 44, and/or 45]
LVNRRPCFSALTPDETYVPKAFDEKLFTFHADICTLPDTEKQIKKQ

SEQ ID NO: 719 - protein, 46 aa, Homo sapiens
  MOD_RES 6..33: note = Carboxymethyl C (2) [6 and/or 33]
  MOD_RES 19..44: note = Glycation (5) [19, 24, 40, 43, and/or 44]
VNRRPCFSALTPDETYVPKAFDEKLFTFHADICTLPDTEKQIKKQT

SEQ ID NO: 720 - protein, 8 aa, Homo sapiens
  MOD_RES 4: note = Deamidation N [4]
  MOD_RES 8: note = Carboxymethyl C [8]
SLVNRRPC

SEQ ID NO: 721 - protein, 6 aa, Homo sapiens
  MOD_RES 3: note = Glycation [3]
HFKGLV

SEQ ID NO: 722 - protein, 4 aa, Homo sapiens
  MOD_RES 3: note = Glycation [3]
HFKG

SEQ ID NO: 723 - protein, 10 aa, Homo sapiens
  MOD_RES 6: note = Carboxymethyl C [6]
QYLQQCPFDE

SEQ ID NO: 724 - protein, 8 aa, Homo sapiens
  MOD_RES 4: note = Carboxymethyl C [4]
LQQCPFDE

SEQ ID NO: 725 - protein, 6 aa, Homo sapiens
  MOD_RES 2: note = Carboxymethyl C (2]
QCPFDE

SEQ ID NO: 726 - protein, 21 aa, Homo sapiens
  MOD_RES 19: note = Carboxymethyl C [19]
TYVPKAFDEKLFTFHADICTL

SEQ ID NO: 727 - protein, 18 aa, Homo sapiens
  MOD_RES 1..18: note = Glycation [?]
TYVPKAFDEKLFTFHADI

SEQ ID NO: 728 - protein, 21 aa, Homo sapiens
  MOD_RES 1..16: note = Glycation (2) [1 and/or 6]
  MOD_RES 15: note = Carboxymethyl C [15]
KAFDEKLFTFHADICTLPDTE

SEQ ID NO: 729 - protein, 20 aa, Homo sapiens
  MOD_RES 14: note = Carboxymethyl C [14]
AFDEKLFTFHADICTLPDTE

SEQ ID NO: 730 - protein, 43 aa, Homo sapiens
  MOD_RES 2..43: note = Glycation (2) [2 ?]
  MOD_RES 19: note = Carboxymethyl C [19]
TYVPKAFDEKLFTFHADICTLPDTEKQIKKQTALVELLKHKPK

SEQ ID NO: 731 - protein, 39 aa, Homo sapiens
  MOD_RES 19: note = Carboxymethyl C [19]
TYVPKAFDEKLFTFHADICTLPDTEKQIKKQTALVELLK

SEQ ID NO: 732 - protein, 45 aa, Homo sapiens
  MOD_RES 18: note = Carboxymethyl C [18]
  MOD_RES 45: note = Glycation [?]
YVPKAFDEKLFTFHADICTLPDTEKQIKKQTALVELLKHKPKATE

SEQ ID NO: 733 - protein, 6 aa, Homo sapiens
  MOD_RES 5: note = Glycation [5]
TYVPKA

SEQ ID NO: 734 - protein, 7 aa, Homo sapiens
  MOD_RES 3: note = Glycation [3]
VPKAFDE

SEQ ID NO: 735 - protein, 27 aa, Homo sapiens
  MOD_RES 1..27: note = Glycation (3) [3?]
  MOD_RES 10: note = Carboxymethyl C [10]
KLFTFHADICTLPDTEKQIKKQTALVE

SEQ ID NO: 736 - protein, 17 aa, Homo sapiens
  MOD_RES 1..17: note = Glycation (2) [2 ?]
TLPDTEKQIKKQTALVE

SEQ ID NO: 737 - protein, 37 aa, Homo sapiens
  MOD_RES 10: note = Carboxymethyl C [10]
KLFTFHADICTLPDTEKQIKKQTALVELLKHKPKATE

SEQ ID NO: 738 - protein, 36 aa, Homo sapiens
  MOD_RES 1..36: note = Glycation (4) [4 ?]
  MOD_RES 10: note = Carboxymethyl C [10]
KLFTFHADICTLPDTEKQIKKQTALVELLKHKPKAT

SEQ ID NO: 739 - protein, 30 aa, Homo sapiens
  MOD_RES 10: note = Carboxymethyl C [10]
KLFTFHADICTLPDTEKQIKKQTALVELLK

SEQ ID NO: 740 - protein, 23 aa, Homo sapiens
  MOD_RES 1..23: note = Glycation (5) [5 ?]
TEKQIKKQTALVELLKHKPKATE

SEQ ID NO: 741 - protein, 31 aa, Homo sapiens
  MOD_RES 1..31: note = Glycation (3) [3 ?]
  MOD_RES 4: note = Carboxymethyl C [4]
ADICTLPDTEKQIKKQTALVELLKHKPKATE

SEQ ID NO: 742 - protein, 38 aa, Homo sapiens
  MOD_RES 1..38: note = Glycation (6) [6 ?]
  MOD_RES 1..34: note = Glycation (7) [1, 17, 20, 21, 30, 32, and/or 34]
  MOD_RES 10: note = Carboxymethyl C [10]
KLFTFHADICTLPDTEKQIKKQTALVELLKHKPKATEE

SEQ ID NO: 743 - protein, 24 aa, Homo sapiens
  MOD_RES 1..24: note = Glycation (4) [4 ?]
TEKQIKKQTALVELLKHKPKATEE

SEQ ID NO: 744 - protein, 23 aa, Homo sapiens
  MOD_RES 1..23: note = Glycation (4) [4 ?]
EKQIKKQTALVELLKHKPKATEE

SEQ ID NO: 745 - protein, 35 aa, Homo sapiens
  MOD_RES 1..35: note = Glycation [?]
  MOD_RES 7: note = Carboxymethyl C [7]
TFHADICTLPDTEKQIKKQTALVELLKHKPKATEE

SEQ ID NO: 746 - protein, 4 aa, Homo sapiens
  MOD_RES 1: note = Glycation [1]
KLFT

SEQ ID NO: 747 - protein, 14 aa, Homo sapiens
  MOD_RES 3: note = Carboxymethyl C [3]
FTFHADICTLPDTE

SEQ ID NO: 748 - protein, 11 aa, Homo sapiens
  MOD_RES 5: note = Carboxymethyl C [5]
HADICTLPDTE

SEQ ID NO: 749 - protein, 9 aa, Homo sapiens
  MOD_RES 3: note = Carboxymethyl C [3]
DICTLPDTE

SEQ ID NO: 750 - protein, 11 aa, Homo sapiens
  MOD_RES 1..11: note = Glycation (4) [2 ?]
KQIKKQTALVE

SEQ ID NO: 751 - protein, 20 aa, Homo sapiens
  MOD_RES 1..20: note = Glycation [?]
KQIKKQTALVELLKHKPKAT

SEQ ID NO: 752 - protein, 19 aa, Homo sapiens
  MOD_RES 1..19: note = Glycation (4) [?]
KQIKKQTALVELLKHKPKA

SEQ ID NO: 753 - protein, 17 aa, Homo sapiens
  MOD_RES 1..17: note = Glycation (4) [4 ?]
KQIKKQTALVELLKHKP

SEQ ID NO: 754 - protein, 16 aa, Homo sapiens
  MOD_RES 1..16: note = Glycation (3) [3 ?]
KQIKKQTALVELLKHK

SEQ ID NO: 755 - protein, 15 aa, Homo sapiens
  MOD_RES 1..14: note = Glycation (4) [1, 4, 5, and/or 14]
KQIKKQTALVELLKH

SEQ ID NO: 756 - protein, 19 aa, Homo sapiens
  MOD_RES 1..19: note = Glycation (2) [2 ?]
IKKQTALVELLKHKPKATE

SEQ ID NO: 757 - protein, 17 aa, Homo sapiens
  MOD_RES 1..17: note = Glycation (3) [3 ?]
KQTALVELLKHKPKATE

SEQ ID NO: 758 - protein, 16 aa, Homo sapiens
  MOD_RES 9..13: note = Glycation (3) [9, 11, and/or 13]
QTALVELLKHKPKATE

SEQ ID NO: 759 - protein, 19 aa, Homo sapiens
  MOD_RES 1..19: note = Glycation [?]
KKQTALVELLKHKPKATEE

SEQ ID NO: 760 - protein, 28 aa, Homo sapiens
  MOD_RES 1..28: note = Glycation (?]
  MOD_RES 28: note = Oxidation M [28]
KQIKKQTALVELLKHKPKATEEQLKTVM

SEQ ID NO: 761 - protein, 26 aa, Homo sapiens
  MOD_RES 1..26: note = Glycation (4) [4 ?]
KQIKKQTALVELLKHKPKATEEQLKT

SEQ ID NO: 762 - protein, 25 aa, Homo sapiens
  MOD_RES 1..25: note = Glycation (5) [5 ?]
KQIKKQTALVELLKHKPKATEEQLK

SEQ ID NO: 763 - protein, 28 aa, Homo sapiens
  MOD_RES 1..28: note = Glycation (?]
  MOD_RES 27: note = Oxidation M [27]
QIKKQTALVELLKHKPKATEEQLKTVME

SEQ ID NO: 764 - protein, 27 aa, Homo sapiens
  MOD_RES 1..27: note = Glycation [?]
  MOD_RES 26: note = Oxidation M [26]
IKKQTALVELLKHKPKATEEQLKTVME

SEQ ID NO: 765 - protein, 24 aa, Homo sapiens
  MOD_RES 9..20: note = Glycation (4) [9, 11, 13, and/or 20]
  MOD_RES 23: note = Oxidation M [23]
QTALVELLKHKPKATEEQLKTVME

SEQ ID NO: 766 - protein, 21 aa, Homo sapiens
  MOD_RES 1..21: note = Glycation (2) [2 ?]
  MOD_RES 20: note = Oxidation M [20]
LVELLKHKPKATEEQLKTVME

SEQ ID NO: 767 - protein, 20 aa, Homo sapiens
  MOD_RES 1..20: note = Glycation [?]
VELLKHKPKATEEQLKTVME

SEQ ID NO: 768 - protein, 5 aa, Homo sapiens
  MOD_RES 1..5: note = Glycation (2) [2 ?]
KQIKK

SEQ ID NO: 769 - protein, 4 aa, Homo sapiens
  MOD_RES 1..4: note = Glycation [?]
KQIK

SEQ ID NO: 770 - protein, 8 aa, Homo sapiens
  MOD_RES 1..8: note = Glycation [?]
KKQTALVE

SEQ ID NO: 771 - protein, 11 aa, Homo sapiens
  MOD_RES 1..11: note = Glycation [?]
LLKHKPKATEE

SEQ ID NO: 772 - protein, 18 aa, Homo sapiens
  MOD_RES 1..18: note = Glycation (2) [2 ?]
  MOD_RES 17: note = Oxidation M [17]
LLKHKPKATEEQLKTVME

SEQ ID NO: 773 - protein, 17 aa, Homo sapiens
  MOD_RES 1..17: note = Glycation (3) [3 ?]
  MOD_RES 2..13: note = Glycation (4) [2, 4, 6, and/or 13]
  MOD_RES 16: note = Oxidation M [16]
LKHKPKATEEQLKTVME

SEQ ID NO: 774 - protein, 9 aa, Homo sapiens
  MOD_RES 5: note = Glycation [5]
  MOD_RES 8: note = Oxidation M [8]
EEQLKTVME

SEQ ID NO: 775 - protein, 21 aa, Homo sapiens
  MOD_RES 1..21: note = Glycation [?]
  MOD_RES 17: note = Oxidation M [17]
  MOD_RES 19: note = Deamidation N [19]
LLKHKPKATEEQLKTVMENFV

SEQ ID NO: 776 - protein, 28 aa, Homo sapiens
  MOD_RES 1..28: note = Glycation (4) [4 ?]
  MOD_RES 17: note = Oxidation M [17]
  MOD_RES 27..28: note = Carboxymethyl C (2) [27 and/or 28]
LLKHKPKATEEQLKTVMENFVAFVDKCC

SEQ ID NO: 777 - protein, 26 aa, Homo sapiens
  MOD_RES 1..26: note = Glycation (3) [3 ?]
LLKHKPKATEEQLKTVMENFVAFVDK

SEQ ID NO: 778 - protein, 28 aa, Homo sapiens
  MOD_RES 1..28: note = Glycation (3) [3 ?]
  MOD_RES 11: note = Oxidation M [11]
  MOD_RES 21..22: note = Carboxymethyl C (2) [21 and/or 22]
KATEEQLKTVMENFVAFVDKCCAADDKE

SEQ ID NO: 779 - protein, 9 aa, Homo sapiens
  MOD_RES 1..9: note = Glycation [?]
LLKHKPKAT

SEQ ID NO: 780 - protein, 4 aa, Homo sapiens
  MOD_RES 3: note = Glycation [3]
LLKH

SEQ ID NO: 781 - protein, 9 aa, Homo sapiens
  MOD_RES 1..9: note = Glycation [?]
LKHKPKATE

SEQ ID NO: 782 - protein, 7 aa, Homo sapiens
  MOD_RES 1..7: note = Glycation [?]
HKPKATE

SEQ ID NO: 783 - protein, 4 aa, Homo sapiens
  MOD_RES 1: note = Glycation [1]
KATE

SEQ ID NO: 784 - protein, 5 aa, Homo sapiens
  MOD_RES 4: note = Glycation [4]
EQLKT

SEQ ID NO: 785 - protein, 9 aa, Homo sapiens
  MOD_RES 4: note = Glycation [4]
EQLKTVMEN

SEQ ID NO: 786 - protein, 19 aa, Homo sapiens
  MOD_RES 1..19: note = Glycation [?]
  MOD_RES 9: note = Deamidation N [9]
  MOD_RES 17..18: note = Carboxymethyl C (2) [17 and/or 18]
EQLKTVMENFVAFVDKCCA

SEQ ID NO: 787 - protein, 30 aa, Homo sapiens
  MOD_RES 17..26: note = Carboxymethyl C (3) [17, 18, and/or 26]
EQLKTVMENFVAFVDKCCAADDKEACFAVE

SEQ ID NO: 788 - protein, 26 aa, Homo sapiens
  MOD_RES 4..23: note = Glycation (3) [4, 16, and/or 23]
  MOD_RES 9: note = Deamidation N [9]
  MOD_RES 17..26: note = Carboxymethyl C (3) [17, 18, and/or 26]
EQLKTVMENFVAFVDKCCAADDKEAC

SEQ ID NO: 789 - protein, 23 aa, Homo sapiens
  MOD_RES 1..23: note = Glycation (2) [2 ?]
  MOD_RES 6: note = Oxidation M [6]
  MOD_RES 16..17: note = Carboxymethyl C (2) [16 and/or 17]
QLKTVMENFVAFVDKCCAADDKE

SEQ ID NO: 790 - protein, 7 aa, Homo sapiens
  MOD_RES 3: note = Glycation [3]
  MOD_RES 6: note = Oxidation M [6]
QLKTVME

SEQ ID NO: 791 - protein, 23 aa, Homo sapiens
  MOD_RES 1..23: note = Glycation (2) [2 ?]
  MOD_RES 6: note = Oxidation M [6]
  MOD_RES 16..17: note = Carboxymethyl C (2) [16 and/or 17]
QLKTVMENFVAFVDKCCAADDKE

SEQ ID NO: 792 - protein, 23 aa, Homo sapiens
  MOD_RES 1..23: note = Glycation [2 ?]
  MOD_RES 6: note = Oxidation M [6]
  MOD_RES 16..17: note = Carboxymethyl [16 and/or 17]
QLKTVMENFVAFVDKCCAADDKE

SEQ ID NO: 793 - protein, 8 aa, Homo sapiens
  MOD_RES 3: note = Glycation [3]
  MOD_RES 6: note = Oxidation M [6]
  MOD_RES 8: note = Deamidation N [8]
QLKTVMEN

SEQ ID NO: 794 - protein, 18 aa, Homo sapiens
  MOD_RES 1..18: note = Glycation [?]
  MOD_RES 16..17: note = Carboxymethyl C (2) [16 and/or 17]
QLKTVMENFVAFVDKCCA

SEQ ID NO: 795 - protein, 17 aa, Homo sapiens
  MOD_RES 6: note = Oxidation M [6]
  MOD_RES 7: note = GIycation [7]
  MOD_RES 8: note = Deamidation N [8]
  MOD_RES 16..17: note = Carboxymethyl C (2) [16 and/or 17]
QLKTVMENFVAFVDKCC

SEQ ID NO: 796 - protein, 22 aa, Homo sapiens
  MOD_RES 7: note = Deamidation N [7]
  MOD_RES 15..16: note = Carboxymethyl C (2) [15 and/or 16]
LKTVMENFVAFVDKCCAADDKE

SEQ ID NO: 797 - protein, 21 aa, Homo sapiens
  MOD_RES 4: note = Oxidation M [4]
  MOD_RES 7: note = Glycation [7]
  MOD_RES 14..15: note = Carboxymethyl C (2) [14 and/or 15]
KTVMENFVAFVDKCCAADDKE

SEQ ID NO: 798 - protein, 19 aa, Homo sapiens
  MOD_RES 1..19: note = Glycation [?]
  MOD_RES 1..19: note = Glycation [?], Glycation (2) [11 and/or 18]
  MOD_RES 2: note = Oxidation M [2]
  MOD_RES 4: note = Deamidation N [4]
  MOD_RES 4: note = Deamidation N [4]
  MOD_RES 11..18: note = GIycation (2) [11 and/or 18]
  MOD_RES 12..13: note = Carboxymethyl C (2) [12 and/or 13]
VMENFVAFVDKCCAADDKE

SEQ ID NO: 799 - protein, 18 aa, Homo sapiens
  MOD_RES 3: note = Deamidation N [3]
  MOD_RES 10..17: note = Glycation (2) [10 and/or 17]
  MOD_RES 11..12: note = Carboxymethyl C (2) [11 and/or 12]
MENFVAFVDKCCAADDKE

SEQ ID NO: 800 - protein, 17 aa, Homo sapiens
  MOD_RES 2..11: note = Deamidation N [2]
  MOD_RES 9..16: note = Glycation (2) [9 and/or 16]
  MOD_RES 10..11: note = Carboxymethyl C (2) [10 and/or 11]
ENFVAFVDKCCAADDKE

SEQ ID NO: 801 - protein, 27 aa, Homo sapiens
  MOD_RES 1..20: note = Glycation (3) [1, 13, and/or 20]
  MOD_RES 6: note = Deamidation N [6]
  MOD_RES 14..23: note = Carboxymethyl C (3) [14, 15, and/or 23]
KTVMENFVAFVDKCCAADDKEACFAVE

SEQ ID NO: 802 - protein, 26 aa, Homo sapiens
  MOD_RES 3: note = Oxidation M [3]
  MOD_RES 7: note = Glycation [7]
  MOD_RES 13..22: note = Carboxymethyl C (3) [13, 14, and/or 22]
TVMENFVAFVDKCCAADDKEACFAVE

SEQ ID NO: 803 - protein, 25 aa, Homo sapiens
  MOD_RES 1..25: note = Glycation (2) [11 and/or 18], Glycation [7 ?]
  MOD_RES 2: note = Oxidation M [2]
  MOD_RES 11..18: note = Glycation (2) [11 and/or 18]
  MOD_RES 12..21: note = Carboxymethyl C (3) [12, 13, and/or 21]
VMENFVAFVDKCCAADDKEACFAVE

SEQ ID NO: 804 - protein, 35 aa, Homo sapiens
  MOD_RES 8: note = Deamidation N [8]
  MOD_RES 16..25: note = Carboxymethyl C (3) [16, 17, and/or 25]
QLKTVMENFVAFVDKCCAADDKEACFAVEGPKLVV

SEQ ID NO: 805 - protein, 41 aa, Homo sapiens
  MOD_RES 1..41: note = Glycation (2) [2 ?]
  MOD_RES 7: note = Deamidation N [7]
  MOD_RES 15..24: note = Carboxymethyl C (3) [15, 16, and/or 24]
LKTVMENFVAFVDKCCAADDKEACFAVEGPKLVVSTQTALA

SEQ ID NO: 806 - protein, 41 aa, Homo sapiens; identical to SEQ ID NO: 805
  MOD_RES 1..41: note = Glycation (2) [2 ?]
  MOD_RES 1..41: note = Glycation (2) [2 ?], Glycation (3) [11, 18, and/or 28]
  MOD_RES 2: note = Oxidation M [2]
  MOD_RES 4: note = Deamidation N [4]
  MOD_RES 12..21: note = Carboxymethyl C (3) [12, 13, and/or 21]

SEQ ID NO: 807 - protein, 38 aa, Homo sapiens
VMENFVAFVDKCCAADDKEACFAVEGPKLVVSTQTALA

SEQ ID NO: 808 - protein, 6 aa, Homo sapiens
  MOD_RES 6: note = Oxidation M [6]
QLKTVM

SEQ ID NO: 809 - protein, 5 aa, Homo sapiens
  MOD_RES 3: note = Glycation [3]
QLKTV

SEQ ID NO: 810 - protein, 6 aa, Homo sapiens
  MOD_RES 2: note = Glycation [2]
LKTVME

SEQ ID NO: 811 - protein, 5 aa, Homo sapiens
  MOD_RES 1: note = Glycation [1]
  MOD_RES 4: note = Oxidation M [4]
KTVME

SEQ ID NO: 812 - protein, 16 aa, Homo sapiens
  MOD_RES 1..16: note = Glycation [?]
  MOD_RES 7: note = Glycation [7]
  MOD_RES 9..10: note = Carboxymethyl C (2) [9 and/or 10]
NFVAFVDKCCAADDKE

SEQ ID NO: 813 - protein, 10 aa, Homo sapiens
  MOD_RES 6: note = Deamidation N [6]
HVKLVNELTE

SEQ ID NO: 814 - protein, 22 aa, Homo sapiens
  MOD_RES 1: note = Deamidation N [1]
  MOD_RES 8..15: note = Glycation (2) [8 and/or 15]
  MOD_RES 9..18: note = Carboxymethyl C (3) [9, 10, and/or 18]
NFVAFVDKCCAADDKEACFAVE

SEQ ID NO: 815 - protein, 19 aa, Homo sapiens
  MOD_RES 1: note = Deamidation N [1]
  MOD_RES 1..19: note = Glycation [?], Glycation (2) [8 and/or 15]
  MOD_RES 9..18: note = Carboxymethyl C (3) [9, 10, and/or 18]
NFVAFVDKCCAADDKEACF

SEQ ID NO: 816 - protein, 17 aa, Homo sapiens
  MOD_RES 1..17: note = Glycation [?]
  MOD_RES 1: note = Deamidation N [1]
  MOD_RES 9..10: note = Carboxymethyl C (2) [9 and/or 10]
NFVAFVDKCCAADDKEA

SEQ ID NO: 817 - protein, 21 aa, Homo sapiens
  MOD_RES 1..21: note = Glycation [?]
  MOD_RES 8..17: note = Carboxymethyl C (3) [8, 9, and/or 17]
FVAFVDKCCAADDKEACFAVE

SEQ ID NO: 818 - protein, 11 aa, Homo sapiens
  MOD_RES 7: note = Carboxymethyl C [7]
ADDKEACFAVE

SEQ ID NO: 819 - protein, 10 aa, Homo sapiens
  MOD_RES 6: note = Carboxymethyl C [6]
DDKEACFAVE

SEQ ID NO: 820 - protein, 8 aa, Homo sapiens
  MOD_RES 1: note = Glycation [1]
  MOD_RES 4: note = Carboxymethyl C [4]
KEACFAVE

SEQ ID NO: 821 - protein, 7 aa, Homo sapiens
  MOD_RES 3: note = Carboxymethyl C [3]
EACFAVE

SEQ ID NO: 822 - protein, 17 aa, Homo sapiens
  MOD_RES 3..10: note = Glycation (2) [3 and/or 10]
  MOD_RES 4..13: note = Carboxymethyl C (3) [4, 5, and/or 13]
VDKCCAADDKEACFAVE

SEQ ID NO: 823 - protein, 15 aa, Homo sapiens
  MOD_RES 2..11: note = Carboxymethyl C (3) [2, 3, and/or 11]
KCCAADDKEACFAVE

SEQ ID NO: 824 - protein, 14 aa, Homo sapiens
  MOD_RES 1..10: note = Carboxymethyl C (3) [1, 2, and/or 10]
  MOD_RES 7: note = Glycation [7]
CCAADDKEACFAVE

SEQ ID NO: 825 - protein, 13 aa, Homo sapiens
  MOD_RES 1..9: note = Carboxymethyl C (2) [1 and/or 9]
CAADDKEACFAVE

SEQ ID NO: 826 - protein, 9 aa, Homo sapiens
  MOD_RES 3: note = Glycation [3]
HVKLVNELT

SEQ ID NO: 827 - protein, 6 aa, Homo sapiens
  MOD_RES 1: note = Deamidation N [1]
NFVAFV

SEQ ID NO: 828 - protein, 14 aa, Homo sapiens
  MOD_RES 8: note = Glycation [8]
  MOD_RES 9..10: note = Carboxymethyl C (2) [9 and/or 10]
NFVAFVDKCCAADD

SEQ ID NO: 829 - protein, 8 aa, Homo sapiens
  MOD_RES 6: note = Deamidation N [6]
HVKLVNEL

SEQ ID NO: 830 - protein, 13 aa, Homo sapiens
  MOD_RES 1: note = Deamidation N [1]
  MOD_RES 8: note = Glycation [8]
  MOD_RES 9..10: note = Carboxymethyl C (2) [9 and/or 10]
NFVAFVDKCCAAD

SEQ ID NO: 831 - protein, 15 aa, Homo sapiens
  MOD_RES 8..9: note = Carboxymethyl C (2) [8 and/or 9]
FVAFVDKCCAADDKE

SEQ ID NO: 832 - protein, 6 aa, Homo sapiens
  MOD_RES 5: note = Glycation [5]
AADDKE

SEQ ID NO: 833 - protein, 5 aa, Homo sapiens
  MOD_RES 4: note = Glycation [4]
ADDKE

SEQ ID NO: 834 - protein, 8 aa, Homo sapiens
  MOD_RES 1: note = Glycation [1]
KLVNELTE

SEQ ID NO: 835 - protein, 12 aa, Homo sapiens
  MOD_RES 1..12: note = Glycation [?]
  MOD_RES 1..12: note = Glycation [?], Glycation (2) [4 and/or 11]
  MOD_RES 5..6: note = Carboxymethyl C (2) [5 6]
FVDKCCAADDKE

SEQ ID NO: 836 - protein, 10 aa, Homo sapiens
  MOD_RES 1..10: note = Glycation [?]
  MOD_RES 3..4: note = Carboxymethyl C (2) [3 and/or 4]
DKCCAADDKE

SEQ ID NO: 837 - protein, 7 aa, Homo sapiens
  MOD_RES 1: note = Carboxymethyl C [1]
CAADDKE

SEQ ID NO: 838 - protein, 6 aa, Homo sapiens
  MOD_RES 2: note = Carboxymethyl C [2]
ACFAVE

SEQ ID NO: 839 - protein, 9 aa, Homo sapiens
  MOD_RES 2: note = Carboxymethyl C [2]
  MOD_RES 9: note = Glycation [9]
ACFAVEGPK

SEQ ID NO: 840 - protein, 8 aa, Homo sapiens
  MOD_RES 2: note = Carboxymethyl C [2]
ACFAVEGP

SEQ ID NO: 841 - protein, 12 aa, Homo sapiens
  MOD_RES 2: note = Carboxymethyl C [2]
  MOD_RES 9: note = Glycation [9]
ACFAVEGPKLVV

SEQ ID NO: 842 - protein, 16 aa, Homo sapiens
  MOD_RES 6: note = Glycation [6]
AVEGPKLVVSTQTALA

SEQ ID NO: 843 - protein, 4 aa, Homo sapiens
  MOD_RES 2: note = Carboxymethyl C [2]
ACFA

SEQ ID NO: 844 - protein, 5 aa, Homo sapiens
  MOD_RES 1: note = Carboxymethyl C [1]
CFAVE

SEQ ID NO: 845 - protein, 17 aa, Homo sapiens
  MOD_RES 4: note = Deamidation N [4]
  MOD_RES 7: note = Glycation [7]
  MOD_RES 13: note = Carboxymethyl C [13]
KLVNELTEFAKTCVADE

SEQ ID NO: 846 - protein, 6 aa, Homo sapiens
  MOD_RES 3: note = Glycation [3]
GPKLVV

SEQ ID NO: 847 - protein, 20 aa, Homo sapiens
  MOD_RES 8: note = Glycation [8]
  MOD_RES 10..19: note = Carboxymethyl C (2) [10 and/or 19]
NELTEFAKTCVADESHAGCE

SEQ ID NO: 848 - protein, 6 aa, Homo sapiens
  MOD_RES 3: note = Glycation [3]
  MOD_RES 6: note = Deamidation N [6]
HVKLVN

SEQ ID NO: 849 - protein, 5 aa, Homo sapiens
  MOD_RES 3: note = Glycation [3]
HVKLV

SEQ ID NO: 850 - protein, 6 aa, Homo sapiens
  MOD_RES 5: note = Deamidation N [5]
VKLVNE

SEQ ID NO: 851 - protein, 5 aa, Homo sapiens
  MOD_RES 1: note = Glycation [1]
  MOD_RES 4: note = Deamidation N [4]
KLVNE

SEQ ID NO: 852 - protein, 11 aa, Homo sapiens
  MOD_RES 8: note = Carboxymethyl C [8]
LTEFAKTCVAD

SEQ ID NO: 853 - protein, 8 aa, Homo sapiens
  MOD_RES 6: note = Glycation [6]
  MOD_RES 8: note = Carboxymethyl C [8]
LTEFAKTC

SEQ ID NO: 854 - protein, 7 aa, Homo sapiens
  MOD_RES 6: note = Glycation [6]
LTEFAKT

SEQ ID NO: 855 - protein, 6 aa, Homo sapiens
  MOD_RES 6: note = Glycation [6]
LTEFAK

SEQ ID NO: 856 - protein, 16 aa, Homo sapiens
  MOD_RES 6..15: note = Carboxymethyl C (2) [6 and/or 15]
EFAKTCVADESHAGCE

SEQ ID NO: 857 - protein, 11 aa, Homo sapiens
  MOD_RES 5: note = Carboxymethyl C [5]
FAKTCVADESH

SEQ ID NO: 858 - protein, 13 aa, Homo sapiens
  MOD_RES 1: note = Glycation [1]
  MOD_RES 3..12: note = Carboxymethyl C (2) [3 and/or 12]
KTCVADESHAGCE

SEQ ID NO: 859 - protein, 12 aa, Homo sapiens
  MOD_RES 2..11: note = Carboxymethyl C (2) [2 and/or 11]
TCVADESHAGCE

SEQ ID NO: 860 - protein, 10 aa, Homo sapiens
  MOD_RES 9: note = Carboxymethyl C [9]
VADESHAGCE

SEQ ID NO: 861 - protein, 8 aa, Homo sapiens
  MOD_RES 7: note = Carboxymethyl C [7]
DESHAGCE

SEQ ID NO: 862 - protein, 7 aa, Homo sapiens
  MOD_RES 6: note = Carboxymethyl C [6]
ESHAGCE

SEQ ID NO: 863 - protein, 21 aa, Homo sapiens
  MOD_RES 1..21: note = Glycation [?]
  MOD_RES 5..14: note = Carboxymethyl C (2) [5 and/or 14]
FAKTCVADESHAGCEKSLHTL

SEQ ID NO: 864 - protein, 16 aa, Homo sapiens
  MOD_RES 3..16: note = Glycation (2) [3 and/or 16]
  MOD_RES 5..14: note = Carboxymethyl C (2) [5 and/or 14]
FAKTCVADESHAGCEK

SEQ ID NO: 865 - protein, 8 aa, Homo sapiens
  MOD_RES 3: note = Glycation [3]
  MOD_RES 5: note = Carboxymethyl C [5]
FAKTCVAD

SEQ ID NO: 866 - protein, 6 aa, Homo sapiens
  MOD_RES 3: note = Glycation [3]
  MOD_RES 5: note = Carboxymethyl C [5]
FAKTCV

SEQ ID NO: 867 - protein, 5 aa, Homo sapiens
  MOD_RES 3: note = Glycation [3]
  MOD_RES 5: note = Carboxymethyl C [5]
FAKTC

SEQ ID NO: 868 - protein, 8 aa, Homo sapiens
  MOD_RES 2: note = Glycation [2]
  MOD_RES 4: note = Carboxymethyl C [4]
AKTCVADE

SEQ ID NO: 869 - protein, 7 aa, Homo sapiens
  MOD_RES 1: note = Glycation [1]
  MOD_RES 3: note = Carboxymethyl C [3]
KTCVADE

SEQ ID NO: 870 - protein, 6 aa, Homo sapiens
  MOD_RES 2: note = Carboxymethyl C [2]
TCVADE

SEQ ID NO: 871 - protein, 5 aa, Homo sapiens
  MOD_RES 1: note = Carboxymethyl C [1]
CVADE

SEQ ID NO: 872 - protein, 6 aa, Homo sapiens
  MOD_RES 5: note = Carboxymethyl C [5]
SHAGCE

SEQ ID NO: 873 - protein, 34 aa, Homo sapiens
  MOD_RES 1..34: note = Oxidation M [?]
  MOD_RES 1..34: note = Glycation [?]
  MOD_RES 5..34: note = Carboxymethyl C (4) [5, 18, 33, and/or 34]
SHAGCEKSLHTLFGDELCKVASLRETYGDMADCC

SEQ ID NO: 874 - protein, 11 aa, Homo sapiens
  MOD_RES 5: note = Carboxymethyl C [5]
SHAGCEKSLHT

SEQ ID NO: 875 - protein, 10 aa, Homo sapiens
  MOD_RES 5: note = Carboxymethyl C [5]
  MOD_RES 7: note = Glycation [7]
SHAGCEKSLH

SEQ ID NO: 876 - protein, 8 aa, Homo sapiens
  MOD_RES 5: note = Carboxymethyl C [5]
  MOD_RES 7: note = Glycation [7]
SHAGCEKS

SEQ ID NO: 877 - protein, 7 aa, Homo sapiens
  MOD_RES 5: note = Carboxymethyl C [5]
  MOD_RES 7: note = Glycation [7]
SHAGCEK

SEQ ID NO: 878 - protein, 5 aa, Homo sapiens
  MOD_RES 5: note = Carboxymethyl C [5]
SHAGC

SEQ ID NO: 879 - protein, 29 aa, Homo sapiens
  MOD_RES 12..28: note = Carboxymethyl C (3) [12, 27, and/or 28]
  MOD_RES 24: note = Oxidation M [24]
KSLHTLFGDELCKVASLRETYGDMADCCE

SEQ ID NO: 880 - protein, 22 aa, Homo sapiens
  MOD_RES 1: note = Oxidation M [1]
  MOD_RES 5..21: note = Carboxymethyl C (3) [5, 20, and/or 21]
  MOD_RES 6: note = Glycation [6]
GDELCKVASLRETYGDMADCCE

SEQ ID NO: 881 - protein, 21 aa, Homo sapiens
  MOD_RES 4..20: note = Carboxymethyl C (3) [4, 19, and/or 20]
  MOD_RES 16: note = Oxidation M [16]
DELCKVASLRETYGDMADCCE

SEQ ID NO: 882 - protein, 20 aa, Homo sapiens
  MOD_RES 1: note = Oxidation M [1]
  MOD_RES 3..19: note = Carboxymethyl C (3) [3, 18, and/or 19]
  MOD_RES 4: note = Glycation [4]
ELCKVASLRETYGDMADCCE

SEQ ID NO: 883 - protein, 28 aa, Homo sapiens
  MOD_RES 1: note = Oxidation M [1]
  MOD_RES 1..28: note = Glycation [?]
  MOD_RES 6..22: note = Carboxymethyl C (3) [6, 21, and/or 22]
FGDELCKVASLRETYGDMADCCEKQEPE

SEQ ID NO: 884 - protein, 8 aa, Homo sapiens
  MOD_RES 1: note = Glycation [1]
KSLHTLFG

SEQ ID NO: 885 - protein, 7 aa, Homo sapiens
  MOD_RES 1: note = Glycation [1]
KSLHTLF

SEQ ID NO: 886 - protein, 6 aa, Homo sapiens
  MOD_RES 1: note = Glycation [1]
KSLHTL

SEQ ID NO: 887 - protein, 14 aa, Homo sapiens
ITPNLAEFAFSLYR

SEQ ID NO: 888 - protein, 10 aa, Homo sapiens
LEVAESEFTH